(19) United States
(12) Patent Application Publication
(10) Pub. No.: US 2023/0227778 A1
(43) Pub. Date: Jul. 20, 2023

(54) COMPOSITIONS AND METHODS OF GENERATING AN IMMUNE RESPONSE

(71) Applicant: Intima Bioscience, Inc., New York, NY (US)

(72) Inventors: Thomas HENLEY, Soham (GB); Modassir CHOUDHRY, New York, NY (US)

(21) Appl. No.: 18/048,227

(22) Filed: Oct. 20, 2022

Related U.S. Application Data
(63) Continuation of application No. PCT/US2021/028436, filed on Apr. 21, 2021.
(60) Provisional application No. 63/013,435, filed on Apr. 21, 2020.
(60) Provisional application No. 63/056,517, filed on Jul. 24, 2020.

(51) Int. Cl. C12N 5/0783

(52) U.S. Cl. CPC C12N 5/0636; C12N 2502/1121; C12N 2501/2301; C12N 2501/2302; C12N 2501/2304; C12N 2501/2307; C12N 2501/2315; C12N 2501/25; C12N 2501/26; C12N 2501/22; C12N 2501/998

(57) ABSTRACT

Provided herein are compositions and methods for use in generating an immune response against a target peptide antigen. Also included, are methods for stabilizing, treating, and eliminating various diseases and conditions associated with target peptide expression.

### CROSS REFERENCE TO RELATED APPLICATIONS

This application is a continuation of, claims priority to, and the benefit of, International Application No. PCT/US2021/028436, filed Apr. 21, 2021, which claims priority to, and the benefit of, U.S. Provisional Patent Application Ser. No. 63/013,435, filed Apr. 21, 2020, and U.S. Provisional Patent Application Ser. No. 63/056,517, filed Jul. 24, 2020, the contents of which are incorporated by reference in their entirety.

### SEQUENCE LISTING

This application contains a Sequence Listing which has been submitted electronically in XML format, which is hereby incorporated by reference in its entirety. Said XML copy, created on Oct. 12, 2022 is named 199827-756301_SL and is 106,496 bytes in size.

### BACKGROUND

Over the past 40 years, there have been recurrent large-scale epidemics from emerging viruses such as HIV, SARS and Middle East respiratory syndrome coronaviruses, 2009 pandemic influenza H1N1 virus, Ebola virus, Zika virus and most recently SARS-CoV-2. In early January 2020, a cluster of cases of pneumonia from a new coronavirus, SARS-CoV-2 (with the disease referred to as COVID-19), was reported in Wuhan, China. This outbreak has spread rapidly, with over 1.2 million reported cases and 64,500 deaths worldwide as of Apr. 4, 2020, with little to no available treatments available to afflicted individuals.

### SUMMARY

In one aspect, provided herein is a method of producing a population of ex vivo antigen specific T cells, said method comprising: (a) isolating T cells from a sample obtained from a subject; (b) isolating dendritic cells or dendritic cell precursors from said sample obtained from said subject; (c) culturing said dendritic cells or dendritic cell precursors with at least a first exogenous peptide to produce a population of antigen presenting dendritic cells or dendritic cell precursors that present at least one of said first exogenous peptide or a derivative thereof, and (d) culturing said isolated T cells from (a) with (i) said population of antigen presenting dendritic cells or precursors thereof from (c); and (ii) an artificial antigen presenting platform that comprises: (1) a first protein that comprises a peptide binding domain of a human leucocyte antigen (HLA) protein that binds a second exogenous peptide, wherein said first protein is attached to a solid support; and (2) a second protein that specifically binds to CD28, wherein said second protein is attached to said solid support; to thereby produce a population of antigen specific T cells that comprises T cells that specifically recognize said first exogenous peptide and T cells that specifically recognize said second exogenous peptide.

In some embodiments, said population of antigen specific T cells comprises CD8+ T cells, CD4+ T cells, or both CD8+ T cells and CD4+ T cells. In some embodiments, said population of antigen specific T cells comprises effector T cells, memory T cells, or both memory T cells and effector T cells. In some embodiments, said T cells that specifically recognize said first exogenous peptide are CD4+ T cells. In some embodiments, said T cells that specifically recognize said second exogenous peptide are CD8+ T cells.

In some embodiments, step (c) comprises culturing said dendritic cells or dendritic cell precursors with a plurality of different exogenous peptides (e.g., at least 2, 3, 4, or 5 different exogenous peptides).

In some embodiments, step (d) comprises culturing said isolated T cells in a medium that comprises a plurality of cytokines. In some embodiments, said plurality of cytokines comprises IL-2, IL-7, or IL-15.

In some embodiments, step (d) further comprises culturing said isolated T cells in a medium that comprises N-Acetyl Cysteine (NAC).

In some embodiments, step (a) further comprises culturing said isolated T cells in a preconditioned medium obtained from an ex vivo culture comprising T cells obtained from a healthy subject not diagnosed with a predetermined condition (e.g., an infection). In some embodiments, step (d) further comprises culturing said isolated T cells in a preconditioned medium obtained from an ex vivo culture comprising T cells obtained from a healthy subject not diagnosed with a predetermined condition (e.g., an infection). In some embodiments, said ex vivo culture comprising T cells obtained from a healthy subject not diagnosed with a predetermined condition (e.g., an infection) have been in culture for at least 12, 24, 48, or 72 hours. In some embodiments, said preconditioned medium comprises a plurality of cytokines that stimulate activation, expansion, or both activation and expansion of said isolated T cells. In some embodiments, said preconditioned medium was cryopreserved and thawed prior to use in said method.

In some embodiments, step (d) comprises culturing said isolated T cells for no more than 96 hours, 80 hours, 72 hours, 48 hours, 36 hours, 24 hours, 18 hours, 12 hours, 10 hours, 9 hours, 8 hours, 7 hours, 6 hours, 5 hours, 4 hours, 3 hours, 2 hours, or 1 hour.

In some embodiments, step (d) comprises culturing said isolated T cells for from about 3-72 hours, 3-48 hours, 3-36 hours 3-24 hours, 3-18 hours, 3-12 hours, 3-6 hours, 6-72 hours, 6-48 hours, 6-36 hours 6-24 hours, 6-18 hours, 6-12 hours, 12-72 hours, 12-48 hours, 12-36 hours 12-24 hours, 12-18 hours, 18-72 hours, 18-48 hours, 18-36 hours 18-24 hours, 24-72 hours, 24-48 hours, or 24-36 hours.

In some embodiments, step (d) comprises culturing said isolated T cells with said artificial antigen presenting platform at a ratio of about 10:1, 15:1, 20:1, 30:1, 40:1, or 50:1.

In some embodiments, after step (d), the population of cells comprises at least a 1 fold, 2 fold, 3 fold, 4 fold, 5 fold, 10 fold, 100 fold, 1000 fold, 10000 fold increase in the number of T cells isolated from said sample. In some embodiments, said population of antigen specific T cells comprises at least about 1.0E+07, 2.0E+07, 3.0E+07, 3.5E+07, 4.0E+07, 5.0E+07, 6.0E+07, 7.0E+07, 8.0E+07, 9.0E+07, or 1.0E+08 cells.

In some embodiments, step (b) comprises isolating dendritic cell precursors. In some embodiments, said dendritic cell precursors comprise immature plasmacytoid dendritic cells, myeloid dendritic cells, monocytes, induced pluripotent stem cells (iPSC), or any combination thereof. In some embodiments, said dendritic cell precursors are not matured ex vivo. In some embodiments, said dendritic cell precursors are matured ex vivo. In some embodiments, said dendritic cell precursors are matured ex vivo by culturing said precursors in a medium that comprises at least one maturation factor (e.g., GMCSF).

In some embodiments, said first exogenous peptide is presented by an HLA class II protein expressed on the surface of said dendritic cells or dendritic cell precursors. In some embodiments, said HLA class II protein is an HLA-DP, HLA-DQ, HLA-DM, HLA-DR, or HLA-DO protein.

In some embodiments, said artificial antigen presenting platform comprises a first protein that comprises a peptide binding domain of an HLA class I protein. In some embodiments, said HLA class I protein is an HLA-A, HLA-B, or HLA-C protein.

In some embodiments, said first exogenous peptide and said second exogenous peptide are each microbial peptides. In some embodiments, said microbial peptides are viral, bacterial, or parasitic peptides.

In some embodiments, said microbial peptides are viral peptides. In some embodiments, said viral peptides are of a virus of order Nidovirales. In some embodiments, said viral peptides are of a virus of family Coronaviridae. In some embodiments, said viral peptides are of a virus of subfamily Orthocoronavirinae. In some embodiments, said viral peptides are of a virus of genus Alphacoronavirus, Betacoronavirus, Gammacoronavirus, and Deltacoronavirus. In some embodiments, said viral peptides are of a virus of genus Betacoronavirus. In some embodiments, said viral peptides are of a virus of subgenus Sarbecovirus. In some embodiments, said viral peptides are of a virus of species severe acute respiratory syndrome-related coronavirus 2. In some embodiments, said viral peptides are of a virus of strain severe acute respiratory syndrome coronavirus 2 (SARS-CoV-2). In some embodiments, said viral peptides are of a virus of a spike protein of severe acute respiratory syndrome coronavirus 2 (SARS-CoV-2). In some embodiments, said viral peptides are from a virus selected from the group consisting of: SARS-CoV-2, SARS-CoV, and MERS-CoV.

In some embodiments, said first protein that comprises a peptide binding domain of a human leucocyte antigen (HLA) protein further comprises an immunoglobulin domain. In some embodiments, said immunoglobulin domain comprises a CH3 region and a CH2 region of an immunoglobulin. In some embodiments, said immunoglobulin domain further comprises a hinge region of an immunoglobulin. In some embodiments, said immunoglobulin domain further comprises a CH1 region of an immunoglobulin. In some embodiments, said HLA protein is fused directly or indirectly to said immunoglobulin domain.

In some embodiments, said peptide binding domain of a human leucocyte antigen (HLA) protein is attached to a synthetic structure on the solid support. In some embodiments, the synthetic structure is an MHC pentamer.

In some embodiments, said second protein comprises an anti-CD28 antibody or functional fragment or functional variant thereof.

In some embodiments, the solid support for instance, as provided in an artificial antigen presenting platform is a bead or nanoparticle.

In some embodiments, said sample is a whole blood sample.

In some embodiments, said subject has been diagnosed with a microbial infection (e.g., a viral infection, e.g., a SARS-CoV-2 infection). In some embodiments, said subject was previously diagnosed with a microbial infection and at the time said sample is obtained from said subject, said subject no longer shows active infection (e.g., in the case of a viral infection as measured by a standard viral nucleic acid assay, or viral protein assay). In some embodiments, said subject is healthy (e.g., has not been diagnosed with a predetermined infection, e.g., a viral infection, e.g., a SARS-CoV-2 infection).

In some embodiments, said method further comprises haplotyping said produced a population of antigen specific T cells. In some embodiments, said method further comprises cryopreserving said produced a population of antigen specific T cells.

In some embodiments, said method further comprises genetically modifying said produced a population of antigen specific T cells to introduce a genomic disruption in at least one HLA gene.

In some embodiments, said genomic disruption inhibits expression of an HLA protein encoded by said at least one HLA gene on the surface of said cell.

In some embodiments, said genomic disruption inhibits expression of an HLA protein encoded by said at least one HLA gene on the surface of said genetically engineered human cell for a period of time sufficient to inhibit generation of an autoimmune response against said population of antigen specific T cells is administered to a non HLA matching subject.

In some embodiments, said HLA gene is an HLA class I cell. In some embodiments, said HLA class I gene is an HLA-A gene, HLA-B gene, HLA-C gene, or β-microglobulin gene. In some embodiments, said HLA class I gene is an β-microglobulin gene.

In some embodiments, said genomic disruption is a double strand break. In some embodiments, said genomic disruption is mediated by an endonuclease. In some embodiments, said endonuclease is a CRISPR endonuclease, a Zinc finger nuclease (ZFN), or a transcription activator-Like Effector Nuclease (TALEN).

In some embodiments, said first protein and said second protein are at a ratio of about 1:20, 1:30, 1:40, 1:50, 1:60, or 1:70.

In one aspect, provided herein is a population of antigen-specific T-cells made by a method described herein.

In one aspect, provided herein is a pharmaceutical composition comprising a population of (i) coronavirus-antigen-specific T-cells, (ii) influenza-antigen-specific T-cells, or (iii) (i) and (ii), made by a method described herein.

In one aspect, provided herein is a method of treating a human subject with a disorder, comprising administering to said human subject, a population of antigen specific T cells produced by a method described herein.

In some embodiments, said disorder is cancer or an infection. In some embodiments, said disorder is an infection. In some embodiments, said infection is a viral infection, bacterial infection, or parasitic infection. In some embodiments, said infection is a viral infection. In some embodiments, said infection is a coronavirus infection, influenza infection, or a combination thereof. In some embodiments, said coronavirus is selected from the group consisting of SARS-CoV-2, SARS-CoV, and MERS-CoV. In some embodiments, said coronavirus is a SARS-CoV-2.

In some embodiments, an influenza infection is of type A or B. In some embodiments, said influenza infection is selected from the group consisting of H1N1, H2N2, H3N2, H5N1, or H7H9.

In some embodiments, said population of antigen specific T cells produced by a method described herein are autologous or allogeneic to said human subject administered said population of antigen specific T cells. In some embodiments, said population of antigen specific T cells produced by a method described herein are allogenic to said human subject administered said population of antigen specific T cells.

In one aspect, provided herein is an artificial antigen presenting platform that comprises: a first protein that comprises a coronavirus peptide binding domain of a human leucocyte antigen (HLA) protein that binds an exogenous peptide, wherein said first protein is attached to a solid support; and a second protein that specifically binds to CD28, wherein said second protein is attached to said solid support.

In some embodiments, said first protein comprises the extracellular domain of said HLA protein. In some embodiments, said HLA protein is an HLA class I protein. In some embodiments, said HLA class I protein is an HLA-A, HLA-B, or HLA-C protein.

In some embodiments, said first protein is an HLA pentamer.

In some embodiments, said first protein comprises a first and a second polypeptide.

In some embodiments, said first polypeptide comprises an HLA-A α2 domain and an HLA-A α3 domain, an HLA-B α2 domain and an HLA-B α3 domain, or an HLA-C α2 domain and an HLA-C α3 domain. In some embodiments, said second polypeptide comprises an HLA-A α1 domain and a β-microglobulin domain, an HLA-B α1 domain and a β-microglobulin domain, or an HLA-C α1 domain and a β-microglobulin domain.

In some embodiments, said HLA protein is an HLA class II protein. In some embodiments, said HLA class II protein is an HLA-DP, HLA-DQ, HLA-DM, HLA-DR, or HLA-DO protein.

In some embodiments, said first protein comprises a first and a second polypeptide. In some embodiments, said first polypeptide comprises an HLA-DP α1 domain and an HLA-DP α2 domain, an HLA-DQ α1 domain and an HLA-DQ α2 domain, an HLA-DR α1 domain and an HLA-DR α2 domain, or an HLA-DM α1 domain and an HLA-DM α2 domain, or an HLA-DO α1 domain and an HLA-DO α2 domain. In some embodiments, said second polypeptide comprises an HLA-DP β1 domain and an HLA-DP β2 domain, an HLA-DQ β1 domain and an HLA-DQ β2 domain, an HLA-DR β1 domain and an HLA-DR β2 domain, or an HLA-DM β1 domain and an HLA-DM β2 domain, or an HLA-DO β1 domain and an HLA-DO β2 domain.

In some embodiments, said HLA protein is an HLA class I protein; and said particle further comprises a third protein that comprises a peptide binding domain of an HLA class II protein, wherein said third protein is conjugated to a solid support.

In some embodiments, said HLA protein is fused to an immunoglobulin polypeptide. In some embodiments, said immunoglobulin polypeptide comprises a CH3 region and a CH2 region of an immunoglobulin. In some embodiments, said immunoglobulin polypeptide further comprises a hinge region of said immunoglobulin. In some embodiments, said immunoglobulin polypeptide further comprises a CH1 region of said immunoglobulin. In some embodiments, said HLA protein is fused directly or indirectly to said immunoglobulin polypeptide.

In some embodiments, said second protein comprises an anti-CD28 antibody or functional fragment or functional variant thereof. In some embodiments, said second protein comprises a CD80 polypeptide or functional fragment or functional variant thereof.

In some embodiments, said first protein further comprises a tag.

In some embodiments, said solid support is a bead or nanoparticle. In some embodiments, said solid support is magnetic.

In some embodiments, said artificial antigen presenting platform is for use in ex vivo or in vitro cell culture.

In some embodiments, said first protein is conjugated to said solid support; and second protein is conjugated to said solid support. In some embodiments, said first protein is covalently attached to said solid support; and second protein is covalently attached to said solid support.

In some embodiments, said coronavirus is selected from the group consisting of: SARS-CoV-2, SARS-CoV, and MERS-CoV. In some embodiments, said coronavirus is SARS-CoV-2.

In some embodiments, said coronavirus peptide is a portion of a SARS-CoV-2 spike (S). In some embodiments, said SARS-CoV-2 peptide comprises an amino acid sequence of about 7-17, 7-15, 7-10, 8-17, 8-15, 8-10, 9-17, 9-15, or 9-10 continuous amino acids of SEQ ID NO: 56. In some embodiments, said SARS-CoV-2 peptide comprises an amino acid sequence of about 7-17, 7-15, 7-10, 8-17, 8-15, 8-10, 9-17, 9-15, or 9-10 continuous amino acids of a peptide encoded by SEQ ID NO: 81. In one aspect, provided herein is a method of expanding coronavirus specific human T cells ex vivo, said method comprising: (a) contacting a population of ex vivo human T cells with said artificial antigen presenting platform described herein; and (b) culturing said T cells in a culture medium for a period of time sufficient to allow for proliferation of said T cells.

In some embodiments, said first polypeptide binds to a T cell receptor (TCR) expressed on the surface of said ex vivo human T cells that is specific for said peptide.

In some embodiments, said second polypeptide binds to a CD28 protein expressed on the surface of said ex vivo human T cells.

In some embodiments, said contacting induces activation of said ex vivo human T cells. In some embodiments, said contacting induces proliferation of said ex vivo human T cells. In some embodiments, said contacting induces differentiation of said ex vivo human T cells into at least one T cell subset.

In some embodiments, said at least one T cell subset is a T effector cell subset. In some embodiments, said at least one T cell subset is a memory T cell subset. In some embodiments, said contacting induces differentiation of said ex vivo human T cells into at least two T cell subsets. In some embodiments, said at least two T cell subsets are a T effector cell subset and a memory T cell subset.

In some embodiments, said culturing comprises culturing said ex vivo human cells for less than about 3, 6, 10, 12, 16, 18, 24, 30, 36, 48, or 72 hours. In some embodiments, said culturing comprises culturing said ex vivo human cells from about 3-72 hours, 3-48 hours, 3-36 hours 3-24 hours, 3-18 hours, 3-12 hours, 3-6 hours, 6-72 hours, 6-48 hours, 6-36 hours 6-24 hours, 6-18 hours, 6-12 hours, 12-72 hours, 12-48 hours, 12-36 hours 12-24 hours, 12-18 hours, 18-72 hours, 18-48 hours, 18-36 hours 18-24 hours, 24-72 hours, 24-48 hours, or 24-36 hours.

In some embodiments, said culture medium is supplemented with at least one cytokine or chemokine.

In some embodiments, said culture medium is supplemented with N-acetyl cysteine (NAC).

In some embodiments, said ex vivo human T cells proliferate to at least 1 fold, 2 fold, 3 fold, 4 fold, 5 fold, 10 fold, 100 fold, 1000 fold, 10000 fold the number of cells prior to said culturing.

In one aspect, provided herein is a composition of ex vivo expanded T cells, wherein said cells are expanded by said method described herein.

In some cases, said T cells and said artificial antigen presenting platform are contacted at a ratio of about 10:1, 15:1, 20:1, 30:1, 40:1, or 50:1.

In some embodiments, said composition comprises ex vivo differentiated effector T cells that specifically bind a target exogenous peptide presented by a human leukocyte antigen (HLA) protein.

In some embodiments, said effector T cells comprise CD4+ T cells, CD8+ T cells, or both CD4+ and CD8+ T cells. In some embodiments, said composition comprises ex vivo differentiated memory T cells that specifically bind said target exogenous peptide presented by said human leukocyte antigen (HLA) protein. In some embodiments, memory T cells express CD45RO on the cell surface. In some embodiments, said composition comprises at least 50%, 60%, 70%, or 75% ex vivo differentiated effector T cells. In some embodiments, said composition comprises at least 25%, 30%, 40% 50%, 60%, 70%, or 75% ex vivo differentiated memory T cells. In some embodiments, said composition comprises at least 2.0×10⁶, 2.0×10⁷, 2.0×10⁸, 3.0×10⁶, 3.0×10⁷, 3.0×10⁸, 4.0×10⁶, 4.0×10⁷, 4.0×10⁸, 5.0×10⁶, 5.0×10⁷, or 5.0×10⁸ ex vivo expanded T cells. In some embodiments, said composition comprises at least 2.0×10⁶, 2.0×10⁷, 2.0×10⁸, 3.0×10⁶, 3.0×10⁷, 3.0×10⁸, 4.0×10⁶, 4.0×10⁷, 4.0×10⁸, 5.0×10⁶, 5.0×10⁷, or 5.0×10⁸ ex vivo expanded T cells that specifically bind a target exogenous peptide presented by a human leukocyte antigen (HLA) protein.

In one aspect, provided herein is a method of producing a population of ex vivo antigen specific T cells, said method comprising: (a) isolating T cells from a sample obtained from a subject; (b) isolating dendritic cells or dendritic cell precursors from said sample obtained from said subject; (c) culturing said dendritic cells or dendritic cell precursors with at least a one exogenous coronavirus peptide to produce a population of antigen presenting dendritic cells or dendritic cell precursors that present said first exogenous peptide; and (d) culturing said isolated T cells from (a) with said antigen presenting dendritic cells or precursors thereof from (c); to thereby produce a population of antigen specific T cells that specifically recognize said at least one exogenous peptide.

In some embodiments, said population of antigen specific T cells comprises CD4+ T cells, CD8+ T cells, or both CD8+ T cells and CD4+ T cells. In some embodiments, said population of antigen specific T cells comprises effector T cells, memory T cells, or both memory T cells and effector T cells.

In some embodiments, step (c) comprises culturing said dendritic cells or dendritic cell precursors with a plurality of different exogenous peptides (e.g., at least 2, 3, 4, or 5 different exogenous peptides).

In some embodiments, step (d) comprises culturing said isolated T cells in a medium that comprises a plurality of cytokines.

In some embodiments, step (d) comprises culturing said isolated T cells in a medium that comprises N-acetyl cysteine (NAC).

In some embodiments, step (a) further comprises culturing said isolated T cells in a preconditioned medium obtained from an ex vivo culture comprising T cells obtained from a healthy subject not diagnosed with a predetermined condition (e.g., an infection). In some embodiments, step (d) further comprises culturing said isolated T cells in a preconditioned medium obtained from an ex vivo culture comprising T cells obtained from a healthy subject not diagnosed with a predetermined condition (e.g., an infection).

In some embodiments, said ex vivo culture comprising T cells obtained from a healthy subject not diagnosed with a predetermined condition (e.g., an infection) has been in culture for at least 12, 24, 48, or 72 hours. In some embodiments, said preconditioned medium comprises a plurality of cytokines that stimulate activation, expansion, or both activation and expansion of said isolated T cells. In some embodiments, said preconditioned medium was cryopreserved and thawed prior to use in said method.

In some embodiments, step (d) comprises culturing said isolated T cells for no more than 96 hours, 80 hours, 72 hours, 48 hours, 36 hours, 24 hours, 18 hours, 12 hours, 10 hours, 9 hours, 8 hours, 7 hours, 6 hours, 5 hours, 4 hours, 3 hours, 2 hours, or 1 hour. In some embodiments, step (d) comprises culturing said isolated T cells for from about 3-72 hours, 3-48 hours, 3-36 hours 3-24 hours, 3-18 hours, 3-12 hours, 3-6 hours, 6-72 hours, 6-48 hours, 6-36 hours 6-24 hours, 6-18 hours, 6-12 hours, 12-72 hours, 12-48 hours, 12-36 hours 12-24 hours, 12-18 hours, 18-72 hours, 18-48 hours, 18-36 hours 18-24 hours, 24-72 hours, 24-48 hours, or 24-36 hours.

In some embodiments, after step (d), the population of cells comprises at least 2 fold, 10 fold, 100 fold, 1000 fold, 10000 fold increase in the number of T cells isolated from said sample.

In some embodiments, said population of antigen specific T cells comprises at least about 1.0E+07, 2.0E+07, 3.0E+07, 3.5E+07, 4.0E+07, 5.0E+07, 6.0E+07, 7.0E+07, 8.0E+07, 9.0E+07, or 1.0E+08 cells.

In some embodiments, step (b) comprises isolating dendritic cell precursors.

In some embodiments, said dendritic cell precursors comprise immature plasmacytoid dendritic cells, myeloid dendritic cells, monocytes, or any combination thereof. In some embodiments, said dendritic cell precursors are not matured ex vivo. In some embodiments, said dendritic cell precursors are matured ex vivo. In some embodiments, said dendritic cell precursors are matured ex vivo by culturing said precursors in a medium that comprises at least one maturation factor (e.g., GMCSF).

In some embodiments, said exogenous peptide is presented by an HLA class II protein expressed on the surface of said dendritic cells or dendritic cell precursors. In some embodiments, said HLA class II protein is an HLA-DP, HLA-DQ, HLA-DM, HLA-DR, or HLA-DO protein.

In some embodiments, said coronavirus is selected from the group consisting of: SARS-CoV-2, SARS-CoV, and MERS-CoV. In some embodiments, said coronavirus is SARS-CoV-2.

In some embodiments, said coronavirus peptide is a portion of a SARS-CoV-2 spike (S). In some embodiments, said SARS-CoV-2 peptide comprises an amino acid sequence of about 7-17, 7-15, 7-10, 8-17, 8-15, 8-10, 9-17, 9-15, or 9-10 continuous amino acids of SEQ ID NO: 56. In some embodiments, said SARS-CoV-2 peptide comprises an amino acid sequence of about 7-17, 7-15, 7-10, 8-17, 8-15, 8-10, 9-17, 9-15, or 9-10 continuous amino acids of a peptide encoded by SEQ ID NO: 81.

In one aspect, provided herein is a population of coronavirus-antigen-specific T-cells made by a method described herein.

In one aspect, provided herein is a pharmaceutical composition comprising a population of antigen specific T cells made by a method described herein.

In one aspect, provided herein is a method of treating a coronavirus infection in a human subject, said method comprising: administering a population of coronavirus antigen specific T cells made by a method described herein.

In some embodiments, said coronavirus is selected from the group consisting of: SARS-CoV-2, SARS-CoV, and MERS-CoV. In some embodiments, said coronavirus infection is a SARS-CoV-2 infection.

In some embodiments, said coronavirus-antigen-specific T-cells are autologous or allogeneic to said human subject.

In one aspect, described herein is a genetically engineered ex vivo human cell that comprises: (a) a genomic disruption in at least one human leukocyte antigen (HLA) gene or at least one transcriptional regulator of an HLA gene; and (b) a nucleic acid encoding a T cell receptor (TCR) that specifically binds to a coronavirus peptide presented by a human leukocyte antigen (HLA) protein expressed on the surface of a cell.

In some embodiments, said nucleic acid encoding said TCR is integrated into the genome of said cell. In some embodiments, said nucleic acid encoding said TCR is integrated into a TRAC or TRBC gene locus in the genome of said cell.

In some embodiments, said genomic disruption inhibits expression of an HLA protein encoded by said at least one HLA gene on the surface of said cell. In some embodiments, said genomic disruption inhibits expression of an HLA protein encoded by said at least one HLA gene on the surface of said genetically engineered human cell for a period of time sufficient to inhibit generation of an autoimmune response against said T cell when said T cell is administered to a non HLA matching subject.

In some embodiments, said HLA gene is an HLA class I cell. In some embodiments, said HLA class I gene is an HLA-A gene, HLA-B gene, HLA-C gene, or β-microglobulin gene. In some embodiments, said HLA class I gene is an β-microglobulin gene.

In some embodiments, said genomic disruption is a double strand break. In some embodiments, said genomic disruption is mediated by an endonuclease. In some embodiments, said endonuclease is a CRISPR endonuclease, a Zinc finger nuclease (ZFN), or a transcription activator-Like Effector Nuclease (TALEN).

In some embodiments, said coronavirus peptide is of a coronavirus selected from the group consisting of SARS-CoV-2, SARS-CoV, and MERS-CoV. In some embodiments, said coronavirus is a SARS-CoV-2. In some embodiments, said coronavirus peptide is of a SARS-CoV-2 spike protein.

In some embodiments, provided herein is a population of said genetically engineered human cell described herein.

In some embodiments, provided herein is a pharmaceutical composition comprising said population of genetically engineered cells described herein.

In some embodiments, provided herein is a method of making a genetically engineered human cell ex vivo, the method comprising: (a) inducing a genomic disruption in at least one human leukocyte antigen (HLA) gene or at least one transcriptional regulator of an HLA gene; and (b) introducing a nucleic acid encoding a T cell receptor (TCR) that specifically binds to a coronavirus peptide presented by a human leukocyte antigen (HLA) protein expressed on the surface of a cell.

In some embodiments, said nucleic acid encoding said TCR is integrated into the genome of said cell. In some embodiments, said nucleic acid encoding said TCR is integrated into a TRAC or TRBC gene locus in the genome of said cell.

In some embodiments, a gamma delta T cell can be utilized in methods provided herein. As opposed to their alpha beta T cell counterparts, gamma delta T cells are not MHC restricted. These cells can be modified to express an exogenous TCR, for example a TCR that targets an antigen peptide provided herein such as one from SARS-CoV-2.

In some embodiments, said genomic disruption inhibits expression of an HLA protein encoded by said at least one HLA gene on the surface of said cell. In some embodiments, said genomic disruption inhibits expression of an HLA protein encoded by said at least one HLA gene on the surface of said method for a period of time sufficient to inhibit generation of an autoimmune response against said T cell when said T cell is administered to a non HLA matching subject.

In some embodiments, said HLA gene is an HLA class I cell. In some embodiments, said HLA class I gene is an HLA-A gene, HLA-B gene, HLA-C gene, or β-microglobulin gene. In some embodiments, said HLA class I gene is an β-microglobulin gene.

In some embodiments, said genomic disruption is a double strand break. In some embodiments, said genomic disruption is mediated by an endonuclease. In some embodiments, said endonuclease is a CRISPR endonuclease, a Zinc finger nuclease (ZFN), or a transcription activator-Like Effector Nuclease (TALEN).

In some embodiments, said coronavirus peptide is of a coronavirus selected from the group consisting of SARS-CoV-2, SARS-CoV, and MERS-CoV. In some embodiments, said coronavirus is a SARS-CoV-2. In some embodiments, said coronavirus peptide is of a SARS-CoV-2 spike protein.

In some embodiments, provided herein is a population of said genetically engineered human cells made by a method described herein.

In some embodiments, provided herein is a pharmaceutical composition comprising said population of genetically engineered cells made by a method described herein.

In some embodiments, provided herein is a method of treating a coronavirus infection in a human subject, said method comprising: administering a population of genetically engineered T cells described herein.

In some embodiments, said coronavirus is selected from the group consisting of: SARS-CoV-2, SARS-CoV, and MERS-CoV. In some embodiments, said coronavirus infection is a SARS-CoV-2 infection.

In some embodiments, said genetically engineered T cells are allogenic to said subject.

In some embodiments, provided herein is a genetically engineered ex vivo human cell that comprises: (a) a genomic disruption in at least one human leukocyte antigen (HLA) gene or at least one transcriptional regulator of an HLA gene; and (b) expresses a T cell receptor (TCR) that specifically binds to a coronavirus peptide presented by a human leukocyte antigen (HLA) protein expressed on the surface of a cell.

In some embodiments, said genomic disruption inhibits expression of an HLA protein encoded by said at least one HLA gene on the surface of said cell.

In some embodiments, said genomic disruption inhibits expression of an HLA protein encoded by said at least one HLA gene on the surface of said genetically engineered human cell for a period of time sufficient to inhibit generation of an autoimmune response against said T cell when said T cell is administered to a non HLA matching subject.

In some embodiments, said HLA gene is an HLA class I cell. In some embodiments, said HLA class I gene is an HLA-A gene, HLA-B gene, HLA-C gene, or β-microglobulin gene. In some embodiments, said HLA class I gene is an β-microglobulin gene.

In some embodiments, said genomic disruption is a double strand break. In some embodiments, said genomic disruption is mediated by an endonuclease. In some embodiments, said endonuclease is a CRISPR endonuclease, a Zinc finger nuclease (ZFN), or a transcription activator-Like Effector Nuclease (TALEN).

In some embodiments, said coronavirus peptide is of a coronavirus selected from the group consisting of SARS-CoV-2, SARS-CoV, and MERS-CoV. In some embodiments, said coronavirus is a SARS-CoV-2. In some embodiments, said coronavirus peptide is of a SARS-CoV-2 spike protein.

In some embodiments, provided herein is a population of said genetically engineered human cell described herein.

In some embodiments, provided herein is a pharmaceutical composition comprising said population of genetically engineered cells described herein.

In some embodiments, provided herein is a method of making a population of genetically engineered human T cells, the method comprising: (a) obtaining a population of T cells from a human subject that express a T cell receptor (TCR) that specifically binds to a coronavirus peptide presented by a human leukocyte antigen (HLA) protein expressed on the surface of a cell; and (b) inducing a genomic disruption in at least one human leukocyte antigen (HLA) gene or at least one transcriptional regulator of an HLA gene in said population of T cells ex vivo.

In some embodiments, said genomic disruption inhibits expression of an HLA protein encoded by said at least one HLA gene on the surface of said cell.

In some embodiments, said genomic disruption inhibits expression of an HLA protein encoded by said at least one HLA gene on the surface of said method for a period of time sufficient to inhibit generation of an autoimmune response against said T cell when said T cell is administered to a non HLA matching subject.

In some embodiments, said HLA gene is an HLA class I cell. In some embodiments, said HLA class I gene is an HLA-A gene, HLA-B gene, HLA-C gene, or β-microglobulin gene. In some embodiments, said HLA class I gene is an β-microglobulin gene.

In some embodiments, said genomic disruption is a double strand break. In some embodiments, said genomic disruption is mediated by an endonuclease. In some embodiments, said endonuclease is a CRISPR endonuclease, a Zinc finger nuclease (ZFN), or a transcription activator-Like Effector Nuclease (TALEN). In some embodiments, said coronavirus peptide is of a coronavirus selected from the group consisting of SARS-CoV-2, SARS-CoV, and MERS-CoV. In some embodiments, said coronavirus is a SARS-CoV-2. In some embodiments, said coronavirus peptide is of a SARS-CoV-2 spike protein.

A population of said genetically engineered human cells made by the method of any one of claims 207-218.

In some embodiments, described herein is a pharmaceutical composition comprising said population of genetically engineered cells made by a method described herein.

In some embodiments, provided herein is a method of treating a coronavirus infection in a human subject, said method comprising: administering a population of genetically engineered T cells described herein.

In some embodiments, said coronavirus is selected from the group consisting of: SARS-CoV-2, SARS-CoV, and MERS-CoV. In some embodiments, said coronavirus infection is a SARS-CoV-2 infection. In some embodiments, said genetically engineered T cells are allogenic to said subject.

Provided herein is a composition that comprises an antigen presenting particle that comprises: a) an antigen peptide bound by a synthetic major histocompatibility complex (MHC); and b) a co-stimulatory moiety. In some cases, the composition further comprises a plurality of human cells. In some cases, the human cells are immune cells. In some cases, the immune cells are T cells, NK cells, dendritic cells, macrophages, Langerhans cells, B cells, or any combination thereof. In some cases, the immune cells are T cells, and wherein the T cells are alpha beta T cells. In some cases, the immune cells are T cells, and the T cells are gamma delta T cells. In some cases, the T cells are matured from induced pluripotent stem cells. In some cases, the immune cells are dendritic cells, and the dendritic cells are primed with the synthetic MHC. In some cases, the composition further comprises N-acetyl cysteine (NAC). In some cases, the composition further comprises a cytokine or chemokine. In some cases, the MHC is class I. In some cases, the MHC is class II. In some cases, the particle further comprises a protein that binds CD3. In some cases, the antigen peptide is from about 15-25 amino acid residues long. In some cases, the human cells and the particle are at a ratio of about 10:1, 15:1, 20:1, 30:1, 40:1, or 50:1. In some cases, the synthetic MHC and the protein are at a ratio of about 1:20, 1:30, 1:40, 1:50, or 1:60. In some cases, the antigen peptide is from a microbe. In some cases, the microbe is viral, bacterial, or parasitic. In some cases, the microbe is viral and comprises: influenza, Epstein-Barr virus (EBV), mega virus, Norwalk virus, coxsackie virus, middle east respiratory syndrome-related coronavirus, severe acute respiratory syndrome-related coronavirus, SARS-Cov-2 virus, hepatitis B, varicella zoster virus, parvovirus, adenovirus, Marburg virus, Ebola virus, Rabies, Smallpox, HIV, Hantavirus, Dengue, Rotavirus, MERS-CoV, mumps virus, cytomegalovirus (CMV), Herpes virus, papillomavirus, chikungunya virus, and any combination thereof. In some cases, the co-stimulatory moiety is from an antigen presenting cell. In some cases, the co-stimulatory moiety is selected from the group consisting of: CD80, CD86, B7RP1, B7-H3, H7-H4, HVEM, CD137L, OX40L, CD70, CD40, GAL9, PDL2, PDL2, NKG2D, CD16, NKp30, NKp44, NKp46, DNAM1, CD96, CD94, KIR, TIGIT, and any combination thereof. In some cases, the synthetic MHC is an MHC trimer or pentamer.

Provided herein is a synthetic particle that comprises an antigen peptide bound by a synthetic major histocompatibility complex (MHC), wherein the antigen peptide comprises: (a) an amino acid sequence of about 7-17, 7-15, 7-10, 8-17, 8-15, 8-10, 9-17, 9-15, or 9-10 continuous amino acids of SEQ ID NO: 42-56; or (b) an amino acid sequence of about 7-17, 7-15, 7-10, 8-17, 8-15, 8-10, 9-17, 9-15, or 9-10 continuous amino acids of a peptide encoded by SEQ ID NO: 81; or c. at least 85% identity to any one of SEQ ID NO: 1-41, 54-55, 59-79 and 81. In some cases, the antigen peptide comprises at least 90%, 95%, 97%, 99%, or 100% sequence identity with any one of SEQ ID NO: 1-41, 54-55, 59-79, or 81. In some cases, a synthetic particle further comprises a co-stimulatory moiety. In some cases, the co-stimulatory moiety is selected from the group consisting of: CD80, CD86, B7RP1, B7-H3, H7-H4, HVEM, CD137L, OX40L, CD70, CD40, GAL9, PDL2, PDL2, NKG2D, CD16, NKp30, NKp44, NKp46, DNAM1, CD96, CD94, KIR, TIGIT, and any combination thereof. In some cases, the synthetic MHC and the co-stimulatory moiety are at a ratio of about 1:20, 1:30, 1:40, 1:50, or 1:60. In some cases a synthetic particle further comprises a second antigen peptide bound by a second synthetic MHC. In some cases, the second antigen peptide is an influenza antigen peptide. In some cases, a synthetic particle further comprises a third antigen peptide bound by a third synthetic MHC. In some cases, the third antigen peptide is an H1N1 antigen peptide. In some cases, the synthetic MHC is an MHC trimer or pentamer. In some cases, the synthetic MHC comprises human MHC chains.

Provided herein is a composition that comprises the synthetic particle and an antigen presenting cell (APC). In some cases, the APC is a dendritic cell. In some cases, the dendritic cell has been primed with the synthetic particle.

Provided herein is a method of generating a virus induced lymphocyte (VIL), the method comprising contacting a T cell with a synthetic particle thereby generating the VIL. In some cases, after the contacting, the VIL comprises antigen peptide specificity as measured by ELISPOT. In some aspects, a method further comprises contacting a second or third time. In some cases, a method further comprises contacting the T cell with a dendritic cell. In some cases, the dendritic cell has been antigen peptide primed by the synthetic particle.

Provided herein is a composition that comprises (a) a synthetic particle; (b) a dendritic cell; and (c) a T cell. In some cases, the dendritic cell is antigen peptide primed by the synthetic particle.

There remains a considerable need for compositions and methods to treat diseases and conditions associated with a pathogenic infection. The present disclosure addresses these needs, and also provides additional advantages applicable for diagnosis, prognosis, and treatment for a wide diversity of diseases and conditions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an exemplary therapy described herein, wherein a COVID-19 patient's T cells (for example VILs) are isolated from whole blood, contacted ex vivo with a particle that is an artificial antigen presenting platform, that has an MHC domain bound to or fused to a SARS-Cov-2 peptide and a T co-stimulatory molecule (e.g., anti-CD28 antibody) on the surface (VIPR), which can stimulate SARS-Cov-2 specific T cells. The cells are rapidly expanded (REP) and reinfused back into the patient.

FIG. 2 shows a schematic of an exemplary particle described herein that has on its surface a protein comprising an MHC portion bound to or fused to a SARS-Cov-2 peptide, and a binding moiety capable of binding a co-stimulatory receptor on the surface of a T cell (e.g., CD80). The particle supports the activation, proliferation, and differentiation of SARS-Cov-2-specific T cells ex vivo, such as effector T cells and memory T cells.

FIG. 3 shows a schematic of three exemplary treatment options available to a SARS-Cov-2 positive subject. Exemplary treatment options include (1) treatment using the subject's autologous T cells that are stimulated ex vivo with autologous anti-SARS-Cov-2 peptide-pulsed dendritic cells. (2) autologous T cells stimulated with a subject particle expressing an HLA-matched HLA-DR-Ig expressing SARS-Cov-2 spike antigen and anti-CD28. (3) allogeneic T cells stimulated with an allogeneic particle carrying a universal HLA-like molecule that expresses SARS-Cov-2 spike antigen.

FIG. 4 shows an exemplary therapy wherein a COVID-19 patient's T cells are isolated from whole blood and cultured with an MHC-I peptide-presenting particle and autologous subject-derived blood dendritic cells pulsed with the peptide. This therapy provides MHC class II antigen presentation via the dendritic cell and MHC-I antigen presentation via the particle thereby expanding both CD4 and CD8 T cells, thus allowing for expansion of therapeutic T cells in a quantity, quality, and temporal time course that allows for a rapid, timely, and therapeutically relevant cellular product.

FIG. 5 shows an exemplary allogeneic platform provided herein whereby lymphocytes are isolated from a recovered COVID-19 subject and contacted with a particle provided herein (for example a VIPR particle), expanded via REP, and cryopreserved for off-the-shelf treatment of many subjects.

FIG. 6 shows flow cytometry plots of dendritic cell isolation. Shown are percent dendritic cells, further subdivided into plasmacytoid DCs (PDCs) and Myeloid DCs (MDCS1 and MDCS2).

FIG. 7 shows flow cytometry plots on day 0 of a reagent validation test to test MART1 and SARS-CoV-2 biotinylated pentamers with 3 different streptavidin secondaries to identify positive cells in PBMCs at Day 0 and test background staining with the reagents.

FIG. 8A shows an experimental schematic for dendritic cell culture testing. FIG. 8B shows flow cytometry plots on day 5 of a dendritic cell culture using Covid Ag1 and MART1 Ag peptides. Tested pools shown are (1) Unfractionated PBMCs; Isolated DCs; DCs+PBMC Feeders; Soluble protein; (2) Stains used were pentamer-bio and a streptavidin secondary approach. FIG. 8C shows flow cytometry plots on day 8 of a dendritic cell culture using Covid Ag1 and MART1 Ag peptides. FIG. 8D shows a summary of percent antigen specific T cells post culture with MART1 peptide loaded DCs.

FIG. 9A shows an experimental schematic for testing SARS-Cov-2 or CMV control antigen loaded particles. Conditions tested were (1) High and low dose of beads; (2) soluble antigen; and (3) Stains used Pentamer-Bio+Streptavidin secondary approach. FIG. 9B shows flow cytometry plots on day 0 of culture. FIG. 9C shows flow cytometry plots on day 3 of culture. FIG. 9D shows flow cytometry plots on day 10 of culture. FIG. 9E shows a summary of day 3 culture data. FIG. 9F shows a summary of day 10 culture data.

FIG. 10A shows an experimental schematic of DC culture testing. Conditions tested are DCs and T cells from a CMV+ donor: (1) DCs pulsed with CMV Ag; Covid Ag-1; MART Ag; (2) Stained with each Pentamer-Bio and analyzed Day 7 and Day 12. FIG. 10B shows day 7 culture flow cytometry plots. FIG. 10C shows day 12 culture flow cytometry plots. FIG. 10D shows a summary of the day 7 plots. FIG. 10E shows a summary of the day 12 plots.

FIG. 11A shows an experimental schematic of testing of two particles, V1 and V2 using a CMV positive donor. Conditions that were tested include: (1) Comparison to soluble matched peptide antigen; (2) V1 particles (CMV Pentamer: CD28); V2 particles (CMV Pentamer: CD3: CD28); and V2 control (CD3:CD28); and (3) Included Covid antigen-1 as a test to see if cells are detectible (also as negative control). FIG. 11B shows flow cytometry data collected on day 6 of culture. FIG. 11C shows flow cytometry data collected on day 12 of culture. FIG. 11D shows flow cytometry data collected on day 15 of culture. A summary of day 6, day 12 and day 15 data is shown in FIG. 11E, FIG. 11F, and FIG. 11G respectively.

FIG. 12A shows an experimental schematic of optimization testing using a CMV positive donor. Conditions that were tested include: (1) Re-dosing of beads onto T cells; (2) Dose of Beads vs T cells; (3) Ration of Pentamer and CD28 on beads; and (4) Addition of N-acetyl cysteine (NAC) to media with beads. FIG. 12B shows flow cytometry data collected on day 2 after 1 dose of CMV pp65 loaded particles. FIG. 12C shows flow cytometry data collected on day 4 after 1 dose of CMV pp65 loaded particles. FIG. 12D shows flow cytometry data collected on day10 after 1 dose of CMV pp65 loaded particles. FIG. 12E shows a summary of the day 4 culture data. FIG. 12F shows a summary of the day 10 culture data. FIG. 12G shows flow cytometry data collected on day 4 after 1 dose of CMV pp65 loaded particles cultured in the presence of NAC. FIG. 12H shows flow cytometry data collected on day 10 after 1 dose of CMV pp65 loaded particles cultured in the presence of NAC. FIG. 12I shows a summary of the day 4 NAC containing culture data. FIG. 12J shows a summary of the day 10 NAC containing culture data. FIG. 12K shows flow cytometry plots acquired on day 10 of culture of a comparative study using utilizing different particle designs: Pentamer/CD28 Ratios: 1:1; 1:10; 1:30 (FIG. 12K). A summary of different particle design day 10 data is shown in FIG. 12L. FIG. 12M shows flow cytometry plots taken on day 10 of culture evaluating T cell to particle ratios: 20:1, 5:1, 1:1, 1:20, and 1:5. FIG. 12N shows a summary of the day 10 assay evaluating different T cell to particle ratios.

FIG. 13 shows a timeline of an exemplary 7-day expansion of antigen-specific CD4 and CD8 T cells utilizing compositions and methods provided herein.

FIG. 14A shows a strategy for the isolation, stimulation and enrichment of CMV antigen-specific T cells from donor PBMCs. FIG. 14B depicts representative flow-cytometric analysis showing proportions of antigen-specific CD8+ and CD4+ T cells identified by Pentamer staining, 7-days after enrichment and expansion with antigen-specific VIPR Particles. FIG. 14C shows a summary of CD8+ data obtained in b, (n=3). FIG. 14D depicts a histogram showing this VIL enrichment is VIPR particles dose-dependent (n=3). FIG. 14E depicts a histogram showing impact on VIL enrichment of MHC-I pentamer and anti-CD28 ratio conjugated to VIPR particles (n=3).

FIG. 15A depicts representative flow-cytometric analysis showing proportions of SARS-CoV-2 antigen-specific CD8+ T cells 7-days after expansion and enrichment with YLQPRTFLL (SEQ ID NO: 72) antigen-specific VIPR Particles. FIG. 15B shows a summary of CD8+ SARS-CoV-2 VIL %, cell number and fold change after VIPR expansion (n=7). FIG. 15C depicts histograms showing total CMV pp65 antigen-specific CD8+ T cell numbers expanded by VIPR particles after 7-days and overall fold change in antigen specific cells (n=3).

FIG. 16A shows the expression of activation markers 4-1BB, OX-40, CD25 and HLA-DR among enriched and expanded SARS-CoV-2-specific CD8+ T cells at day 7 (n=8). FIG. 16B shows the expression of checkpoint genes PD-1, LAG-3 and TIGIT among enriched and expanded SARS-CoV-2-specific CD8+ T cells at day 7 (n=8). FIG. 16C shows the expression of activation markers 4-1BB, OX-40, CD25 and HLA-DR among enriched and expanded CMV-specific CD8+ T cells (n=4). FIG. 16D shows the expression of checkpoint genes PD-1, LAG-3 and TIGIT among enriched and expanded CMV-specific CD8+ T cells (n=4).

FIG. 17A shows representative flow-cytometric analysis showing expression of CD45RO, CD45RA and CD62L among enriched and expanded CMV-specific CD8+ T cells at day-7. FIG. 17B shows a summary of naïve, central memory and effector memory T cells subsets from data obtained in FIG. 17A, (n=4). FIG. 17C shows representative flow-cytometric analysis showing expression of CD45RO, CD45RA and CD62L among enriched and expanded SARS-CoV-2-specific CD8+ T cells at day-7. 17D shows a summary of naïve, central memory and effector memory T cells subsets from data obtained in FIG. 17C, (n=8).

FIG. 18A shows representative flow-cytometric analysis showing expression of IFN-γ and TNF-a among enriched and expanded SARS-COV-2 CD8+ T cells at day-7, after 6-hour stimulation with specific peptide antigen, and proportions of IFN-γ/TNF-a expressing cells also expressing IL-2. FIG. 18B shows a summary of the data obtained as in FIG. 18A for each cytokine (n=7). FIG. 18C shows representative proportion of SARS-CoV-2 CD8+ T cells expressing 1, 2 or 3 cytokines. FIG. 18D shows extended analysis of SARS-CoV-2 VIL polycytokine function as SPICE representation. FIG. 18E shows representative flow-cytometric analysis showing expression of IFN-γ and TNF-a among enriched and expanded CMV-specific CD8+ T cells at day-7, after 6-hour stimulation with specific peptide antigen, and proportions of IFN-γ/TNF-a expressing cells also expressing IL-2. FIG. 18F shows a summary of the data obtained as in FIG. 18E for each cytokine (n=3). FIG. 18G shows representative proportion of CMV CD8+ T cells expressing 1, 2 or 3 cytokines. FIG. 18H shows extended analysis of CMV-specific VIL polycytokine function as SPICE representation.

FIG. 19 shows a schematic for a COVID-19 cell therapy in which PMBCs are collected from the blood of HLA-typed hospitalized patients and total T cells isolated. T cells are stimulated with HLA-matched MHC-I/MHC-II antigen-specific SARS-CoV-2 VIPR beads to enrich and expand CD4+ and CD8+ T cells with TCRs specific for the SARS-CoV-2 antigen epitopes. These antigen-specific VIL expand at an average of 1,000-fold prior to adoptive transfer back to HLA-matched patients to mediate a T cell immune response to support the eradication of the SARS-CoV-2 virus and to engender protective immunity against repeat infection.

### DETAILED DESCRIPTION

Compositions and methods provided herein can be utilized to generate antigen-peptide specific T cells to reduce or eliminate sources of foreign peptides, such as viruses, bacteria, parasites, and cancer cells. The compositions and methods provided herein can increase the number or percentage of antigen peptide-specific T cells for use as a therapeutic, for example in an autologous or allogeneic adoptive cellular therapy. In an exemplary autologous setting, a SARS-Cov-2 positive subject undergoes apheresis to isolate circulating T cells. Those T cells are cultured with a particle expressing the SARS-Cov-2 spike peptide and a CD28 costimulatory agent such that anti-SARS-Cov-2 specific T cells are expanded in large numbers for adoptive immunotherapy. Similar techniques can be utilized to generate allogeneic T cells for HLA-matched or HLA-partially matched recipients.

The practice of some embodiments disclosed herein employ, unless otherwise indicated, conventional techniques of immunology, biochemistry, chemistry, molecular biology, microbiology, cell biology, genomics and recombinant DNA, which are within the skill of the art.

Unless defined otherwise, all technical and scientific terms used herein have the same meaning as is commonly understood by one of skill in the art.

The term “a” and “an” refers to one or to more than one (i.e., to at least one) of the grammatical object of the article. By way of example, “an element” means one element or more than one element.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “artificial antigen presenting cell” and “artificial antigen presenting platform” as used herein and in U.S. Provisional Patent Application Ser. No. 63/013,435 and U.S. Provisional Patent Application Ser. No. 63/056,517 are synonymous in that both refer to an “artificial antigen presenting platform” as described herein. The skilled artisan will appreciate that “artificial antigen presenting cells” and “artificial antigen presenting platforms” do not refer to naturally-occurring antigen presenting cells.

As used herein, “major histocompatibility complex” (MHC) refers to a group of highly polymorphic glycoproteins or portions thereof encoded by MHC class I and MHC class II genes. MHC is a series of cell surface proteins used by the body to recognize foreign molecules. These proteins bind antigens and then display the antigens on their surface so that the antigens are recognized by immune cells, such as T-cells. There are three major class I MHC haplotypes (A, B, and C), three major MHC class II haplotypes (DR, DP, and DQ), newly identified MHC haplotypes and subsets thereof are also provided. The MHC in humans is also known as the human leukocyte antigen (HLA) complex. Class I MHC proteins may further comprise other elements such as molecules which assist in antigen presenting such as TAP and tapasin. The MHC complex can refer to entire proteins, functional and non-functional portions of MHC proteins, and any modified versions thereof. Class I MHC proteins, generally, comprises three domains, labeled a1, a2, and a3. The a1 domain functions to attach the MHC to the b-microglobulin, a3 functions is a transmembrane domain which anchors the protein into the cell membrane, and the groove between the a1 and a2 submits functions as the peptide presenting domain. On the other hand, class II MHC proteins typically have two domains, each with two classes of protein subunits, a and b. The first domain comprises a1 and a2 subunits while the second domain comprises b1 and b2 subunits. The a2 and b2 form the transmembrane domain of the protein anchoring the MHC to the cellular membrane with the a1 and b1 subunits forming the peptide binding groove.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “Virus Induced Lymphocyte” (VIL) as used herein, refers to a cell generated using any of the compositions or methods provided herein. For example, a VIL can comprise peptide-specificity to any of the peptides or fragments thereof provided herein. A VIL can be generated using any of the particles, antigen presenting cells, and expansion methods described. A VIL can be generated from any type of cell.

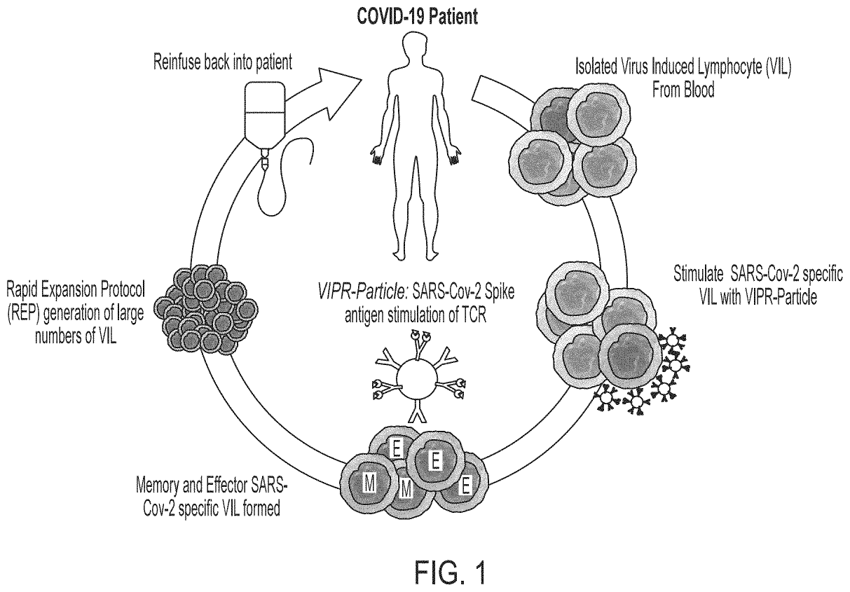

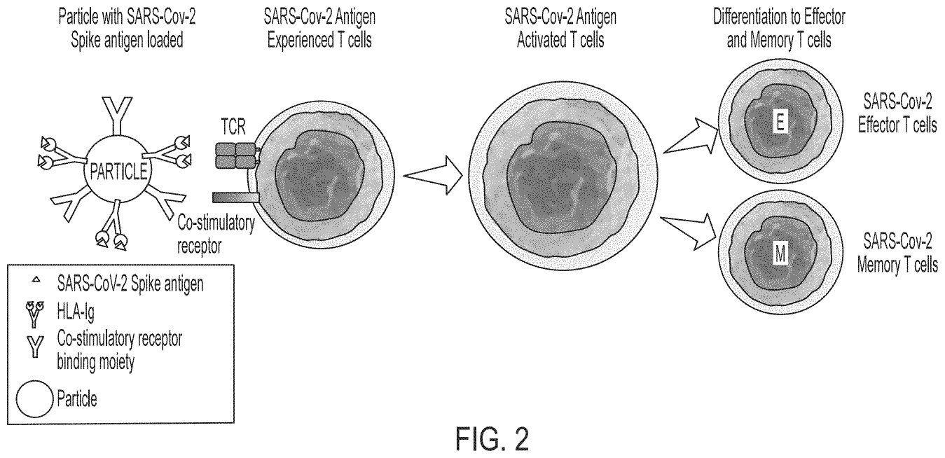

Compositions and methods provided herein can be used to treat, stabilize, and/or prevent conditions such as infections or cancers. Compositions comprising a solid support, such as a particle, that displays an antigen-peptide from a source such as a virus can be used to generate increased numbers of antigen peptide-specific T cells for use in therapy, FIG. 1.

### Artificial Antigen Presenting Platforms

In some embodiments, provided herein are artificial antigen presenting platforms. In some embodiments, the artificial antigen presenting platforms comprise a solid support (e.g., a particle described herein) and a peptide binding moiety fused (e.g., as described herein) or bound to a peptide (e.g., as described herein). Non-solid supports may also be used. The terms “artificial antigen presenting cell” and “artificial antigen presenting platform” (aAPP) as used herein and in U.S. Provisional Patent Application Ser. No. 63/013,435 and U.S. Provisional Patent Application Ser. No. 63/056,517 are synonymous in that both refer to an “artificial antigen presenting platform” (aAPP) as described herein. The skilled artisan will appreciate that “artificial antigen presenting cells” and “artificial antigen presenting platforms” do not refer to naturally-occurring antigen presenting cells. In some embodiments, said peptide binding moiety binds to a protein (e.g., a TCR) expressed on the surface of a T cell that specifically recognizes said peptide. In some embodiments, said antigen presenting cell comprises a co-stimulatory moiety (e.g., as described herein). In some embodiments, said co-stimulatory moiety binds to a protein expressed on the surface of a T cell and enhances activation and/or expansion of said T cell. In some cases, said peptide is a microbial peptide (e.g., a viral peptide). In some cases, the peptide is associated with a peptide binding domain of a human leucocyte antigen (HLA) protein or fragment thereof. In some cases, said artificial antigen presenting platform comprises additional attachments, such as a costimulatory protein or fragment thereof such that when the artificial antigen presenting platform contacts a cell in the population, the cell is appropriately stimulated.

In some cases, an artificial antigen presenting platform comprises a synthetic MHC. A synthetic MHC can be a synthetic structure that comprises MHC peptide chains that correspond to the soluble form of the normally membrane-bound protein. The number of synthetic MHCs comprised on the aAPP can be modulated. A synthetic MHC can be a trimer, tetramer, heptamer, pentamer, hexamer, octamer, megamer, or any combination thereof. In some cases, an aAPP comprises an MHC pentamer.

For class I synthetic MHCs, the construct can be similar to or identical to the soluble form of an MHC. The synthetic MHC can be derived from the native form by deletion of the transmembrane and cytoplasmic domains. For class I proteins, the soluble form can include the α1, α2 and α3 domains of the a chain. For class II proteins the soluble form can include the α1 and α2 or β1 and β2 domains of the α chain or β chain, respectively. Not more than about 10, usually not more than about 5, preferably none of the amino acids of the transmembrane domain will be included in a subject synthetic MHC. The deletion may extend as much as about 10 amino acids into the α3 domain. Preferably none of the amino acids of the α3 domain will be deleted. The deletion will be such that it does not interfere with the ability of the α3 domain to fold into a functional disulfide bonded structure. The class Iβ chain, β2m, lacks a transmembrane domain in its native form, and does not have to be truncated. The above deletion is likewise applicable to class II subunits. It may extend as much as about 10 amino acids into the α2 or β2 domain, preferably none of the amino acids of the α2 or β2 domain will be deleted. The deletion will be such that it does not interfere with the ability of the α2 or β2 domain to fold into a functional disulfide bonded structure.

A synthetic MHC can comprise MHC peptide complexes in a plane at one end of their meric coiled-coil core. Synthetic MHCs can be held together via linker peptides. Such linker may e.g. be a repeat of glycine residues, interspersed with prolines or serines, for flexibility and solubility, (GGPGG)n or (GGSGG)n with n typically ranging between 1 and 6. It will be appreciated that other linkers, which are flexible, have sufficient solubility and do not form significant secondary structure will also be suitable for this purpose. In general, polypeptide linkers of 1 to 30, preferably 3 to 20 and most preferably 3 to 10 amino acids in length may be used. For non-peptidic linkers their length may be adjusted accordingly. Non-peptidic linkers can also include PEG or poly ethylene oxide (PEO) repeats. In some cases, a linker peptide comprises at least about 1-3, 1-5 1-10, 5-10, 5-15, 5-20, 10-15, 10-20, 15-20, 15-30, or up to about 10-50 residues.

In some cases, a synthetic MHC can further comprise a tag and/or purification domain. The additional domain(s) may e.g. be provided on the multivalent entity or the antigen peptide, but may also be present on the MHC alpha and/or beta chains. A tag can be located at any position of the pentamer. In some cases, the tag is found proximate to the MHC peptide complex. In some cases, the tag is found distal to the MHC peptide complex. The MHC pentamer can comprise at least 1, 2, 3, 4, 5, 6, or up to 8 tags. The tags can be identical. In some cases, the tags are different. Suitable tags comprise: flow cytometry tags (R-PE, APC or biotin Pentamer Fluorotag), Biotin, streptavin, degradation tags. fluorescent tags, biodegradable tags, bioinert tags, and any combination thereof.

In some cases, a synthetic MHC or variant or mimic thereof can be affixed through covalent or non-covalent binding to an aAPP. In an aspect, a synthetic MHC is bound to an aAPP at the multivalent end or distal to the multivalent end. In some cases, a synthetic MHC can be affixed through a covalent binding. For example, a covalent binding can be between biotin molecules that have been added to the synthetic MHC (such as a pentamer), and streptavidin molecules that are coated on a particle, such as a solid support including a bead. An exemplary bead can be a M-280 Epoxy microbead. The Biotinylated synthetic MHCs can be incubated at 4C with the particles, in a PBS buffer, with agitation for any period of time including but not limited to 30 min, 45 min, 1 hour, 2 hours, 3 hours, 5 hours, 8 hours, 12 hours, or up to about 24 hours. This can be sufficient to promote binding of the biotin to the streptavidin, before washing and magnetic purification of the beads from the unbound protein.

### Solid Support

In some cases, a composition provided herein can comprise a solid support. A solid support can be at least partially solid. Non-limiting examples include particles, beads, biodegradable particles, sheets, gels, filters, membranes (e. g. nylon membranes), fibers, capillaries, needles, microtiter strips, tubes, plates or wells, combs, pipette tips, micro arrays, chips, slides, or indeed any at least partially solid surface material. The solid or semi-solid support can be labelled, if this is desired. The support can also have scattering properties or sizes, which enable discrimination among supports of the same nature, e.g. particles of different sizes or scattering properties, color or intensities. Conveniently the support can be made of glass, silica, latex, plastic or any polymeric material. The support can also be made from a biodegradable material. Generally speaking, the nature of the support is not critical and as such variety of materials can be used. In some cases, the solid support can comprise a hydrophobic or hydrophilic phase.

In some embodiments, the solid support is a particle. In some cases, a particle is at least partially magnetic. Several types of magnetic particles could be prepared: ferromagnetic particles, superparamagnetic particles and paramagnetic particles. Methods to prepare superparamagnetic particles are described in U.S. Pat. No. 4,770,183. In an aspect, provided herein is a ferromagnetic particle or a particle that comprises a ferromagnetic phase. A ferromagnetic particle can be susceptible to magnetic fields and may be capable of retaining magnetic properties when the field is removed. Ferromagnetism occurs only when unpaired electrons in the material are contained in a crystalline lattice thus permitting coupling of the unpaired electrons. In another aspect, a particle can be superparamagnetic or comprise a superparamagnetic phase. Superparamagnetic particles can be highly magnetically susceptible, for example they become strongly magnetic when placed in a magnetic field, but, like paramagnetic materials, can rapidly lose their magnetism. Superparamagnetism occurs in ferromagnetic materials when the crystal diameter is decreased to less than a critical value. In an aspect, a particle can be paramagnetic or comprise a paramagnetic phase. A paramagnetic particle can comprise materials that have a diminished magnetic susceptibility and when the field is removed quickly lose their weak magnetism. They are characterized by containing unpaired electrons which are not coupled to each other through an organized matrix. Paramagnetic materials can be ions in solution or gases but can also exist in organized particulate form. Examples of paramagnetic ions include, without limitation, Au (II), Gd (III), Eu (III), Dy (III), Pr (III), Pa (IV), Mn (II), Cr (III), Co (III), Fe (III), Cu (II), Ni (II), Ti (III), and V (IV). Exemplary paramagnetic materials include, without limitation, magnesium, molybdenum, lithium, tantalum, and iron oxide. Paramagnetic beads suitable for magnetic enrichment are commercially 20 available (DYNABEADS™, MACS MICROBEADS™, Miltenyi Biotec). In some embodiments, a particle is an iron dextran bead (e.g., dextran-coated iron-oxide bead). A particle can also be or comprise a diamagnetic material. Diamagnetic refers to materials which do not acquire magnetic properties even in the presence of a magnetic field, i.e., they have no appreciable magnetic susceptibility. In some cases, a particle is an M-280 Epoxy microbead.

Particles may comprise, without limitation, iron, gold, cobalt, nickel, gadolinium, dysprosium, praseodymium, europium, manganese, protactinium, chromium, copper, titanium, or vanadium. Particles can be made, for example, out of metals such as iron, nickel, aluminum, copper, zinc, cadmium, titanium, zirconium, tin, lead, chromium, manganese and cobalt; metal oxides and hydrated oxides such as aluminum oxide, chromium oxide, iron oxide, zinc oxide, and cobalt oxide; metal silicates such as of magnesium, aluminum, zinc, lead, chromium, copper, iron, cobalt, and nickel; alloys such as bronze, brass, stainless steel, and so forth. Particles can also be made of non-metal or organic materials such as cellulose, ceramics, glass, nylon, polystyrene, rubber, plastic, or latex. In some embodiments, particles comprise a combination of a metal and a non-metal or organic compound, for example, methacrylate- or styrene-coated metals and silicate coated metals. The base material can be doped with an agent to alter its physical or chemical properties. For example, rare earth oxides can be included in aluminosilicate glasses to create a paramagnetic glass material with high density (see White & Day, Key Engineering Materials Vol. 94-95, 181-208, 1994). In some embodiments, nanoparticles comprise or consist of biodegradable organic materials, such as cellulose, dextran, and the like. Suitable commercially available particles include, for example, nickel particles (Type 123, VM 63, 18/209A, 10/585A, 347355 and HDNP sold by Novamet Specialty Products, Inc., Wyckoff, N.J.; 08841R sold by Spex, Inc.; 01509BW sold by Aldrich), stainless steel particles (P316L sold by Ametek), zinc dust (Aldrich), palladium particles (D13A17, John Matthey Elec.), and TiO2, SiO₂, or MnO2 particles (Aldrich).

In certain aspects, for example, where magnetic properties are not required, beads can be made of nonmetal or organic (e.g., polymeric) materials such as cellulose, ceramics, glass, nylon, polystyrene, rubber, plastic, or latex.

In some embodiments, a particle that serves as a solid support includes, but is not limited to Quantum Dots (Q-dots). A quantum dot can be a nanocrystal made of semiconductor materials that is small enough to exhibit quantum mechanical properties. Specifically, its excitons are confined in all three spatial dimensions. The electronic properties of these materials are intermediate between those of bulk semiconductors and of discrete molecules. Quantum dots can be engineered to be sensitive to energy in the infrared region, the visible spectrum, and even ultraviolet range through changes in size and composition. Further, they can be designed to be either photoluminescent or photovoltaic, producing either light or energy, respectively.

In some embodiments, quantum dot materials include, but are not limited to, carbon, colloidal gold, germanium, indium arsenide, indium antimonide, gallium arsenide, gallium nitride, cadmium/selenium/telluride, lead, lead oxide, lead sulfide, lead selenide, indium gallium phosphide, silicon, colloidal silver, mercury cadmium telluride, iron, iron oxide, cobalt, graphene, lanthanum, cerium, strontium carbonate, manganese, manganese oxide, nickel oxide, platinum, lithium, lithium titanate, tantalum, copper, palladium, molybdenum, boron carbide, silicon carbide, titanium carbide, tungsten oxide, aluminum, niobium, thulium, aluminum nitride, tin, aluminum oxide, tin oxide, antimony, dysprosium, paseodynium, antinmony oxide, erbium, rhenium, barium, ruthenium, beryllium, samarium, bismuth oxide, boron, gadolinium, boron nitride, vanadium oxide, strontium, ytterbium, zirconium, diamond (C), Silicon (Si), germanium (Ge), silicon carbide (SiC), silicon-germanium (SiGe), aluminium antimonide (AlSb), aluminium arsenide (AlAs), aluminium nitride (AlN), aluminium phosphide (AlP), boron nitride (BN), boron phosphide (BP), boron arsenide (BAs), gallium antimonide (GaSb), gallium arsenide (GaAs), gallium nitride 41 (GaN), gallium phosphide (GaP), indium antimonide (InSb), indium arsenide (InAs), indium nitride (InN), indium phosphide (InP), aluminium gallium arsenide (AlGaAs, AlxGai-xAs), indium gallium arsenide (InGaAs, InxGai.xAs), indium gallium phosphide (InGaP), aluminum indium arsenide (AlInAs), aluminum indium antimonide (AllnSb), gallium arsenide nitride (GaAsN), gallium arsenide phosphide (GaAsP), aluminum gallium nitride (AlGaN), aluminum gallium phosphide (AlGaP), indium gallium nitride (InGaN), indium arsenide antimonide (InAsSb), indium gallium antimonide (InGaSb), aluminum gallium indium phosphide (AlGalnP, also InAlGaP, InGaAlP, AlInGaP), aluminum gallium arsenide phosphide (AlGaAsP), indium gallium arsenide phosphide (InGaAsP), aluminum indium arsenide phosphide (AlInAsP), aluminum gallium arsenide nitride (AlGaAsN), indium gallium arsenide nitride (InGaAsN), indium aluminium arsenide nitride (InAlAsN), gallium arsenide antimonide nitride (GaAsSbN), gallium indium nitride arsenide antimonide (GalnNAsSb), gallium indium arsenide antimonide phosphide (GalnAsSbP), cadmium selenide (CdSe), cadmium sulfide (CdS), cadmium telluride (CdTe), zinc oxide (ZnO), zinc selenide (ZnSc), zinc sulfide (ZnS), zinc telluride (ZnTc), cadmium zinc telluride (CdZnTe, “CZT”), mercury cadmium telluride (HgCdTc), mercury zinc telluride (HgZnTe), mercury zinc selenide (HgZnSe), cuprous chloride (CuCl), lead selenide (PbSe), lead sulfide (PbS), lead telluride (PbTe), tin sulfide (SnS), tin telluride (SnTe), lead tin telluride (PbSnTe), thallium tin telluride (TSnTes), thallium germanium telluride (TlGeTes), bismuth telluride (BiTe), cadmium phosphide (Cd), cadmium arsenide (Cd3As2), cadmium antimonide (Cd3Sb2), zinc phosphide (Zn3P2), zinc arsenide (Zn3As2), zinc antimonide (Zn3Sb2), lead(II) iodide (Pbl2), molybdenum disulfide (MOS2), gallium selenide (GaSe), tin sulfide (SnS), bismuth sulfide (Bi2Ss), copper indium gallium selenide (CIGS), platinum silicide (PtSi), bismuth(III) iodide (BiL), mercury(II) iodide (Hgl2), thallium(I) bromide (TlBr), titanium dioxide: anatase (TiO₂), copper(I) oxide (CU20), copper(II) oxide (CuO), uranium dioxide (UO2), uranium trioxide (UO3), and the like. In some embodiments, suitable materials for quantum dots include organic semiconductors comprising pentacene, anthracene and rubrene. In some embodiments, suitable materials for quantum dots include magnetic semiconductors such as manganese-doped indium arsenide and gallium arsenide, manganese-doped indium antimonide, manganese- and iron-doped indium oxide, 42 manganese doped zinc oxide, and chromium doped aluminum nitride, iron-doped tin dioxide, n-type Cobalt-doped zinc oxide, cobalt-doped titanium dioxide (both rutile and anatase), chromium-doped rutile, Iron-doped rutile and iron-doped anatase, nickel-doped anatase, and manganese-doped tin dioxide.

Quantum dots can be formed using a variety of techniques. For example, quantum dots can be formed by creating a region of a first material having a first band gap surrounded by a second material of a second band gap, wherein the second band gap is larger than the first band gap. Exemplary quantum dots produced by such a process include, but are not limited to, a cadmium selenide (CdSe) core surrounded by a zinc selenide (ZnS) shell. Alternatively, self-assembled quantum dots nucleate spontaneously under certain conditions during molecular beam epitaxy (MBE) and metallorganic vapor phase epitaxy (MOVPE), when a material is grown on a substrate to which it is not lattice matched. The resulting strain between the grown layer and the substrate produces coherently strained islands on top of a two-dimensional “wetting-layer.” The islands can be subsequently surrounded by a shell to form the quantum dot. Individual quantum dots can also be created from two-dimensional electron or hole gases present in remotely doped quantum wells or semiconductor heterostructures. In this case, a surface is coated with a thin layer of photoresist. A lateral pattern is then defined in the resist by electron beam lithography. This pattern can then be transferred to the electron or hole gas by etching, or by depositing metal electrodes (lift-off process) that allow the application of external voltages between the electron gas and the electrodes. Quantum dots can also be formed in quantum well structures due to monolayer fluctuations in the well's thickness. Alternatively, quantum dots can be produced by Ultrasonic Aerosol Pyrolysis (UAP).

In some embodiments, quantum dots include an inner semiconductor core formed of, for example, indium/gallium/phosphide, silicon, gallium arsenide, cadmium telluride, copper indium gallium selenide, indium gallium nitride, carbon, colloidal gold, colloidal silver, or organic materials such as polymer-fullerene heterojunctions (e.g., P3HT+C60), organic nanocrystal solar cells (e.g., cadmium selenide or cadmium 43 telluride), dye sensitized cells (e.g., dye and titanium oxide or nobelium oxide), or a tandem cell (e.g., copper-phthalocyanin+C60); a shell, formed of, for example, zinc selenide or other suitable material; a coating, formed of, for example, PEG lipids or other suitable material; and biofunctional material, formed of, for example, biotin, streptavidin, adhesion proteins, vitamins, organic or inorganic compounds, carbohydrates, aptamers, amino acids, lipids, hyaluronic acid, or other suitable proteins.

Subject particles can comprise a coating. In some cases, a particle is at least partially coated. A particle can comprise a coating that comprises a hydrophilic polymer, an amphiphilic copolymer, an amphiphilic block copolymer, or combinations thereof. In some cases, a polymer may be a homopolymer, copolymer, or block copolymer. In some cases, a polymer can be biocompatible. Examples of biocompatible polymers include, without limitation: polyetherglycols, polyethylene glycol (PEG), methoxy-poly(ethylene glycol), proteins, gelatin, albumin, peptides, DNA, RNA, polysaccharides, dextran, polyvinyl alcohol, polyvinyl pyrrolidone, polyvinyltriazole, N-oxide of polyvinylpyridine, N-(2-hydroxypropyl) methacrylamide (HPMA), polyortho esters, polyglycerols, polyacrylamide, polyoxazolines (e.g., methyl or ethyl poly(2-oxazolines)), polyacroylmorpholine, and copolymers, modified versions thereof, derivatives thereof, or combinations thereof. In an aspect, a coating can comprise a polysaccharide or protein coating. A polysaccharide can be from a starch, glycogen, cellulose, or chitin. Suitable polysaccharides include dextran, dextrin, pullulan, synthetic polysaccharides, vinyl polymers, poly ethylene glycol, poly propylene glycol, derivatized cellulosics, strep-tactin and poly-streptavidin. Polysaccharides may be dextran or different variants of dextrans, such as carboxy methyl dextran, dextran polyaldehyde, and cyclodextrins. An example of a synthetic polysaccharide is e.g. ficoll. Vinyl polymers include, but are not limited to, poly(acrylic acid), poly(acrylamides), poly(acrylic esters), poly(methyl methacrylate), poly(maleic acid), poly(acrylamide), poly(methacrylic acid) and poly(vinylalcohol). Polymeric backbones consisting of derivatized cellulosics include, but are not limited to, derivatized cellulosics including carboxymethyl cellulose, carboxymethyl hydroxyethyl cellulose and hydroxy-ethyl cellulose.

A particle can comprise any number of coatings. In an aspect, a particle comprises from about 1, 2, 3, 4, 5, 6, 7, or 8 coatings. The coatings can be the same or can be different.

In some embodiments, a particle is avidin coated. In some embodiments, a particle is dextran coated. Dextran coated particles are made using any known process. For example, magnetic iron-dextran particles can be prepared by mixing 10 ml of 50% (w/w) aqueous Dextran T-40 (Pharmacia) with an equal volume of an aqueous solution containing 1.51 g FeCl3-6H2O and 0.64 g FeCl2-4H2O. While stirring, the mixture is titrated to pH 10-11 by the drop-wise addition of 7.5% (v/v) NH4OH heated to 60-65° C. in a water bath for 15 minutes. Aggregates are then removed by 3 cycles of centrifugation in a low-speed clinical centrifuge at 600×g for 5 minutes. The iron-dextran particles are separated from unbound dextran by gel filtration chromatography on Sephacryl-300. Five ml of the reaction mixture is then applied to a 2.5×33 cm column and eluted with 0.1 M sodium acetate and 0.15 M NaCl at pH 6.5. The purified ferromagnetic iron-dextran particles collected in the void volume will have a concentration of 7-10 mg/ml as determined by dry weight analysis. Molday and Mackenzie (1982) Journal of Immunological Methods 52:353-367. Also see (Xiangiao (2003) China Particuology Vol. 1, No. 2, 76-79). In an aspect, a particle is an iron-dextran paramagnetic particle. A particle can also be an avidin-coated quantum dot nanocrystal.

The coating can have a thickness (e.g., the average distance from the outside surface of the core magnetic particle to the outside surface of the coating) of from about 1 nm to about 500 nm, e.g., from about 1 nm to about 5 nm, from about 5 nm to about 10 nm, from about 10 nm to about 15 nm, from about 15 nm to about 20 nm, from about 20 nm to about 25 nm, from about 25 nm to about 30 nm, from about 30 nm to about 40 nm, from about 40 nm to about 50 nm, from about 50 nm to about 60 nm, from about 60 nm to about 70 nm, from about 70 nm to about 80 nm, from about 80 nm to about 90 nm, from about 90 nm to about 100 nm, from about 100 nm to about 125 nm, from about 125 nm to about 150 nm, from about 150 nm to about 175 nm, from about 175 nm to about 200 nm, from about 200 nm to about 225 nm, from about 225 nm to about 250 nm, from about 250 nm to about 275 nm, from about 275 nm to about 300 nm. The ratio of the thickness of the coating to the diameter of the magnetic core particle is from about 1:1 to about 1:1000, e.g., from about 1:1 to about 1:1.5, from about 1:1.5 to about 1:2, from about 1:2 to about 1:2.5, from about 1:2.5 to about 1:5, from about 1:5 to about 1:10, from about 1:10 to about 1:25, from about 1:25 to about 1:50, from about 1:50 to about 1:100, from about 1:100 to about 1:250, from about 1:250 to about 1:500, from about 1:500 to about 1:750, or from about 1:750 to about 1:1000.

Molecules can be directly attached to particles by adsorption or by direct chemical bonding, including covalent bonding. See, Hermanson, BIOCONJUGATE TECHNIQUES, Academic Press, New York, 1996, which is hereby incorporated by reference in its entirety. A molecule itself can be directly activated with a variety of chemical functionalities, including nucleophilic groups, leaving groups, or electrophilic groups. Activating functional groups include alkyl and acyl halides, amines, sulfhydryls, aldehydes, unsaturated bonds, hydrazides, isocyanates, isothiocyanates, ketones, and other groups known to activate for chemical bonding. Alternatively, a molecule can be bound to a bead through the use of a small molecule-coupling reagent. Non-limiting examples of coupling reagents include carbodiimides, maleimides, n-hydroxysuccinimide esters, bischloroethylamines, bifunctional aldehydes such as glutaraldehyde, anyhydrides and the like. In other embodiments, a molecule can be coupled to a bead through affinity binding such as a biotin-streptavidin linkage or coupling, as is well known in the art. For example, streptavidin can be bound to a bead by covalent or non-covalent attachment, and a biotinylated molecule can be synthesized using methods that are well known in the art. If covalent binding to a bead is contemplated, the support can be coated with a polymer that contains one or more chemical moieties or functional groups that are available for covalent attachment to a suitable reactant, typically through a linker. For example, amino acid polymers can have groups, such as the ε-amino group of lysine, available to couple a molecule covalently via appropriate linkers. Activation chemistries can be used to allow the specific, stable attachment of molecules to the surface of bead. There are numerous methods that can be used to attach proteins to functional groups. For example, the common cross-linker glutaraldehyde can be used to attach protein amine groups to an aminated bead surface in a two-step process. The resultant linkage is hydrolytically stable. Other methods include use of cross-linkers containing n-hydrosuccinimido (NHS) esters which react with amines on proteins, cross-linkers containing active halogens that react with amine-, sulfhydryl-, or histidine-containing proteins, cross-linkers containing epoxides that react with amines or sulfhydryl groups, conjugation between maleimide groups and sulfhydryl groups, and the formation of protein aldehyde groups by periodate oxidation of pendant sugar moieties followed by reductive amination.

The attachment of specific proteins to a particle surface can be accomplished by direct coupling of the protein or by using indirect methods. Certain proteins will lend themselves to direct attachment or conjugation while other proteins or antibodies retain better functional activity when coupled to a linker or spacer protein such as anti-mouse IgG or streptavidin. If desired, linkers or attachment proteins can be used.

Many different type of small particles (nanoparticles or micron-sized particles) are commercially available from several different manufacturers including: Bangs Laboratories (Fishers, Ind.); Promega (Madison, Wis.); Dynal Inc. (Lake Success, N.Y.); Advanced Magnetics Inc. (Surrey, U.K.); CPG Inc. (Lincoln Park, N.J.); Cortex Biochem (San Leandro, Calif.); European Institute of Science (Lund, Sweden); Ferrofluidics Corp. (Nashua, N.H.); FeRx Inc.; (San Diego, Calif.); Immunicon Corp.; (Huntingdon Valley, Pa.); Magnetically Delivered Therapeutics Inc. (San Diego, Calif.); Miltenyi Biotec GmbH (USA); Microcaps GmbH (Rostock, Germany); PolyMicrospheres Inc. (Indianapolis, Ind.); Scigen Ltd. (Kent, U.K.); Seradyn Inc.; (Indianapolis, Ind.); and Spherotech Inc. (Libertyville, Ill.). Such particles can be made using conventional techniques, such as grinding and milling, emulsion polymerization, block copolymerization, and microemulsion.

The configuration of particles can vary from being irregular in shape to being spherical and/or from having an uneven or irregular surface to having a smooth surface. Particles may be of uniform or variable size. Particle size distribution can be conveniently determined, for example, using dynamic light scattering. A subject particle can be of any size. In an aspect, a particle is micron-sized. In another aspect, a particle is nano-sized. In an aspect, a particle is from about 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm in diameter. In some embodiments, nanoparticles have a mean particle diameter of 25-30 nm, 2535 nm, 25-40 nm, 25-45 nm, 25-50 nm, 25-55 nm, 25-60 nm, 25-70 nm, 25-75 nm, 25-80 nm, 25-90 nm, 25-95 nm, 25-100 nm, 25-125 nm, 25-150 nm, 25-200 nm, 25300 nm, 25-400 nm, 30-35 nm, 35-40 nm, 35-45 nm, 35-50 nm, 35-55 nm, 35-60 nm, 35-70 nm, 35-75 nm, 35-80 nm, 35-90 nm, 35-95 nm, 35-100 nm, 35-125 nm, 35-150 nm, 35-200 nm, 35-300 nm, 35-400, 35-500 nm, 40-45 nm, 35-50 nm, 45-55 nm, 4560 nm, 45-70 nm, 45-75 nm, 45-80 nm, 45-90 nm, 45-95 nm, 45-100 nm, 45-125 nm, 45-150 nm, 45-200 nm, 45-300 nm, 45-400, 45-500 nm, 50-55 nm, 50-60 nm, 50-70 nm, 50-75 nm, 50-80 nm, 50-90 nm, 50-95 nm, 50-100 nm, 50-125 nm, 50-150 nm, 50-200 nm, 50-300 nm, 50-400, 50-500 nm, 55-60 nm, 55-70 nm, 55-75 nm, 55-80 31 nm, 55-90 nm, 55-95 nm, 55-100 nm, 55-125 nm, 55-150 nm, 55-200 nm, 55-300 nm, 55-400, 55-500 nm, 60-70 nm, 60-75 nm, 60-80 nm, 60-90 nm, 60-95 nm, 60-100 nm, 60-125 nm, 60-150 nm, 60-200 nm, 60-300 nm, 60-400, 60-500 nm, 65-70 nm, 65-75 nm, 65-80 nm, 65-90 nm, 65-95 nm, 65-100 nm, 65-125 nm, 65-150 nm, 65-200 nm, 65-300 nm, 65-400, 65-500 nm, 70-75 nm, 70-80 nm, 70-90 nm, 70-95 nm, 70-100 nm, 70-125 nm, 70-150 nm, 70-200 nm, 70-300 nm, 70-400, 70-500 nm, 75-80 nm, 75-90 nm, 75-95 nm, 75-100 nm, 75-125 nm, 75-150 nm, 75-200 nm, 75-300 nm, 75400, 75-500 nm, 80-90 nm, 80-95 nm, 80-100 nm, 80-125 nm, 80-150 nm, 80-200 nm, 80-300 nm, 80-400, 80-500 nm, 85-90 nm, 85-95 nm, 85-100 nm, 85-125 nm, 85150 nm, 85-200 nm, 85-300 nm, 85-400, 85-500 nm, 90-95 nm, 90-100 nm, 90-125 nm, 90-150 nm, 90-200 nm, 90-300 nm, 90-400, 90-500 nm, 100-125 nm, 100-150 nm, 100-200 nm, 100-300 nm, 100-400, 100-500 nm, 125-150 nm, 125-200 nm, 125300 nm, 125-400, 125-500 nm, 150-200 nm, 150-300 nm, 150-400, 150-500 nm, 175-200 nm, 175-300 nm, 175-400, 175-500 nm, 200-300 nm, 200-400, 200-500 nm, 300-400, 300-500 nm, or 400-500 nm.

The density of particles can be selected such that the particles will differentially settle through a sample suspension more rapidly than cells. Thus, particles preferably are composed of a high-density material to facilitate cell separation and manipulation of the particles. Use of such particles permits the particles to settle under gravity to facilitate their separation from antigen-specific T cells, T cell precursors, B cell precursors, B cells, or other cells.

A particle can comprise proteins or portions thereof associated to or attached thereto. In some cases, a protein or fragment thereof that is associated to or attached to a particle is associated with T cell activation. T-cell activation involves interactions involving both T-cell receptor (TCR) signaling and costimulation, such as CD28 costimulation. A TCR interacts with foreign antigen in the context of self-MHC, which provides “signal 1.” However, signal 1 by itself may be insufficient to enable T-cell activation. “Signal 2” is provided when a co-stimulatory receptor, such as CD28, which is constitutively expressed on T cells, binds to B7-1 (CD80) and B7-2 (CD86) molecules that are expressed on antigen-presenting cells (APCs). An APC is a cell that displays an antigen peptide complexed with major histocompatibility complexes (MHCs) or portion thereof on their surfaces; this process is known as antigen presentation. T cells may recognize these complexes using their T cell receptors (TCRs). T cells utilize fragments of antigens to be phagocytosed by APCs such as dendritic cells, with eventual antigen processing and presentation by the APCs. T cells then interact with the APCs to receive both signals 1 and 2 for appropriate T-cell activation, which leads to cytokine production and proliferation as well as active killing of a pathogen, such as a virus. Particles provided herein can comprise any number of proteins or fragments thereof associated with T cell activation.

In some cases, a protein or fragment thereof provides a stimulatory signal to a T cell, such as a first signal. A first signal can be a cognate antigenic peptide presented in the context of major histocompatibility complex (MHC) that binds the T cell receptor (TCR). In an aspect, a protein or fragment thereof provides a second signal. A second signal can be provided by a co-stimulatory receptor that modulates or is capable of modulating a T cell response. In some cases, a subject particle comprises a signal 1 that is delivered by a chimeric MHC-immunoglobulin dimer (MHC-Ig) loaded with a specific peptide or fragment thereof, and signal 2 is either B7.1 (the natural ligand for the T cell receptor CD28) or an activating antibody against CD28. Both proteins or fragments thereof can be chemically coupled to the surface of a subject particle to create artificial antigen presenting platforms (aAPP). An aAPP can be a surrogate for an endogenous APC. aAPPs can display antigen complexed with major histocompatibility complexes (MHCs) or a portion thereof on their surfaces to present a target peptide antigen to T cells so that they may recognize these complexes using their T cell receptors (TCRs).

The ratio of particular proteins on a particle can be varied to increase the effectiveness of the particle in peptide presentation. For example, optimal ratios of peptide (Signal 1) to anti-CD28 or anti-CD3 (Signal 2) can be modulated. Particles are coupled with peptide and anti-CD28 or anti-CD3 at a variety of ratios, such as 200:1, 100:1, 50:1, 40:1, 30:1, 10:1, 3:1, 1:1, 0.3:1; 0.1:1, 0.03:1, 1:5, 1:10, 1:20, 1:30, 1:40, 1:50, 1:75, 1:100, or 1:200. The total amount of protein coupled to the supports is kept constant (for example, at 150 mg/ml of particles) or can be varied. Because effector functions such as cytokine release and growth may have differing requirements for Signal 1 versus Signal 2 than T cell activation and differentiation, these functions can be assayed separately.

T cells: particle ratios can also be modulated in compositions and methods provided herein. Suitable T cell: particle ratios comprise: 100:1, 50:1, 40:1, 30:1, 20:1, 10:1, 5:1, 3:1, 1:1, 1:5, 1:10, 1:20, 1:30, 1:40, 1:50, or 1:100.

Use of subject particles for stimulating immune cells, such as T cells can generate increased populations of effector T cells, memory T cells, and both effector T cells and memory T cells. Particles can be used to present peptide in the context of MHC I, MHC II, or both in order to stimulate CD8, CD4, or both CD8 and CD4 T cells. A combinatorial approach can yield a greater response to an unwanted source expressing a peptide antigen such as a virus, other pathogen, or cancer cell. For example, after contact with a particle T cells can differentiate into memory T cells and effector T cells. Memory T cells are antigen peptide-specific T cells that remain long-term after an infection has been eliminated. Memory T cells can be quickly converted into large numbers of effector T cells upon re-exposure to the specific source expressing an invading antigen peptide thereby providing a rapid response to past infection. Memory T cells are either CD4+ or CD8+ depending on the type of antigen encountered. The memory T cells include subtypes such as, central memory T cells (Tcm cells), effector memory T cells (Tem cells and Temra cells). They also typically express CD45RO cell surface protein. Any one of these subtypes of T cells can be expanded by way of contacting a T cells with a subject peptide expressed on a particle.

In some cases, a CD4 memory T cell expansion, resulting from contact with a particle, can lead to a “vaccine booster” shot effect in enhancing active immunity in subjects recovering from COVID19 or any viral illness. For example, this vaccine booster can be superior to a passive immunity antibody-mediated convalescent serum strategy.

### Peptides

Provided herein are, inter alia, compositions and methods utilizing a peptide or fragment thereof. In some cases, the peptide is antigenic. In other cases, the peptide is not antigenic. Peptides provided herein may essentially come from any source. A source may include, but is not limited to, a human, a virus, a bacterium, a parasite, a plant, a fungus, or a tumor. In some cases, a peptide or fragment thereof is a viral, bacterial, or a parasitic peptide.

### Viral Peptides

In some cases, the source is a virus. In some embodiments, the virus is a DNA virus (i.e. the genome of the virus is DNA). Exemplary DNA viruses include, but are not limited to: Adenoviruses, Herpesvirus, Poxvirus, Papovaviruses, Hepadnaviruses, or any combination thereof. In another aspect, a peptide source is a DNA virus and is any one of: Human adenoviruses, Herpes simplex, Varicella zoster, Epstein-Barr virus, cytomegalovirus, Kaposi's sarcoma, Vaccinia virus, Human parvovirus, Papilloma virus, Hepatitis B virus, or any combination thereof. In some cases, the virus is an RNA virus, Exemplary RNA viruses, include, but are not limited to Orthomyxoviruses, Paramyxoviruses, Coronavirus, Picomaviruses, Reoviruses, Togaviruses, Flaviviruses, Arenaviruses, Phabdoviruses, Retroviruses, or any combination thereof. Exemplary RNA viruses can also include: Infuenza virus, Mumps, measles, respiratory syncytial virus, Common cold viruses, Polio, coxsackie, hepatitis A, rhinovirus, Rotavirus, reovirus, Rubella, arthropod-bome encephaltis, Arthopod-bome viruses (yellow fever, dengue fever), Lymphocytic choriomeningitis, Lassa fever, Rabies, Human T-cell leukemia virus, HIV, or any combination thereof.

In some cases a viral peptide is from a virus that causes a respiratory infection such as influenza, parainfluenza, MERS, SARS, coronavirus, rhinovirus or respiratory syncytial virus. The infection may be a pandemic infection, such as pandemic influenza, MERS, SARS, SARS-COV-2, or other similar infections. In some cases, a viral peptide is from a coronavirus and is SARS-CoV-2, SARS-CoV, and/or MERS-CoV. In some cases, a viral peptide can be from a variant of SARS-CoV-2. In some cases, a variant of SARS-CoV-2 can comprise B.1.1.7 (or the U.K. variant), B.1.1.207, Cluster 5, B.1.351 (or RSA variant), P.1 (or Brazil variant), B.1.617 (or India variant), B.1525, NS3, WIV04/2019, or CAL.20C. In some embodiments, the B.1.617 variant includes a mutation in a spike protein comprising at least one of E154K, E484Q, L452R, P681R, Q1071H, or any combination thereof. In some cases, a variant of SARS-CoV-2 can comprise lineage A.1, A.2, A.3, A.4, A.5, A.6, B.1, B.2, B.3, B.4, B.5, B.6, B.7, B.8, B.9, B.10, B.11, B.12, B.13, B.14, B.15, or B.16.

In some cases, an influenza antigen peptide may be utilized. In influenza viral antigen may be human or non-human. In some cases, an influenza viral antigen that is used originates from type A, B, C, and/or D. In some cases, an influenza viral antigen is A(H1N1), A(H3N2), B(Victoria), or B(Yamagata). In some cases, an influenza viral antigen can be from a non-human species, such as swine, bird, bat, bovine, canine, horse, poultry, feline, monkey and the like.

In some embodiments, a viral peptide can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (EBOV) (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus (CMV)), chikungunya, Hantavirus, Hypoviridae, Iridoviridae, Leviviridae, Lipothrixviridae, Microviridae, Orthomyxoviridae (e.g., Influenza virus A, such as H1N1 strain, and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue DINS1, Dengue D1NS2, and Dengue D1NS3. Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, JC virus, west nile virus, cytomeglavirus, Epstein-Barr, rotavirus, rhinovirus, adenovirus, coxsackieviruses, equine encephalitis, BK virus, malaria, MuLV, VSV, HTLV, Japanese encephalitis, yellow fever, Rift Valley fever, and lymphocytic choriomeningitis.

In some embodiments, a viral peptide can be derived from one or more viruses from the Orthomyxovirus family, for example, the Influenza virus A, Influenza virus B, Influenza virus C, Isavirus, Thogotovirus and Quaranjavirus. Exemplary influenza A virus subtypes include H1N1, H1N2, H3N2, H3N1, H5N1, H2N2, and H7N7. Exemplary influenza virus antigens include one or more proteins or glycoproteins such as hemagglutinin, such as HA1 and HA2 subunits, neuraminidase, viral RNA polymerase, such as one or more of PB1, PB2 PA and PB1-F2, reverse transcriptase, capsid protein, non-structured proteins, such as NS 1 and NEP, nucleoprotein, matrix proteins, such as M1 and M2 and pore proteins. In some embodiments, Influenza A virus antigens include one or more of the Hemagglutinin (HA) or Neuraminidase (NA) glycoproteins or fragments of the HA or NA, including the antigenic sites of the Hemagglutinin HA1 glycoprotein. In an exemplary embodiment, MDNPs include RNA encoding the influenza A/WSN/33 HA protein.

In some embodiments, the viral antigen in derived from one or more viruses from the genus Ebolavirus, for example, the Zaire ebolavirus (EBOV), Sudan ebolavirus (SUDV), Tai Forest ebolavirus (TAFV), Reston ebolavirus (RESTV), and Bundibugyo ebolavirus (BDBV). In an exemplary embodiment, MDNPs include RNA, such as repRNA, encoding the Zaire ebolavirus glycoprotein (GP), or one or more fragments of the Zaire ebolavirus glycoprotein (GP).

In some embodiments, the viral antigen in derived from one or more viruses from the genus Flavivirus, for example, the Zika virus (ZIKV).

In some cases, a peptide or fragment thereof can have from about 50%, 60%, 70%, 75%, 80%, 85%, 88%, 90%, 93%, 95%, 96%, 97%, 98%, 99%, or 100% identity to SEQ ID NO: 15 from Table 1.

TABLE 1
Exemplary viral peptides and corresponding MHC alleles

| SEQ | | | | | | MHC |
|---|---|---|---|---|---|---|
| ID | | | IEDB | | | Allele |
| No | | Protein | ID | Peptide | MHC Allele | Class |
| 1 | HIV1 | Gag | 59613 | SLYNTVATL | HLA-A*02:01/HLA- | I |
| | | | | | A*02:02/HLA- | |
| | | | | | A*02:03/HLA- | |
| | | | | | A*02:06/HLA- | |
| | | | | | A*02:11/HLA- | |
| | | | | | A*02:19/HLA-A2/HLA- | |
| | | | | | B*15:01/HLA- | |
| | | | | | A*02:05/HLA- | |
| | | | | | A*02:14/HLA- | |
| | | | | | A*68:02/HLA- | |
| | | | | | A*69:01/HLA-B*07:02 | |
| 2 | HIV1 | Gag | 33250 | KRWIILGLNK | HLA-B*27:05/HLA- | I |
| | | | | | B*27:03/HLA- | |
| | | | | | A*03:01/HLA- | |
| | | | | | B*27:02/HLA- | |
| | | | | | DRB1*01:01/Mamu- | |
| | | | | | B*017:04/HLA- | |
| | | | | | DRB1*01:03 | |
| 3 | HIV1 | Gag | 21635 | GPGHKARVL | HLA-B*07:02/H2- | I |
| | | | | | Dd/HLA-A*01:01/HLA- | |
| | | | | | A*02:01/HLA- | |
| | | | | | A*03:01/HLA- | |
| | | | | | A*11:01/HLA- | |
| | | | | | A*31:01/HLA- | |
| | | | | | A*69:01/HLA- | |
| | | | | | B*15:01/HLA- | |
| | | | | | B*27:05/HLA- | |
| | | | | | B*40:01/HLA-B*58:01 | |
| 4 | HIV1 | Gag | 29804 | KAFSPEVIPMF | HLA-B*57:01/HLA- | I |
| | | | | | B*57:03 | |
| 5 | HIV1 | Gag | 69360 | VLAEAMSQV | HLA-A*02:01/HLA- | I |
| | | | | | A*02:02/HLA- | |
| | | | | | A*02:03/HLA- | |
| | | | | | A*02:06/HLA- | |
| | | | | | A*68:02/HLA- | |
| | | | | | A*02:11/HLA- | |
| | | | | | A*02:19/HLA-A*69:01 | |
| 6 | HIV1 | Gag | 131070 | SLFNTVATL | HLA-A*02:01 | I |
| 7 | HIV1 | Nef | 56620 | RYPLTFGWCF | HLA-A*24:02 | I |
| 8 | HIV1 | Nef | 5295 | AVDLSHFLK | HLA-A*11:01/HLA- | I |
| | | | | | A*03:01/HLA- | |
| | | | | | A*01:01/HLA- | |
| | | | | | A*02:01/HLA-A*24:02 | |
| 9 | HIV1 | Nef | 52760 | QVPLRPMTYK | HLA-A*03:01/HLA- | I |
| | | | | | A*11:01/HLA- | |
| | | | | | A*02:01/HLA- | |
| | | | | | A*31:01/HLA- | |
| | | | | | A*33:01/HLA- | |
| | | | | | A*68:01/HLA- | |
| | | | | | A*01:01/HLA- | |
| | | | | | A*02:02/HLA- | |
| | | | | | A*02:05/HLA-A*24:02 | |
| 10 | HIV1 | Nef | 102046 | RYPLTFGW | HLA-A*24:02 | I |
| 11 | HIV1 | Nef | 193060 | RFPLTFGWCF | HLA-A*24:02 | I |
| 12 | HIV1 | Envelope | 53935 | RGPGRAFVTI | H2-Dd/H2-Db/H2- | I |
| | | glycoprotein | | | Ld/H2-Kb/H2-Kd/HLA- | |
| | | gp160 | | | A*02:01/HLA- | |
| | | | | | A*02:01/HLA-A2/HLA- | |
| | | | | | A*02:02/HLA- | |
| | | | | | A*02:03/HLA- | |
| | | | | | A*02:06/HLA-A*68:02 | |
| 13 | HIV1 | Env gp160 | 32201 | KLTPLCVTL | HLA-A*02:01/HLA- | I |
| | | | | | A*02:02/HLA- | |
| | | | | | A*02:03/HLA- | |
| | | | | | A*02:06/HLA- | |
| | | | | | A*02:11/HLA- | |
| | | | | | A*02:19/HLA- | |
| | | | | | A*68:02/HLA-A*69:01 | |
| 14 | HIV1 | Env gp160 | 54226 | RIQRGPGRAFV | HLA- | II |
| | | | | TIGK | DQA1*03:01/DQB1*03: | |
| | | | | | 02 | |
| 15 | HIV1 | Env gp160 | 53114 | RAIEAQQHL | HLA-C*12:02/HLA- | I |
| | | | | | B*40:01/HLA- | |
| | | | | | A*31:01/HLA- | |
| | | | | | A*69:01/HLA- | |
| | | | | | B*07:02/HLA- | |
| | | | | | B*15:01/HLA- | |
| | | | | | B*58:01/HLA- | |
| | | | | | C*03:01/HLA- | |
| | | | | | A*01:01/HLA- | |
| | | | | | A*02:01/HLA- | |
| | | | | | A*03:01/HLA- | |
| | | | | | A*11:01/HLA-B*27:05 | |
| 16 | HIV1 | Env gp160 | 54226 | RIQRGPGRAFV | HLA-DQA1*03:01/ | II |
| | | | | TIGK | DQB1*03:02 | |
| 17 | HIV1 | Env gp160 | 67245 | TVYYGVPVWK | HLA-A*11:01/HLA- | I |
| | | | | | A*03:01/HLA- | |
| | | | | | A*31:01/HLA- | |
| | | | | | A*68:01/HLA-A*33:01 | |
| 18 | HIV1 | Env gp160 | 54730 | RLRDLLLIVTR | HLA-A*11:01/HLA- | I |
| | | | | | A*03:01/HLA- | |
| | | | | | A*01:01/HLA- | |
| | | | | | A*02:01/HLA- | |
| | | | | | A*24:02/HLA- | |
| | | | | | A*33:03/HLA-A*07:02 | |
| 19 | Zaire | Nucleoprotein | 16888 | FLSFASLFL | HLA-A*02:01/HLA- | I |
| | ebola | | | | A*24:02/HLA- | |
| | virus | | | | B*15:01/HLA- | |
| | | | | | C*03:03/HLA- | |
| | | | | | A*03:01/HLA- | |
| | | | | | A*25:01/HLA- | |
| | | | | | A*26:01/HLA- | |
| | | | | | A*80:01/HLA- | |
| | | | | | A*18:01/HLA- | |
| | | | | | B*18:01/HLA- | |
| | | | | | B*27:03/HLA- | |
| | | | | | B*46:01/HLA-B*57:01 | |
| 20 | Zaire | Nucleoprotein | 17527 | FQQTNAMVT | HLA-A*02:01/HLA- | I |
| | ebola | | | | B*15:01 | |
| | virus | | | | | |
| 21 | Zaire | Nucleoprotein | 32188 | KLTEAITAA | HLA-A*02:02/HLA- | I |
| | ebola | | | | B*15:01 | |
| | virus | | | | | |
| 22 | Zaire | Nucleoprotein | 54673 | RLMRTNFLI | HLA-A*02:01/HLA- | I |
| | ebola | | | | A*24:02/HLA- | |
| | virus | | | | A*03:01/HLA- | |
| | | | | | A*11:01/HLA- | |
| | | | | | A*08:01/HLA- | |
| | | | | | B*15:01/HLA- | |
| | | | | | B*07:02/HLA- | |
| | | | | | A*25:01/HLA- | |
| | | | | | A*26:01/HLA- | |
| | | | | | B*18:01/HLA-B*46:01 | |
| 23 | Zaire | Nucleoprotein | 75566 | YQNNLEEI | HLA-A*24:02/HLA- | I |
| | ebola | | | | A*02:01/HLA-B*15:01 | |
| | virus | | | | | |
| 24 | Zaire | Envelope | 91144 | ATDVPSATK | HLA-A*11:01/HLA- | I |
| | ebola | glycoprotein | | | A*01:01/HLA- | |
| | virus | | | | A*03:01/HLA-A*24:02 | |
| 25 | Zaire | Envelope | 54480 | RLASTVIYR | HLA-A*03:01/HLA- | I |
| | ebola | glycoprotein | | | A*11:01/HLA- | |
| | virus | | | | A*02:01/HLA- | |
| | | | | | A*31:01/HLA- | |
| | | | | | A*02:03/HLA- | |
| | | | | | A*02:12/HLA- | |
| | | | | | A*02:19/HLA- | |
| | | | | | A*23:01/HLA- | |
| | | | | | A*24:03/HLA- | |
| | | | | | A*25:01/HLA- | |
| | | | | | A*26:01/HLA- | |
| | | | | | A*68:02/HLA- | |
| | | | | | A*69:01/HLA- | |
| | | | | | A*80:01/HLA- | |
| | | | | | B*15:01/HLA- | |
| | | | | | B*15:17/HLA- | |
| | | | | | B*18:01/HLA- | |
| | | | | | B*27:03/HLA- | |
| | | | | | B*39:01/HLA- | |
| | | | | | B*46:01/HLA- | |
| | | | | | B*51:01/HLA-B*57:01 | |
| 26 | Zaire | Envelope | 66646 | TTIGEWAFW | HLA-A*24:02/HLA- | I |
| | ebola | glycoprotein | | | A*32:07/HLA- | |
| | virus | | | | A*32:15/HLA- | |
| | | | | | A*68:23/HLA- | |
| | | | | | B*15:42/HLA- | |
| | | | | | B*45:06/HLA- | |
| | | | | | B*58:01/HLA- | |
| | | | | | B*83:01/HLA- | |
| | | | | | C*04:01/HLA- | |
| | | | | | A*02:01/HLA- | |
| | | | | | A*02:03/HLA- | |
| | | | | | A*02:11/HLA- | |
| | | | | | A*02:12/HLA- | |
| | | | | | A*02:16/HLA- | |
| | | | | | A*02:19/HLA- | |
| | | | | | A*03:01/HLA- | |
| | | | | | A*26:01/HLA- | |
| | | | | | A*68:02/HLA- | |
| | | | | | A*69:01/HLA- | |
| | | | | | A*80:01/HLA- | |
| | | | | | B*15:01/HLA- | |
| | | | | | B*18:01/HLA- | |
| | | | | | B*27:03/HLA- | |
| | | | | | B*39:01/HLA-B*46:01 | |
| 27 | Zaire | Envelope | 91362 | GFRSGVPPK | HLA-A*03:01/HLA- | I |
| | ebola | glycoprotein | | | A*11:01/HLA-B*15:01 | |
| | virus | | | | | |
| 28 | Zaire | Envelope | 91766 | NQDGLICGL | HLA-A*02:01 | I |
| | ebola | glycoprotein | | | | |
| | virus | | | | | |
| 29 | Zika | Genome | 569587 | IGVSNRDFV | H2-Db/H2-Kb | I |
| | Virus | polyprotein | | | | |
| 30 | Zika | Genome | 741567 | IRCIGVSNRDF | HLA-DRB1*01:01/HLA- | II |
| | Virus | polyprotein | | VEGMSGGTW | DTB1*03:01//HLA- | |
| | | | | | DTB1*04:01/HLA- | |
| | | | | | DTB1*07:01/HLA- | |
| | | | | | DTB1*15:01/HLA- | |
| | | | | | DTB5*01:01/HLA- | |
| | | | | | DTB1*11:O1 | |
| 31 | Zika | Genome | 741871 | QPENLEYRIML | HLA-DRB5*01:01/HLA- | II |
| | Virus | polyprotein | | SVHGSQHSG | DRB1*03:01/HLA- | |
| | | | | | DRB1*04:01/HLA- | |
| | | | | | DRB1*07:01/HLA- | |
| | | | | | DRB1*11:01/HLA- | |
| | | | | | DRB1*15:01/HLA- | |
| | | | | | DRB5*01:01 | |
| 32 | Zika | Genome | 741599 | KGVSYSLCTA | HLA-DRB1*01:01/HLA- | II |
| | Virus | polyprotein | | AFTFTKIPAE | DRB1*04:01/HLA- | |
| | | | | | DRB1*07:01/HLA- | |
| | | | | | DRB1*11:01/HLA- | |
| | | | | | DRB1*15:01/HLA- | |
| | | | | | DRB5*01:01/HLA- | |
| | | | | | DRB1*03:01 | |
| 33 | Zika | Genome | 741402 | FEATVRGAKR | HLA-DRB1*01:01/HLA- | II |
| | Virus | polyprotein | | MAVLGDTAW | DRB1*03:01/HLA- | |
| | | | | D | DRB1*07:01/HLA- | |
| | | | | | DRB1*11:01/HLA- | |
| | | | | | DRB1*15:01/HLA- | |
| | | | | | DRB5*01:01//HLA- | |
| | | | | | DRB1*04:01 | |
| 34 | Zika | Genome | 741533 | HRSGSTIGKAF | HLA-DRB1*01:01/HLA- | II |
| | Virus | polyprotein | | EATVRGAKR | DRB1*04:01/HLA- | |
| | | | | | DRB1*11:01/HLA- | |
| | | | | | DRB1*15:01/HLA- | |
| | | | | | DRB5*01:01/HLA- | |
| | | | | | DRB1*03:01/HLA- | |
| | | | | | DRB1*07:01 | |
| 35 | Influenza | Hemagglutinin | 125913 | ELLVLLENERT | HLA-DRB1*04:01 | II |
| | A | | | LDYHDS | | |
| | (H1N1, | | | | | |
| | 2009) | | | | | |
| 36 | Influenza | Hemagglutinin | 125913 | ELLVLLENERT | HLA-DRB1*04:01 | II |
| | A | | | LDYHDS | | |
| 37 | Influenza | Matrix protein | 67496 | TYVLSIIPSGPL | HLA-DRB1*04:01 | II |
| | A | 1 | | KAEIAQRL | | |
| 38 | Influenza | Matrix protein | 124495 | LYKKLKREITF | HLA-A*24:02 | I |
| | A | 1 | | | | |
| 39 | Influenza | Neuraminidase | 126100 | GFEMIWDPNG | HLA-DRB1*04:01 | II |
| | A | | | WTGTDN | | |
| 40 | Influenza | Neuraminidase | 126167 | GQASYKIFRIE | HLA-DRB1*04:01 | II |
| | A | | | KGKIVK | | |
| 41 | Influenza | Neuraminidase | 126199 | GWAIYSKDNS | HLA-DRB1*04:01 | II |
| | A | | | VRIGSKG | | |

### SAPS-CoV-2

In some embodiments, the peptide or fragment thereof is from a virus (a i.e. a viral peptide). A virus can be riboviria. A virus can be nidovirales. A virus can be comnidovirineae. A virus can be coronaviridae. A virus can be orthocoronavirinae. A virus can be betacoronavirus. In some cases, a viral peptide is derived from a viral protein of a virus of genus Alphacoronavirus, Betacoronavirus, Gammacoronavirus, and Deltacoronavirus. A virus can be sarbecovirus. A virus can be severe acute respiratory syndrome-related coronavirus. A virus can be severe acute respiratory syndrome coronavirus 2 (SARS-Cov-2) (also referred to herein as COVID-19).

In some cases, a peptide or fragment thereof is from a SARS-CoV-2 virus. SARS-Cov-2 is an enveloped positive strand single strand RNA virus. In some cases, a peptide or fragment thereof is encoded by a gene from SARS-Cov-2. SARS-Cov-2 may have any number of genes, for example (5′ to 3′): replicase ORF1ab, spike (S), envelope (E), membrane (M) and nucleocapsid (N). Any one of the S, E, M, N, or combination thereof may be utilized as a subject peptide or fragment thereof in compositions and methods provided herein. In some cases, a peptide or fragment thereof can have from about 50%, 60%, 70%, 75%, 80%, 85%, 88%, 90%, 93%, 95%, 96%, 97%, 98%, 99%, or 100% identity to any one of SEQ ID NO: 42-SEQ ID NO: 55 from Table 2. In some cases, a peptide or fragment thereof comprises from about 7-17, 7-15, 7-10, 8-17, 8-15, 8-10, 9-17, 9-15, or 9-10 continuous amino acids from a sequence in Table 2. In some cases, a peptide or fragment thereof comprises from about 7-17, 7-15, 7-10, 8-17, 8-15, 8-10, 9-17, 9-15, or 9-10 continuous amino acids of SEQ ID NO: 44.

TABLE 2
Severe acute respiratory syndrome-related
coronavirus 2 protein genome. Accession: NC_004718.3
Depicted is the positive strand of the SARS-Cov-2 virus.

| SEQ | | | | | | | | |
|---|---|---|---|---|---|---|---|---|
| ID | | | Gene | | Locus | Protein | | Protein |
| NO | Start | Stop | ID | Locus | tag | product | Length | Name |
| 42 | 265 | 21485 | 1489680 | orf1ab | sars1 | NP_ | 7073 | orf1ab |
| | | | | | | 828849.2 | | polyprotein |
| | | | | | | | | (pp1ab) |

MESLVLGVNEKTHVQLSLPVLQVRDVLVRGFGDSVEEALSEAREFILKNG
TCGLVELEKGVLPQLEQPYVFIKRSDALSTNHGHKVVELVAEMDGIQYGR
SGITLGVLVPHVGETPIAYRNVLLRKNGNKGAGGHSYGIDLKSYDLGDEL
GTDPIEDYEQNWNTKHGSGALRELTRELKGGAVTRYVDNNFCGPDGYPLD
CIKDFLARAGKSMCTLSEQLDYIESKRGVYCCRDHEHEIAWFTERSDKSY
EHQTPFEIKSAKKFDTFKGECPKFVFPLNSKVKVIQPRVEKKKTEGFMGR
IRSVYPVASPQECNNMHLSTLMKCNHCDEVSWQTCDFLKATCEHCGTENL
VIEGPTTCGYLPTNAVVKMPCPACQDPETGPEHSVADYHNHSNIETRLRK
GGRTRCFGGCVFAYVGCYNKRAYVVVPRASADIGSGHTGITGDNVETLNE
DLLEILSRERVNINIVGDFHLNEEVAIILASFSASTSAFIDTIKSLDYKS
FKTIVESCGNYKVTKGKPVKGAWNIGQQRSVLTPLCGFPSQAAGVIRSIF
ARTLDAANHSIPDLQRAAVTILDGISEQSLRLVDAMVYTSDLLTNSVIIM
AYVTGGLVQQTSQWLSNLLGTTVEKLRPIFEWIEAKLSAGVEFLKDAWEI
LKFLITGVFDIVKGQIQVASDNIKDCVKCFIDVVNKALEMCIDQVTIAGA
KLRSLNLGEVFIAQSKGLYRQCIRGKEQLQLLMPLKAPKEVTFLEGDSHD
TVLTSEEVVLKNGELEALETPVDSFTNGAIVGTPVCVNGLMLLEIKDKEQ
YCALSPGLLATNNVFRLKGGAPIKGVTFGEDTVWEVQGYKNVRITFELDE
RVDKVLNEKCSVYTVESGTEVTEFACVVAEAVVKTLQPVSDLLTNMGIDL
DEWSVATFYLFDDAGEENFSSRMYCSFYPPDEEEEDDAECEEEEIDETCE
HEYGTEDDYQGLPLEFGASAETVRVEEEEEEDWLDDTTEQSEIEPEPEPT
PEEPVNQFTGYLKLTDNVAIKCVDIVKEAQSANPMVIVNAANIHLKHGGG
VAGALNKATNGAMQKESDDYIKLNGPLTVGGSCLLSGHNLAKKCLHVVGP
NLNAGEDIQLLKAAYENFNSQDILLAPLLSAGIFGAKPLQSLQVCVQTVR
TQVYIAVNDKALYEQVVMDYLDNLKPRVEAPKQEEPPNTEDSKTEEKSVV
QKPVDVKPKIKACIDEVTTTLEETKFLTNKLLLFADINGKLYHDSQNMLR
GEDMSFLEKDAPYMVGDVITSGDITCVVIPSKKAGGTTEMLSRALKKVPV
DEYITTYPGQGCAGYTLEEAKTALKKCKSAFYVLPSEAPNAKEEILGTVS
WNLREMLAHAEETRKLMPICMDVRAIMATIQRKYKGIKIQEGIVDYGVRF
FFYTSKEPVASIITKLNSLNEPLVTMPIGYVTHGFNLEEAARCMRSLKAP
AVVSVSSPDAVTTYNGYLTSSSKTSEEHFVETVSLAGSYRDWSYSGQRTE
LGVEFLKRGDKIVYHTLESPVEFHLDGEVLSLDKLKSLLSLREVKTIKVF
TTVDNTNLHTQLVDMSMTYGQQFGPTYLDGADVTKIKPHVNHEGKTFFVL
PSDDTLRSEAFEYYHTLDESFLGRYMSALNHTKKWKFPQVGGLTSIKWAD
NNCYLSSVLLALQQLEVKFNAPALQEAYYRARAGDAANFCALILAYSNKT
VGELGDVRETMTHLLQHANLESAKRVLNVVCKHCGQKTTTLTGVEAVMYM
GTLSYDNLKTGVSIPCVCGRDATQYLVQQESSFVMMSAPPAEYKLQQGTF
LCANEYTGNYQCGHYTHITAKETLYRIDGAHLTKMSEYKGPVTDVFYKET
SYTTTIKPVSYKLDGVTYTEIEPKLDGYYKKDNAYYTEQPIDLVPTQPLP
NASFDNFKLTCSNTKFADDLNQMTGFTKPASRELSVTFFPDLNGDVVAID
YRHYSASFKKGAKLLHKPIVWHINQATTKTTFKPNTWCLRCLWSTKPVDT
SNSFEVLAVEDTQGMDNLACESQQPTSEEVVENPTIQKEVIECDVKTTEV
VGNVILKPSDEGVKVTQELGHEDLMAAYVENTSITIKKPNELSLALGLKT
IATHGIAAINSVPWSKILAYVKPFLGQAAITTSNCAKRLAQRVFNNYMPY
VFTLLFQLCTFTKSTNSRIRASLPTTIAKNSVKSVAKLCLDAGINYVKSP
KFSKLFTIAMWLLLLSICLGSLICVTAAFGVLLSNFGAPSYCNGVRELYL
NSSNVTTMDFCEGSFPCSICLSGLDSLDSYPALETIQVTISSYKLDLTIL
GLAAEWVLAYMLFTKFFYLLGLSAIMQVFFGYFASHFISNSWLMWFIISI
VQMAPVSAMVRMYIFFASFYYIWKSYVHIMDGCTSSTCMMCYKRNRATRV
ECTTIVNGMKRSFYVYANGGRGFCKTHNWNCLNCDTFCTGSTFISDEVAR
DLSLQFKRPINPTDQSSYIVDSVAVKNGALHLYFDKAGQKTYERHPLSHF
VNLDNLRANNTKGSLPINVIVFDGKSKCDESASKSASVYYSQLMCQPILL
LDQALVSDVGDSTEVSVKMFDAYVDTFSATFSVPMEKLKALVATAHSELA
KGVALDGVLSTFVSAARQGVVDTDVDTKDVIECLKLSHHSDLEVTGDSCN
NFMLTYNKVENMTPRDLGACIDCNARHINAQVAKSHNVSLIWNVKDYMSL
SEQLRKQIRSAAKKNNIPFRLTCATTRQVVNVITTKISLKGGKIVSTCFK
LMLKATLLCVLAALVCYIVMPVHTLSIHDGYTNEIIGYKAIQDGVTRDII
STDDCFANKHAGFDAWFSQRGGSYKNDKSCPVVAAIITREIGFIVPGLPG
TVLRAINGDFLHFLPRVFSAVGNICYTPSKLIEYSDFATSACVLAAECTI
FKDAMGKPVPYCYDTNLLEGSISYSELRPDTRYVLMDGSIIQFPNTYLEG
SVRVVTTFDAEYCRHGTCERSEVGICLSTSGRWVLNNEHYRALSGVFCGV
DAMNLIANIFTPLVQPVGALDVSASVVAGGIIAILVTCAAYYFMKFRRVF
GEYNHVVAANALLFLMSFTILCLVPAYSFLPGVYSVFYLYLTFYFTNDVS
FLAHLQWFAMFSPIVPFWITAIYVFCISLKHCHWFFNNYLRKRVMFNGVT
FSTFEEAALCTFLLNKEMYLKLRSETLLPLTQYNRYLALYNKYKYFSGAL
DTTSYREAACCHLAKALNDFSNSGADVLYQPPQTSITSAVLQSGFRKMAF
PSGKVEGCMVQVTCGTTTLNGLWLDDTVYCPRHVICTAEDMLNPNYEDLL
IRKSNHSFLVQAGNVQLRVIGHSMQNCLLRLKVDTSNPKTPKYKFVRIQP
GQTFSVLACYNGSPSGVYQCAMRPNHTIKGSFLNGSCGSVGFNIDYDCVS
FCYMHHMELPTGVHAGTDLEGKFYGPFVDRQTAQAAGTDTTITLNVLAWL
YAAVINGDRWFLNRFTTTLNDFNLVAMKYNYEPLTQDHVDILGPLSAQTG
IAVLDMCAALKELLQNGMNGRTILGSTILEDEFTPFDVVRQCSGVTFQGK
FKKIVKGTHHWMLLTFLTSLLILVQSTQWSLFFFVYENAFLPFTLGIMAI
AACAMLLVKHKHAFLCLFLLPSLATVAYFNMVYMPASWVMRIMTWLELAD
TSLSGYRLKDCVMYASALVLLILMTARTVYDDAARRVWTLMNVITLVYKV
YYGNALDQAISMWALVISVTSNYSGVVTTIMFLARAIVFVCVEYYPLLFI
TGNTLQCIMLVYCFLGYCCCCYFGLFCLLNRYFRLTLGVYDYLVSTQEFR
YMNSQGLLPPKSSIDAFKLNIKLLGIGGKPCIKVATVQSKMSDVKCTSVV
LLSVLQQLRVESSSKLWAQCVQLHNDILLAKDTTEAFEKMVSLLSVLLSM
QGAVDINRLCEEMLDNRATLQAIASEFSSLPSYAAYATAQEAYEQAVANG
DSEVVLKKLKKSLNVAKSEFDRDAAMQRKLEKMADQAMTQMYKQARSEDK
RAKVTSAMQTMLFTMLRKLDNDALNNIINNARDGCVPLNIIPLTTAAKLM
VVVPDYGTYKNTCDGNTFTYASALWEIQQVVDADSKIVQLSEINMDNSPN
LAWPLIVTALRANSAVKLQNNELSPVALRQMSCAAGTTQTACTDDNALAY
YNNSKGGRFVLALLSDHQDLKWARFPKSDGTGTIYTELEPPCRFVTDTPK
GPKVKYLYFIKGLNNLNRGMVLGSLAATVRLQAGNATEVPANSTVLSFCA
FAVDPAKAYKDYLASGGQPITNCVKMLCTHTGTGQAITVTPEANMDQESF
GGASCCLYCRCHIDHPNPKGFCDLKGKYVQIPTTCANDPVGFTLRNTVCT
VCGMWKGYGCSCDQLREPLMQSADASTFLNRVCGVSAARLTPCGTGTSTD
VVYRAFDIYNEKVAGFAKFLKTNCCRFQEKDEEGNLLDSYFVVKRHTMSN
YQHEETIYNLVKDCPAVAVHDFFKFRVDGDMVPHISRQRLTKYTMADLVY
ALRHFDEGNCDTLKEILVTYNCCDDDYFNKKDWYDFVENPDILRVYANLG
ERVRQSLLKTVQFCDAMRDAGIVGVLTLDNQDLNGNWYDFGDFVQVAPGC
GVPIVDSYYSLLMPILTLTRALAAESHMDADLAKPLIKWDLLKYDFTEER
LCLFDRYFKYWDQTYHPNCINCLDDRCILHCANFNVLFSTVFPPTSFGPL
VRKIFVDGVPFVVSTGYHFRELGVVHNQDVNLHSSRLSFKELLVYAADPA
MHAASGNLLLDKRTTCFSVAALTNNVAFQTVKPGNFNKDFYDFAVSKGFF
KEGSSVELKHFFFAQDGNAAISDYDYYRYNLPTMCDIRQLLFVVEVVDKY
FDCYDGGCINANQVIVNNLDKSAGFPFNKWGKARLYYDSMSYEDQDALFA
YTKRNVIPTITQMNLKYAISAKNRARTVAGVSICSTMTNRQFHQKLLKSI
AATRGATVVIGTSKFYGGWHNMLKTVYSDVETPHLMGWDYPKCDRAMPNM
LRIMASLVLARKHNTCCNLSHRFYRLANECAQVLSEMVMCGGSLYVKPGG
TSSGDATTAYANSVFNICQAVTANVNALLSTDGNKIADKYVRNLQHRLYE
CLYRNRDVDHEFVDEFYAYLRKHFSMMILSDDAVVCYNSNYAAQGLVASI
KNFKAVLYYQNNVFMSEAKCWTETDLTKGPHEFCSQHTMLVKQGDDYVYL
PYPDPSRILGAGCFVDDIVKTDGTLMIERFVSLAIDAYPLTKHPNQEYAD
VFHLYLQYIRKLHDELTGHMLDMYSVMLTNDNTSRYWEPEFYEAMYTPHT
VLQAVGACVLCNSQTSLRCGACIRRPFLCCKCCYDHVISTSHKLVLSVNP
YVCNAPGCDVTDVTQLYLGGMSYYCKSHKPPISFPLCANGQVFGLYKNTC
VGSDNVTDFNAIATCDWTNAGDYILANTCTERLKLFAAETLKATEETFKL
SYGIATVREVLSDRELHLSWEVGKPRPPLNRNYVFTGYRVTKNSKVQIGE
YTFEKGDYGDAVVYRGTTTYKLNVGDYFVLTSHTVMPLSAPTLVPQEHYV
RITGLYPTLNISDEFSSNVANYQKVGMQKYSTLQGPPGTGKSHFAIGLAL
YYPSARIVYTACSHAAVDALCEKALKYLPIDKCSRIIPARARVECFDKFK
VNSTLEQYVFCTVNALPETTADIVVFDEISMATNYDLSVVNARLRAKHYV
YIGDPAQLPAPRTLLTKGTLEPEYFNSVCRLMKTIGPDMFLGTCRRCPAE
IVDTVSALVYDNKLKAHKDKSAQCFKMFYKGVITHDVSSAINRPQIGVVR
EFLTRNPAWRKAVFISPYNSQNAVASKILGLPTQTVDSSQGSEYDYVIFT
QTTETAHSCNVNRFNVAITRAKIGILCIMSDRDLYDKLQFTSLEIPRRNV
ATLQAENVTGLFKDCSKIITGLHPTQAPTHLSVDIKFKTEGLCVDIPGIP
KDMTYRRLISMMGFKMNYQVNGYPNMFITREEAIRHVRAWIGFDVEGCHA
TRDAVGTNLPLQLGFSTGVNLVAVPTGYVDTENNTEFTRVNAKPPPGDQF
KHLIPLMYKGLPWNVVRIKIVQMLSDTLKGLSDRVVFVLWAHGFELTSMK
YFVKIGPERTCCLCDKRATCFSTSSDTYACWNHSVGFDYVYNPFMIDVQQ
WGFTGNLQSNHDQHCQVHGNAHVASCDAIMTRCLAVHECFVKRVDWSVEY
PIIGDELRVNSACRKVQHMVVKSALLADKFPVLHDIGNPKAIKCVPQAEV
EWKFYDAQPCSDKAYKIEELFYSYATHHDKFTDGVCLFWNCNVDRYPANA
IVCRFDTRVLSNLNLPGCDGGSLYVNKHAFHTPAFDKSAFTNLKQLPFFY
YSDSPCESHGKQVVSDIDYVPLKSATCITRCNLGGAVCRHHANEYRQYLD
AYNMMISAGFSLWIYKQFDTYNLWNTFTRLQSLENVAYNVVNKGHFDGHA
GEAPVSIINNAVYTKVDGIDVEIFENKTTLPVNVAFELWAKRNIKPVPEI
KILNNLGVDIAANTVIWDYKREAPAHVSTIGVCTMTDIAKKPTESACSSL
TVLFDGRVEGQVDLFRNARNGVLITEGSVKGLTPSKGPAQASVNGVTLIG
ESVKTQFNYFKKVDGIIQQLPETYFTQSRDLEDFKPRSQMETDFLELAMD
EFIQRYKLEGYAFEHIVYGDFSHGQLGGLHLMIGLAKRSQDSPLKLEDFI
PMDSTVKNYFITDAQTGSSKCVCSVIDLLLDDFVEIIKSQDLSVISKVVK
VTIDYAEISFMLWCKDGHVETFYPKLQASQAWQPGVAMPNLYKMQRMLLE
KCDLQNYGENAVIPKGIMMNVAKYTQLCQYLNTLTLAVPYNMRVIHFGAG
SDKGVAPGTAVLRQWLPTGTLLVDSDLNDFVSDADSTLIGDCATVHTANK
WDLIISDMYDPRTKHVTKENDSKEGFFTYLCGFIKQKLALGGSIAVKITE
HSWNADLYKLMGHFSWWTAFVTNVNASSSEAFLIGANYLGKPKEQIDGYT
MHANYIFWRNTNPIQLSSYSLFDMSKFPLKLRGTAVMSLKENQINDMIYS
LLEKGRLIIRENNRVVVSSDILVNN

| | | | | | | | | |
|---|---|---|---|---|---|---|---|---|
| 43 | 265 | 13413 | 1489680 | orf1ab | sars1 | NP_ | 4382 | orf1a |
| | | | | | | 828850.1 | | polyprotein |
| | | | | | | | | (pp1a) |

MESLVLGVNEKTHVQLSLPVLQVRDVLVRGFGDSVEEALSEAREHLKNGT
CGLVELEKGVLPQLEQPYVFIKRSDALSTNHGHKVVELVAEMDGIQYGRS
GITLGVLVPHVGETPIAYRNVLLRKNGNKGAGGHSYGIDLKSYDLGDELG
TDPIEDYEQNWNTKHGSGALRELTRELNGGAVTRYVDNNFCGPDGYPLDC
IKDFLARAGKSMCTLSEQLDYIESKRGVYCCRDHEHEIAWFTERSDKSYE
HQTPFEIKSAKKFDTFKGECPKFVFPLNSKVKVIQPRVEKKKTEGFMGRI
RSVYPVASPQECNNMHLSTLMKCNHCDEVSWQTCDFLKATCEHCGTENLV
IEGPTTCGYLPTNAVVKMPCPACQDPEIGPEHSVADYHNHSNIETRLRKG
GRTRCFGGCVFAYVGCYNKRAYWVPRASADIGSGHTGITGDNVETLNEDL
LEILSRERVNINIVGDFHLNEEVAIILASFSASTSAFIDTIKSLDYKSFK
TIVESCGNYKVTKGKPVKGAWNIGQQRSVLTPLCGFPSQAAGVIRSIFAR
TLDAANHSIPDLQRAAVTILDGISEQSLRLVDAMVYTSDLLTNSVIIMAY
VTGGLVQQTSQWLSNLLGTTVEKLRPIFEWIEAKLSAGVEFLKDAWEILK
FLITGVFDIVKGQIQVASDNIKDCVKCFIDVVNKALEMCIDQVTIAGAKL
RSLNLGEVFIAQSKGLYRQCIRGKEQLQLLMPLKAPKEVTFLEGDSHDTV
LTSEEVVLKNGELEALETPVDSFTNGAIVGTPVCVNGLMLLEIKDKEQYC
ALSPGLLATNNVFRLKGGAPIKGVTFGEDTVWEVQGYKNVRITFELDERV
DKVLNEKCSVYTVESGTEVTEFACVVAEAVVKTLQPVSDLLTNMGIDLDE
WSVATFYLFDDAGEENFSSRMYCSFYPPDEEEEDDAECEEEEIDETCEHE
YGTEDDYQGLPLEFGASAETVRVEEEEEEDWLDDTTEQSEIEPEPEPTPE
EPVNQFTGYLKLTDNVAIKCVDIVKEAQSANPMVIVNAANIHLKHGGGVA
GALNKATNGAMQKESDDYIKLNGPLTVGGSCLLSGHNLAKKCLHVVGPNL
NAGEDIQLLKAAYENFNSQDILLAPLLSAGIFGAKPLQSLQVCVQTVRTQ
VYIAVNDKALYEQVVMDYLDNLKPRVEAPKQEEPPNTEDSKTEEKSVVQK
PVDVKPKIKACIDEVTTTLEETKFLTNKLLLFADINGKLYHDSQNMLRGE
DMSFLEKDAPYMVGDVITSGDITCVVIPSKKAGGTTEMLSRALKKVPVDE
YITTYPGQGCAGYTLEEAKTALKKCKSAFYVLPSEAPNAKEEILGTVSWN
LREMLAHAEETRKLMPICMDVRAIMATIQRKYKGIKIQEGIVDYGVRFFF
YTSKEPVASIITKLNSLNEPLVTMPIGYVTHGFNLEEAARCMRSLKAPAV
VSVSSPDAVTTYNGYLTSSSKTSEEHFVETVSLAGSYRDWSYSGQRTELG
VEFLKRGDKIVYHTLESPVEFHLDGEVLSLDKLKSLLSLREVKTIKVFTT
VDNTNLHTQLVDMSMTYGQQFGPTYLDGADVTKIKPHVNHEGKTFFVLPS
DDTLRSEAFEYYHTLDESFLGRYMSALNHTKKWKFPQVGGLTSIKWADNN
CYLSSVLLALQQLEVKFNAPALQEAYYRARAGDAANFCALILAYSNKTVG
ELGDVRETMTHLLQHANLESAKRVLNVVCKHCGQKTTTLTGVEAVMYMGT
LSYDNLKTGVSIPCVCGRDATQYLVQQESSFVMMSAPPAEYKLQQGTFLC
ANEYTGNYQCGHYTHITAKETLYRIDGAHLTKMSEYKGPVTDVFYKETSY
TTTIKPVSYKLDGVTYTEIEPKLDGYYKKDNAYYTEQPIDLVPTQPLPNA
SFDNFKLTCSNTKFADDLNQMTGFTKPASRELSVTFFPDLNGDVVAIDYR
HYSASFKKGAKLLHKPIVWHINQATTKTTFKPNTWCLRCLWSTKPVDTSN
SFEVLAVEDTQGMDNLACESQQPTSEEVVENPTIQKEVIECDVKTTEVVG
NVILKPSDEGVKVTQELGHEDLMAAYVENTSITIKKPNELSLALGLKTIA
THGIAAINSVPWSKILAYVKPFLGQAAITTSNCAKRLAQRVFNNYMPYVF
TLLFQLCTFTKSTNSRIRASLPTTIAKNSVKSVAKLCLDAGINYVKSPKF
SKLFTIAMWLLLLSICLGSLICVTAAFGVLLSNFGAPSYCNGVRELYLNS
SNVTTMDFCEGSFPCSICLSGLDSLDSYPALETIQVTISSYKLDLTILGL
AAEWVLAYMLFTKFFYLLGLSAIMQVFFGYFASHFISNSWLMWFIISIVQ
MAPVSAMVRMYIFFASFYYIWKSYVHIMDGCTSSTCMMCYKRNRATRVEC
TTIVNGMKRSFYVYANGGRGFCKTHNWNCLNCDTFCTGSTFISDEVARDL
SLQFKRPINPTDQSSYIVDSVAVKNGALHLYFDKAGQKTYERHPLSHFVN
LDNLRANNTKGSLPINVIVFDGKSKCDESASKSASVYYSQLMCQPILLLD
QALVSDVGDSTEVSVKMFDAYVDTFSATFSVPMEKLKALVATAHSELAKG
VALDGVLSTFVSAARQGVVDTDVDTKDVIECLKLSHHSDLEVTGDSCNNF
MLTYNKVENMTPRDLGACIDCNARHINAQVAKSHNVSLIWNVKDYMSLSE
QLRKQIRSAAKKNNIPFRLTCATTRQVVNVITTKISLKGGKIVSTCFKLM
LKATLLCVLAALVCYIVMPVHTLSIHDGYTNEIIGYKAIQDGVTRDIIST
DDCFANKHAGFDAWFSQRGGSYKNDKSCPVVAAIITREIGFIVPGLPGTV
LRAINGDFLHFLPRVFSAVGNICYTPSKLIEYSDFATSACVLAAECTIFK
DAMGKPVPYCYDTNLLEGSISYSELRPDTRYVLMDGSIIQFPNTYLEGSV
RVVTTFDAEYCRHGTCERSEVGICLSTSGRWVLNNEHYRALSGVFCGVDA
MNLIANIFTPLVQPVGALDVSASVVAGGIIAILVTCAAYYFMKFRRVFGE
YNHVVAANALLFLMSFTILCLVPAYSFLPGVYSVFYLYLTFYFTNDVSFL
AHLQWFAMFSPIVPFWITAIYVFCISLKHCHWFFNNYLRKRVMFNGVTFS
TFEEAALCTFLLNKEMYLKLRSETLLPLTQYNRYLALYNKYKYFSGALDT
TSYREAACCHLAKALNDFSNSGADVLYQPPQTSITSAVLQSGFRKMAFPS
GKVEGCMVQVTCGTTTLNGLWLDDTVYCPRHVICTAEDMLNPNYEDLLIR
KSNHSFLVQAGNVQLRVIGHSMQNCLLRLKVDTSNPKTPKYKFVRIQPGQ
TFSVLACYNGSPSGVYQCAMRPNHTIKGSFLNGSCGSVGFNIDYDCVSFC
YMHHMELPTGVHAGTDLEGKFYGPFVDRQTAQAAGTDTTITLNVLAWLYA
AVINGDRWFLNRFTTTLNDFNLVAMKYNYEPLTQDHVDILGPLSAQTGIA
VLDMCAALKELLQNGMNGRTILGSTILEDEFTPFDVVRQCSGVTFQGKFK
KIVKGTHHWMLLTFLTSLLILVQSTQWSLFFFVYENAFLPFTLGIMAIAA
CAMLLVKHKHAFLCLFLLPSLATVAYFNMVYMPASWVMRIMTWLELADTS
LSGYRLKDCVMYASALVLLILMTARTVYDDAARRVWTLMNVITLVYKVYY
GNALDQAISMWALVISVTSNYSGVVTTIMFLARAIVFVCVEYYPLLFITG
NTLQCIMLVYCFLGYCCCCYFGLFCLLNRYFRLTLGVYDYLVSTQEFRYM
NSQGLLPPKSSIDAFKLNIKLLGIGGKPCIKVATVQSKMSDVKCTSVVLL
SVLQQLRVESSSKLWAQCVQLHNDILLAKDTTEAFEKMVSLLSVLLSMQG
AVDINRLCEEMLDNRATLQAIASEFSSLPSYAAYATAQEAYEQAVANGDS
EVVLKKLKKSLNVAKSEFDRDAAMQRKLEKMADQAMTQMYKQARSEDKRA
KVTSAMQTMLFTMLRKLDNDALNNIINNARDGCVPLNIIPLTTAAKLMVV
VPDYGTYKNTCDGNTFTYASALWEIQQVVDADSKIVQLSEINMDNSPNLA
WPLIVTALRANSAVKLQNNELSPVALRQMSCAAGTTQTACTDDNALAYYN
NSKGGRFVLALLSDHQDLKWARFPKSDGTGTIYTELEPPCRFVTDTPKGP
KVKYLYFIKGLNNLNRGMVLGSLAATVRLQAGNATEVPANSTVLSFCAFA
VDPAKAYKDYLASGGQPITNCVKMLCTHTGTGQAITVTPEANMDQESFGG
ASCCLYCRCHIDHPNPKGFCDLKGKYVQIPTTCANDPVGFTLRNTVCTVC
GMWKGYGCSCDQLREPLMQSADASTFLNGFAV

| | | | | | | | | |
|---|---|---|---|---|---|---|---|---|
| 44 | 21492 | 25259 | 1489668 | S | sars2 | NP_ | 1255 | E2 |
| | | | | | | 828851.1 | | glycoprotein |
| | | | | | | | | precursor |

MFIFLLFLTLTSGSDLDRCTTFDDVQAPNYTQHTSSMRGVYYPDEIFRSD
TLYLTQDLFLPFYSNVTGFHTINHTFGNPVIPFKDGIYFAATEKSNVVRG
WVFGSTMNNKSQSVIIINNSTNVVIRACNFELCDNPFFAVSKPMGTQTHT
MIFDNAFNCTFEYISDAFSLDVSEKSGNFKHLREFVFKNKDGFLYVYKGY
QPIDVVRDLPSGFNTLKPIFKLPLGINITNFRAILTAFSPAQDIWGTSAAA
YFVGYLKPTTFMLKYDENGTITDAVDCSQNPLAELKCSVKSFEIDKGIYQ
TSNFRVVPSGDVVRFPNITNLCPFGEVFNATKFPSVYAWERKKISNCVAD
YSVLYNSTFFSTFKCYGVSATKLNDLCFSNVYADSFVVKGDDVRQIAPGQ
TGVIADYNYKLPDDFMGCVLAWNTRNIDATSTGNYNYKYRYLRHGKLRPF
ERDISNVPFSPDGKPCTPPALNCYWPLNDYGFYTTTGIGYQPYRVVVLSF
ELLNAPATVCGPKLSTDLIKNQCVNFNFNGLTGTGVLTPSSKRFQPFQQF
GRDVSDFTDSVRDPKTSEILDISPCAFGGVSVITPGTNASSEVAVLYQDV
NCTDVSTAIHADQLTPAWRIYSTGNNVFQTQAGCLIGAEHVDTSYECDIP
IGAGICASYHTVSLLRSTSQKSIVAYTMSLGADSSIAYSNNTIAIPTNFS
ISITTEVMPVSMAKTSVDCNMYICGDSTECANLLLQYGSFCTQLNRALSG
IAAEQDRNTREVFAQVKQMYKTPTLKYFGGFNFSQILPDPLKPTKRSFIE
DLLFNKVTLADAGFMKQYGECLGDINARDLICAQKFNGLTVLPPLLTDDM
IAAYTAALVSGTATAGWTFGAGAALQIPFAMQMAYRFNGIGVTQNVLYEN
QKQIANQFNKAISQIQESLTTTSTALGKLQDVVNQNAQALNTLVKQLSSN
FGAISSVLNDILSRLDKVEAEVQIDRLITGRLQSLQTYVTQQLIRAAEIR
ASANLAATKMSECVLGQSKRVDFCGKGYHLMSFPQAAPHGVVFLHVTYVP
SQERNFTTAPAICHEGKAYFPREGVFVFNGTSWFITQRNFFSPQIITTDN
TFVSGNCDVVIGIINNTVYDPLQPELDSFKEELDKYFKNHTSPDVDLGDI
SGINASVVNIQKEIDRLNEVAKNLNESLIDLQELGKYEQYIKWPWYVWLG
FIAGLIAIVMVTILLCCMTSCCSCLKGACSCGSCCKFDEDDSEPVLKGVK
LHYT

| | | | | | | | |
|---|---|---|---|---|---|---|---|
| 45 | 25268 | 26092 | 1489669 | sars3a | NP_ | 274 | hypothetical |
| | | | | | 828852.2 | | protein |
| | | | | | | | sars3a |

MDLFMRFFTLRSITAQPVKIDNASPASTVHATATIPLQASLPFGWLVIGV
AFLAVFQSATKIIALNKRWQLALYKGFQFICNLLLLFVTIYSHLLLVAAG
MEAQFLYLYALIYFLQCINACRIIMRCWLCWKCKSKNPLLYDANYFVCWH
THNYDYCIPYNSVTDTIVVTEGDGISTPKLKEDYQIGGYSEDRHSGVKDY
VVVHGYFTEVYYQLESTQITTDTGIENATFFIFNKLVKDPPNVQIHTIDG
SSGVANPAMDPIYDEPTTTTSVPL

| | | | | | | | |
|---|---|---|---|---|---|---|---|
| 46 | 25689 | 26153 | 1489670 | sars3b | NP_ | 154 | hypothetical |
| | | | | | 828853.1 | | protein |
| | | | | | | | sars3b |

MMPTTLFAGTHITMTTVYHITVSQIQLSLLKVTAFQHQNSKKTTKLVVIL
RIGTQVLKTMSLYMAISPKFTTSLSLHKLLQTLVLKMLHSSSLTSLLKTH
RMCKYTQSTALQELLIQQWIQFMMSRRRLLACLCKHKKVSTNLCTHSFRK
KQVR

| | | | | | | | | |
|---|---|---|---|---|---|---|---|---|
| 47 | 26117 | 26347 | 1489671 | E | sars4 | NP_ | 76 | protein |
| | | | | | | 828854.1 | | E |

MYSFVSEETGTLIVNSVLLFLAFVVFLLVTLAILTALRLCAYCCNIVNVS
LVKPTVYVYSRVKNLNSSEGVPDLLV

| | | | | | | | | |
|---|---|---|---|---|---|---|---|---|
| 48 | 26398 | 27063 | 1489672 | M | sars5 | NP_ | 221 | matrixprotein |
| | | | | | | 828855.1 | | |

MADNGTITVEELKQLLEQWNLVIGFLFLAWIMLLQFAYSNRNRFLYIIKL
VFLWLLWPVTLACFVLAAVYRINWVTGGIAIAMACIVGLMWLSYFVASFR
LFARTRSMWSFNPETNILLNVPLRGTIVTRPLMESELVIGAVIIRGHLRM
AGHSLGRCDIKDLPKEITVATSRTLSYYKLGASQRVGTDSGFAAYNRYRI
GNYKLNTDHAGSNDNIALLVQ

| | | | | | | | |
|---|---|---|---|---|---|---|---|
| 49 | 27074 | 27265 | 1489673 | sars6 | NP_ | 63 | hypothetical |
| | | | | | 828856.1 | | protein |
| | | | | | | | sars6 |

MFHLVDFQVTIAEILIIIMRTFRIAIWNLDVIISSIVRQLFKPLTKKNYS
ELDDEEPMELDYP

| | | | | | | | |
|---|---|---|---|---|---|---|---|
| 50 | 27273 | 27641 | 1489674 | sars7a | NP_ | 122 | hypothetical |
| | | | | | 828857.1 | | protein |
| | | | | | | | sars7a |

MKIILFLTLIVFTSCELYHYQECVRGTTVLLKEPCPSGTYEGNSPFHPLA
DNKFALTCTSTHFAFACADGTRHTYQLRARSVSPKLFIRQEEVQQELYS
PLFLIVAALVFLILCFTIKRKTE

| | | | | | | | |
|---|---|---|---|---|---|---|---|
| 51 | 27638 | 27772 | 1489675 | sars7b | NP_ | 44 | hypothetical |
| | | | | | 849175.1 | | protein |
| | | | | | | | sars7b |

MNELTLIDFYLCFLAFLLFLVLIMLIIFWFSLEIQDLEEPCTKV

| | | | | | | | |
|---|---|---|---|---|---|---|---|
| 52 | 27779 | 27898 | 1489676 | sars8a | NP_ | 39 | hypothetical |
| | | | | | 849176.1 | | protein |
| | | | | | | | sars8a |

MKLL1VLTCISLCSCICTVVQRCASNKPHVLEDPCKVQH

| | | | | | | | |
|---|---|---|---|---|---|---|---|
| 53 | 27864 | 28118 | 1489677 | sars8b | NP_ | 84 | hypothetical |
| | | | | | 849177.1 | | protein |
| | | | | | | | sars8b |

MCLKILVRYNTRGNTYSTAWLCALGKVLPFHRWHTMVQTCTPNVTINCQD
PAGGALIARCWYLHEGHQTAAFRDVLVVLNKRTN

| | | | | | | | | |
|---|---|---|---|---|---|---|---|---|
| 54 | 28120 | 29388 | 1489678 | N | sars9a | NP_ | 422 | nucleocapsid |
| | | | | | | 828858.1 | | protein |

MSDNGPQSNQRSAPRITFGGPTDSTDNNQNGGRNGARPKQRRPQGLPNNT
ASWFTALTQHGKEELRFPRGQGVPINTNSGPDDQIGYYRRATRRVRGGDG
KMKELSPRWYFYYLGTGPEASLPYGANKEGIVWVATEGALNTPKDHIGTR
NPNNNAATVLQLPQGTTLPKGFYAEGSRGGSQASSRSSSRSRGNSRNSTP
GSSRGNSPARMASGGGETALALLLLDRLNQLESKVSGKGQQQQGQTVTKK
SAAEASKKPRQKRTATKQYNVTQAFGRRGPEQTQGNFGDQDLIRQGTDYK
HWPQIAQFAPSASAFFGMSRIGMEVTPSGTWLTYHGAIKLDDKDPQFKDN
VILLNKHIDAYKTFPPTEPKKDKKKKTDEAQPLPQRQKKQPTVTLLPAAD
MDDFSRQLQNSMSGASADSTQA

| | | | | | | | |
|---|---|---|---|---|---|---|---|
| 55 | 28130 | 28426 | 1489679 | sars9b | NP_ | 98 | hypothetical |
| | | | | | 828859.1 | | protein |
| | | | | | | | sars9b |

MDPNQTNVVPPALHLVDPQIQLTITRMEDAMGQGQNSADPKVYPIILRLG
SQLSLSMARRNLDSLEARAFQSTPIVVQMTKLATTEELPDEFVVVTAK

In some cases, a peptide comprises at least a fragment of a SARS-Cov-2 viral spike protein. In some cases, a peptide comprises a conserved domain of a SARS-Cov-2 virus structural protein, a polyprotein protease, a spike protein, or combinations thereof. In some cases, a peptide or fragment thereof can have from about 50%, 60%, 70%, 75%, 80%, 85%, 88%, 90%, 93%, 95%, 96%, 97%, 98%, 99%, or 100% identity to SEQ ID NO: 56 from Table 3. In some cases, a peptide or fragment thereof comprises from about 7-17, 7-15, 7-10, 8-17, 8-15, 8-10, 9-17, 9-15, or 9-10 continuous amino acids from a sequence in Table 3. In some cases, a peptide or fragment thereof comprises from about 7-17, 7-15, 7-10, 8-17, 8-15, 8-10, 9-17, 9-15, or 9-10 continuous amino acids of SEQ ID NO: 56. In some cases, an epitope of SEQ ID NO: 56 can be utilized.

TABLE 3
SARS-CoV-2 full length spike glycoprotein

| SEQ | |
|---|---|
| ID | |
| NO | Sequence |
| 56 | MGILPSPGMPALLSLVSLLSVLLMGCVAET |
| | GTQCVNLTTRTQLPPAYTNSFTRGVYYPDK |
| | VFRSSVLHSTQDLFLPFFSNVTWFHAIHVS |
| | GTNGTKRFDNPVLPFNDGVYFASTEKSNII |
| | RGWIFGTTLDSKTQSLLIVNNATNVVIKVC |
| | EFQFCNDPFLGVYYHKNNKSWMESEFRVYS |
| | SANNCTFEYVSQPFLMDLEGKQGNFKNLRE |
| | FVFKNIDGYFKIYSKHTPINLVRDLPQGFS |
| | ALEPLVDLPIGINITRFQTLLALHRSYLTP |
| | GDSSSGWTAGAAAYYVGYLQPRTFLLKYNE |
| | NGTITDAVDCALDPLSETKCTLKSFTVEKG |
| | IYQTSNFRVQPTESIVRFPNITNLCPFGEV |
| | FNATRFASVYAWNRKRISNCVADYSVLYNS |
| | ASFSTFKCYGVSPTKLNDLCFTNVYADSFV |
| | IRGDEVRQIAPGQTGKIADYNYKLPDDFTG |
| | CVIAWNSNNLDSKVGGNYNYLYRLFRKSNL |
| | KPFERDISTEIYQAGSTPCNGVEGFNCYFP |
| | LQSYGFQPTNGVGYQPYRVVVLSFELLHAP |
| | ATVCGPKKSTNLVKNKCVNFNFNGLTGTGV |
| | LTESNKKFLPFQQFGRDIADTTDAVRDPQT |
| | LEILDITPCSFGGVSVITPGTNTSNEVAVL |
| | YQDVNCTEVPVAIHADQLTPTWRVYSTGSN |
| | VFQTRAGCLIGAEHVNNSYECDIPIGAGIC |
| | ASYQTQTNSPSGAGSVASQSIIAYTMSLGA |
| | ENSVAYSNNSIAIPTNFTISVTTEILPVSM |
| | TKTSVDCTMYICGDSTECSNLLLQYGSFCT |
| | QLNRALTGIAVEQDKNTQEVFAQVKQIYKT |
| | PPIKDFGGFNFSQILPDPSKPSKRSFIEDL |
| | LFNKVTLADAGFIKQYGDCLGDIAARDLIC |
| | AQKFNGLTVLPPLLTDEMIAQYTSALLAGT |
| | ITSGWTFGAGAALQIPFAMQMAYRFNGIGV |
| | TQNVLYENQKLIANQFNSAIGKIQDSLSST |
| | ASALGKLQDVVNQNAQALNTLVKQLSSNFG |
| | AISSVLNDILSRLDPPEAEVQIDRLITGRL |
| | QSLQTYVTQQLIRAAEIRASANLAATKMSE |
| | CVLGQSKRVDFCGKGYHLMSFPQSAPHGVV |
| | FLHVTYVPAQEKNFTTAPAICHDGKAHFPR |
| | EGVFVSNGTHWFVTQRNFYEPQIITTDNTF |
| | VSGNCDVVIGIVNNTVYDPLQPELDSFKEE |
| | LDKYFKNHTSPDVDLGDISGINASVVNIQK |
| | EIDRLNEVAKNLNESLIDLQELGKYEQYIK |
| | GSGRENLYFQGGGGSGYIPEAPRDGQAYVR |
| | KDGEWVLLSTFLGHHHHHHHH |

Compositions and methods provided herein can be utilized to prevent or reduce uptake of a virus, for instance, a SARS-Cov-2 virus by reducing or preventing infection via the human ACE2 receptor. In some cases, administration of subject cells generated using a compositions or method provided therein are effective to reduce or prevent infection of a SARS-Cov-2 virus by at least about 0.5 fold, 1 fold, 2 fold, 3 fold, 4 fold, 5 fold, 6 fold, 7 fold, 8 fold, 9 fold, 10 fold, 20 fold, 30 fold, 40 fold, 50 fold, 60 fold, 80 fold, 100 fold, 120 fold, 140 fold, 160 fold, 180 fold, or up to about 200 fold.

Suitable methods to determine the presence of an anti-SARS-Cov-2 virus response include evaluating humoral immunity, cellular immunity, or combinations thereof. Suitable assays include but are not limited to ELISA, pseudoviral neutralization test, anti-S protein antibodies, inhibition and neutralization of hemagglutination, opsonophagocytosic capacity, surface plasmon resonance, and the like. An ELISA can be performed on serum or cellular supernatant.

### Bacterial Peptides

In some embodiments, the peptide is from a bacterial source or is a variant or derivative of a peptide from a bacterial source. In some embodiments, the bacterial source is Gram positive (+)ve cocci, Gram negative (−)ve cocci, Gram positive (+)ve bacilli, Gram negative (−)ve bacilli, Anaerobic bacteria, Spirochetes, Mycobacteria, Rickettsia, Chlamydia, Mycoplasmas, or any combination thereof. In some embodiments, said bacteria is selected from any one of: Staphylococcus aureus, Streptococcus pneumoniae, S, pyogenes, Neisseria gonorrhoeae, N. meningitidis, Corynebacteria, Bacillus anthracis, Listeria monocytogenes, Salmonella, Shigella, Campylobacter, Vibrio, Yersiria, Pasteurella, Pseudomonas, Brucella, Hemophilus, Legionella, Bordetella, Clostridium tetani, C. botulinum, C. perfringens, Treponema pallidum, Borrelia burgdorferi, Leptospira interrogans, Mycobacterium tuberculosis, M. leprae, M. avium, Rickettsia prowazekii, Chlamydia trachomatis, or Mycoplasma pneumoniae, or any combination thereof.

Additional exemplary bacterial peptides can originate from any bacteria including, but not limited to: Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

### Fungal, Protozoa, and Parasitic Peptides

In some embodiments, a peptide is isolated from a fungus or a variant or derivative of a peptide isolate form a fungus. In some embodiments, said fungus is selected from the group consisting of cases, a pathogen can also be a fungi that is selected from the group consisting of Candida albicans, Cryptococcus neoformans, Aspergillus, Histoplasma capsulatum, Coccidioides immitis, and Pneumocystis carinii. A pathogen can also be a protozoan that is selected from the group consisting of Entamoeba histolytica, Giardia, Leishmania, Plasmodium, Trypanosoma, Toxoplasma gondii, and Cryptosporidium. In some cases, said peptide is a parasitic worm peptide. In some embodiments, a peptide is from a worm selected from the group consisting of Trichuris trichura, Trichinella spiralis, Enterobius vermicularis, Ascaris lumbricoides, Ancylostoma, Strongyloides; Filaria, Onchocerca volvulus, Loa, Dracuncula medinensis; Schistosoma, and Clonorchis sinensis.

Exemplary parasite peptides can original from a parasite including but not limited to: Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

In some embodiments, a peptide is from a protozoan, such as one or more protozoans from the genus Toxoplasma, for example T. gondii and species from a related genus, such as Neospora, Hammondia, Frenkelia, Isospora and Sarcocystis. Exemplary peptides derived from T. gondii include the GRA6, ROP2A, ROP18, SAG1, SAG2A and AMA1 gene products.

### Cancer Peptides

In some cases, a peptide is associated with a cancer or tumor. Exemplary peptides can be neoantigens. In some embodiments, the peptide or fragment thereof is selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (ela2), bcr-abl p210 (b2a2), bcr-abl p210 (b3a2), BING-4, CAG-3, CAIX, CAMEL, CISH, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, RORI, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WTi, α-folate receptor, and κ-light chain.

In some cases, a peptide comprises a neoantigen peptide. For example, a neoantigen can be a peptide that arises from polypeptide generated from genomic sequence that comprises an E805G mutation in ERBB2IP. Neoantigen and neoepitopes can be identified by whole-exome sequencing. A neoantigen and neoepitope peptide can be expressed on a subject particle. In some cases, a gene that can comprise a mutation that gives rise to a neoantigen or neoepitope peptide can be ABL1, ACOl 1997, ACVR2A, AFP, AKT1, ALK, ALPPL2, ANAPC1, APC, ARID1A, AR, AR-v7, ASCL2, β2M, BRAF, BTK, C15ORF40, CDH1, CLDN6, CNOT1, CT45A5, CTAG1B, DCT, DKK4,EEF1B2, EEFIDP3, EGFR, EIF2B3, env, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGEC1, MEK, MLANA, MLL2, MMP13, MSH3, MSH6, MYC, NDUFC2, NRAS, NY-ESO, PAGE2, PAGE5, PDGFRa, PIK3CA, PMEL, pol protein, POLE, PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, XPOT.

In some cases, the peptide(s) or fragment(s) thereof are derived from a polypeptide, a polypeptide generated from a nucleic acid sequence, or a neoantigen derived from at least one of A1CF, ABI1, ABL1, ABL2, ACKR3, ACSL3, ACSL6, ACVR1, ACVR1B, ACVR2A, AFDN, AFF1, AFF3, AFF4, AKAP9, AKT1, AKT2, AKT3, ALDH2, ALK, AMER1, ANK1, APC, APOBEC3B, AR, ARAF, ARHGAP26, ARHGAP5, ARHGEF10, ARHGEF10L, ARHGEF12, ARID1A, ARID1B, ARID2, ARNT, ASPSCR1, ASXL1, ASXL2, ATF1, ATIC, ATM, ATP1A1, ATP2B3, ATR, ATRX, AXIN1, AXIN2, B2M, BAP1, BARD1, BAX, BAZ1A, BCL10, BCL11A, BCL11B, BCL2, BCL2L12, BCL3, BCL6, BCL7A, BCL9, BCL9L, BCLAF1, BCOR, BCORL1, BCR, BIRC3, BIRC6, BLM, BMP5, BMPR1A, BRAF, BRCA1, BRCA2, BRD3, BRD4, BRIP1, BTG1, BTK, BUB1B, C15orf65, CACNAID, CALR, CAMTA1, CANT1, CARD11, CARS, CASP3, CASP8, CASP9, CBFA2T3, CBFB, CBL, CBLB, CBLC, CCDC6, CCNB1IP1, CCNC, CCND1, CCND2, CCND3, CCNE1, CCR4, CCR7, CD209, CD274, CD28, CD74, CD79A, CD79B, CDC73, CDH1, CDH10, CDH11, CDH17, CDK12, CDK4, CDK6, CDKN1A, CDKN1B, CDKN2A, CDKN2C, CDX2, CEBPA, CEP89, HCHD7, CHD2, CHD4, CHEK2, CHIC2, CHST11, CIC, CIITA, CLIP1, CLP1, CLTC, CLTCL1, CNBD1, CNBP, CNOT3, CNTNAP2, CNTRL, COL1A1, COL2A1, COL3A1, COX6C, CPEB3, CREB1, CREB3L1, CREB3L2, CREBBP, CRLF2, CRNKL1, CRTC1, CRTC3, CSF1R, CSF3R, CSMD3, CTCF, CTNNA2, CTNNB1, CTNND1, CTNND2, CUL3, CUX1, CXCR4, CYLD, CYP2C8, CYSLTR2, DAXX, DCAF12L2, DCC, DCTN1, DDB2, DDIT3, DDR2, DDX10, DDX3X, DDX5, DDX6, DEK, DGCR8, DICER1, DNAJB1, DNM2, DNMT1, DNMT3A, DROSHA, EBF1, ECT2L, EED, EGFR, EIF1AX, EIF3E, EIF4A2, ELF3, ELF4, ELK4, ELL, ELN, EML4, EP300, EPAS1, EPHA3, EPHA7, EPS15, ERBB2, ERBB3, ERBB4, ERC1, ERCC2, ERCC3, ERCC4, ERG, ESR1, ETNK1, ETV1, ETV4, ETV5, ETV6, EWSR1, EXT1, EXT2, EZH2, EZR, FAM131B, FAM135B, FAM46C, FAM47C, FANCA, FANCC, FANCD2, FANCE, FANCF, FANCG, FAS, FAT1, FAT3, FAT4, FBLN2, FBXO11, FBXW7, FCGR2B, FCRL4, FEN1, FES, FEV, FGFR1, FGFR1OP, FGFR2, FGFR3, FGFR4, FH, FHIT, FIP1L1, FKBP9, FLCN, FLI1, FLNA, FLT3, FLT4, FNBP1, FOXA1, FOXL2, FOXO1, FOXO3, FOXO4, FOXP1, FOXR1, FSTL3, FUBP1, FUS, GAS7, GATA1, GATA2, GATA3, GLI1, GMPS, GNA11, GNAQ, GNAS, GOLGA5, GOPC, GPC3, GPC5, GPHN, GRIN2A, GRM3, H3F3A, H3F3B, HERPUD1, HEY1, HIF1A, HIP1, HIST1H3B, HIST1H4I, HLA-A, HLF, HMGA1, HMGA2, HNF1A, HNRNPA2B1, HOOK3, HOXA11, HOXA13, HOXA9, HOXC11, HOXC13, HOXD11, HOXD13, HRAS, HSP90AA1, HSP90AB1, ID3, IDH1, IDH2, IGF2BP2, IKBKB, IKZF1, IL2, IL21R, IL6ST, IL7R, IRF4, IRS4, ISX, ITGAV, ITK, JAK1, JAK2, JAK3, JAZF1, JUN, KAT6A, KAT6B, KAT7, KCNJ5, KDM5A, KDM5C, KDM6A, KDR, KDSR, KEAP1, KIAA1549, KIF5B, KIT, KLF4, KLF6, KLK2, KMT2A, KMT2C, KMT2D, KNL1, KNSTRN, KRAS, KTN1, LARP4B, LASP1, LCK, LCP1, LEF1, LEPROTL1, LHFPL6, LIFR, LMNA, LMO1, LMO2, LPP, LRIG3, LRP1B, LSM14A, LYL1, LZTR1, MAF, MAFB, MALT1, MAML2, MAP2K1, MAP2K2, MAP2K4, MAP3K1, MAP3K13, MAPK1, MAX, MB21D2, MDM2, MDM4, MDS2, MECOM, MED12, MEN1, MET, MGMT, MITF, MKL1, MLF1, MLH1, MLLT1, MLLT10, MLLT11, MLLT3, MLLT6, MN1, MNX1, MPL, MSH2, MSH6, MSI2, MSN, MTCP1, MTOR, MUC1, MUC16, MUC4, MUTYH, MYB, MYC, MYCL, MYCN, MYD88, MYH11, MYH9, MYO5A, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB2, NFKBIE, NIN, NKX2-1, NONO, NOTCH1, NOTCH2, NPM1, NR4A3, NRAS, NRG1, NSD1, NSD2, NSD3, NT5C2, NTHL1, NTRK1, NTRK3, NUMA1, NUP214, NUP98, NUTM1, NUTM2A, NUTM2B, OLIG2, OMD, P2RY8, PABPC1, PAFAH1B2, PALB2, PATZ1, PAX3, PAX5, PAX7, PAX8, PBRM1, PBX1, PCBP1, PCM1, PD-1, PDCD1LG2, PDGFB, PDGFRA, PDGFRB, PDL1, PER1, PHF6, PHOX2B, PICALM, PIK3CA, PIK3CB, PIK3R1, PIM1, PLAG1, PLCG1, PML, PMS1, PMS2, POLD1, POLE, POLG, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKAR1A, PRKCB, PRPF40B, PRRX1, PSIP1, PTCH1, PTEN, PTK6, PTPN11, PTPN13, PTPN6, PTPRB, PTPRC, PTPRD, PTPRK, PTPRT, PWWP2A, QKI, RABEP1, RAC1, RAD17, RAD21, RAD51B, RAF1, RALGDS, RANBP2, RAP1GDS1, RARA, RB1, RBM10, RBM15, RECQL4, REL, RET, RFWD3, RGPD3, RGS7, RHOA, RHOH, RMI2, RNF213, RNF43, ROBO2, ROS1, RPL10, RPL22, RPL5, RPN1, RSPO2, RSPO3, RUNX1, RUNX1T1, S100A7, SALL4, SBDS, SDC4, SDHA, SDHAF2, SDHB, SDHC, SDHD, SEPT5, SEPT6, SEPT9, SET, SETBP1, SETD1B, SETD2, SF3B1, SFPQ, SFRP4, SGK1, SH2B3, SH3GL1, SHTN1, SIRPA, SIX1, SIX2, SKI, SLC34A2, SLC45A3, SMAD2, SMAD3, SMAD4, SMARCA4, SMARCB1, SMARCD1, SMARCE1, SMC1A, SMO, SND1, SNX29, SOCS1, SOX2, SOX21, SOX9, SPECC1, SPEN, SPOP, SRC, SRGAP3, SRSF2, SRSF3, SS18, SS18L1, SSX1, SSX2, SSX4, STAG1, STAG2, STAT3, STAT5B, STAT6, STIL, STK11, STRN, SUFU, SUZ12 SYK, TAF15, TAL1, TAL2, TBL1XR1, TBX3, TCEA1, TCF12, TCF3, TCF7L2, TCL1A, TEC, TERT, TET1, TET2, TFE3, TFEB, TFG, TFPT, TFRC, TGFBR2, THRAP3, TLX1, TLX3, TMEM127, TMPRSS2, TNC, TNFAIP3, TNFRSF14, TNFRSF17, TOP1, TP53, TP63, TPM3, TPM4, TPR, TRAF7, TRIM24, TRIM27, TRIM33, TRIP11, TRRAP, TSC1, TSC2, TSHR, U2AF1, UBR5, USP44, USP6, USP8, VAV1, VHL, VTI1A, WAS, WDCP, WIF1, WNK2, WRN, WT1, WWTR1, XPA, XPC, XPO1, YWHAE, ZBTB16, ZCCHC8, ZEB1, ZFHX3, ZMYM2, ZMYM3, ZNF331, ZNF384, ZNF429, ZNF479, ZNF521, ZNRF3, ZRSR2, or any combination thereof.

### Allergens and Environmental Peptides

In some embodiments, a peptide is from an allergen or the environment. Exemplary sources of peptides include but are not limited to: pollen allergens (tree-, herb, weed-, and grass pollen allergens), insect allergens (inhalant, saliva and venom allergens), animal hair and dandruff allergens, and food allergens.

In some cases, a peptide is from the environment. Exemplary environmental peptides can originate from: pollen allergens from trees, grasses and herbs originate from the taxonomic orders of Fagales, Oleales, Finales and platanaceae including i.a. birch (Betula), alder (Alnus), hazel (Corylus), hombeam (Carpinus) and olive (Olea), cedar Cryptomeriaand Juniper us), Plane tree (Platanus), the order of Poales including i.e. grasses of the genera Lolium, Phleum, Poa, Cynodon, Dactylis, Holcus, Phalaris, Secal, and Sorghum, the orders oi Aster ales and Urticales including i.a. herbs of the genera Ambrosia, Artemisia, and Parietaria. Other allergen antigen peptides that may be used include those that originate from house dust mites of the genus Dermatophagoides and Euroglyphus, storage mite e.g. Lepidoglyphys, Glycyphagus and Tyrophagus, those from cockroaches, midges and fleas, e.g., Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

Exemplary food peptides can originate from cow's milk (e.g., lactose), eggs, nuts, shellfish, fish, and legumes (peanuts and soybeans), fruits and vegetables such as tomatoes.

In some cases, any of the aforementioned peptides can be used in a method provided herein to desensitize a cell, such as an immune cell.

### Peptide Length

In some cases, a peptide or fragment thereof, provided herein, can be of any length of amino acid residues. In an aspect, a peptide presented by a peptide binding domain, for example on an MHC molecule (either class I or class II), is from about 1-3 aa, 2-4, 3-5, 4-6, 5-7, 6-8, 7-9, 8-10, 9-11, 10-12, 11-13, 12-14, 13-15, 14-16, 15-17, 16-18, 17-19, 18-20, 19-21, 20-22, 21-23, 22-24, 23-25, 24-26, 25-27, 26-28, 27-29, 28-30, 29-31, 30-32, or up to about 31-33 aa in length. In an aspect, a peptide or fragment thereof presented by a peptide binding domain, for example on a MHC molecule (either class I or class II), is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or up to about 40 aa in length. In some embodiments, a peptide or fragment thereof can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500 or more amino acid residues in length. In some cases, a peptide or fragment thereof can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length. In some embodiments, a peptide or fragment thereof has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids. In some cases, a peptide or fragment thereof has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids. In some cases, a peptide or fragment thereof is selected from Table 1-Table 3, Table 5, or Table 6 and can comprise any number of residues of SEQ ID NO: 1-SEQ ID NO: 56 or SEQ ID NO: 59-79.

### Peptide Libraries

In some embodiments, a library of peptides is provided herein. The library in particular cases comprises a mixture of peptides (“pepmixes”) that can span part or all of the same antigen peptide. Pepmixes may be from commercially available peptide libraries made up of peptides that are about 7-15 amino acids long and overlap one another. In some cases, they may be generated synthetically. Examples include those from JPT Technologies (Springfield, Va.) or Miltenyi Biotec (Auburn, Calif.). In particular embodiments, the peptides are at least 7, 8, 9, 10, 1 1, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example. The mixture of different peptides may include any ratio of the different peptides, although in some embodiments each particular peptide is present at substantially the same numbers in the mixture as another particular peptide.

### Peptide Binding Domains

In some embodiments, solid supports (e.g., particles) described herein have on their surface a protein that comprises a peptide binding domain. In some embodiments, the solid support comprises a plurality of the peptide binding domains that are the same. In some embodiments, the solid support comprises a plurality of a first peptide binding domain and a plurality of a second peptide binding domain, wherein the first and second peptide binding domains are different. In some embodiments, the solid support comprises a plurality of the peptide binding domains that bind the same peptide. In some embodiments, the solid support comprises a plurality of a first peptide binding domain and a plurality of a second peptide binding domain, wherein the first peptide binding domain binds a first peptide and said second peptide binding domain binds a second peptide, and wherein said first and second peptide are different.

A peptide binding domain can comprise at least a portion of a major histocompatibility complex (MHC). The MHC comprises 3 subgroups based on the structure and function of the encoded proteins: Class I, Class II, and Class III. In humans, the class I region on an MHC allele contains the genes encoding the HLA molecules HLA-A, HLA-B, and HLA-C. These molecules are expressed on the surfaces of almost all immune cells and can play a role in processing and presenting subject peptides or fragments thereof. HLA class I molecules present peptide or fragments thereof to immune cells (CD8+ T cells). Most of these peptides originate from the breakdown of normal cellular proteins (“self”). However, if foreign peptide fragments are presented (e.g., from a viral pathogen), CD8+ T cells can recognize the peptides as “non-self” and can be activated to release inflammatory cytokines and launch an immune response to attack the viral pathogen or foreign body.

Major histocompatibility complex class II (MHC-II) molecules present peptides to e.g., CD4+ T cells. Their expression is typically directed to professional antigen-presenting cells (APCs), including dendritic cells (DCs), monocytes/macrophages and B lymphocytes. APCs are cells that display an antigen peptide complexed with MHC or HLA on their surfaces. T cells may recognize these complexes using their T cell receptors (TCRs). In humans, HLA-DR, HLA-DQ, and HLA-DP molecules are the three classical MHC-II molecules. Both glycoproteins a and R chains of the MHC-II are generated in the endoplasmic reticulum (ER) and are associated with the invariant chain (Ii), a chaperone molecule which can stabilize the conformation of the MHC dimer, avoiding the binding of endogenous peptides in the groove of MHC-II molecules. The activation of naïve CD4⁺ T cells can be originated by the interaction of T Cell Receptors (TCRs) with specific pMHC-II complexes presented by professional APCs. To fully prime CD4⁺ T cells, antigenic signal is reinforced by costimulatory molecule interactions with APCs and by cytokines secreted in the local milieu. The main costimulatory molecule expressed by T cells is CD28, which interacts with CD80 and CD86 on APCs. CD40 molecule on APCs that binds CD40L on activated T cells is also utilized for CD4⁺ T cell responses, at least, in part, by amplifying APC activation.

In some cases, an MHC can be human or non-human. In some cases, an MHC is non-human and can be from a non-human subject, including but not limited to a mouse, rat, guinea pig, swine, cattle, horse, sheep, goat, dog, monkey, cat, chicken, and the like. In some cases, a monkey is a rhesis macaque.

In some cases, an MHC is human and comprises a Human Leukocyte Antigen (HLA). Human MHC class I and class II proteins (human leukocyte antigens, HLAs) are each expressed from three gene regions (MHC class I: HLA-A, —B, —C; MHC class II: HLA-DR, -DP, -DQ). An HLA or a polypeptide thereof can be class I or class II. In some cases, an HLA class I polypeptide includes but is not limited to HLA-A, HLA-B, HLA-C, or can be a non-classical HLA such as HLA-G. In another case, an HLA is HLA-A and includes but is not limited to HLA-A1, HLA-A2, HLA-A3, HLA-A9, HLA-A10, HLA-A11 HLA-A19, HLA-A23, HLA-A24, HLA-A25, HLA-A26, HLA-A28, HLA-A30, HLA-A31, HLA-A32, HLA-A33, HLA-A34 HLA-A36 HLA-A43, HLA-A66, HLA-68, HLA-A69, HLA-A74, or HLA-A80. In another case, an HLA is HLA-B and includes but is not limited to HLA-B5, HLA-B7, HLA-B8, HLA-B12, HLA-B13, HLA-B14, HLA-B15, HLA-B-16, HLA-B17, HLA-B18, HLA-B21, HLA-B22, HLA-B27, HLA-B35, HLA-B37, HLA-B38, HLA-B39, HLA-B40, HLA-B41, HLA-B42, HLA-B46, HLA-B47, HLA-B48, HLA-B49, HLA-B50, HLA-B51, HLA-B52, HLA-B53, HLA-B54, HLA-B55, HLA-B56, HLA-B57, HLA-B58, HLA-B59, HLA-B60, HLA-B61, HLA-B62, HLA-B63, HLA-B64, HLA-B65, HLA-B67, HLA-B70, HLA-B71, HLA-B73, HLA-B75, HLA-B76, HLA-B77, HLA-B78, HLA-B81, HLA-B82, or HLA-B83. In some cases, an HLA is HLA-C and includes but is not limited to HLA-Cw1, HLA-Cw2, HLA-Cw3, HLA-Cw4, HLA-Cw5, HLA-Cw6, HLA-Cw7, HLA-Cw8, HLA-Cw9, or HLA-Cw10.

Any component of the aforementioned MHC and HLA can be utilized in compositions provided herein. For example, MHC class I molecular complexes comprise at least two fusion proteins. A first fusion protein comprises a first MHC class I a chain and a first immunoglobulin heavy chain, and a second fusion protein comprises a second MHC class I a chain and a second immunoglobulin heavy chain. The first and second immunoglobulin heavy chains associate to form the MHC class I molecular complex, which comprises two MHC class I peptide binding clefts. The immunoglobulin heavy chain can be the heavy chain of an IgM, IgD, IgG1, IgG3, IgG2β, IgG2α, IgE, or IgA. In some cases, an IgG heavy chain is used to form MHC class I molecular complexes. If multivalent MHC class I molecular complexes are desired, IgM or IgA heavy chains can be used to provide pentavalent or tetravalent molecules, respectively. MHC class I molecular complexes with other valencies can also be constructed, using multiple immunoglobulin heavy chains.

MHC class II molecular complexes comprise at least four fusion proteins. Two first fusion proteins comprise (i) an immunoglobulin heavy chain and (ii) an extracellular domain of an MHC class 11 chain. Two second fusion proteins comprise (i) an immunoglobulin κ or κ light chain and (ii) an extracellular domain of an MHC class IIα chain. The two first and the two second fusion proteins associate to form the MHC class II molecular complex. The extracellular domain of the MHC class 11 chain of each first fusion protein and the extracellular domain of the MHC class IIα chain of each second fusion protein form an MHC class II peptide binding cleft. The immunoglobulin heavy chain can be the heavy chain of an IgM, IgD, IgG3, IgG1, IgG2β, IgG2α, IgE, or IgA. Preferably, an IgG1 heavy chain is used to form divalent molecular complexes comprising two antigen binding clefts. Optionally, a variable region of the heavy chain can be included. IgM or IgA heavy chains can be used to provide pentavalent or tetravalent molecular complexes, respectively. Molecular complexes with other valencies can also be constructed, using multiple immunoglobulin chains.

Compositions provided herein can comprise fusion proteins of components of MHC or HLA polypeptides or fragments thereof. To generate fusion proteins, MHC or HLA components can comprise linkers. For example, in some cases, a class I or II molecular complex can comprise a peptide linker inserted between an immunoglobulin chain and an extracellular domain of an MHC class I or II polypeptide. The length of the linker sequence can vary, depending upon the flexibility required to regulate the degree of antigen binding and receptor cross-linking. Constructs can also be designed such that the extracellular domains MHC class I or II polypeptides are directly and covalently attached to the immunoglobulin molecules without an additional linker region.

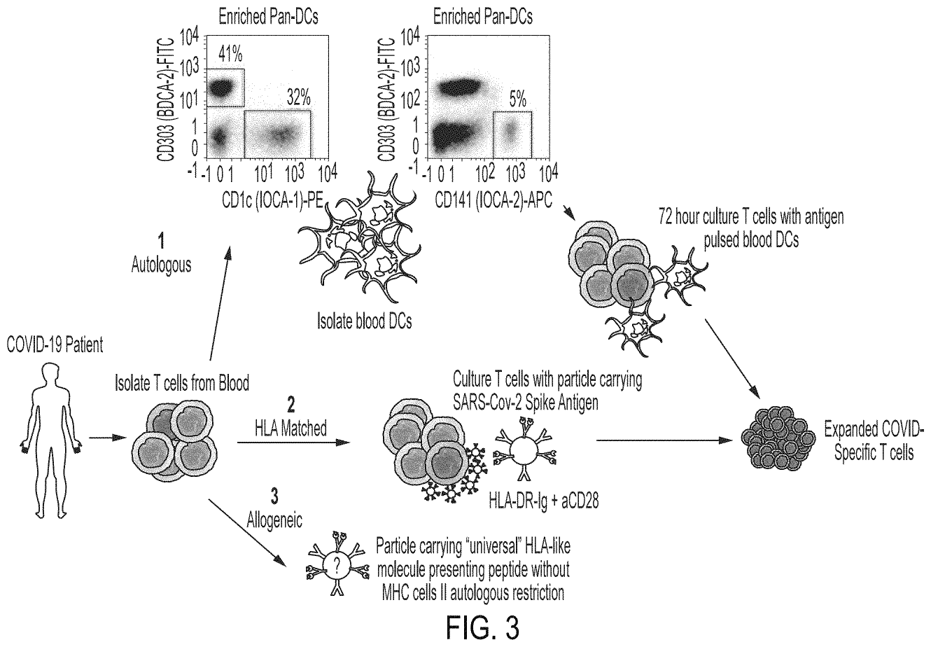

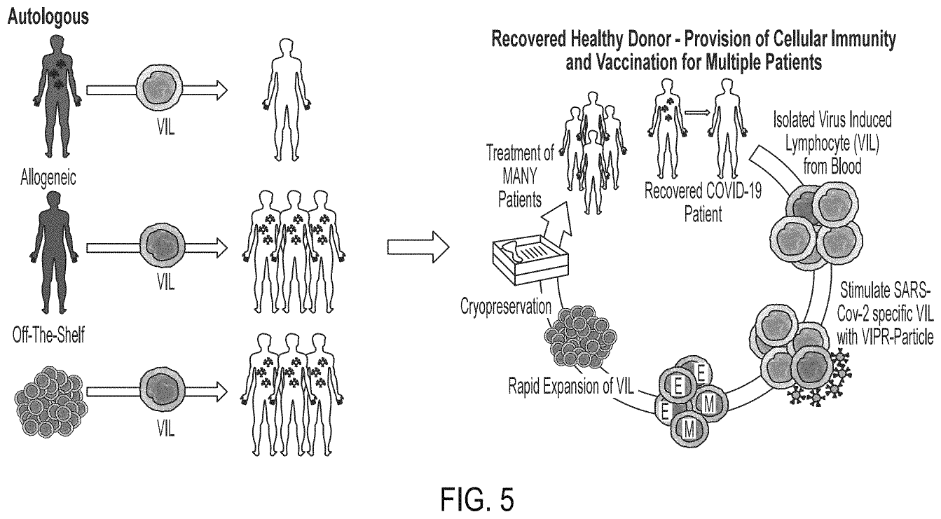

In some cases, a universal peptide binding domain can be utilized. A universal peptide binding domain functions without MHC or HLA restriction and can be utilized in an allogeneic setting. A universal peptide binding domain can also be utilized as an off-the-shelf therapeutic in combination with compositions comprising particles provided herein. Various modalities of treatment are provided herein comprising autologous, allogeneic, and universal techniques, FIG. 3 and FIG. 5.

In some cases, a peptide binding domain comprises a linker. If a linker region is included, this region can contain at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or up to about 30 amino acids. In some cases, the linker is about 5 and not more than 20 amino acids; in some cases, the linker is less than 10 amino acids. Generally, the linker consists of short glycine/serine spacers, but any amino acid can be used. A preferred linker for connecting an immunoglobulin heavy chain to an extracellular domain of an MHC class II β chain is GLY-GLY-GLY-THR-SER-GLY (SEQ ID NO: 57). A preferred linker for connecting an immunoglobulin light chain to an extracellular domain of an MHC class IIα chain is GLY-SER-LEU-GLY-GLY-SER (SEQ ID NO: 58).

In some cases, a composition comprises a first polypeptide that comprises an HLA-A α2 domain and an HLA-A α3 domain, an HLA-B α2 domain and an HLA-B α3 domain, or an HLA-C α2 domain and an HLA-C α3 domain. In some cases, a second polypeptide comprises an HLA-A α1 domain and a β-microglobulin domain, an HLA-B α1 domain and a β-microglobulin domain, or an HLA-C α1 domain and a β-microglobulin domain.

In some cases, an HLA is class II and is HLA-DR, HLA-DQ, or HLA-DP. In some cases, a HLA class II is HLA-DPA1, HLA-DPB1, HLA-DQA1, HLA-DQB1, HLA-DRA, or HLA-DRB1. In some cases, an HLA is HLA-DR and includes but is not limited to HLA-DR1, HLA-DR15, HLA-DR16, HLA-DR3, HLA-DR4, HLA-DR5, HLA-DR6, HLA-DR7, HLA-DR8, HLA-DR9, HLA-DR10, HLA-DR11, HLA-DR12, HLA-DR13, HLA-DR14, HLA-DR51, HLA-DR52, or HLA-DR53. In some cases, an HLA is HLA-DQ and includes but not limited to HLA-DQ1, HLA-DQ2, HLA-DQ3, HLA-DQ4, HLA-DQ5, or HLA-DQ6.

In some cases, a first polypeptide comprises an HLA-DP α1 domain and an HLA-DP α2 domain, an HLA-DQ α1 domain and an HLA-DQ α2 domain, an HLA-DR α1 domain and an HLA-DR α2 domain, or an HLA-DM α1 domain and an HLA-DM α2 domain, or an HLA-DO α1 domain and an HLA-DO α2 domain. In some cases, a second polypeptide comprises an HLA-DP β1 domain and an HLA-DP β2 domain, an HLA-DQ β1 domain and an HLA-DQ β2 domain, an HLA-DR β1 domain and an HLA-DR β2 domain, or an HLA-DM β1 domain and an HLA-DM β2 domain, or an HLA-DO β1 domain and an HLA-DO β2 domain.

In some cases, a peptide binding domain can be an MHC-Ig dimer. In some cases, an MHC-Ig dimer comprises a flexible hinge region. MHC-Ig dimers can provide MHC dimerization which may confer increased MHC/TCR interactions as compared to an MHC monomer. In some cases, a composition comprises an HLA-Class I or Class II Ig or portion thereof. Subject Igs can comprise heavy chains, light chains, and combinations thereof. The immunoglobulin heavy chain can be the heavy chain of an IgM, IgD, IgG3, IgG1, IgG2p, IgG2a,, IgE, or IgA. In some cases, a protein or polypeptide can comprise additional immunoglobulin regions. Exemplary immunoglobulin regions can be a VH, VL, CH, CL, hinge, Fab, Fc, constant region, variable region, antigen binding site, FcR, carbohydrates, and any combination thereof. An immunoglobulin region can comprise a CH3 domain and/or CH2 domain of an immunoglobulin. In some cases, an immunoglobulin region can further comprise a hinge region of an immunoglobulin. In some cases, an immunoglobulin region further comprises a CH1 domain of an immunoglobulin. Additionally, a peptide binding domain of an HLA protein can be fused directly or indirectly to an immunoglobulin region.

In some cases, the length of the linker sequence can vary, depending upon the flexibility required to regulate the degree of peptide binding and cross-linking. Constructs can also be designed such that the extracellular domains of TCR polypeptides are directly and covalently attached to the immunoglobulin molecules without an additional linker region. If a linker region is included, this region can contain at least 3 and not more than about 30 amino acids. In some cases, the linker is about 5 and not more than 20 amino acids; in some cases, the linker is less than 10 amino acids.

### Co-Stimulatory Moiety

Provided herein are co-stimulatory moieties. In some embodiments, a particle described herein comprises a co-stimulatory moiety attached to the surface. In some embodiments, said particle comprises a plurality of co-stimulatory moieties attached to the surface.

In some embodiments, a co-stimulatory moiety comprises a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some cases, a particle comprises at least about 1, 2, 3, 4, 5, 6, 7, 8, 9, 10, or more co-stimulatory moieties.

A particle can express ligands for T cell receptor and/or costimulatory molecules. A particle can be used to activate and expand T cells for adoptive cellular therapy. A particle can be modified to express a protein associated with T cell activation. A particle can be engineered to express any protein associated with T cell expansion.

In some cases, a particle composition provided herein comprises a protein from a costimulatory molecule. A protein from a costimulatory molecule can be selected from the group consisting of CD80 (B7-1), CD86 (B7-2), B7-H3, ICOSL (ICOS), 4-1BBL, CD27, CD30, OX40L (OX40), B7h (B7RP-1), CD40, LIGHT, CD70 (CD27), Tim 3 (Galectin 9), Tim 4 (Tim 1), ICAM (LFA1), CD40 (CD40L), B7 (CD28), HVEM (BTLA or CD160) an antibody that specifically binds to CD28, an antibody that binds CD3, an antibody that specifically binds to HVEM, an antibody that specifically binds to CD40L, an antibody that specifically binds to OX40, and an antibody that specifically binds to 4-1BB.

In some cases, compositions comprising a particle that expresses a peptide and a costimulatory protein, can be used to expand immune cells such as CD4 or CD8 T cells. In some cases, the addition of a co-stimulatory protein increases the proliferation or expansion of a population of cells by at least about 5%, 10%, 20%, 30%, 40%, 50%, 60%, 70%, 80%, 90%, or up to about 100% more than a comparable population that undergoes a contacting step with a particle lacking the co-stimulatory protein. In another case, the proliferation or expansion of a population of cells increases by about 1 fold, 2 fold, 3 fold, 4 fold, 5 fold, 10 fold, 20 fold, 40 fold, 60 fold, 100 fold, 200 fold, 300 fold, 400 fold, or up to about 500 fold more than a comparable population that undergoes a contacting step with a particle lacing the co-stimulatory protein.

### Methods of Activating T Cells Ex Vivo Utilizing an Artificial Antigen Presenting Platform

In some cases, cells can be activated or expanded by co-culturing with an antigen presenting cell. In some cases, an APC is artificial, which is herein referred to as an “artificial antigen presenting platform.” An artificial antigen presenting platform (aAPP) can express ligands for a T cell receptor and a costimulatory receptor. aAPPs can be generated using a variety of means. For example, an aAPP can be engineered to express peptides provided herein and/or costimulatory proteins provided herein. An aAPP can be a bead, a cell, a protein, an antibody, a cytokine, or any combination. An aAPP can deliver signals associated with stimulation and proliferation of immune cells. For example, an aAPP can deliver a signal 1, signal, 2, signal 3 or any combination. A signal 1 can be an antigen recognition signal. For example, signal 1 can be ligation of a TCR by a peptide-MHC complex expressed by an aAPP, binding of agonistic antibodies directed towards CD3 that can lead to activation of the CD3 signal-transduction complex, or both. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof. Cytokines can be expressed by aAPPs or can be added exogenously into a culture media.

In some cases, an artificial antigen presenting platform (aAPP) can be used to activate and/or expand a cell population. In some cases, a K562 cell can be used for expansion. A K562 cell can also be used for stimulation. A K562 cell can be a human erythroleukemic cell line. A K562 cell can be engineered to express proteins described herein. In some cases, K562 cells do not endogenously express HLA class I, II, or CD1d molecules but can express ICAM-1 (CD54) and LFA-3 (CD58). K562 can be engineered to deliver a signal 1 to T cells. For example, K562 cells can be engineered to express HLA class I and/or HLA class II. In some cases, K562 cells can be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

In some cases, an aAPP can be a particle. In some cases, a particle is a bead. In some cases, a bead is a polystyrene bead. A spherical polystyrene bead can be coated with antibodies against CD3 and CD28 and be used for T cell activation. A bead can be utilized at any cell to bead ratio. For example, a 3 to 1 bead to cell ratio at 1 million cells per milliliter can be used. An aAPP can also be a rigid spherical particle, a polystyrene latex microbeads, a magnetic nano-or micro-particles, a nanosized quantum dot, a 4, poly(lactic-co-glycolic acid) (PLGA) microsphere, a nonspherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof.

In some cases, an aAPP can expand CD4 T cells. For example, an aAPP can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells. In some cases, the use of aAPPs can be combined with exogenously introduced cytokines for T cell activation, expansion, or any combination. Cells can also be expanded in vivo, for example in the subject's blood after administration of transplanted cells into a subject. In some cases, a CD4 memory T cell expansion, resulting from contact with an APC such as a particle, aAPP, APC, or dendritic cell, can lead to a “vaccine booster” shot effect in enhancing active immunity in subjects recovering from COVID19 or any viral illness. For example, this vaccine booster can be superior to a passive immunity antibody-mediated convalescent serum strategy. In some cases, the superiority can be from about 1 fold, 3 fold, 5 fold, 7 fold, 9 fold, 13 fold, 15 fold, 17 fold, 19 fold, 21 fold, or about 50 fold greater than a comparable method.

### Peptide-Pulsed Antigen-Presenting Cells and Methods of Activating T Cells Ex Vivo to Generate a Virus Induced Lymphocyte

In some cases, a composition provided herein comprises an antigen presenting cell (APC). Antigen presenting cells include but are not limited to dendritic cells, B lymphocytes, monocytes, macrophages and the like. In some cases, an APC is a Dendritic Cell (DC). DCs are potent activators of T cells and have been shown to be involved in immune responses elicited by a wide array of immunotherapeutic approaches. In some cases, a DC can be utilized as an antigen peptide delivery vehicle.

In some embodiments, a DC is pulsed with a SARS-Cov-2 peptide. A SARS-Cov-2 peptide can be selected from any of the sequences of Table 1-Table 3, Table 5, or Table 6, modified versions thereof, fragments thereof, truncations thereof, portions thereof, derivatives thereof, or any combination thereof. In some cases, a peptide comprises a sequence of a SARS-COV-2 spike glycoprotein. A DC can be modified to express any number of SARS-Cov-2 peptides from Table 1-Table 3, Table 5, or Table 6, modified versions thereof, fragments thereof, truncations thereof, portions thereof, derivatives thereof, or any combination thereof. In some embodiments, at least 2, at least 3, at least 4, at least 5, at least 6, at least 7, or up to about 8 different peptides can be expressed on a DC. In some embodiments, the peptides can comprise overlapping sequences. In some embodiments, the peptides do not have overlapping sequences. In some cases, peptides from Table 1-Table 3, Table 5, or Table 6, modified versions thereof, fragments thereof, truncations thereof, portions thereof, derivatives thereof, or any combination thereof can be modified to increase antigenicity. In some cases, peptides from Table 1 or other peptides provided herein can be modified to be extra-antigenic or sub-antigenic as compared to their wildtype counterpart.

In some cases, a subject peptide can be contacted with an APC, such as a dendritic cell. A subject peptide can be delivered to an APC via gene transduction, endocytosis of particulate preparations, or endocytosis of whole proteins or portions thereof. In some cases, APCs can be incubated directly with preprocessed, synthetic peptides.

In some cases, an APC is a DC. A DC can exist in both immature and mature states. Different states of differentiation are associated with different abilities to process and present peptide antigens. For example, prior to encountering a foreign antigen peptide, dendritic cells express very low levels of MHC class II and co-stimulatory molecules on their cell surface. These immature dendritic cells can be ineffective at presenting antigen to T helper cells. Once a dendritic cell's pattern-recognition receptors recognize a pathogen-associated molecular pattern, peptide antigen is phagocytosed and the dendritic cell becomes activated, upregulating the expression of MHC class II molecules. It also upregulates several co-stimulatory molecules utilized for T cell activation, including CD40 and B7. The latter can interact with CD28 on the surface of a CD4+ T cell. The dendritic cell is then a fully mature professional APC. In some cases, immature dendritic cells are matured to form mature dendritic cells. Mature DC lose the ability to take up antigen and display up-regulated expression of costimulatory cell surface molecules and various cytokines. Specifically, mature DC express higher levels of MHC class I and II antigens than immature dendritic cells, and mature dendritic cells are generally identified as being CD80+, CD83+, CD86+, and CD14−. In some cases, greater MHC expression leads to an increase in antigen density on the DC surface, while up regulation of costimulatory molecules CD80 and CD86 strengthens the T cell activation signal through the counterparts of the costimulatory molecules, such as CD28 on the T cells. In some cases, a DC is not matured. For example, a DC can be isolated from blood as an immature plasmacytoid and/or myeloid DC.

In some cases, a DC is immature. In other cases, a DC is mature. In some cases, a method comprise differentiation a DC. Various methods of differentiating DCs exist. In some cases, a DC can be contacted with a cytokine. A cytokine cocktail that can be utilized to mature a DC can comprise IL-1β, TNF-α, IL-6, GM-CSF, and/or PGE2. Additionally, ex vivo differentiation can involve culturing dendritic cell precursors, or populations of cells having dendritic cell precursors, in the presence of one or more differentiation agents. Suitable differentiating agents can be, for example, cellular growth factors (e.g., cytokines such as (GM-CSF), Interleukin 4 (IL-4), Interleukin 13 (IL-13), and/or combinations thereof). In certain embodiments, the monocytic dendritic cells precursors are differentiated to form monocyte-derived immature dendritic cells.

The dendritic cell precursors can be cultured and differentiated in suitable culture conditions. Suitable tissue culture media include AIM-V®, RPMI 1640, DMEM, X-VIVO 15®, and the like. The tissue culture media can be supplemented with serum, amino acids, vitamins, N-acetyl cysteine (NAC), cytokines, such as GM-CSF and/or IL-4, divalent cations, and the like, to promote differentiation of the cells. In certain embodiments, the dendritic cell precursors can be cultured in the serum-free media. Such culture conditions can optionally exclude any animal-derived products. A typical cytokine combination in a typical dendritic cell culture medium is about 500 units/ml each of GM-CSF (50 ng/ml) and IL-4 (10 ng/ml). Dendritic cell precursors, when differentiated to form immature dendritic cells, are phenotypically similar to skin Langerhans cells. Immature dendritic cells typically are CD14− and CD11c+, express low levels of CD86 and CD83, and are able to capture soluble antigens via specialized endocytosis. In some cases, immature DC can express elevated levels of CD86. In some cases, a DC comprising population can be mixed in terms of CD14 and CD11C expression. In some cases, a majority of cells are CD11c positive. In some cases, there can be a distinct subpopulation that are CD11c negative and CD 14 positive.

In some cases, an APC is a macrophage. A macrophage can be stimulated by T cell secretion of interferon gamma or exogenous interferon gamma. After this activation, macrophages are able to express MHC class II and co-stimulatory molecules, including the B7 complex and can present phagocytosed peptide fragments to helper T cells, thereby stimulating them. An APC can also be a B cell. B cells can internalize antigen that binds to their B cell receptor and present it to helper T cells. Unlike T cells, B cells can recognize soluble antigen for which their B cell receptor is specific. They can then process the antigen and present peptides using MHC class II molecules. When a T helper cell with a TCR specific for that peptide binds, the B cell marker CD40 binds to CD40L on the T cell surface. When activated by a T cell, a B cell can undergo antibody isotype switching, affinity maturation, as well as formation of memory cells. In some cases, any one of a DC, macrophage, or B cell can be peptide pulsed with a subject peptide and utilized to stimulate an immune cell, such as a CD4 (MHC II) or CD8 T cell (MHC I).

Antigen presenting cells can be isolated from any source by any means. In some cases, an APC is a DC that can be isolated from a patient's blood. In some cases, a hematopoietic stem cell is isolated. In some cases, a myeloid lymphoid precursor, granulocyte monocyte precursor, erythrocyte megakaryocyte precursor, granulocyte precursor, monocyte precursor, myeloid-derived DC precursor, monocyte, pre-DC, lymphoid derived DC precursor, b-cell precursor, NK-cell precursor, thymic lymphoid precursor, or any combination thereof are isolated. In some cases, a DC is isolated as an immature plasmacytoid and/or myeloid DC. In some cases, isolating an at least partially differentiated cell can reduce a preparation time by about 1 day, 2 days, 3 days, 4 days, 5 days, 6 days, 7 days, 8 days, 9 days, 10 days, 11 days, 12 days, 13 days, 14 days, or about 15 days.

In some cases, the peptide is contacted to the APC for about 30 minutes, 1 hour, 1.5 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 24 hours, 2 days, 3 days, 4 days, 5 days, 6 days, or up to about 7 days. In some cases, immature DC are typically contacted with effective amounts of a nucleic acid composition and/or an antigen peptide composition for at most, at least, or about 1, 2, 3, 4, 5, 6, 7, 8, 9, 10, 11, 12, to 10, 11, 12, 13, 14, 15, 16, 17, 18, 19, 20, 21, 22, 23, or 24 minutes, hours, or days. The immature dendritic cells can be cultured and matured in suitable maturation culture conditions. Suitable tissue culture media comprise AIM-V®, RPMI 1640, DMEM, X-VIVO 15®, and the like. The tissue culture media can be supplemented with amino acids, vitamins, cytokines, such as GM-CSF, IL-4, IL-10, Flt3L, TLR8L, TNF-α, PGE2, IL-7, N-acetyl cysteine (NAC), divalent cations, and combinations thereof, to promote maturation of the cells.

Any amount of peptide can be utilized during a contacting step with an APC. In some cases, from about 2 μg/ml, 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 60 μg/ml, 70 μg/ml, 80 μg/ml, 90 μg/ml, 100 μg/ml, 150 μg/ml, or up to about 200 μg/ml. Additionally, in some cases, mature dendritic cells can be prepared (i.e., matured) by contacting the immature dendritic cells with effective amounts or concentrations of a nucleic acid composition and an antigen peptide composition. Effective amounts of nucleic acid composition typically range from at most, at least, or about 0.01, 0.1, 1, 5, 10, to 10, 15, 20, 50, 100 ng or mg of nucleic acid per culture dish or per cell, including all values and ranges there between. Effective amounts of tumor antigen composition typically range from at most, at least, or about 0.01, 0.1, 1, 5, 10, to 10, 15, 20, 50, 100 ng or mg of protein per culture dish or per cell. In certain aspects 0.001 ng of tumor antigen/cell to 1 μg of tumor antigen/million cells) can be used. The tumor antigen composition can optionally be heat inactivated or treated (e.g., exposed to protease) prior to contact with dendritic cells. Maturing the immature dendritic cells with a nucleic acid composition and an antigen peptide composition primes the mature dendritic cells for a type 1 (Th-1) response.

In some cases, provided herein is also a method of producing a population of ex vivo peptide-specific T cells. In some cases, a method comprises isolating T cells or T cell precursors from a hematopoietic sample obtained from a subject; isolating dendritic cells or dendritic cell precursors from the blood sample obtained from the subject; contacting the dendritic cells or dendritic cell precursors with at least one peptide to produce peptide antigen-presenting dendritic cells that present at least one antigen-peptide; and contacting the isolated T cells or T cell precursors from with the peptide pulsed antigen-presenting dendritic cells from to produce peptide-antigen-specific T-cells that recognize said at least one peptide; to thereby produce a population of peptide antigen-specific T-cells.

In some cases, a hybrid approach may be utilized whereby immature DC or at least partially immature DCs are pulsed with antigen peptide, contacted with a population of cells that comprises a T cells, and cocultured with a maturing factor. A hybrid approach may be a way to perform in situ maturation to potentially reduce a preparation time. In some cases, a hybrid approach can reduce a preparation time by about 1 day, 2 days, 3 days, 4 days, 5 days, 6 days, 7 days, 8 days, 9 days, 10 days, 11 days, 12 days, 13 days, 14 days, or about 15 days as compared to a comparable method absent the hybrid approach.

In some cases, T cells that are contacted with an APC, aAPP, particle, or any combination thereof are contacted for no more than about 4 days, 96 hours, 80 hours, 72 hours, 48 hours, 36 hours, 24 hours, 18 hours, 12 hours, 10 hours, 9 hours, 8 hours, 7 hours, 6 hours, 5 hours, 4 hours, 3 hours, 2 hours, or 1 hour. In some cases, T cells are contacted and/or cultured with an APC, aAPP, particle or any combination thereof from about 3-72 hours, 3-48 hours, 3-36 hours 3-24 hours, 3-18 hours, 3-12 hours, 3-6 hours, 6-72 hours, 6-48 hours, 6-36 hours 6-24 hours, 6-18 hours, 6-12 hours, 12-72 hours, 12-48 hours, 12-36 hours 12-24 hours, 12-18 hours, 18-72 hours, 18-48 hours, 18-36 hours 18-24 hours, 24-72 hours, 24-48 hours, or 24-36 hours.

In some cases, T cells that are contacted with an APC can be fed during an incubation period. In some cases, T cells are fed daily, every other day, every third day, every fourth day, or every fifth day. In some cases, T cells can also under a restimulation with a subject APC, such as a DC. In some cases, a restimulation occurs at 3 days, 5 days, 7 days, 12 days, 14 days after primary stimulation of peptide-pulsed DC as indicated. Restimulations can be repeated. In some cases, a restimulation is repeated every 2-4 days, 8-10 days, or 5-10 days.

In some cases, a population of cells comprises at least about a 1 fold, 2 fold, 10 fold, 50 fold, 100 fold, 200 fold, 400 fold, 600 fold, 800 fold, 1000 fold, 10000 fold increase in the number of T cells isolated from a sample after a contacting step. For example, a population can comprise at least about 1.0E+07, 2.0E+07, 3.0E+07, 3.5E+07, 4.0E+07, 5.0E+07, 6.0E+07, 7.0E+07, 8.0E+07, 9.0E+07, or 1.0E+08 cells after a contacting step.

In some cases, T cells can be evaluated after being contacted with a subject APC. T cells can be evaluated for functionality, peptide antigen specificity, phenotype, proliferative potential, exhaustion, or any combination thereof. Cells can be evaluated using flow cytometry, ELISA, western blot, microscopic analysis, or any combination thereof. In some cases, supernatants are collected 24-48 h after stimulation for analysis via ELISA. In some cases, T cells are analyzed to determine their induced phenotype and cytokine production. To determine the frequency of peptide-specific CD8+ T cells, an ELISPOT assay can be performed. An ELISPOT assay can be performed from about 3-5 days after every restimulation. In an aspect, a population of cells that is functional via ELISPOT can be utilized in a pharmaceutical composition provided herein to administer to a subject.

In some cases, a method comprises contacting a population of immune cells with an antigen presenting cell, such as a dendritic cell and/or a particle. In some cases, a combination approach whereby both an APC and particle are utilized can stimulate both CD8 and CD4 T cells. For example, inclusion of both DCs and a particle as a combinatorial method for stimulation of both CD8 T cells by MHC-I antigen presenting particles, and CD4 T cells by autologous or allogeneic (HLA-matched) MHC-II antigen presenting DCs can yield a more potent immune activation. In some cases, an off-the-shelf cellular composition is utilized whereby cells are genetically modified to reduce and/or eliminate immunogenicity. In some cases, a combinatorial approach of stimulating an immune cell can increase an immune response by about 1 fold, 2 fold, 3 fold, 4 fold, 5 fold, 6 fold, 7 fold, 8 fold, 9 fold, 10 fold, 20 fold, 30 fold, 40 fold, 50 fold, 100 fold, 150 fold, or up to about 200 fold as compared to a comparable approach whereby a single agent is used, particle alone or APC alone. Relevant immune responses include those described herein such as cytokine production, cytotoxicity, proliferation, expansion, differentiation, stimulation, and the like.

In some cases, a method can also be utilized to generate an anti-peptide immune response in both CD8 and CD4 T cells. For example, a particle can be utilized to stimulate CD8 T cells and an APC can be utilized to stimulate CD4 T cells against a source expressing a target peptide. A therapy provided herein using a particle for MHC Class I peptide presentation and an autologous dendritic cell to stimulate MHC class I and/or MHC class II can generate a viral induced lymphocyte (VIL).

### Genomic Modification

Provided herein can also be a peptide-specific cell that comprises a genomic modification. A genomic modification can be in at least one of an MHC encoding gene, HLA encoding gene, a T cell receptor, or any combination thereof. In some cases, an allogeneic T cell lacks expression of a functional T cell receptor (TCR), a human leukocyte antigen (HLA), e.g., HLA class I and/or HLA class II, and combinations thereof. Genomic modifications can allow for off-the-shelf usage of compositions provided herein.

In embodiments, an agent, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), Argonaut, can be used to introduce a genomic disruption into a cell.

### CRISPR System

In some cases, a CRISPR system can be used to introduce a genomic disruption into a cell. There are at least five types of CRISPR systems which all incorporate RNAs and Cas proteins. Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA. The general mechanism and recent advances of CRISPR system are discussed in Cong, L. et al, “Multiplex genome engineering using CRISPR systems,” Science, 339(6121): 819-823 (2013); Fu, Y. et al., “High-frequency off-target mutagenesis induced by CRISPR-Cas nucleases in human cells,” Nature Biotechnology, 31, 822-826 (2013); Chu, V T et al. “Increasing the efficiency of homology-directed repair for CRISPR-Cas9-induced precise gene editing in mammalian cells,” Nature Biotechnology 33, 543-548 (2015); Shmakov, S. et al, “Discovery and functional characterization of diverse Class 2 CRISPR-Cas systems,” Molecular Cell, 60, 1-13 (2015); Makarova, K S et al, “An updated evolutionary classification of CRISPR-Cas systems,”, Nature Reviews Microbiology, 13, 1-15 (2015). Site-specific cleavage of a target DNA occurs at locations determined by both 1) base-pairing complementarity between the guide RNA and the target DNA (also called a protospacer) and 2) a short motif in the target DNA referred to as the protospacer adjacent motif (PAM). For example, an engineered cell can be generated using a CRISPR system, e.g., a type II CRISPR system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

A CRISPR system can be introduced to a cell or to a population of cells using any means. In some embodiments, a CRISPR system may be introduced by electroporation or nucleofection. Electroporation can be performed for example, using the Neon® Transfection System (ThermoFisher Scientific) or the AMAXA® Nucleofector (AMAXA® Biosystems). Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some embodiments, the electroporation pulse voltage, the electroporation pulse width, number of pulses, cell density, and tip type may be adjusted to optimize transfection efficiency and/or cell viability.

### Cas Protein

In some cases, a vector can be operably linked to an enzyme-coding sequence encoding a CRISPR enzyme, such as a Cas protein (CRISPR-associated protein). In some embodiments, a nuclease or a polypeptide encoding a nuclease is from a CRISPR system (e.g., CRISPR enzyme).

In some embodiments, the CRISPR enzyme directs cleavage of one or both strands at a target sequence. In some embodiments, the CRISPR enzyme mediates cleavage of both strands at a target DNA sequence (e.g., creates a double strand break in a target DNA sequence).

Non-limiting examples of Cas proteins can include Cas1, CaslB, Cas2, Cas3, Cas4, Cas5, Cas6, Cas7, Cas8, Cas9 (also known as Csn1 or Csxl2), CaslO, Csy1, Csy2, Csy3, Cse1, Cse2, Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csxl7, Csxl4, CsxlO, Csxl6, CsaX, Csx3, Csxl, CsxlS, Csf1, Csf2, CsO, Csf4, Cpf 1, c2c1, c2c3, Cas9HiFi, homologues thereof, or modified versions thereof. In some embodiments, a catalytically dead Cas protein can be used (e.g., catalytically dead Cas9 (dCas9)). An unmodified CRISPR enzyme can have DNA cleavage activity, such as Cas9. In some embodiments, a nuclease is Cas9. In some embodiments, a polypeptide encodes Cas9. In some embodiments, a nuclease or a polypeptide encoding a nuclease is catalytically dead. In some embodiments, a nuclease is a catalytically dead Cas9 (dCas9). In some embodiments, a polypeptide encodes a catalytically dead Cas9 (dCas9). A Cas protein can be a high fidelity Cas protein such as Cas9HiFi.

While S. pyogenes Cas9 (SpCas9) is commonly used as a CRISPR endonuclease for genome engineering, it may not be the best endonuclease for every target excision site. For example, the PAM sequence for SpCas9 (5′ NGG 3′) is abundant throughout the human genome, but a NGG sequence may not be positioned correctly to target a desired gene for modification. In some embodiments, a different endonuclease may be used to target certain genomic targets. In some embodiments, synthetic SpCas9-derived variants with non-NGG PAM sequences may be used. Additionally, other Cas9 orthologues from various species have been identified and these “non-SpCas9s” bind a variety of PAM sequences that could also be useful for the present disclosure. For example, the relatively large size of SpCas9 (approximately 4 kb coding sequence) means that plasmids carrying the SpCas9 cDNA may not be efficiently expressed in a cell. Conversely, the coding sequence for Staphylococcus aureus Cas9 (SaCas9) is approximately 1 kilo base shorter than SpCas9, possibly allowing it to be efficiently expressed in a cell. Similar to SpCas9, the SaCas9 endonuclease is capable of modifying target genes in mammalian cells in vitro and in mice in vivo.

Alternatives to S. pyogenes Cas9 may include RNA-guided endonucleases from the Cpf 1 family that display cleavage activity in mammalian cells. Unlike Cas9 nucleases, the result of Cpfl-mediated DNA cleavage is a double-strand break with a short 3′ overhang. Cpfl's staggered cleavage pattern may open up the possibility of directional gene transfer, analogous to traditional restriction enzyme cloning, which may increase the efficiency of gene editing. Like the Cas9 variants and orthologues described above, Cpfl may also expand the number of sites that can be targeted by CRISPR to AT-rich regions or AT-rich genomes that lack the NGG PAM sites favored by SpCas9.

A vector that encodes a CRISPR enzyme comprising one or more nuclear localization sequences (NLSs), such as at least 1, 2, 3, 4, 5, 6, 7, 8, 9, 10, NLSs can be used. For example, a CRISPR enzyme can comprise at least 1, 2, 3, 4, 5, 6, 7, 8, 9, 10, NLSs at or near the ammo-terminus, at least 1, 2, 3, 4, 5, 6, 7, 8, 9, 10, NLSs at or near the carboxyl-terminus, or any combination of these (e.g., one or more NLS at the ammo-terminus and one or more NLS at the carboxyl terminus). When more than one NLS is present, each can be selected independently of others, such that a single NLS can be present in more than one copy and/or in combination with one or more other NLSs present in one or more copies. The NLS can be located anywhere within the polypeptide chain, e.g., near the N- or C-terminus. For example, the NLS can be within or within about 1, 2, 3, 4, 5, 10, 15, 20, 25, 30, 40, 50 amino acids along a polypeptide chain from the N- or C-terminus. Sometimes the NLS can be within or within about 50 amino acids or more, e.g., 100, 200, 300, 400, 500, 600, 700, 800, 900, or 1000 amino acids from the N- or C-terminus.

Any functional concentration of Cas protein can be introduced to a cell. For example, 15 micrograms of Cas mRNA can be introduced to a cell. In other cases, a Cas mRNA can be introduced from 0.5 micrograms to 100 micrograms. A Cas mRNA can be introduced from 0.5, 5, 10, 15, 20, 25, 30, 35, 40, 45, 50, 55, 60, 65, 70, 75, 80, 85, 90, 95, or 100 micrograms.

In some embodiments, a dual nickase approach may be used to introduce a double stranded break or a genomic break. Cas proteins can be mutated at known amino acids within either nuclease domains, thereby deleting activity of one nuclease domain and generating a nickase Cas protein capable of generating a single strand break. A nickase along with two distinct guide RNAs targeting opposite strands may be utilized to generate a double strand break (DSB) within a target site (often referred to as a “double nick” or “dual nickase” CRISPR system). This approach can increase target specificity because it is unlikely that two off-target nicks will be generated within close enough proximity to cause a DSB.

Guiding Polynucleic Acids (gRNA or gDNA)

A guiding polynucleic acid (or a guide polynucleic acid) can be DNA (gDNA) or RNA (gRNA). A guiding polynucleic acid can be single stranded or double stranded. In some embodiments, a guiding polynucleic acid can contain regions of single stranded areas and double stranded areas. A guiding polynucleic acid can also form secondary structures.

In some embodiments, said guide nucleic acid is a gRNA. In some embodiments, said gRNA comprises a guide sequence that specifies a target site and guides an RNA/Cas complex to a specified target DNA for cleavage. Site-specific cleavage of a target DNA occurs at locations determined by both 1) base-pairing complementarity between a gRNA and a target DNA (also called a protospacer) and 2) a short motif in a target DNA referred to as a protospacer adjacent motif (PAM). Similarly, a gRNA can be specific for a target DNA and can form a complex with a nuclease to direct its nucleic acid-cleaving activity.

In some embodiments, said gRNA comprises two RNAs, e.g., CRISPR RNA (crRNA) and transactivating crRNA (tracrRNA). In some embodiments, said gRNA comprises a single-guide RNA (sgRNA) formed by fusion of a portion (e.g., a functional portion) of crRNA and tracrRNA. In some embodiments, said gRNA comprises a dual RNA comprising a crRNA and a tracrRNA. In some embodiments, said gRNA comprises a crRNA and lacks a tracrRNA. In some embodiments, said crRNA hybridizes with a target DNA or protospacer sequence.

In some embodiments, said gRNA targets a nucleic acid sequence of or of about 20 nucleotides. In some embodiments, said gRNA targets a nucleic acid sequence of or of about 5, 10, 15, 16, 17, 18, 19, 20, 21, 22, 23, 24, 25, 30 or more nucleotides. In some embodiments, said gRNA binds a genomic region from about 1 base pair to about 20 base pairs away from a PAM. In some embodiments, said gRNA binds a genomic region from about 1, 2, 3, 4, 5, 6, 7, 8, 9, 10, 11, 12, 13, 14, 15, 16, 17, 18, 19, or up to about 20 base pairs away from a PAM. In some embodiments, said gRNA binds a genomic region within about 1, 2, 3, 4, 5, 6, 7, 8, 9, or 10 base pairs away from a PAM.

A guide RNA can also comprise a dsRNA duplex region that forms a secondary structure. For example, a secondary structure formed by a guide RNA can comprise a stem (or hairpin) and a loop. The length of a loop and a stem can vary. For example, a loop can range from about 3 to about 10 nucleotides in length, and a stem can range from about 6 to about 20 base pairs in length. A stem can comprise one or more bulges of 1 to about 10 nucleotides. The overall length of a second region can range from about 16 to about 60 nucleotides in length. For example, a loop can be or can be about 4 nucleotides in length and a stem can be or can be about 12 base pairs. A dsRNA duplex region can comprise a protein-binding segment that can form a complex with an RNA-binding protein, such as a RNA-guided endonuclease, e.g., Cas protein.

In some embodiments, a Cas protein, such as a Cas9 protein or any derivative thereof, is pre-complexed with a gRNA to form a ribonucleoprotein (RNP) complex. In some embodiments, the RNP complex is introduced into a cell to mediate editing.

In some embodiments, a gRNA is modified. The modifications can comprise chemical alterations, synthetic modifications, nucleotide additions, and/or nucleotide subtractions. The modifications can also enhance CRISPR genome engineering. A modification can alter chirality of a gRNA. In some embodiments, chirality may be uniform or stereopure after a modification. In some embodiments, the modification enhances stability of said gRNA.

In some embodiments, the modification is a chemical modification. A modification can be selected from 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′ deoxyribonucleoside analog pyrimidine, ribonucleoside analog, 2′-O-methyl ribonucleoside analog, and sugar modified analogs, wobble/universal bases, fluorescent dye label, 2′ fluoro RNA, 2′ O-methyl RNA, methylphosphonate, phosphodiester DNA, phosphodiester RNA, phosphothioate DNA, phosphorothioate RNA, UNA, pseudouridine-5′-triphosphate, and 5-methylcytidine-5′-triphosphate, and any combination thereof.

In some embodiments, said modification comprise a phosphorothioate internucleotide linkage. In some embodiments, said gRNA comprises from 1 to 10, 1 to 5, or 1-3 phosphorothioate. In some embodiments, said gRNA comprises 1, 2, 3, 4, 5, 6, 7, 8, 9, 10, 11, 12, 13, 14, 15, 16, 17, 18, 19, or 20 phosphorothioates linkages. In some embodiments, said gRNA comprises phosphorothioate internucleotide linkages at the N terminus, C terminus, or both N terminus and C terminus. For example, in some embodiments, said gRNA comprises phosphorothioates internucleotide linkages between the N terminal 3-5 nucleotides, the C terminal 3-5 nucleotides, or both.

In some embodiments, the modification is a 2′-O-methyl phosphorothioate addition. In some embodiments, said gRNA comprises 1-10, 1-9, 1-8, 1-7, 1-6, 1-5, 1-4, 1-3, or 1-2 2′-O-methyl phosphorothioates. In some embodiments, said gRNA comprises from 1 to 10, 1 to 5, or 1-3 2′-O-methyl phosphorothioates. In some embodiments, said gRNA comprises 1, 2, 3, 4, 5, 6, 7, 8, 9, 10, 11, 12, 13, 14, 15, 16, 17, 18, 19, or 20 2′-O-methyl phosphorothioates. In some embodiments, said gRNA comprises 2′-O-methyl phosphorothioate internucleotide linkages at the N terminus, C terminus, or both N terminus and C terminus. For example, in some embodiments, said gRNA comprises 2′-O-methyl phosphorothioate internucleotide linkages between the N terminal 3-5 nucleotides, the C terminal 3-5 nucleotides, or both.

A gRNA can be introduced at any functional concentration. In some embodiments, 0.5 micrograms to 100 micrograms of said gRNA is introduced into a cell. In some embodiments, 0.5, 5, 10, 15, 20, 25, 30, 35, 40, 45, 50, 55, 60, 65, 70, 75, 80, 85, 90, 95, or 100 micrograms of said gRNA is introduced into a cell.

### Other Endonucleases

Other endonuclease based gene editing systems known in the art can be used to make an engineered cell described herein. For example, zinc finger nuclease systems and TALEN systems.

ZFNs are targeted nucleases comprising a nuclease fused to a zinc finger DNA binding domain. A “zinc finger DNA binding domain” or “ZFBD” is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFN designs and binding data. A “selected” zinc finger domain is a domain not found in nature whose production results primarily from an empirical process such as phage display, interaction trap or hybrid selection. The most recognized example of a ZFN in the art is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-di-residues (RVD). The most recognized example of a TALEN in the art is a fusion polypeptide of the FokI nuclease to a TAL effector DNA binding domain.

Another example of a targeted nuclease that finds use in the methods described herein is a targeted Spoil nuclease, a polypeptide comprising a Spoil polypeptide having nuclease activity fused to a DNA binding domain, e.g., a zinc finger DNA binding domain, a TAL effector DNA binding domain, etc. that has specificity for a DNA sequence of interest. Additional examples of targeted nucleases suitable for the present invention include, but are not limited to Bxbl, phiC31, R4, PhiBTi, and WO/SPBc/TP901-1, whether used individually or in combination.

Any one of the aforementioned methods comprising genomically editing via use of an endonuclease can result in a genomic disruption. The genomic disruption can be sufficient to result in reduced or eliminated protein expression. In some cases, a genomic disruption can also refer to the incorporation of an exogenous transgene into the cellular genome. In such cases, an exogenous transgene can also be detected. The genomic disruption can be detected in at least about 10%, 20%, 30%, 40%, 50%, 60%, 70%, 80%, 90% or 100% of cells tested. Detection can be performed by evaluating the disruption at the genomic level via sequencing, at the mRNA level, or protein level. Suitable methods include PCR, qPCR, flow cytometry, imaging, ELISA, NGS, and any combination thereof. In some cases, protein expression can be reduced by about 1 fold, 2 fold, 3 fold, 5 fold, 10 fold, 20 fold, 30 fold, 50 fold, 70 fold, 100 fold, 125 fold, 150 fold, 200 fold, 250 fold, 300 fold, 350 fold, 500 fold, or up to about 1000 fold as compared to a comparable method that lacks the use of the gene editing, such as with CRISPR.

In some embodiments, compositions and methods provided herein can be used in the autologous setting. In other embodiments, compositions and methods can be used in the allogeneic or xenogeneic setting. For example, compositions that comprise peptide-specific cells can be administered to a diseased subject, a recovered subject, or a healthy subject. In some cases, a subject can be of a different species. In some cases, cells are cryopreserved.

In some embodiments, a peptide-specific cell can be MHC-typed. In some embodiments, a subject can be tissue typed to determine their MHC or HLA type. This allogenic approach can be used to generate a bank of large numbers of cells over a longer expansion duration, during which the cells and a receiving subject can be MHC-typed and matched. Potential recipients can be tissue matched to the same MHC haplotype, thus providing an allogenic platform for the treatment of many patients (one donor, many patients). In some cases, an intended recipient is partially MHC matched. In other cases, an intended recipient is fully MHC matched. In some cases, there is at least about 1 mismatch, 2 mismatches, 3 mismatches, 4 mismatches, 5 mismatches, 6 mismatches, or up to about 10 mismatches. In some cases, an MHC mismatch is class I. In other cases, an MHC mismatch is class II. An MHC mismatch can also be in both MHC I and MHC II.

Various means of tissue typing are known in the art including but not limited to PCR and sequence based typing. Sequence based typing methods include but are not limited to DNA-based HLA typing methods using molecular techniques, such as sequence-specific oligonucleotide probe hybridization (SSOP), sequence-specific primer amplification (SSP), sequencing-based typing (SBT), and reference strand-based conformation analysis (RSCA). A subject can be typed at any time, for example before, during, and/or after administration of a composition, comprising for example peptide-specific cell.

In some cases, cellular growth factors can be utilized in combination with compositions and methods provided herein. Growth factors, as defined herein, are molecules capable of promoting survival, growth and/or differentiation of cells, either in culture or in the intact tissue, through specific effects on a transmembrane receptor. Growth factors can include polypeptides and non-polypeptide factors. Cellular growth factors can contribute to proliferation and/or differentiation of cells. In some cases, the compositions comprising particles can be combined with exogenously introduced cytokines for enhanced T cell activation, expansion, differentiation, or combinations thereof. In some cases, a growth factor can be selected to skew a population of cells towards a particular phenotype. In some cases, a growth factor can be selected to skew a population of cells towards a memory T cells, CD4, CD8, and combinations thereof. Examples of growth factors include cytokines (e.g., interleukins, interferons), lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha, TGF-beta, TGF-beta1, TGF-beta2, and TGF-beta3; insulin-like growth factor-I and —II; erythropoietin (EPO); Flt-3L; stem cell factor (SCF); osteoinductive factors; interferons (IFNs) such as IFN-α, IFN-β, IFN-γ; colony stimulating factors (CSFs) such as macrophage-CSF (M-CSF); granulocyte-macrophage-CSF (GM-CSF); granulocyte-CSF (G-CSF); macrophage stimulating factor (MSP); interleukins (ILs) such as IL-1, IL-1a, IL-1b, IL-1RA, IL-18, IL-2, IL-3, IL-4, IL-5, IL-6, IL-7, IL-8, IL-9, IL-10, IL-11, IL-12, IL-13, IL-14, IL-15, IL-16, IL-17, IL-20; a tumor necrosis factor such as CD154, LT-beta, TNF-alpha, TNF-beta, 4-1BBL, APRIL, CD70, CD153, CD178, GITRL, LIGHT, OX40L, TALL-1, TRAIL, TWEAK, TRANCE; and other polypeptide factors including LIF, oncostatin M (OSM) and kit ligand (KL). Cytokine receptors refer to the receptor proteins which bind cytokines. Cytokine receptors may be both membrane-bound and soluble. In some cases, a subject particle can express a cytokine.

In some cases, compositions provided herein comprise additional proteins added thereto. A cytokine can be any cytokine. A cytokine can be IL-2, IL-7, IL-12, IL-15, IL-21, or any combination thereof. In some cases, IL-2 can be utilized to further stimulate immune cells either before, during, after, or any combination thereof of a contacting step with a subject particle and/or APC. In some cases, a cytokine cocktail can be IL-2, IL-7, and IL-15. A concentration of a cytokine can be about 6000 IU/mL. A concentration of a cytokine can also be about 100 IU/mL, 200 IU/mL, 300 IU/mL, 400 IU/mL, 500 IU/mL, 600 IU/mL, 700 IU/mL, 800 IU/mL, 900 IU/mL, 1000 IU/mL, 2000 IU/mL, 3000 IU/mL, 4000 IU/mL, 5000 IU/mL, 6000 IU/mL, 7000 IU/mL, 8000 IU/mL, 9000 IU/mL, or up to about 10000 IU/mL. In some cases, a cytokine is IL-2 and is utilized in the range of 600-10000 IU/mL. In some variations of the methods, an incubation of cells with a growth factor is carried out at about 37° C. for about 10 min, 15 min, 20 min, 25 min, 30 min, 35 min, 40 min, 45 min, 50 min, 55 min, 1 hr., 5 hrs. 10 hrs., 15 hrs., 20 hrs., 1 day, 2 days, 3 days, 4 days, 5 days, 6 days, or up to about 7 days.

Contacting the cells with a composition of the can occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors, such as those previously described, to which the cells are responsive. In some cases, a media is preconditioned. Preconditioned media can comprise supernatant from a second cellular culture, for example a cellular culture from a healthy subject. In some cases, supernatant taken from a T cell culture after several days can contain secreted cytokines and/or growth factors that can be used to supplement fresh media during the activation of virus-specific T cells from a subject. This initial presence of growth factors, prior to the production of these from the subject's cells may help to speed up the initial entry of these cells into proliferation.

In some cases, cells can be scaled up to yields achieved by standard rapid expansion protocols (REP). In some cases, an average fold expansion can be from 500 to 2000. An average fold expansion of genetically modified TILs can be from 500 to 600, from 600 to 700, from 700 to 800, from 800 to 900, from 900 to 1000, from 1000 to up to 2000 fold. A REP can be performed in any suitable containment system for example a bag, a flask, a plate, or G-Rex flask.

In some cases, provided is a method of enriching antigen-peptide specific T cells in a polyclonal T cell population. In various embodiments, the present compositions and methods provide for about 100-10,000 fold expansion, or more, of T cells. For example, cells can comprise from about 100, or about 300, or about 500, or about 700, or about 1000, or about 2000, or about 3000, or about 4000, or about 5000, or about 7500, or about 10000 fold expansion in the span of, for example, less than about one month, or less than about three weeks, or less than about two weeks, or less than about one week.

In some cases, cellular compositions can undergo pre-infusion testing prior to an administration or concurrent with an administration. Pre-infusion or pre-administration testing can be performed to ensure a cellular product is functional, sterile, and capable of functioning post-infusion. Pre-infusion testing can comprise determining a phenotype, cytotoxicity, memory/stemness, exhaustion, bone marrow migration, ELISA, and any combination thereof. In an aspect, a pre-administration testing can comprise performing an in vitro or an in vivo assay. In an aspect, a level of cytotoxicity may be determined in a population of engineered cells. For example, a population of cells can be evaluated by FACs for expression of any one of: CD3, CD4, CD8, CD45RO, CCR7, CD45RA, CD62L (L-selectin), CD27, CD28, and IL-7Ra, CD95, IL-2RP, CXCR3, and LFA-1. In an aspect, functional testing can also comprise a co-culture assay, cytotoxicity assay, ELISA (for example to quantify interleukin-2 (IL-2), and/or IFN-γ section), or ELISPOT assays.

In some embodiments, cellular activity of cells generated by subject methods can be determined. In an aspect, cellular activity comprises cytokine release by the cells. In some embodiments, the cellular activity comprises release of intercellular molecules, metabolites, chemical compounds or combinations thereof. Cytokine release by the immune cell can comprise the release of IL-1, IL-2, IL-4, IL-5, IL-6, IL-13, IL-17, IL-21, IL-22, IFNγ, TNFα, CSF, TGFβ, granzyme, and the like. In some embodiments, cytokine release may be quantified using ELISA, flow cytometry, western blot, and the like. In some embodiments, the cells generated using methods provided herein can generate from about 1 fold, 2 fold, 3 fold, 4 fold, 5 fold, 6 fold, 7 fold, 8 fold, 9 fold, 10 fold, 11 fold, 12 fold, 13 fold, 14, fold 15 fold, 20 fold, 30 fold, 40 fold, 50 fold, 60 fold, 70 fold, 80 fold, 90 fold, 100 fold, 150 fold, 200 fold, 250 fold, or over 300 fold more cytokine in response to contact with a peptide as compared to a comparable cell absent contacting. In some embodiments, cells generated using methods and compositions provided herein can generate from about 1 fold, 2 fold, 3 fold, 4 fold, 5 fold, 6 fold, 7 fold, 8 fold, 9 fold, 10 fold, 11 fold, 12 fold, 13 fold, 14, fold 15 fold, 20 fold, 30 fold, 40 fold, 50 fold, 60 fold, 70 fold, 80 fold, 90 fold, 100 fold, 150 fold, 200 fold, 250 fold, or over 300 fold more cytokine in response to contact with a peptide as compared to a comparable cell absent the contacting. In some embodiments, cytokine release can be quantified, in vitro or in vivo.

In some embodiments, activity of subject cells comprises cytotoxicity of a target pathogen, such as a virus. In an aspect, a cell or population cells can induce death of a target cell or pathogen. Killing of a target can be useful for a variety of applications, including, but not limited to, treating a disease or disorder in which a cell population or virus is desired to be eliminated or its proliferation desired to be inhibited. Cytotoxicity can refer to the killing of the target. Cytotoxicity can also refer to the release of cytotoxic cytokines, for example IFNγ or granzyme, by subject cells. In some embodiments, cytotoxicity can be quantified by a cytotoxicity assay including, a co-culture assay, ELISPOT, chromium release cytotoxicity assay, and the like. In some embodiments, cells provided herein can kill from about 5%, 10%, 15%, 20%, 25%, 30%, 35%, 40%, 45%, 50%, 55%, 60%, 65%, 70%, 75%, 80%, 85%, 90%, 95%, or up to about 100% targets after contacting with a subject particle or antigen presenting cell as compared to a control cell absent the contacting. In some embodiments, cytotoxicity can be determined in vitro or in vivo.

Provided herein can also be a method comprising prophylactic therapy. In some cases, a subject may be administered a composition to boost CD4 memory T cell expansion, CD8 cytolytic T cell expansion, or combinations thereof before, during, and/or after an infection, for instance a viral infection. For example, CD4 memory expansion can lead to a “vaccine booster” shot effect in enhancing active immunity in subjects recovering from a viral infection, such as COVID19, superior to a passive immunity antibody-mediated convalescent serum therapeutic strategy or a strategy that lacks prophylactic therapy.

### Pharmaceutical Compositions

Compositions and methods provided herein can utilize pharmaceutical compositions. In various embodiments of the aspects herein, methods of the disclosure are performed in a subject. A subject can be a human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

In some cases, the methods of the present disclosure may comprise obtaining one or more cells from a subject. In an aspect, cells can be primary cells. Primary cells can be primary lymphocytes. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. For example, any T cell lines can be used. Alternatively, the cell can be derived from a healthy donor, from a patient diagnosed with cancer, or from a patient diagnosed with an infection, such as a viral infection. In another case, cells can be part of a mixed population of cells which present different phenotypic characteristics. A cell can also be obtained from a cell therapy bank. A selection can include at least one of: magnetic separation, flow cytometric selection, antibiotic selection. Cells can be any blood cells, such as peripheral blood mononuclear cell (PBMC), lymphocytes, monocytes or macrophages. In some cases, an apheresis can be a leukapheresis. Leukapheresis can be a procedure in which blood cells are isolated from blood. During a leukapheresis, blood can be removed from a needle in an arm of a subject, circulated through a machine that divides whole blood into red cells, plasma and lymphocytes, and then the plasma and red cells are returned to the subject through a needle in the other arm. In some cases, cells are isolated after an administration of a treatment regime and cellular therapy. For example, an apheresis can be performed in sequence or concurrent with a cellular administration. In some cases, an apheresis is performed prior to and up to about 6 weeks following administration of a cellular product. In some cases, an apheresis is performed −3 weeks, −2 weeks, −1 week, 0, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, or up to about 10 years after an administration of a cellular product. In some cases, cells acquired by an apheresis can undergo testing for specific lysis, cytokine release, metabolomics studies, bioenergetics studies, intracellular FACs of cytokine production, ELISA-spot assays, and lymphocyte subset analysis. In some cases, samples of cellular products or apheresis products can be cryopreserved for retrospective analysis of infused cell phenotype and function.

An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Suitable cells include primary cells. In some cases, human cells, In other cases, nonhuman cells. Cells can be immune cells. Cells can be stem cells. Non-limiting examples of cells include, but are not limited to, lymphoid cells, such as B cell, T cell (Cytotoxic T cell, Natural Killer T cell, Regulatory T cell, T helper cell), alpha beta T cells, gamma delta T cells, Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells, and any combination thereof.

In some cases, an immune cell comprises a lymphocyte. In some embodiments, the lymphocyte is a natural killer cell (NK cell). In some embodiments, the lymphocyte is a T cell. In some cases, an immune cell is a dendritic cell. T cells can be obtained from a number of sources, including peripheral blood mononuclear cells, bone marrow, lymph node tissue, spleen tissue, umbilical cord, and tumors. In some embodiments, any number of T cell lines available can be used. Immune cells such as lymphocytes (e.g., cytotoxic lymphocytes) can preferably be autologous cells, although heterologous cells can also be used. T cells can be obtained from a unit of blood collected from a subject using any number of techniques, such as Ficoll separation. Cells from the circulating blood of an individual can be obtained by apheresis or leukapheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media, such as phosphate buffered saline (PBS), for subsequent processing steps. After washing, the cells can be resuspended in a variety of biocompatible buffers, such as Ca-free, Mg-free PBS. Alternatively, the undesirable components of the apheresis sample can be removed, and the cells directly resuspended in culture media. Samples can be provided directly by the subject, or indirectly through one or more intermediaries, such as a sample collection service provider or a medical provider (e.g. a physician or nurse). In some embodiments, isolating T cells from peripheral blood leukocytes can include lysing the red blood cells and separating peripheral blood leukocytes from monocytes by, for example, centrifugation through, e.g., a PERCOL™ gradient.

A specific subpopulation of T cells, such as CD4+ or CD8+ T cells, can be further isolated by positive or negative selection techniques. Negative selection of a T cell population can be accomplished, for example, with a combination of antibodies directed to surface markers unique to the cells negatively selected. One suitable technique includes cell sorting via negative magnetic immunoadherence, which utilizes a cocktail of monoclonal antibodies directed to cell surface markers present on the cells negatively selected. For example, to isolate CD4+ cells, a monoclonal antibody cocktail can include antibodies to CD14, CD20, CD11b, CD16, HLA-DR, and CD8. The process of negative selection can be used to produce a desired T cell population that is primarily homogeneous. In some embodiments, a composition comprises a mixture of two or more (e.g. 2, 3, 4, 5, or more) different kind of T-cells.

In some cases, a cell may be a cell that is positive or negative for a given factor. In some cases, a cell may be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin-cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1positive cell, a lectin-like receptor G1 negative cell, or an differentiated or de-differentiated cell thereof. In some cases, a cell can be any immune cells including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells. The T cells can also be selected from a bulk population, for example, selecting T cells from whole blood. The T cells can also be expanded from a bulk population. The T cells can also be skewed towards particular populations and phenotypes. For example, the T cells can be skewed to phenotypically comprise, CD45RO (−), CCR7(+), CD45RA (+), CD62L (+), CD27(+), CD28(+) and/or IL-7Ra (+). Suitable cells can be selected that comprise one of more markers selected from a list comprising: CD45RO (−), CCR7(+), CD45RA (+), CD62L (+), CD27(+), CD28(+) and/or IL-7Ra (+). The examples of factors expressed by cells is not intended to be limiting, and a person having skill in the art will appreciate that a cell may be positive or negative for any factor known in the art. In some cases, a cell may be positive for two or more factors. For example, a cell may be CD4+ and CD8+. In some cases, a cell may be negative for two or more factors. For example, a cell may be CD25−, CD44−, and CD69−. In some cases, a cell may be positive for one or more factors, and negative for one or more factors. For example, a cell may be CD4+ and CD8−.

In some cases, a cell can be a stem cell. Stem cells can give rise to a variety of somatic cells and thus have in principle the potential to serve as an endless supply of therapeutic cells of virtually any type. The re-programmability of stem cells also allows for additional engineering to enhance the therapeutic value of the reprogrammed cell. In any of the methods of the present disclosure, one or more cells may be derived from a stem cell. Non-limiting examples of stem cells include embryonic stem cells, adult stem cells, tissue-specific stem cells, neural stem cells, allogenic stem cells, totipotent stem cells, multipotent stem cells, pluripotent stem cells, induced pluripotent stem cells, hematopoietic stem cells, epidermal stem cells, umbilical cord stem cells, epithelial stem cells, or adipose-derived stem cells. In one example, a cell may be hematopoietic stem cell-derived lymphoid progenitor cells. In another example, a cell may be embryonic stem cell-derived T cell. In yet another example, a cell may be an induced pluripotent stem cell (iPSC)-derived T cell.

In some cases, an iPSC cell can be differentiated into a T cell. Various methods of differentiation are known and can be employed in methods provided herein. Exemplary methods can be found in Vizcardo R, Masuda K, Yamada D, et al. Regeneration of human tumor antigen-specific T cells from iPSCs derived from mature CD8(+) T cells. Cell Stem Cell. 2013; 12(1):31− 36 and/or Themeli M, Kloss C C, Ciriello G, et al. Generation of tumor-targeted human T lymphocytes from induced pluripotent stem cells for cancer therapy. Nat Biotechnol. 2013; 31(10):928-933.

In some cases, an immune cell is a member of an enriched population of cells. One or more desired cell types can be enriched by any suitable method, non-limiting examples of which include treating a population of cells to trigger expansion and/or differentiation to a desired cell type, treatment to stop the growth of undesired cell type(s), treatment to kill or lyse undesired cell type(s), purification of a desired cell type (e.g. purification on an affinity column to retain desired or undesired cell types on the basis of one or more cell surface markers). In some embodiments, the enriched population of cells is a population of cells enriched in cytotoxic lymphocytes selected from cytotoxic T cells (also variously known as cytotoxic T lymphocytes, CTLs, T killer cells, cytolytic T cells, CD8+ T cells, and killer T cells), natural killer (NK) cells, and lymphokine-activated killer (LAK) cells.

In some cases, a pharmaceutical composition comprises a population of coronavirus specific human T cells that comprises: a population of ex vivo differentiated coronavirus specific effector T cells that specifically bind a coronavirus peptide presented by an human leukocyte antigen (HLA) protein; and a population of ex vivo differentiated coronavirus specific memory T cells that specifically bind said coronavirus peptide presented by said human leukocyte antigen (HLA) protein. In some cases, the coronavirus peptide is a portion of a coronavirus spike (S) or nucleocapsid (NP) protein. In some cases, effector T cells and memory T cells are expanded ex vivo or in vivo.

In some cases, a pharmaceutical composition comprises a virus induced lymphocyte.

A cell utilized in methods and compositions can be a primary cell. In some cases, a cell is expanded, for example, cultures of primary cells can be passaged 0 times, 1 time, 2 times, 4 times, 5 times, 10 times, 15 times or more. Cells can be unicellular organisms. Cells can be grown in culture.

Conditions appropriate for T cell culture can include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-21, IL-15, TGF beta, and TNF alpha or any other additives for the growth of cells. Other additives for the growth of cells include, but are not limited to, surfactant, plasmanate, and reducing agents such as N-acetyl-cysteine and 2− mercaptoethanol. Media can include RPMI 1640, A1 M-V, DMEM, MEM, α-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some cases, an 865 mL bottle of RPMI may have 100 mL of human serum, 25 mL of Hepes 1M, 10 mL of Penicillin/streptomycin at 10,000U/mL and 10,000 μg/mL, and 0.2 mL of gentamycin at 50 mg/mL. After addition of additives an RPMI media may be filtered using a 0.2 μm×1L filter and stored at 4° C. In some embodiments, antibiotics, e.g., penicillin and streptomycin, are included only in experimental cultures but not in cultures of cells that are to be infused into a subject. In some cases, human serum can be thawed in a 37° C. water bath, and then heat inactivated (e.g., at 56° C. for 30 min for 100 mL bottle). The sera can be filtered through a 0.8 μm and 0.45 μm filter prior to addition of medium. In some cases, a sera free media can be utilized.

Cells can be maintained under conditions necessary to support growth; for example, an appropriate temperature (e.g., 37° C.) and atmosphere (e.g., air plus 5% CO2). In some instances, T cells that have been exposed to varied stimulation times may exhibit different characteristics. In some cases, a soluble or immobilized antibody against human CD3, CD28, CD2, or any combination thereof may be used during a culturing step.

Cellular compositions described herein can be cryopreserved. A cryopreservation can be performed in, for example, a Cryostor CS10 at 5% DMSO final concentration. A cryopreservation can be at a freeze density from about 7.5×10⁷ cells/mL to about 1.5×10⁸ cells/mL. A freeze density can be from about 1×10⁷ cells/mL, 1.5×10⁷ cells/mL, 2×10⁷ cells/mL, 2.5×10⁷ cells/mL, 3×10⁷ cells/mL, 3.5×10⁷ cells/mL, 4×10⁷ cells/mL, 4.5×10⁷ cells/mL, 5×10⁷ cells/mL, 5.5×10⁷ cells/mL, 6×10⁷ cells/mL, 6.5×10⁷ cells/mL, 7×10⁷ cells/mL, 7.5×10⁷ cells/mL, 8×10⁷ cells/mL, 8.5×10⁷ cells/mL, 9×10⁷ cells/mL, 9.5×10⁷ cells/mL, 1×10⁸ cells/mL, 1.5×10⁸ cells/mL, 2×10⁸ cells/mL, 2.5×10⁸ cells/mL, 3×10⁸ cells/mL, 3.5×10⁸ cells/mL, 4×10⁸ cells/mL, 4.5×10⁸ cells/mL, 5×10⁸ cells/mL, 5.5×10⁸ cells/mL, 6×10⁸ cells/mL, 6.5×10⁸ cells/mL, 7×10⁸ cells/mL, 7.5×10⁸ cells/mL, or up to about 8×10⁸ cells/mL.

### Methods of Treatment

Compositions described herein can be administered before, during, or after the occurrence of a disease or condition, and the timing of administering the composition containing a compound can vary. For example, the pharmaceutical compositions can be used as a prophylactic and can be administered continuously to subjects with a propensity to conditions or diseases in order to prevent the occurrence of the disease or condition. The pharmaceutical compositions can be administered to a subject during or as soon as possible after the onset of the symptoms. The administration of the molecules can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A composition can be administered as soon as is practicable after the onset of a disease or condition is detected or suspected, and for a length of time necessary for the treatment of the disease, such as, for example, from about 1 month to about 3 months. The length of treatment can vary for each subject.

Pharmaceutical compositions containing cells described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The molecules can be packed together or separately, in a single package or in a plurality of packages. One or all of the therapeutic agents can be given in multiple doses. If not simultaneous, the timing between the multiple doses may vary to as much as about a month.

In some aspects, a condition is a viral infection. In some cases, the viral infection is from a coronavirus. In some cases, the coronavirus is SARS-Cov-2. A subject can be positive for COVID-19 or suspected of being positive. On average, it can take from about 5-6 days from infection to the time symptoms present. In some cases, an asymptomatic period can extend for at least 1 week, at least 2 weeks, at least 3 weeks, or in some cases a subject may never present symptoms or have undetected symptoms. In some cases, a subject comprises symptoms of COVID-19 such as fever, fatigue, dry cough, aches, pain, nasal congestion, runny nose, sore throat, diarrhea, breathing difficulties, shortness of breath, confusion, inability to arouse, bluish lips or face, tremors, cytokine release syndrome, organ failure, to name a few.

In some cases, a subject can be tested for presence or absence of a disease or condition provided herein. In some cases, a blood tests and/or cultures may be done. Polymerase chain reaction (PCR) techniques may be used to make many copies of the viral genetic material. Blood may also be tested for antigens, which are proteins on or in viruses that trigger the body's defense. Blood may also be tested for antibodies to viruses. In some cases, a sample of blood or other tissues can be examined with an electron microscope. Exemplary testing platforms can include: antibody tests, viral antigen detection test, viral or bacterial culture, viral DNA or RNA detection, host antibody detection, hemagglutination assay, sequencing, RT-PCR, ELISA, serology, electron microscopy, immunofluorescence, immunoperoxidase, and the like. Different types of samples are used for a viral test, including blood, urine, stool (feces), organ tissue, spinal fluid, and saliva. The type of sample used for the test depends on the type of infection that may be present.

In some cases, a subject undergoes testing for a viral infection. In some cases, a method comprises testing for COVID-19. Testing for COVID-19 can be performed by reverse transcription polymerase chain reaction (RT-PCR). Suitable samples for testing comprise nasopharynx, throat, stool, and blood. In some cases, detection of SARS-CoV-2 RNA in blood may be a marker of severe illness.

In some cases, a subject with COVID-19 presents with lymphopenia, neutrophilia, elevated serum alanine aminotransferase and aspartate aminotransferase levels, elevated lactate dehydrogenase, high CRP, and high ferritin levels. In some cases, elevated D-dimer and lymphopenia can be associated with mortality. In some cases, a subject with critical illness may have high plasma levels of inflammatory makers. In some cases, compositions provided herein can be combined with supportive management of the most common complications of severe COVID-19 including but not limited to pneumonia, hypoxemic respiratory failure/ARDS, sepsis and septic shock, cardiomyopathy and arrhythmia, acute kidney injury, and complications from prolonged hospitalization including secondary bacterial infections, thromboembolism, gastrointestinal bleeding, and critical illness polyneuropathy/myopathy.

In some cases, an immunity assay can also be performed in a recovered or recovering subject. A person who has recently been infected by a virus will produce antibodies in their bloodstream that specifically recognize that virus, known as humoral immunity. Two types of antibodies are detected. The first called IgM is highly effective at neutralizing viruses but is only produced by the cells of the immune system for a few weeks. The second, called, IgG is produced indefinitely. Therefore, the presence of IgM in the blood of the host is used to test for acute infection, whereas IgG indicates an infection sometime in the past. Both types of antibodies can be measured when tests for immunity are performed.

Antibody testing has become widely available. It can be done for individual viruses (e.g. using an ELISA assay) but in automated panels that can screen for many viruses at once are becoming increasingly common. Antibody responses can be evaluated against any component of a pathogen. In some cases, when the pathogen comprises SARS-Cov-2 an antibody response can be evaluated against any one of: the receptor binding domain (RBD), the prefusion S ectodomain (S), and the nucleocapsid (N), and combinations thereof. Additionally, the presence of various immune responses can be evaluated such as antibody-dependent complement deposition (ADCD), antibody-dependent cellular phagocytosis (ADCP), antibody-dependent neutrophil phagocytosis (ADNP), and antibody-dependent NK cell degranulation (NK CD107a) and cytokine secretion (NK MIP1β, NK IFNγ).

One application of any of the compositions or methods provided herein can be to temper immunogenicity associated with gene therapy vectors. WT AAV genomes can persist for years in host cells, either episomally or integrated within the host DNA, and be reactivated by a helper virus or a genotoxic reagent. Seroprevalence studies have indicated that initial exposure to WT AAV often occurs early during childhood, when humoral and cellular immune responses directed against the AAV capsid might be mounted. As such, memory AAV-specific T and B cells might persist lifelong and be recalled upon rAAV-mediated gene transfer. To address these concerns, compositions and methods provided herein can be utilized to expand AAV-specific CD4+ regulatory T cells that express the transcription factor Foxp3+ (Tregs) from the blood of treated subjects. This could offer a method by which Tregs can be either be isolated, expanded, or induced through ex vivo cytokine culture to then provide a modality to suppress the AAV-mediated immune response taking place in vivo in response to the gene therapy vector.

Suitable gene therapy vectors are not limited to AAV but can also comprise those from any one of: adenoviruses, alphaviruses, flaviviruses, herpes simplex viruses (HSV), measles viruses, rhabdoviruses, retroviruses, lentiviruses, Newcastle disease virus (NDV), and/or poxviruses. In some cases, a gene therapy vector is from AAV. In the case that a gene therapy vector is from AAV, an exemplary vector can be of a serotype selected from the group consisting of: AAV1, AAV2, AAV3, AAV4, AAV5, AAV6, AAV7, AAV8, AAV9, AAV10, AAV11, AAV 12, and any combination thereof.

In some cases, use of any of the compositions or methods provided herein to temper immunogenicity to a gene therapy vector, can result in reduced or absent immunogenicity to a gene therapy vector by at least about 1 fold, 2 fold, 3 fold, 5 fold, 10 fold, 20 fold, 40 fold, 60 fold, 80 fold, 100 fold, 150 fold, 200 fold, 300 fold, 500 fold, 1000 fold, or up to about 3000 fold, In some cases, use of any of the compositions or methods provided herein allows for repeated administration of gene therapy vectors. Any number of repeated administrations can be performed, including but not limited to 2, 3, 4, 5, 6, 7, 8, 9, or up to about 10 administrations. More than 10 administrations can be performed and can be repeated for the life of a subject at various frequencies such as daily, weekly, bimonthly, monthly, semi-annually, or yearly.

Compositions provided herein can be administered by a route of administration selected from the group consisting of intravenous administration, intra-arterial administration, subcutaneous administration, intradermal administration, intralymphatic administration, and intra-tumoral administration. In some cases, multiple routes of administration are utilized.

In some embodiments, administering comprises infusing a cellular composition comprising from about 1×10²/kg body weight. In some embodiments, the administering comprises infusing from about 1×10³/kg body weight. In some embodiments, the administering comprises infusing from about 1×10⁴/kg body weight. In some aspects, an administering comprises infusing from about 1×10⁵/kg body weight. In some aspects, an administering comprises infusing from about 3×10⁵/kg body weight. In some aspects, an administering comprises infusing from about 1×10⁵/kg body weight to about 3×10⁵/kg body weight. In some aspects, an administering comprises infusing from about 0.5×10⁵/kg body weight to about 1×10⁵/kg body weight. In some aspects, an administering comprises infusing from about 1×10⁴/kg body weight to about 4×10⁵/kg body weight. In some aspects, an administering comprises infusing from about 0.5×10⁵/kg body weight to about 1×10⁵/kg body weight. In some aspects, an administering comprises infusing from about 0.5×10⁵/kg body weight to about 1.5×10⁵/kg body weight. In some embodiments, the administering comprises infusing from about 1×10³/kg body weight.

In some embodiments, a total of about 5×10¹⁰ cells are administered to a subject. In some embodiments, a subject can be administered a total concentration or a dose (cells/kg body weight) with at least about 1×10⁶ cells, at least about 2×10⁶ cells, at least about 3×10⁶ cells, at least about 4×10⁶ cells, at least about 5×10⁶ cells, at least about 6×10⁶ cells, at least about 6×10⁶ cells, at least about 8×10⁶ cells, at least about 9×10⁶ cells, 1×10⁷ cells, at least about 2×10⁷ cells, at least about 3×10⁷ cells, at least about 4×10⁷ cells, at least about 5×10⁷ cells, at least about 6×10⁷ cells, at least about 6×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 6×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 6×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 6×10¹¹ cells, at least about 6×10¹¹ cells, at least about 8×10¹¹ cells, at least about 9×10¹¹ cells, or at least about 1×10¹² cells are administered to a subject or dosed according to body weight (cells/kg body weight).

In some cases, a subject is administered a pharmaceutical composition that comprises at least about 2.0×10⁶, 2.0×10⁷, 2.0×10⁸, 3.0×10⁶, 3.0×10⁷, 3.0×10⁸, 4.0×10⁶, 4.0×10⁷, 4.0×10⁸, 5.0×10⁶, 5.0×10⁷, or 5.0×10⁸ ex vivo expanded T cells. In some cases, the ex vivo expanded T cells specifically bind a target exogenous peptide presented by a human leukocyte antigen (HLA) protein or fragment thereof.

Subjects to be treated with compositions provided herein can be human or non-human. A subject can be mammalian. Subjects can also be of any age. A subject can be an adult (from 18 years of age), teenager, child, infant, or a fetus. In some cases, a subject is non-human and can be any one of: dog, cat, monkey, cow, pig, bat, bird, and combinations thereof.

### Kits

Any of the compositions described herein may be comprised in a kit. In a non-limiting example, a particle, a cellular composition, a peptide, a protein associated with costimulation, a library of pepmixes, and any combination thereof may be comprised in a kit, any type of cells may be provided in the kit, and/or reagents for manipulation of peptides and/or cells may be provided in the kit. The components are provided in suitable container means.

The kits may comprise a suitably aliquoted composition. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EXAMPLES

### Example 1: T Cell and Dendritic Cell Isolation

### Whole Blood Isolation

100-400 mL of whole blood from a COVID-19 patient is collected. Blood is aliquoted into 50 mL conical tubes and centrifuged at 1300 rpm for 10 min (brake OFF). After centrifugation, the buffy coat is removed and diluted to 50 mL in sterile PBS. 20 mL of Ficoll-Paque is aliquoted in 50 mL conical tubes. 30 mL of PBS-diluted buffy coat is overlaid over each of the Ficoll-Paque aliquots. The mixture is centrifuged at 1800 rpm 20 min (brake OFF). 15− 20 mL of PBMC layer is collected and dilute to 50 mL in sterile PBS and centrifuged at 1300 rpm for 10 min. Cell pellets are resuspended in 5 mL MACS Buffer and filtered through a 70 μm filter, collecting all PBMCs in a 50 mL conical. Cells are counted with a hemocytometer.

### Apheresis Isolation for Leukopak

Mononuclear cells are collected using a FDA-approved apheresis cell separator system, using continuous-flow centrifugation for separation of blood into different components. Approximately 3 blood volumes is processed over approximately 196 minutes in a full-size leukapheresis donation. Approximately 1.5 blood volumes is processed over approximately 100 minutes in a half-pak leukapheresis donation. The collection of cells is performed at a collect rate range of about 0.8-1.2 mL/min. Dendritic cells can be isolated from additional sources as described in Table 4.

TABLE 4
Considerations Regarding Isolation of Dendritic Cells for ex vivo CD4 T cell Expansion

| | | Whole blood | Buffy coat | LRSC | Leukopak |
|---|---|---|---|---|---|
| Erythrocytes | Absolute | 5 × 10⁹ | 4 × 10¹¹ cells/buffy | 5 × 10¹⁰ | 8 × 10⁹ cells/half |
| | numbers | cells/ml. | coat | cells/LRSC | Leukopak |
| | Ratio to PBMCs | 2500 | 400 | 50 | 1.2 |
| PBMCs | Absolute | 2 × 10⁶ | 1 × 10⁹ cells/buffy | 1 × 10⁹ | 7 × 10⁹cells/half |
| | numbers | cells/mL | coat | cells/LRSC | Leukopak |
| | Ratio to PBMCs | 1 | 1 | 1 | 1 |
| Leukocytes | Absolute | 5 × 10⁶ | 2 × 10⁹ cells/buffy | 1 × 10⁹ | 7 × 10⁹ cells/half |
| | numbers | cells/mL | coat | cells/LRSC | Leukopak |
| | Ratio to PBMCs | 2.5 | 2 | 1 | 1 |

### Negative Selection of T Cells

Non-target cells, i.e., monocytes, neutrophils, eosinophils, B cells, stem cells, dendritic cells, NK cells, granulocytes, or erythroid cells are labeled by using a cocktail of biotin-conjugated antibodies. The cocktail contains antibodies against CD14, CD15, CD16, CD19, CD34, CD36, CD56, CD123, and CD235a (Glycophorin A). Subsequently, non-target cells are magnetically labelled with the Pan T Cell MicroBead Cocktail (Miltenyi Biotec). Isolation of highly pure T cells is achieved by depletion of magnetically labelled cells.

Alternatively, T cells can be isolated using StemCell Technologies, EasySep Human T cell Isolation Kit (Cat. #17951).

### Dendritic Cell Isolation

In an autologous setting, the isolation was performed in a two-step procedure. A subject's dendritic cells (DCs) were used to expand CD4+ T cells using MHC-II restricted antigen peptides presented by the subject's own DCs. DCs were isolated from the subject's PBMC sample used to extract the patients T cells, using a blood DC magnetic isolation kit. In short, blood Pan DCs were isolated using the Miltenyi Blood Dendritic Cell isolation kit II, followed by CD3+ T cell isolation using the Stem Cell T Cell enrichment kit. The dendritic cell isolation is performed as described below:

Centrifuge PBMCs at 300×g for 10 minutes.

Aspirate supernatant completely.

Resuspend cell pellet in 300 μL of MACS buffer (per 10⁸ total cells)

Add 100 μL of FcR Blocking Reagent and 100 μL of Non-DC Depletion Cocktail per 10⁸ total cells.

Mix well and incubate for 15 minutes in the refrigerator (2-8° C.).

Wash cells by adding 5-10 mL of buffer per 10⁸ cells and centrifuge at 300×g for 10 minutes.

Aspirate supernatant completely.

Resuspend cell pellet in buffer: 500 μL for up to 1.25×108 cells

Place LD Column in the magnetic field of a suitable MACS Separator.

Prepare column by rinsing with 2 mL of buffer.

Apply cell suspension onto the column.

Collect unlabeled cells that pass through and wash column with 2×1 mL of buffer.

Collect total effluent; this is the unlabeled pre-enriched dendritic cell fraction.

Perform washing steps by adding buffer two times. Only add new buffer when the column reservoir is empty.

Proceed to isolation of dendritic cells.

Centrifuge cell suspension at 300×g for 10 minutes.

Aspirate supernatant completely.

Resuspend cell pellet in 400 μL of buffer.

Add 100 μL of DC Enrichment Cocktail.

Mix well and incubate for 15 minutes in the refrigerator (2-8° C.).

Wash cells by adding 5-10 mL of buffer and centrifuge at 300×g for 10 minutes.

Aspirate supernatant completely.

Resuspend up to 10⁸ cells in 500 μL of buffer.

Proceed to magnetic separation

Place MS Column in the magnetic field of a suitable MACS Separator.

Prepare column by rinsing with 500 μL of buffer.

Apply cell suspension onto the column. Collect flow-through containing unlabeled cells.

Wash column with 3×500 μL of buffer. Collect unlabeled cells that pass through and combine with the flow-through from step 3. Note: Perform washing steps by adding buffer aliquots only when the column reservoir is empty.

Remove column from the separator and place it on a suitable collection tube.

Pipette 500 μL of buffer onto the column. Immediately flush out the magnetically labelled cells by firmly pushing the plunger into the column.

To increase the purity of dendritic cells, the eluted fraction can be enriched over a second MS or LS Column. Repeat the magnetic separation procedure as described in steps 1 to 6 by using a new column.

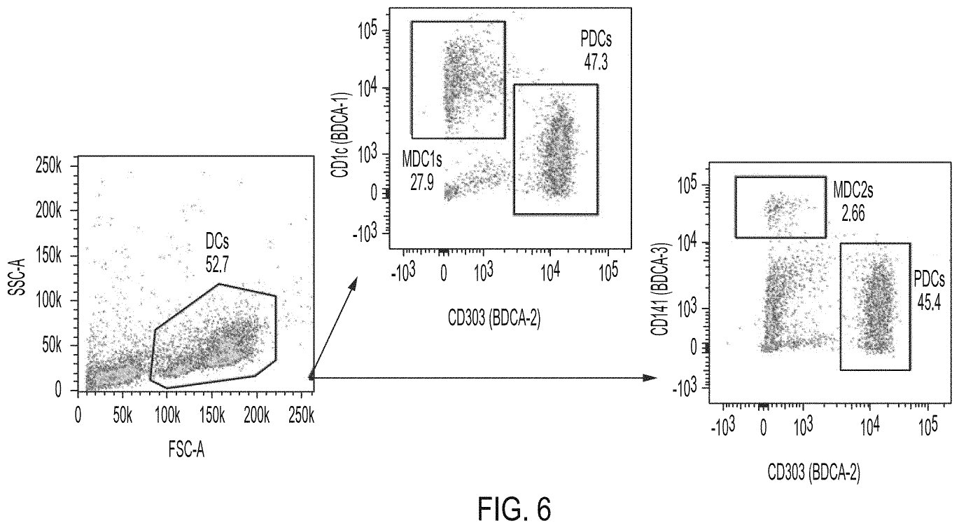

Approximately 1% of PBMCs are dendritic cells, further subdivided into plasmacytoid DCs (PDCs) and Myeloid DCs (MDCS1 and MDCS2, as shown in FIG. 6.

Alternatively, B cells and monocytes are magnetically labeled and depleted using a cocktail of CD19 and CD14 MicroBeads. Subsequently, the pre-enriched dendritic cells in the non-magnetic flow-through fraction are magnetically labeled and enriched using a cocktail of antibodies against the dendritic cell markers CD304 (BDCA-4/Neuropilin-1), CD141 (BDCA-3), and CD1c (BDCA-1). The highly pure enriched cell fraction comprises plasmacytoid dendritic cells, CD1c (BDCA-1)+ type-1 myeloid dendritic cells (MDC1s), and CD1c (BDCA-1)-CD141 (BDCA-3)bright type-2 myeloid dendritic cells (MDC2s).

B cells and monocytes are depleted in advance because a subpopulation of B cells expresses CD1c (BDCA-1), and monocytes express CD141 (BDCA-3) at low levels.

### Dendritic Cell Maturation

In the case where dendritic cell maturation is needed, the following cytokines are used for DC stimulation and are added sequentially for day 0, GM-CSF (R&D Systems; 1,000 U/mL), IL-4 (R&D Systems; 500 U/mL), IL-10 (R&D Systems; 10 ng/mL), and Flt3L (R&D Systems; 50 ng/mL); for day 1, TLR8L (ssRNA40, Invivogen; 0.5 μg/mL), TNF-α (R&D Systems; 1,000 U/mL), PGE2 (Merck Calbiochem; 1 μM), and IL-7 (R&D Systems; 0.5 ng/mL); and for day 2, IL-2 (Proleukin, Novartis; 100 U/mL), IL-15 (R&D Systems; 25 ng/mL), and IL-7 (R&D Systems; 5 ng/mL). Cytokines are added by replacing the half medium volume with AIM-V and 10% human serum containing the abovementioned cytokines at the indicated final concentrations calculated for the whole culture volume. When IL-10 is added at day 0, it may not be further added at day 1. Half medium is replenished every 2-3 days with AIM-V and 10% human serum, supplemented with 100 U/mL IL-2, 25 ng/mL IL-15, and 5 ng/mL IL-7.

Alternatively, dendritic cells can be cultured overnight with maturation factors as previously described. Dendritic cells were seeded overnight with maturation factors in complete media (X-VIVO 15 media with 10% human serum) supplemented with GM-CSF 100 ng/ml and 50 ng/ml IL-4 and incubated overnight.

### Biotin Pentamer and Streptavidin FITC Reagent Test

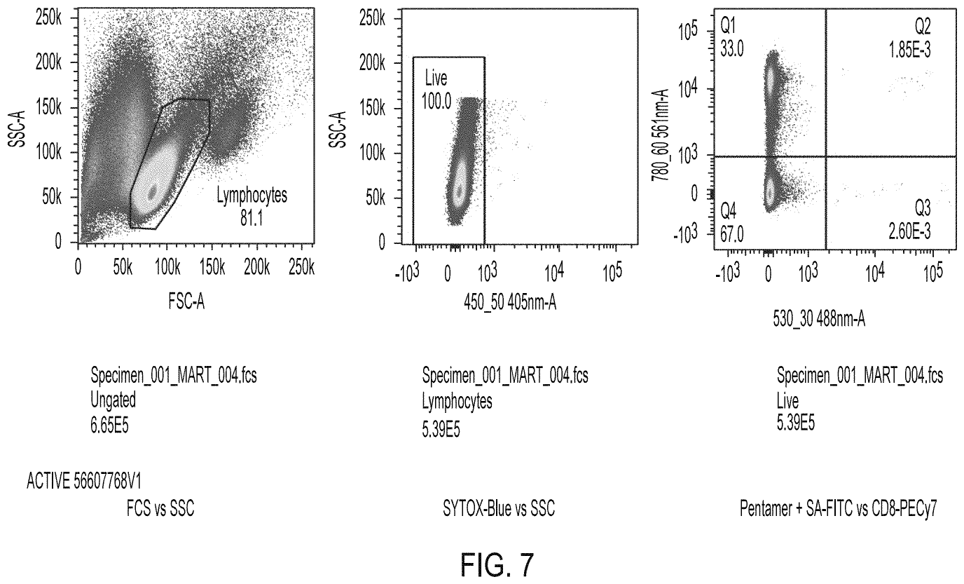

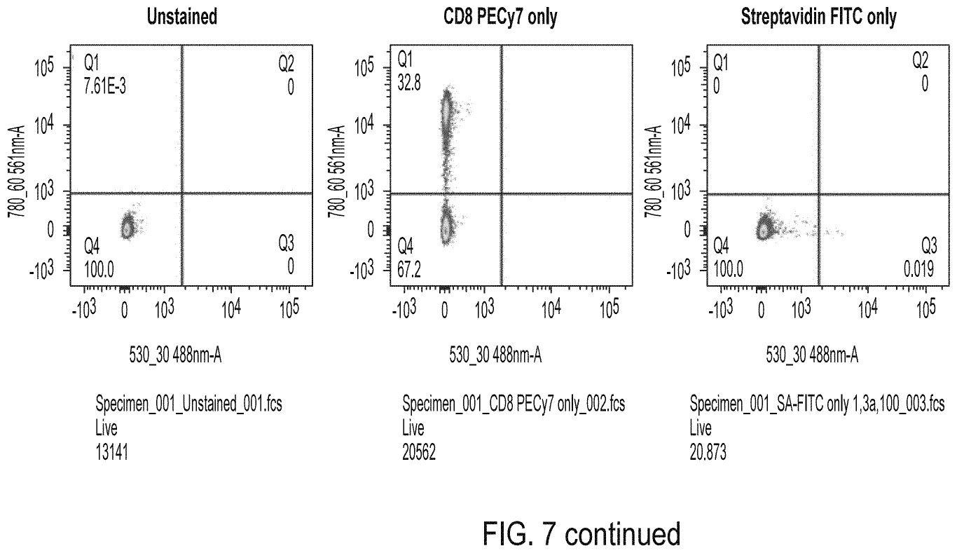

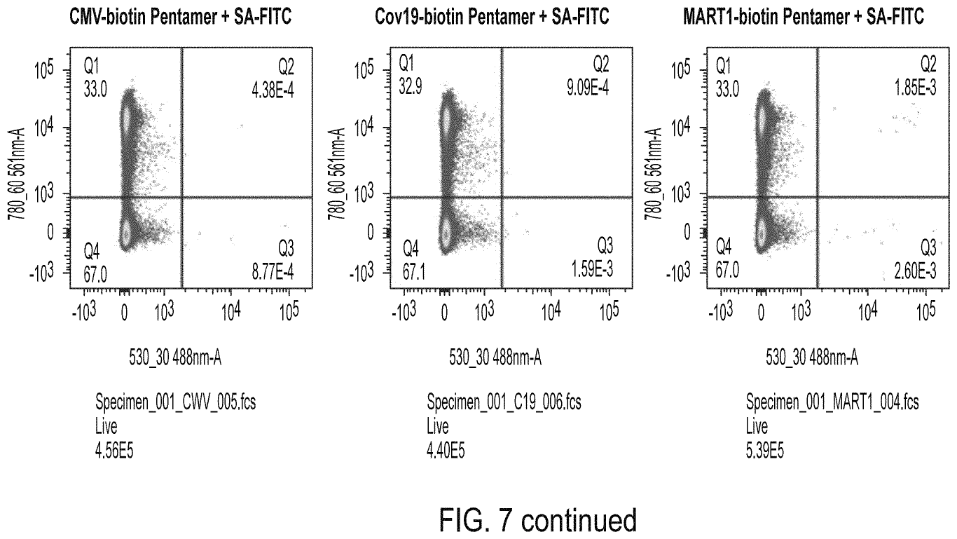

PBMCs were isolated from normal healthy donors and stained for CD8 T cells and for TCRs against the CMV pp65 antigen (NLVPMVATV, SEQ ID NO: 70), MART1 antigen (ELAGIGILTV, SEQ ID NO: 71) and the SARS-CoV2 predicted antigen (FIAGLIAIV, SEQ ID NO: 59) using the matched biotinylated Pentamers. Pentamers were visualized via incubating the stain PBMCs with a streptavidin conjugated to FITC prior to flow cytometry FIG. 7. Wildtype cells are HLA-A*02:01.

Results show that the SARS-Cov-2 immunogen was found to have reduced immunogenicity. A small number of MART1 positive cells were identified in PMBCs. The biotinylated pentamers yielded low background staining.

### Example 2: Generating COVID-19 Spike Peptide Loaded Dendritic Cells

DCs are resuspended at 1×10⁶ cells/ml normal saline with 1% human albumin. DCs are pulsed with 10 μg/ml of COVID-19 spike peptide for HLA-A2 and/or HLA-DR for 4 h at room temperature. Control DCs are pulsed with a control antigen (e.g. Tetanaus Toxin). Efficient endocytosis is confirmed using FITC-dextran or DQ red BSA.

Alternatively, peptides were added to the DCs in the morning by replacing with complete media containing 20 ug/ml of peptide antigen and incubated for 2 hours. After incubation, 50% of the media on the DCs was replaced with T cells at a ratio of 1:10 (DCs:T Cells) cultured in complete T cell media containing 10% human serum, IL-2 (6000 IU/ml), IL-7 (5 ng/ml) and IL-15 (5 ng/ml), N acetyl cysteine (10 mM). Peptide antigens that were utilized are as follows: NLVPMVATV (SEQ ID NO: 70), immunodominant A*02:01 epitope from CMV pp65; ELAGIGILTV (SEQ ID NO: 71), A*02:01 restricted MelanA/MART-1 epitope; YLQPRTFLL (SEQ ID NO: 72), A*02:01 restricted SARS-CoV-2 S protein 288-296 residues of SEQ ID NO: 56; and PKYVKQNTLKLAT (SEQ ID NO: 73), Influenza DRB1*01:01 restricted immunodominant epitope.

Exemplary SARS-Cov-2 peptides that can be utilized are provided in Table 5 or Table 6. In some cases, a peptide or fragment thereof can have from about 50%, 60%, 70%, 75%, 80%, 85%, 88%, 90%, 93%, 95%, 96%, 97%, 98%, 99%, or 100% identity to a peptide from Table 5, Table 6, any one of SEQ ID NO: 70-73, 80; or a peptide or fragment encoded by SEQ ID NO: 81.

TABLE 5
SARS-CoV-2 derived T cell epitopes obtained
using positive T cell assays that are
identical in SARS-CoV-2 spike.

| SEQ | | | | MHC |
|---|---|---|---|---|
| ID | IEDB | Peptide | | Allele |
| NO | ID | Epitope | MHC Allele | Class |
| 59 | 16156 | FIAGLIAIV | HLA-A*02:01/ | I |
| | | | HLA-A*02:02/ | |
| | | | HLA-A*02:03/ | |
| | | | HLA-A*02:06/ | |
| | | | HLA-A*68:02/ | |
| | | | HLA-A2 | |
| 60 | 2801 | ALNTLVKQL | HLA-A*02:01 | I |
| 61 | 36724 | LITGRLQSL | HLA-A2/ | I |
| | | | HLA-A*02:01 | |
| 62 | 44814 | NLNESLIDL | HLA-A*02:01 | I |
| 63 | 54680 | RLNEVAKNL | HLA-A*02:01 | I |
| 64 | 69657 | VLNDILSRL | HLA-A*02:01 | I |
| 65 | 71663 | VVFLHVTYV | HLA-A*02:01/ | I |
| | | | HLA-A*02:02/ | |
| | | | HLA-A*02:03/ | |
| | | | HLA-A*02:06/ | |
| | | | HLA-A*68:02 | |
| 66 | 100048 | GAALQIPFA | HLA-DRA*01:01 | II |
| | | MQMAYRF | DRB1*07:01 | |
| 67 | 100300 | MAYRFNGIG | HLA-DRB1*04:01 | II |
| | | VTQNVLY | | |
| 68 | 100428 | QLIRAAEIR | HLA-DRB1*04:01 | II |
| | | ASANLAATK | | |
| 69 | 50311 | QALNTLVKQ | HLA-DRB1*04:01 | II |
| | | LSSNFGAI | | |

TABLE 6
Additional SARS-CoV-2 peptides

| SEQ | | | | MHC |
|---|---|---|---|---|
| ID | | Peptide | MHC | Allele |
| NO | Protein | Epitope | Allele | Class |
| 74 | Replicase | TTDPSFLG | A*01:01 | I |
| | polyprotein | RY | | |
| | lab | | | |
| 75 | Replicase | PTDNYITTY | A*01:01 | I |
| | polyprotein | | | |
| | lab | | | |
| 76 | N protein | LLLDRLNQL | A*02:01 | I |
| 77 | S-protein | YLQPRTFLL | A*02:01 | I |
| 78 | S-protein | RLQSLQTYV | A*02:01 | I |
| 79 | nucleocapsid | MEVTPSGTWL | B*40:01 | I |

GenBank: QHD43423.2
SEQ ID NO: 80
MSDNGPQNQRNAPRITFGGPSDSTGSNQNGERSGARSKQR
RPQGLPNNTASWFTALTQHGKEDLKFPRGQGVPINTNSSP
DDQIGYYRRATRRIRGGDGKMKDLSPRWYFYYLGTGPEAG
LPYGANKDGIIWVATEGALNTPKDHIGTRNPANNAAIVLQ
LPQGTTLPKGFYAEGSRGGSQASSRSSSRSRNSSRNSTPG
SSRGTSPARMAGNGGDAALALLLLDRLNQLESKMSGKGQQ
QQGQTVTKKSAAEASKKPRQKRTATKAYNVTQAFGRRGPE
QTQGNFGDQELIRQGTDYKHWPQIAQFAPSASAFFGMSRI
GMEVTPSGTWLTYTGAIKLDDKDPNFKDQVILLNKHIDAY
KTFPPTEPKKDKKKKADETQALPQRQKKQQTVTLLPAADL
DDFSKQLQQSMSSADSTQA
cDNA of the RSA Spike protein without
the furin cleaveg site
SEQ ID NO: 81
ATGGCATTCGTGTTTCTGGTGCTGCTGCCTCTGGTGTCCA
GCCAGTGCGTGAACTTCACCACCAGAACACAGCTGCCTCC
AGCCTACACCAACAGCTTTACCAGAGGCGTGTACTACCCC
GACAAGGTGTTCAGATCCAGCGTGCTGCACTCTACCCAGG
ACCTGTTCCTGCCTTTCTTCAGCAACGTGACCTGGTTCCA
CGCCATCCACGTGTCCGGCACCAATGGCACCAAGAGATTC
GCCAATCCTGTGCTGCCCTTCAACGACGGGGTGTACTTTG
CCAGCACCGAGAAGTCCAACATCATCAGAGGCTGGATCTT
CGGCACCACACTGGACAGCAAGACCCAGAGCCTGCTGATC
GTGAACAACGCCACCAACGTGGTCATCAAAGTGTGCGAGT
TCCAGTTCTGCAACGACCCCTTCCTGGGCGTCTACTACCA
CAAGAACAACAAGAGCTGGATGGAAAGCGAGTTCCGGGTG
TACAGCAGCGCCAACAACTGCACCTTCGAGTACGTGTCCC
AGCCTTTCCTGATGGACCTGGAAGGCAAGCAGGGCAACTT
CAAGAACCTGCGCGAGTTCGTGTTCAAGAACATCGACGGC
TACTTCAAGATCTACAGCAAGCACACCCCTATCAACCTCG
TGCGGGGACTGCCTCAGGGCTTTTCTGCTCTGGAACCCCT
GGTGGATCTGCCCATCGGCATCAACATCACCCGGTTTCAG
ACCCTGCACCGGTCCTATCTGACACCCGGCGATTCTTCTA
GCGGATGGACAGCTGGCGCCGCTGCCTACTATGTGGGATA
CCTGCAGCCTCGGACCTTCCTGCTGAAGTACAACGAGAAC
GGCACCATCACCGACGCCGTGGATTGTGCTCTGGATCCTC
TGAGCGAGACAAAGTGCACCCTGAAGTCCTTCACCGTGGA
AAAGGGCATCTACCAGACCAGCAACTTCCGGGTGCAGCCC
ACCGAATCCATCGTGCGGTTCCCCAATATCACCAATCTGT
GCCCCTTCGGCGAGGTGTTCAATGCCACCAGATTCGCCTC
TGTGTACGCCTGGAACCGGAAGCGGATCAGCAATTGCGTG
GCCGACTACTCCGTGCTGTACAACTCCGCCAGCTTCAGCA
CCTTCAAGTGCTACGGCGTGTCCCCTACCAAGCTGAACGA
CCTGTGCTTCACAAACGTGTACGCCGACAGCTTCGTGATC
CGGGGAGATGAAGTGCGGCAGATTGCCCCTGGACAGACCG
GCAATATCGCCGACTACAACTACAAGCTGCCCGACGACTT
CACCGGCTGTGTGATTGCCTGGAACAGCAACAACCTGGAC
TCCAAAGTCGGCGGCAACTACAATTACCTGTACCGGCTGT
TCCGGAAGTCCAATCTGAAGCCCTTCGAGCGGGACATCTC
CACCGAGATCTATCAGGCCGGCAGCACCCCTTGCAATGGC
GTGAAGGGCTTTAACTGCTACTTCCCACTGCAGTCCTACG
GCTTCCAGCCAACATACGGCGTGGGCTACCAGCCTTACAG
AGTGGTGGTGCTGAGCTTCGAGCTGCTGCATGCTCCTGCC
ACAGTGTGCGGCCCTAAGAAAAGCACCAATCTCGTGAAGA
ACAAATGCGTCAACTTCAATTTCAACGGCCTGACCGGCAC
CGGCGTGCTGACAGAGAGCAACAAGAAGTTCCTGCCATTC
CAGCAGTTCGGCCGGGACATTGCCGATACCACAGATGCTG
TCAGAGATCCCCAGACACTGGAAATCCTGGACATCACCCC
ATGCAGCTTCGGCGGAGTGTCTGTGATCACCCCTGGCACC
AACACCAGCAATCAGGTGGCAGTGCTGTACCAGGGCGTCA
ACTGTACAGAGGTGCCAGTGGCCATTCACGCCGATCAGCT
GACCCCTACTTGGCGGGTGTACTCCACAGGCAGCAATGTG
TTCCAGACCAGAGCCGGCTGTCTGATCGGAGCCGAGCACG
TGAACAATAGCTACGAGTGCGACATCCCCATCGGCGCTGG
CATCTGTGCCAGCTACCAGACACAGACAAACAGCCCCGGC
GGCAGCGGATCTGTGGCCAGCCAGAGCATCATTGCCTACA
CAATGTCTCTGGGCGTCGAGAACAGCGTGGCCTACTCCAA
CAACTCTATCGCTATCCCCACCAATTTCACCATCAGCGTG
ACCACAGAGATCCTGCCTGTGTCCATGACCAAGACCAGCG
TGGACTGCACCATGTACATCTGCGGCGATAGCACCGAGTG
CTCCAACCTGCTGCTGCAGTACGGCAGCTTCTGCACCCAG
CTGAATAGAGCCCTGACCGGAATCGCCGTGGAACAGGACA
AGAACACCCAAGAGGTGTTCGCCCAAGTGAAGCAGATCTA
CAAGACCCCTCCTATCAAGGACTTCGGCGGCTTCAACTTC
AGCCAGATTCTGCCCGATCCTAGCAAGCCCAGCAAGCGGA
GCTTCATCGAGGACCTGCTGTTCAACAAAGTGACACTGGC
CGACGCCGGCTTCATCAAGCAGTATGGCGATTGTCTGGGC
GACATTGCAGCCCGGGATCTGATTTGCGCCCAGAAGTTTA
ACGGACTGACCGTGCTGCCTCCTCTGCTGACCGATGAGAT
GATCGCCCAGTACACATCTGCCCTGCTGGCCGGCACAATC
ACAAGCGGCTGGACATTTGGAGCTGGCGCTGCCCTGCAGA
TCCCCTTTGCTATGCAGATGGCCTACCGGTTCAACGGCAT
CGGAGTGACCCAGAATGTGCTGTACGAGAACCAGAAGCTG
ATCGCCAACCAGTTCAACAGCGCCATCGGCAAGATCCAGG
ACAGCCTGAGCAGCACAGCCAGCGCTCTGGGAAAACTGCA
GGACGTGGTCAACCAGAACGCCCAGGCTCTGAATACCCTG
GTCAAGCAGCTGTCCTCCAACTTCGGCGCCATCAGCTCTG
TGCTGAACGATATCCTGAGCAGACTGGACCCTCCTGAAGC
CGAGGTGCAGATCGACAGACTGATCACCGGAAGGCTGCAG
TCCCTGCAGACCTACGTTACCCAGCAGCTGATCAGAGCCG
CCGAGATTAGAGCCTCTGCCAATCTGGCCGCCACCAAGAT
GTCTGAGTGTGTGCTGGGCCAGAGCAAGAGAGTGGACTTT
TGCGGCAAGGGCTACCACCTGATGAGCTTCCCTCAGTCTG
CACCACACGGCGTGGTGTTTCTGCACGTGACATACGTGCC
CGCTCAAGAGAAGAACTTCACAACAGCCCCTGCCATCTGC
CACGACGGCAAAGCCCACTTTCCTAGAGAAGGCGTGTTCG
TGTCCAACGGCACCCATTGGTTCGTGACCCAGCGGAACTT
CTACGAGCCCCAGATCATCACCACCGACAACACCTTCGTG
TCTGGCAACTGCGACGTCGTGATCGGCATTGTGAACAATA
CCGTGTACGACCCTCTGCAGCCCGAGCTGGACAGCTTCAA
AGAGGAACTGGATAAGTACTTTAAGAACCACACAAGCCCC
GACGTGGACCTGGGCGATATCAGCGGAATCAATGCCAGCG
TCGTGAACATCCAGAAAGAGATCGACCGGCTGAACGAGGT
GGCCAAGAATCTGAACGAGAGCCTGATCGACCTGCAAGAA
CTGGGGAAGTACGAGCAGTACATCAAGTGGCCTTGGTACA
TCTGGCTGGGCTTTATCGCCGGACTGATTGCCATCGTGAT
GGTCACAATCATGCTGTGCTGTATGACCAGCTGCTGTAGC
TGCCTGAAGGGCTGTTGCAGCTGTGGCTCCTGCTGCAAGT
TCGACGAGGACGACAGTGAGCCGGTGCTTAAGGGCGTAAA
ACTTCATTACACTTCCGGATGA.

### Example 3: Culture of T Cells with COVID-19 Spike Peptide Loaded Dendritic Cells

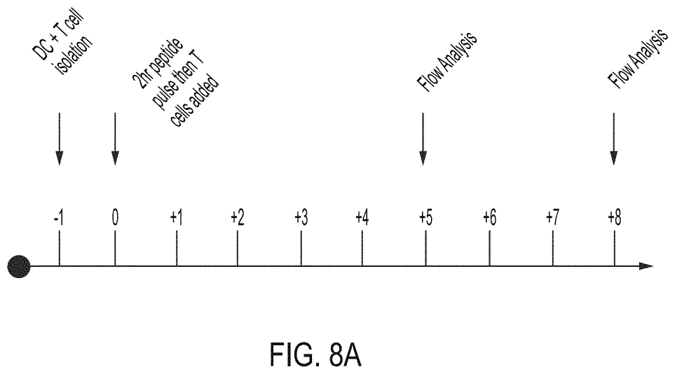

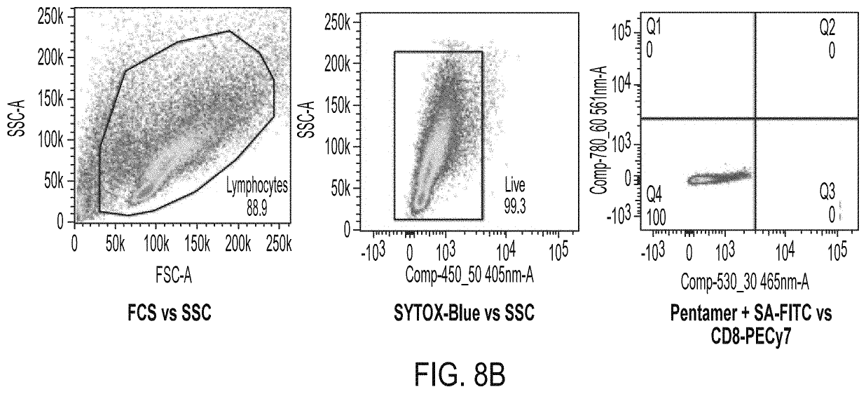

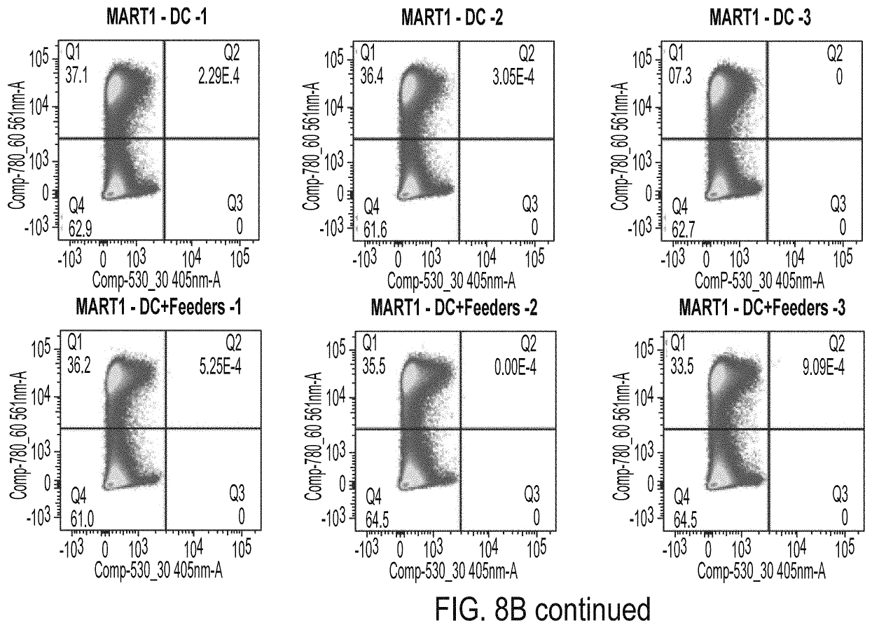

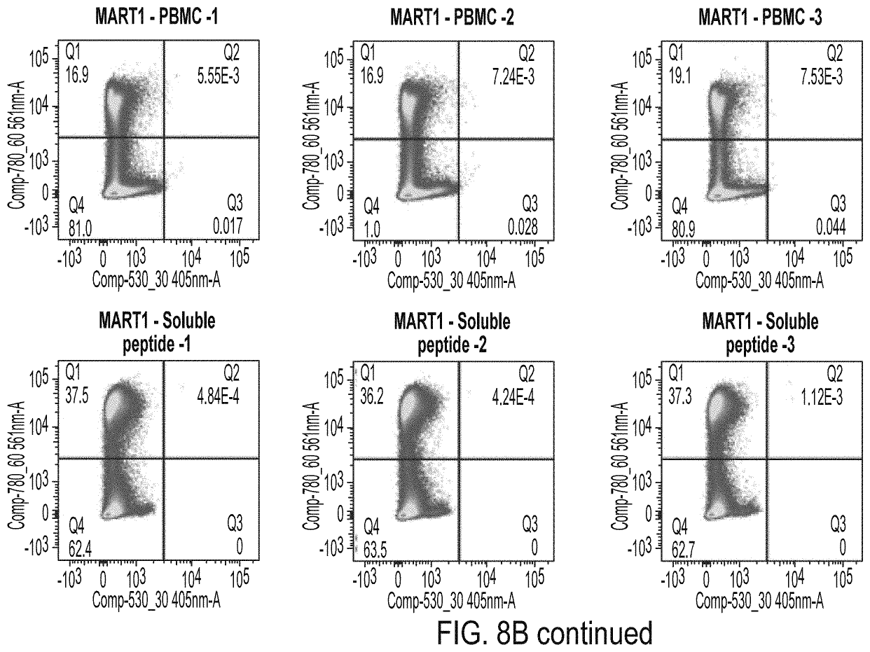

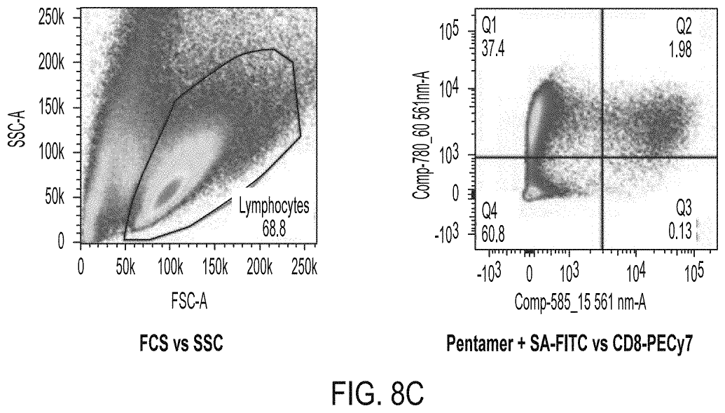

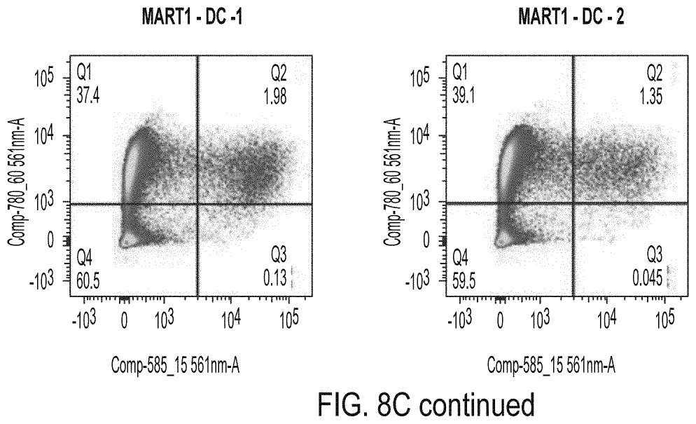

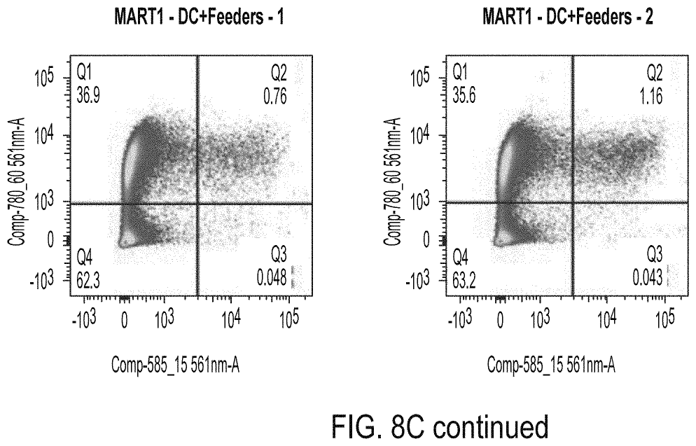

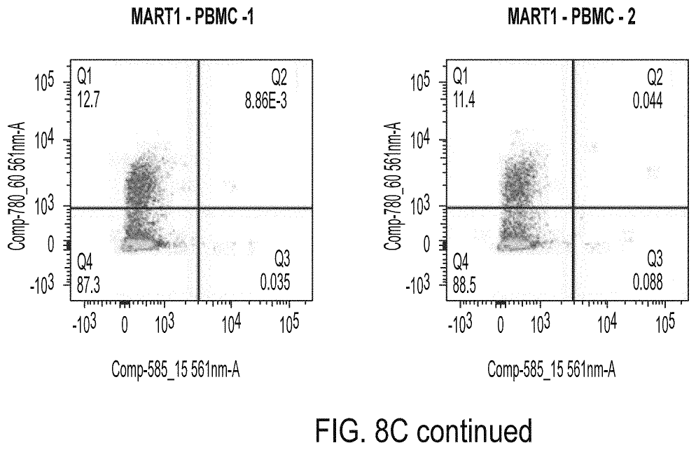

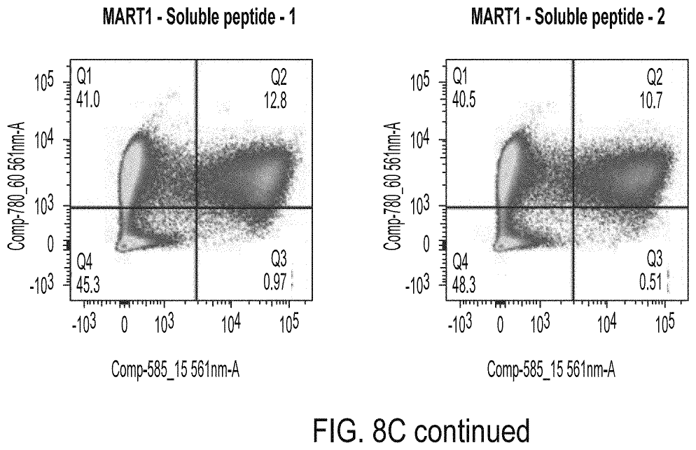

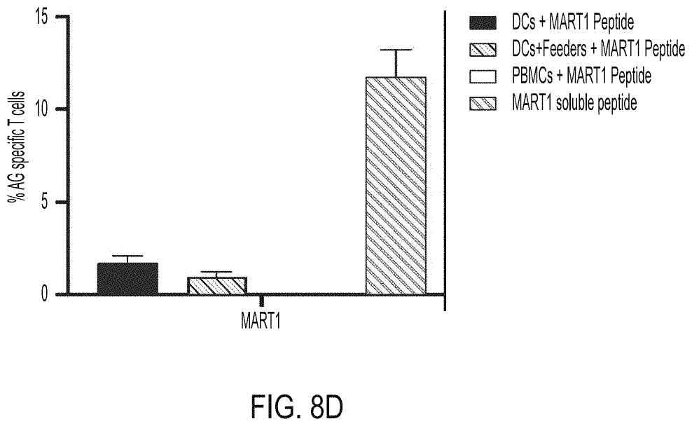

Cultures from an HLA-A*02:01 COVID+ donor were established to test different DC conditions for the expansion of MART1 positive cells with 2×10⁵ T cells: DCs+ T cells; DCs with PBMC feeders; PBMCs alone; soluble protein alone, FIG. 8A.

In short, T cells were stimulated with peptide loaded autologous DCs (pulsed with 20 ug of matched peptide antigen) at ratios between 1:10 to 1:20 for 72 hours in media supplemented with 1000 U/ml IL4 (R&D Systems, Minneapolis, Minn.) and 10 ng/ml IL7 (PeproTech, Rocky Hill, N.J.). MART peptide of SEQ ID NO: 71 and SARS-CoV-2 peptide of SEQ ID NO: 59. Flow cytometry was set up on day 5 of culture to evaluate biotinylated pentamers and SA-FITC, FIG. 8B. A flow cytometry assay was also performed on day 8 or 9 of culture, FIG. 8C. A summary of percent antigen specific T cells post culture is shown at FIG. 8D for MART1 peptide loaded DCs. Flow cytometry plots were gated using the markers shown in Table 7.

Results show that (1) Rapidly matured DCs from unfractionated PBMCs (acDCs) are less efficient than alternative strategies. (2) MART1 soluble peptide seems highly immunogenic, leading to best expansion of MART1-specific T cells (this may be the case for the MART1 and CMV pp65 antigens, the response to specific SARS-CoV-2 peptides will need to be evaluated). (3) Best cellular response appears to be with isolated DCs (without any additional feeders).

TABLE 7
Summary of flow cytometry analysis of viral-specific T cells

| Color | Marker | Fluorochrome | Species | Laser | Filter |
|---|---|---|---|---|---|
| 1 | CD4 | APC efluor780 | Mouse | 640 | 780/60 |
| 2 | CD8 | PeCy7 | Mouse | 561 | 780/60 |
| 3 | Antigen Pentamer | Streptavidin-PE | N/A | 561 | 585/15 |
| 4 | CD69 | FITC | Mouse | 488 | 530/30 |
| 5 | CD45RA | APC efluor780 | Mouse | 640 | 670/30 |
| 6 | CD45RO | Pe-Cy5 | Mouse | 561 | 670/30 |
| 7 | IFNg | BUV395 | Mouse | 355 | 379/28 |
| 8 | IL-2 | eFluor450 | Rat | 450 | 450/50 |
| 9 | TNFa | PerCP-Cy5.5 | Mouse | 488 | 670/30 |
| 10 | Dead cells | 7-AAD | N/A | 561 | 710/50 |
| 11 | Dead cells | SYTOX Blue | N/A | 405 | 450/40 |

### Example 4: Nano-aAPP Particle Synthesis

Iron-Dextran aAPP

Nanoscale iron-dextran aAPP were manufactured in one of two ways. 2 μM biotinylated MHC-lg dimer and an equimolar concentration of biotinylated anti-CD28 antibody were incubated with 100 μl of anti-biotin Miltenyi Microparticles (Miltenyl Biotec) for at least 1 hour with gentle agitation at 4° C. Unbound protein was washed using a MS magnetic enrichment column (Miltenyl Biotec). Particle concentration was measured by absorbance at 405 nm using a Beckman Coulter AD340 plate reader.

Alternatively, MHC-lg dimer and B7.1-lg were directly chemically coupled to biodegradable particles (Miltenyl Biotec). Total protein content was assessed by Bradford assay. Unless otherwise stated, “iron-dextran aAPP” refers to particles directly chemically coupled to MHC and B7.1, rather than anti-biotin coupling.

Quantum Dot aAPP

Nanoscale quantum dot aAPP were manufactured by incubating 5 μm biotinylated MHC-1 g dimer and an equimolar concentration of biotinylated anti-CD28 antibody with 100 μL of 1 μM streptavidin coated quantum dots (Life Technologies) for 2 hours at 4° C. Quantum dots were washed and concentrated using a Sartorius Vivaspin membrane with a 300,000 molecular weight cutoff. Quantum dot concentration was measured by absorbance at 405 nm using a Beckman Coulter AD340 plate reader.

Microbead aAPP

In a first step, pentamers are affixed to microbeads. In brief, M-280 Epoxy microbeads (BD bioscience) are modified to affix an MHC (I or II) pentamer (Proimmune) through covalent binding between biotin molecules that have been added to the pentamer, and streptavidin molecules that are coated on the M-280 Epoxy microbead. The biotinylated pentamers are incubated at 4C with the microbeads, in a PBS buffer, with agitation for 1 hour. This is sufficient to promote binding of the biotin to the streptavidin, before washing and magnetic purification of the beads from the unbound protein. Alternatively, MHC-II tetramers are affixed to M280 Streptavidin Epoxy microbeads (BD bioscience) and biotinylated anti-human CD28 antibody (BD bioscience).

In a second step, pentamer and/or tetramers were combined with anti-CD28 antibody in a pre-decided molar ratio (e.g. 1:30) and incubate with at excess with 100 ul of M280 beads (approx. 6×10⁷ beads). The mixture was incubated at 4° C. for 1 hour with frequent agitation. The particles were centrifuged for 5 minutes at 4° C. at 14,000×g. The microbeads were washed in PBS two times while the microbeads remained inside the magnet to secure the microbeads along the inner bottom side of the tube. Microbeads were resuspended at a density of 5×10⁸ microbeads per ml in PBS and stored at 4° C.

### Example 5: Preparation of MHC-Ig Dimers

Soluble MHC-lg dimers, K^{b}-lg and D^{b}-lg, were prepared and loaded with peptide as described (Schneck J P, Slansky J E, O'Herrin S M, Greten T F. Monitoring antigen-specific T cells using MHC-Ig dimmers. Chapter 17. Current protocols in immunology/edited by John E. Coligan et al. 2001 Unit 17.2). Briefly, K^{b}-lg molecules were loaded with COVID-19 spike peptide by stripping at alkaline condition (pH 11.5), and then refolded in the presence of 50 fold excess COVID-19 spike peptide. D^{b}-lg molecules were stripped under mildly acidic conditions (pH 6.5) and refolded in the presence of 50 fold molar excess peptide and 2-fold molar excess of human 02-microglobulin. Human A2-lg was passively loaded in the presence of excess M1 peptide (Chiu Y-L, Schneck J P, Oelke M. HLA-Ig based artificial antigen presenting cells for efficient ex vivo expansion of human CTL. Journal of visualized experiments: JoVE. 2011; (50):1-5). Peptides SIY (SIYRYYGL (SEQ ID NO: 85), synthetic), SIIN (SIINFEKL (SEQ ID NO: 86), derived from ovalbumin protein), GP100 (KVPRNQDWL (SEQ ID NO: 87), from melanocyte GP100 protein) ASN (ASNENMETH (SEQ ID NO: 88), from influenza A nucleoprotein), and M1 (GILGFVFTL (SEQ ID NO: 89), from influenza A M1 protein) were purchased from Genscript (Piscataway, N.J.). Protein concentration was determined after labeling by size exclusion high performance liquid chromatography (HPLC).

### Example 6: Stimulation of SARS-Cov2 Specific T Cells with Particles to Generate an Antigen-Specific T Cell Such as a Virus Inducted Lymphocyte (VIL)

T cells are diluted to 1×10⁶/mL, aliquoted into 5 mL cultures in a 6 well plate, and contacted with about 40 μg/mL particles (HLA-matched or universal/allogeneic) for 24 hours at 37° C. After 24 hour incubation, the particles are removed, and the cultures are fed every 2-3 days with fresh medium containing 50 IU/ml rIL-2.

After 24 hours, the VILs are analyzed by flow cytometry for expression of memory T and effector T cell markers. T cells are stained with fluorochrome-conjugated monoclonal antibodies (CD3, CD4, CD8, CD25, CD45RA, CD45RO, CD127, CD62, and CCR7) in FACS Wash Buffer (Dulbecco's phosphate buffered saline 1× with 1% bovine serum albumin) for 30 min on ice for surface staining. Dead cells are excluded using AQUA live/dead dye from Invitrogen. Effector T cells can comprise any one of CD25+, CD45RA−, CD45RO+, CD127−, CD62−, CCR7−) and Effector memory T can comprise any one of CD25−, CD45RA−, CD45RO+, CD127+, CD62L+, CCR7+.

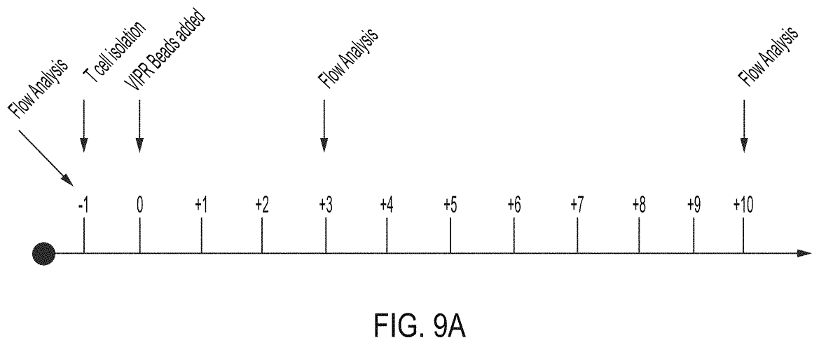

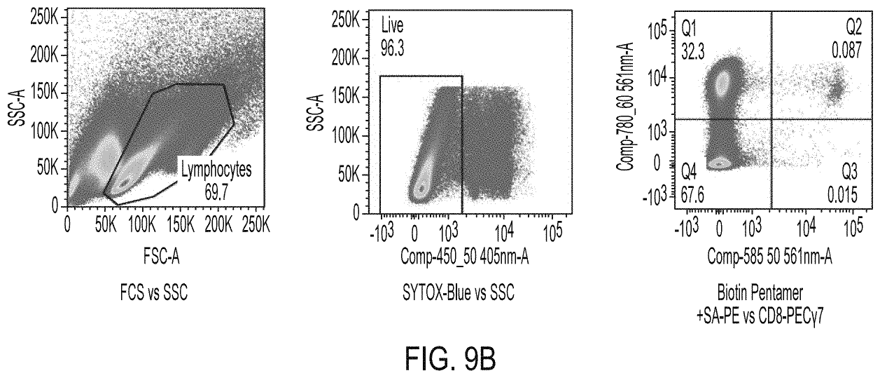

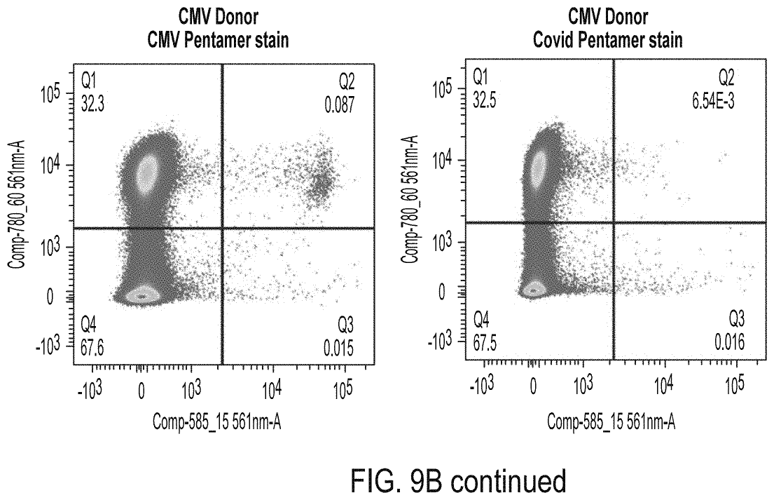

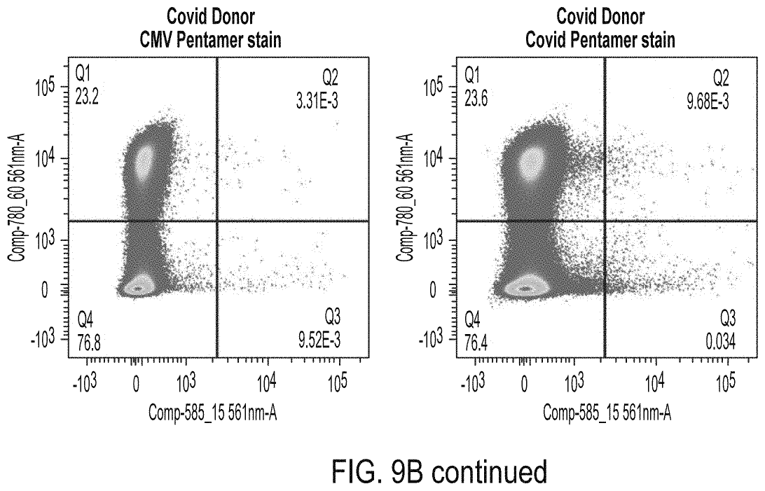

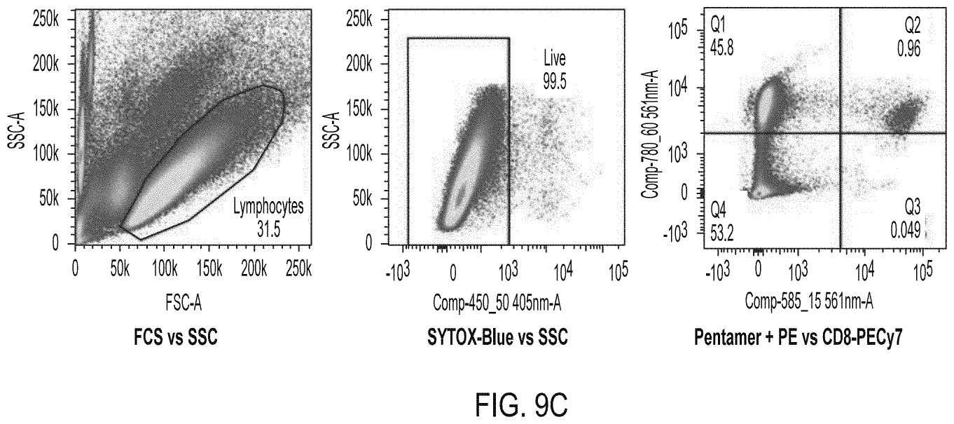

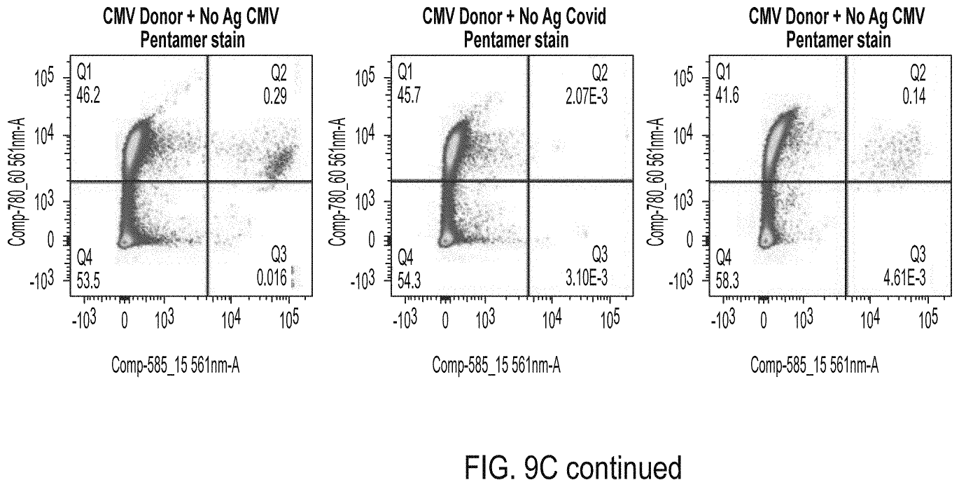

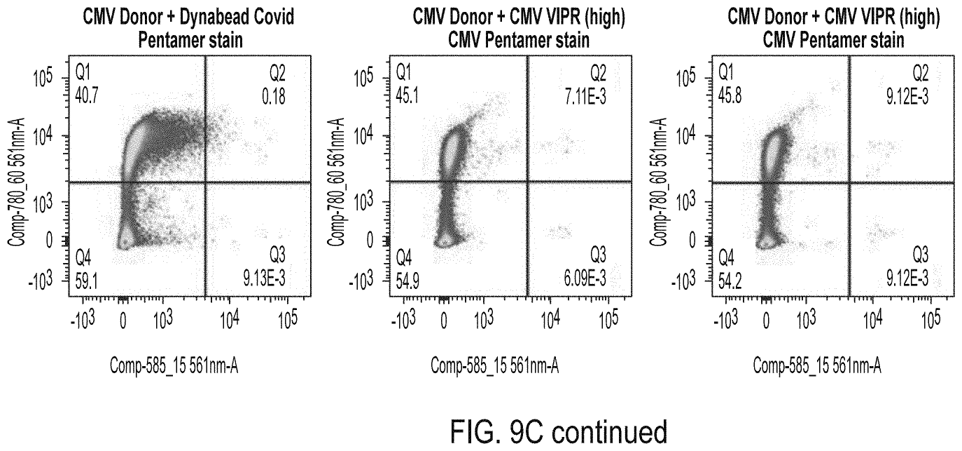

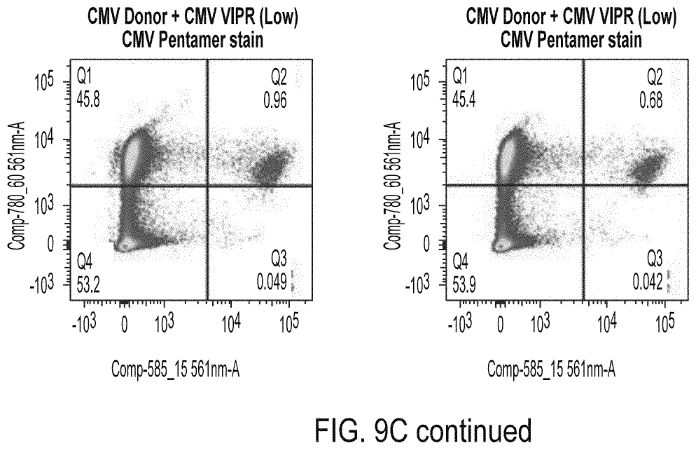

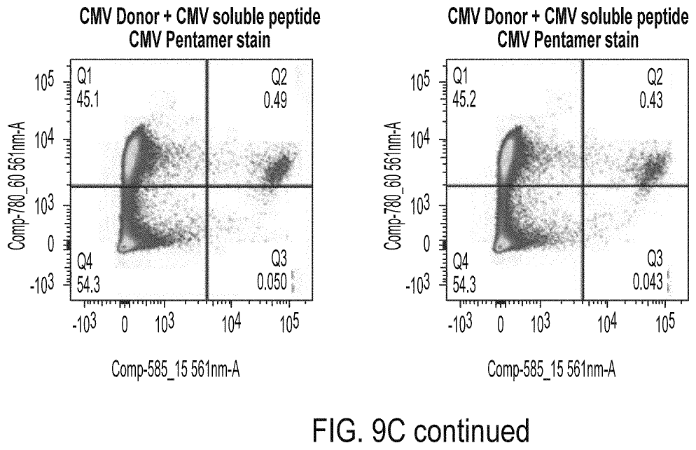

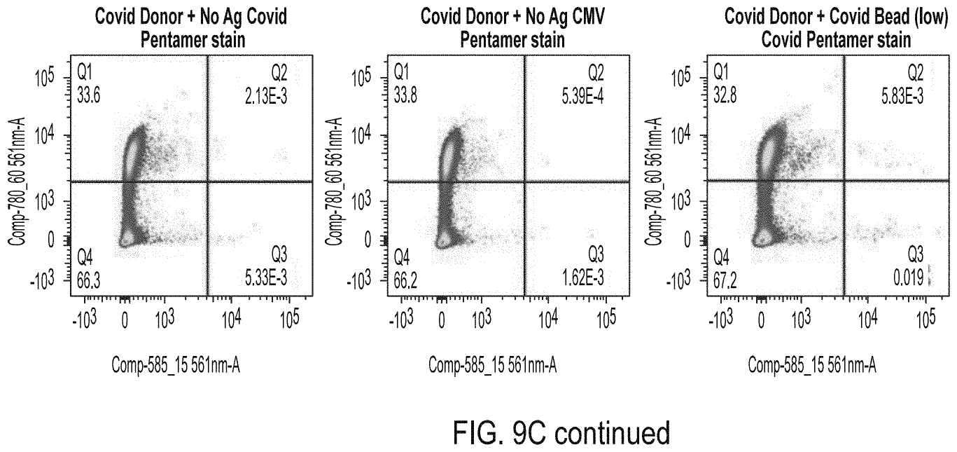

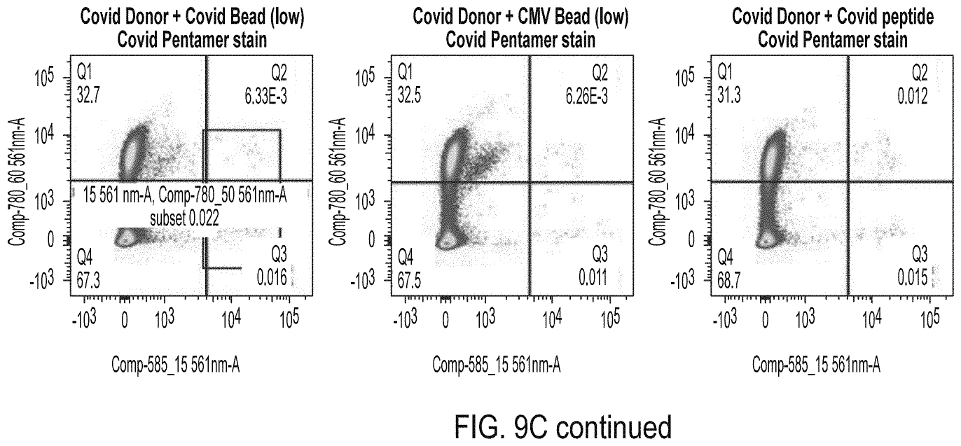

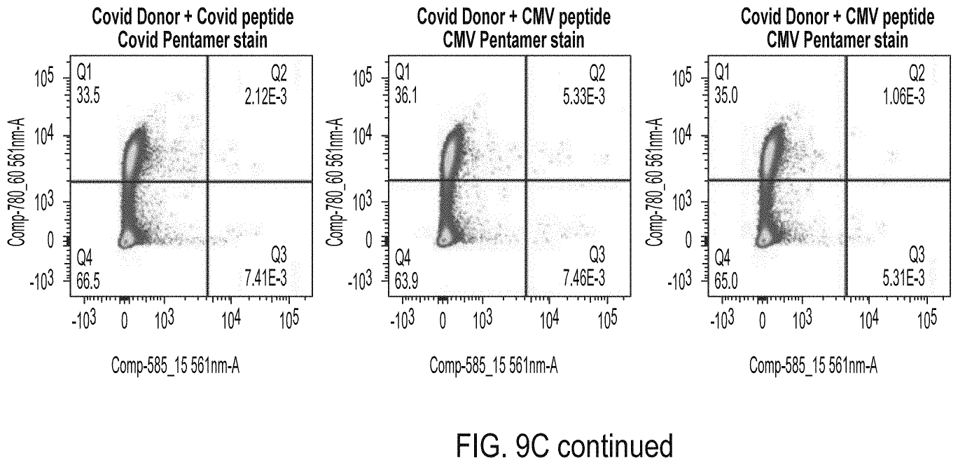

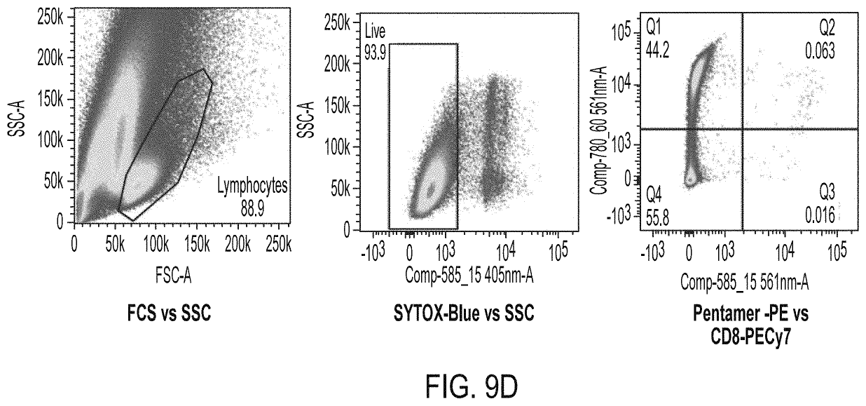

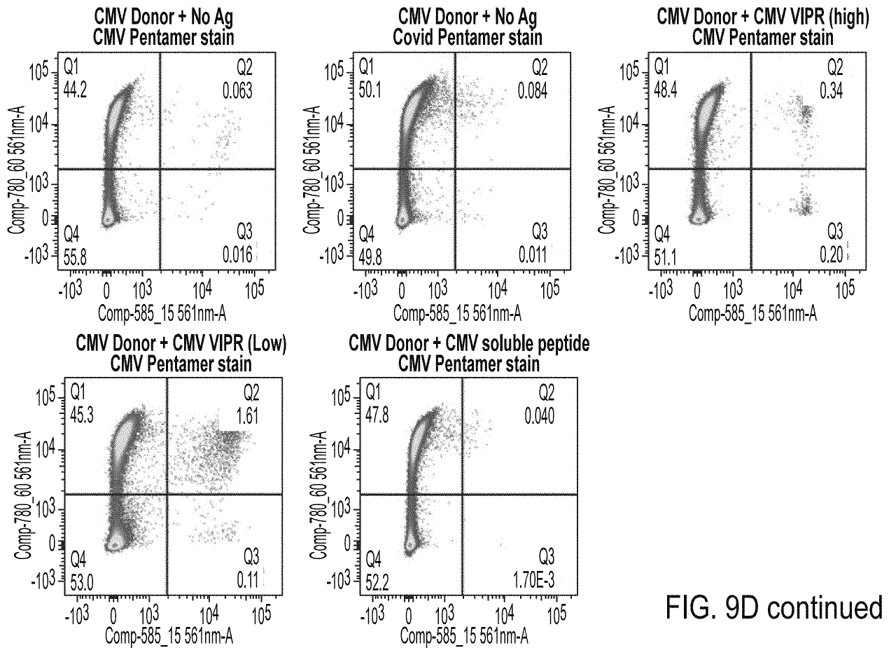

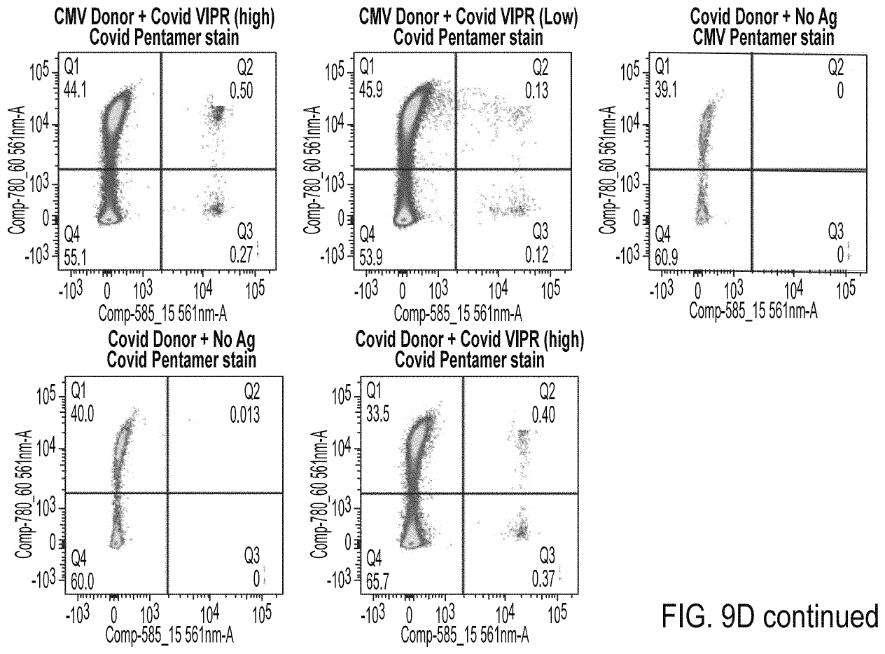

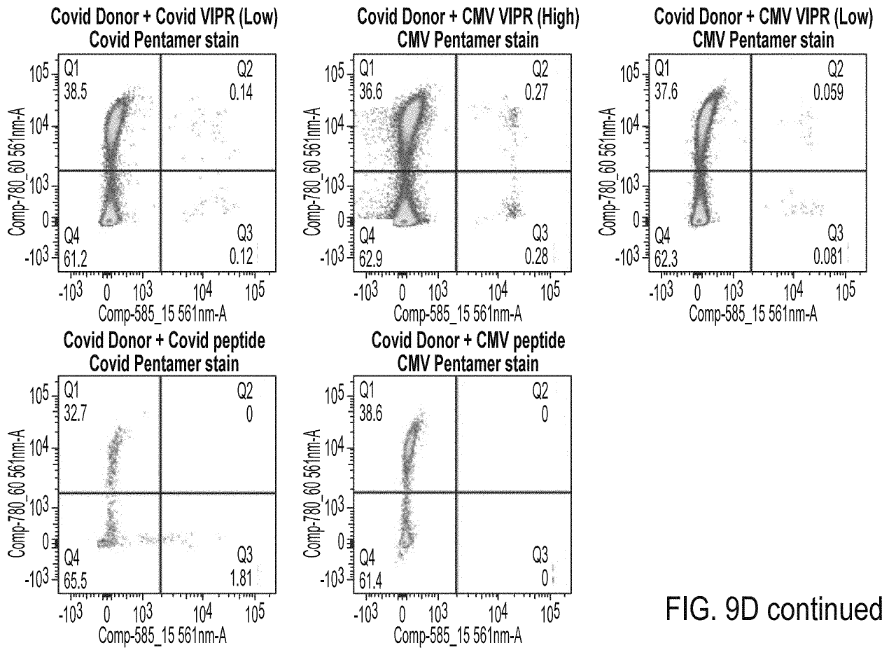

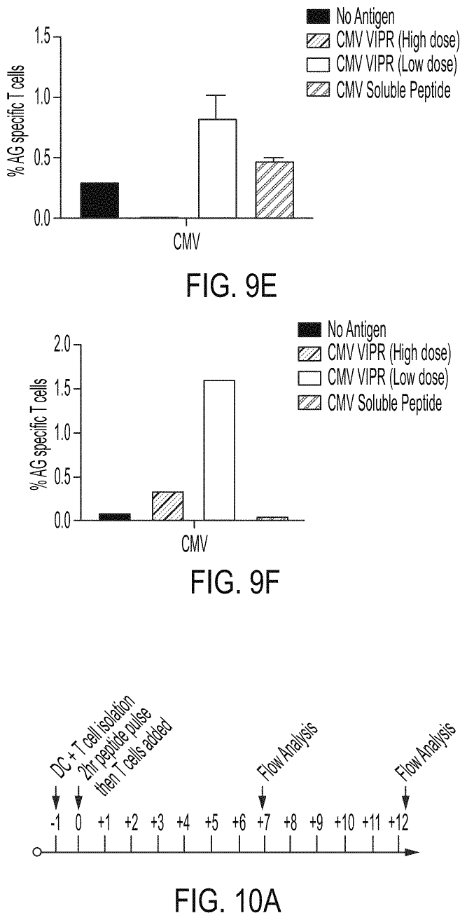

Alternatively, SARS-Cov-2 (SEQ ID NO: 59) or CMV peptide (SEQ ID NO: 70) loaded particles can be cultured with T cells from a COVID-19 positive donor or a CMV positive donor (HLA-A*02:01) respectively, as shown in FIG. 9A. Media was replaced every 2-3 days with complete T cell media containing 10% human serum, IL-2 (6000 IU/ml), IL-7 (5 ng/ml) and IL-15 (5 ng/ml), N acetyl cysteine (10 mM). An additional dose of particles can be added every 2 days where appropriate. Flow cytometry of Biotinylated Pentamers+SA-PE on day 0 (FIG. 9B), day 3 (FIG. 9C), and day 10 (FIG. 9D) are shown. A summary of results for days 3 and 10 are shown in FIG. 9E and FIG. 9F respectively.

Results show that the low dose (5 particles per T cell) appear much better at expanding the T cells than the higher (50 particles per T cell) dose. Fold expansion with low dose V1 CMV particles is: 2.8-fold expansion at Day 3 and 25.5-fold expansion at Day 10. Additionally, soluble peptide can expand CMV+ T cells, in this assay however the particles are more efficient at expanding T cells.

### Example 7: Stimulation of T Cells with SARS-Cov2 Specific Particles and Dendritic Cells to Generate a Virus Inducted Lymphocyte (VIL)

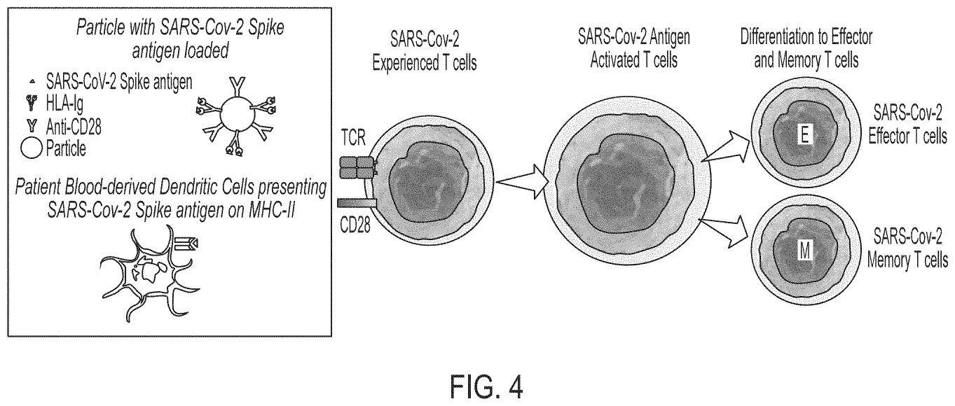

T cells are diluted to 1×10⁶/mL, aliquoted into 5 mL cultures in a 6 well plate, and contacted with about 40 μg/mL particles expressing peptide in the context of HLA class I and peptide-pulsed dendritic cells (expressing HLA class II) for 24 hours at 37° C. thereby expanding both CD4 and CD8 T cells, FIG. 4. After 24 hour incubation, the particles are removed, and the cultures are fed every 2-3 days with fresh medium containing 50 IU/ml rIL-2. After 24 hours, the VILs are analyzed as previously described.

### Example 8: Stimulation of T Cells with SARS-Cov2 Specific Particles to Generate a Virus Inducted Lymphocyte (VIL)

### SARS-CoV2 FIAGLIAIV Antigen (SEQ ID NO: 59)

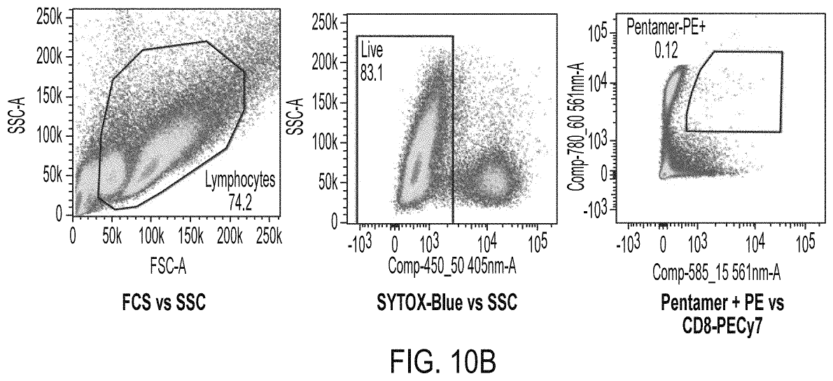

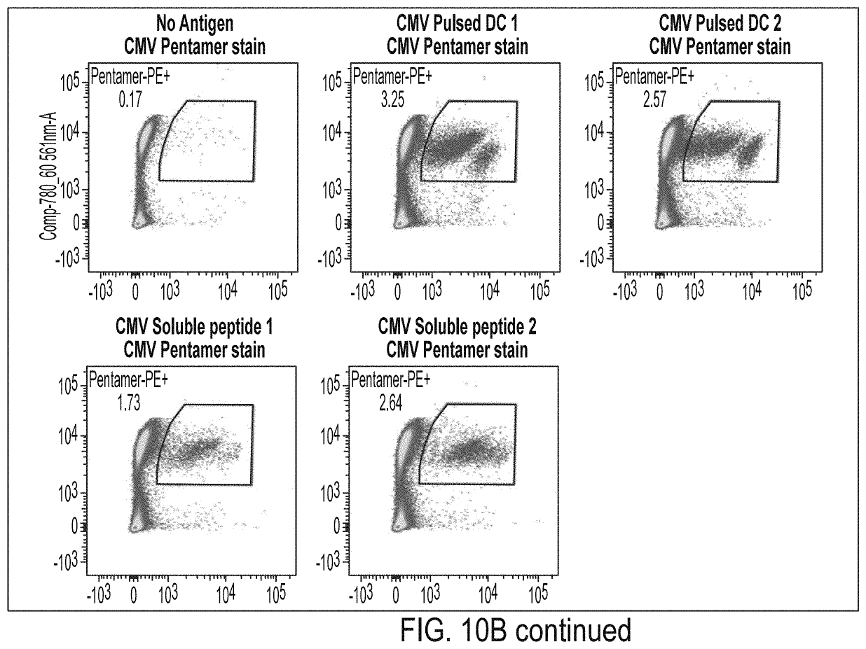

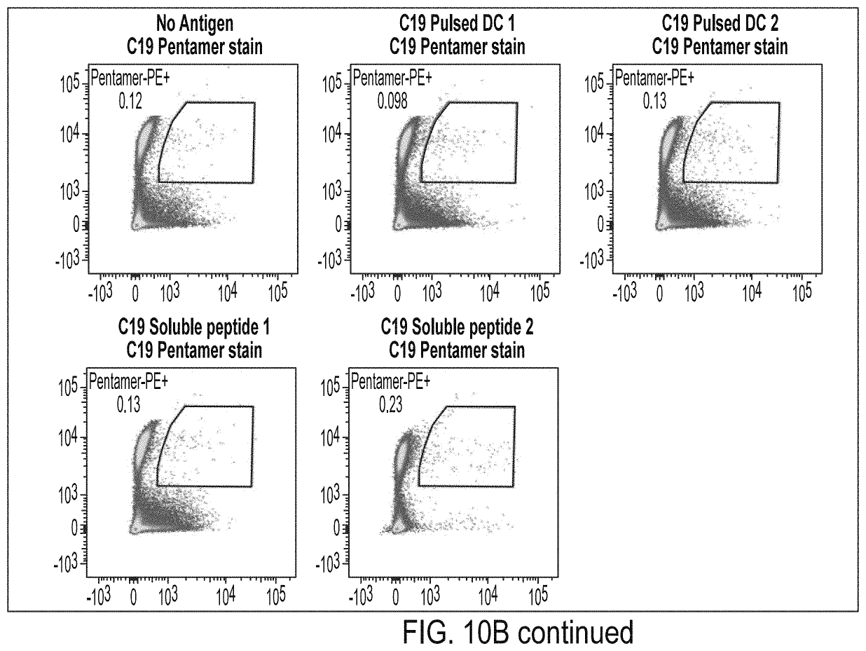

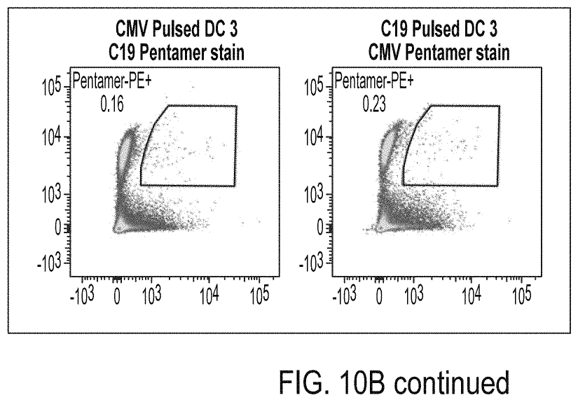

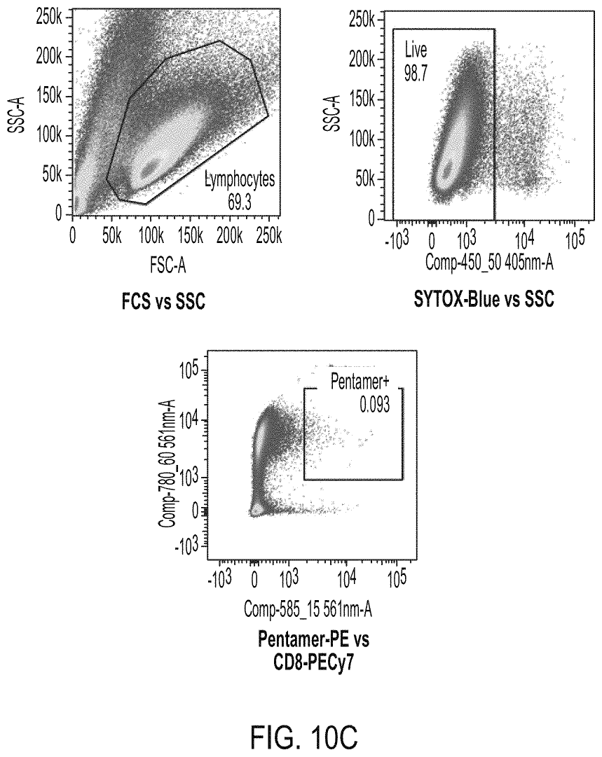

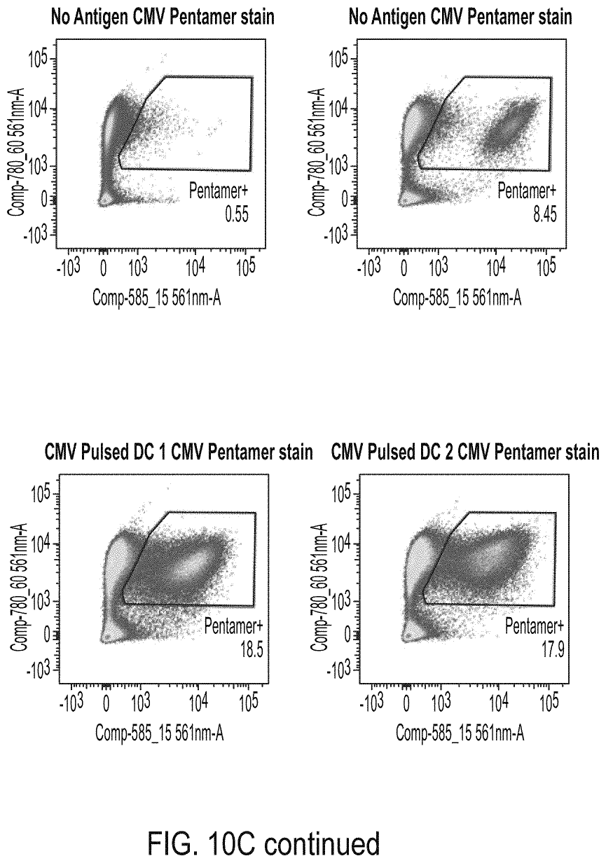

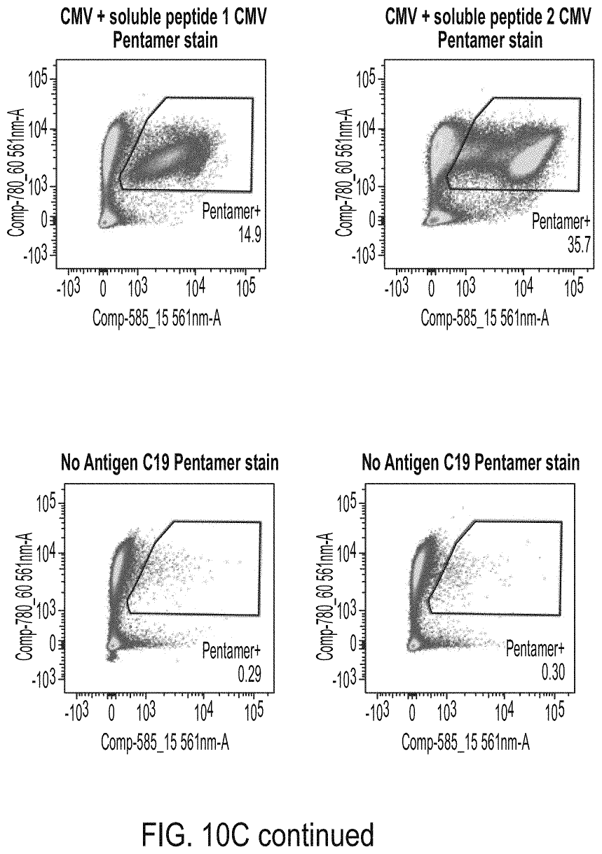

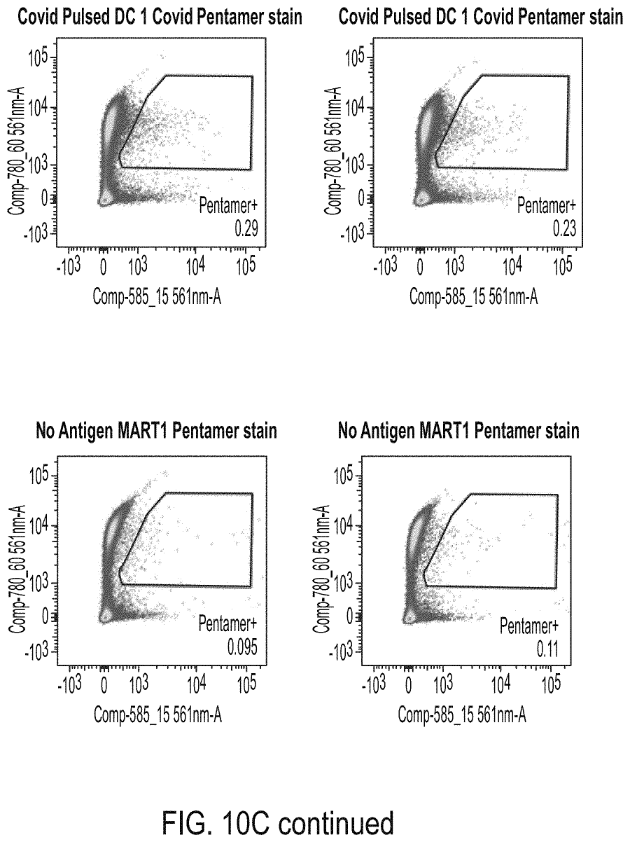

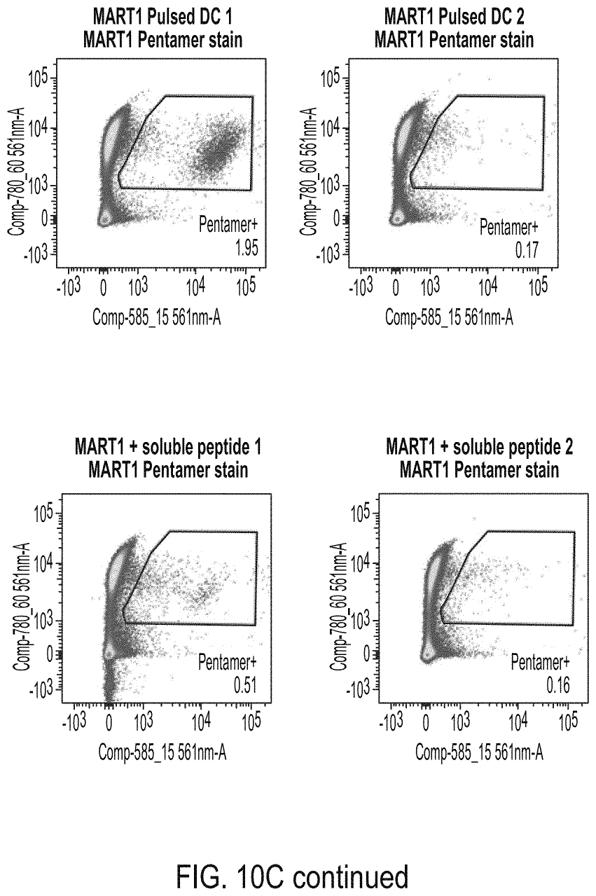

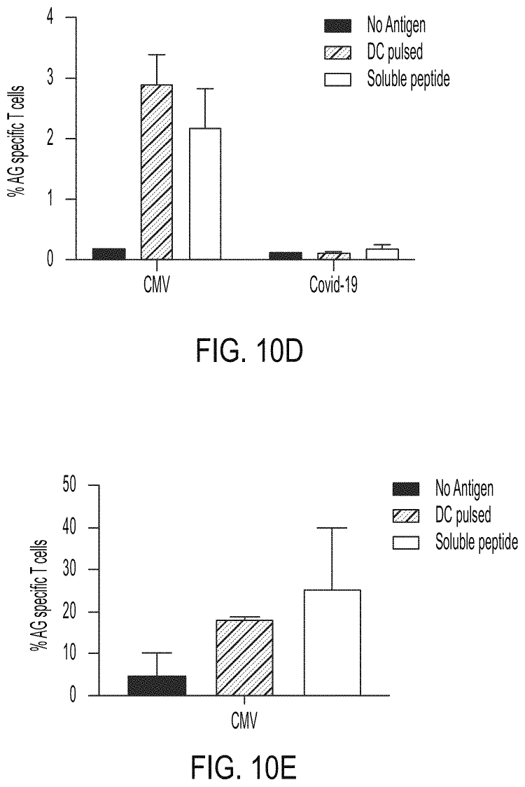

Alternatively, stimulation can also be performed with antigen peptide loaded DCs alone utilizing a CMV positive donor that is HLA-A*02:01. Peptides that were tested include: CMV pp65 70% peptide of SEQ ID NO: 70, SARS-CoV-2 70% peptide of SEQ ID NO: 59, and MART1 70% peptide of SEQ ID NO: 71. Conditions that were tested are DCs and T cells from a CMV+ donor: (1) DCs pulsed with CMV Ag; Covid Ag-1; MART Ag; (2) Stained with each Pentamer-Bio and analyzed Day 7 and Day12. Briefly, DCs were pulsed with 20 ug/ml peptide for 2 hours (no wash) with the addition of 2×10⁵ T Cells in the presence of 6000 U/mL of IL-2. Flow cytometry utilizing MHCI-Pentamer PE was performed on day 7 (FIG. 10B), and day 12 (FIG. 10C). A summary of day 7 and day 12 data is shown in FIG. 10D and FIG. 10E respectively.

CMV pulsed DC T cell expansion shows that by day 7, the DCs outperform soluble peptide for CMV specific expansions leading to a 17-fold expansion of T cells. By day 12 all cells have expanded, and the soluble peptide cultures have overtaken the DCs.

### SARS-CoV2 YLQPRTFLL (SEQ ID NO: 72)

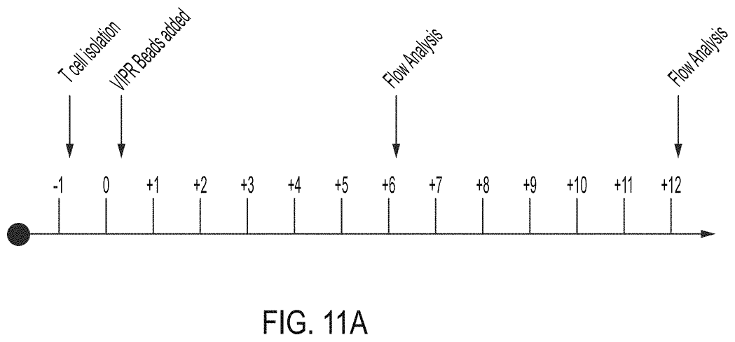

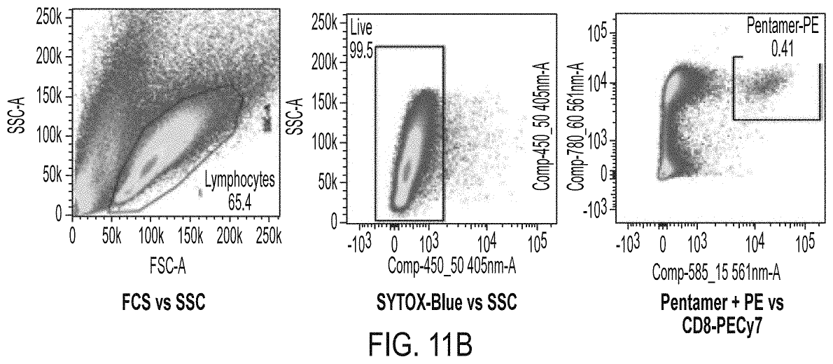

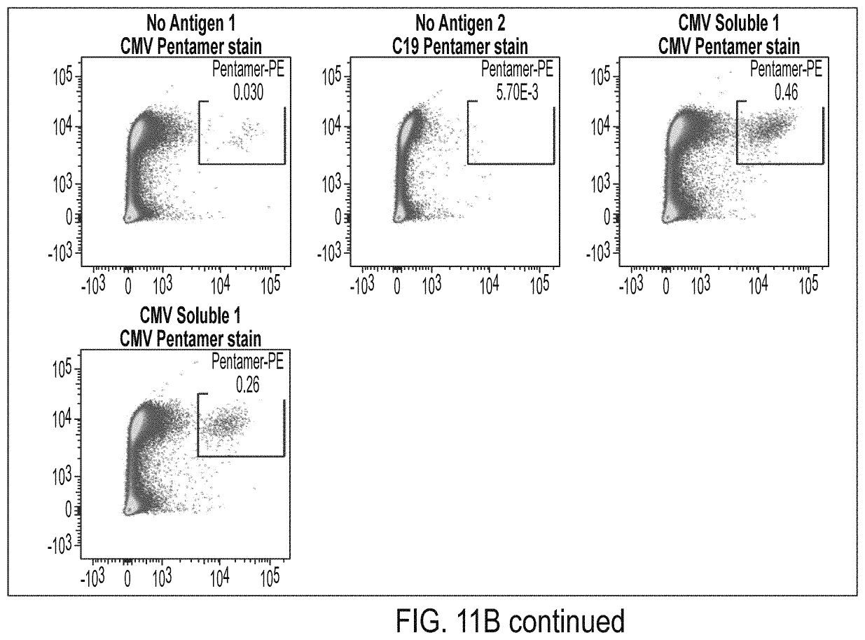

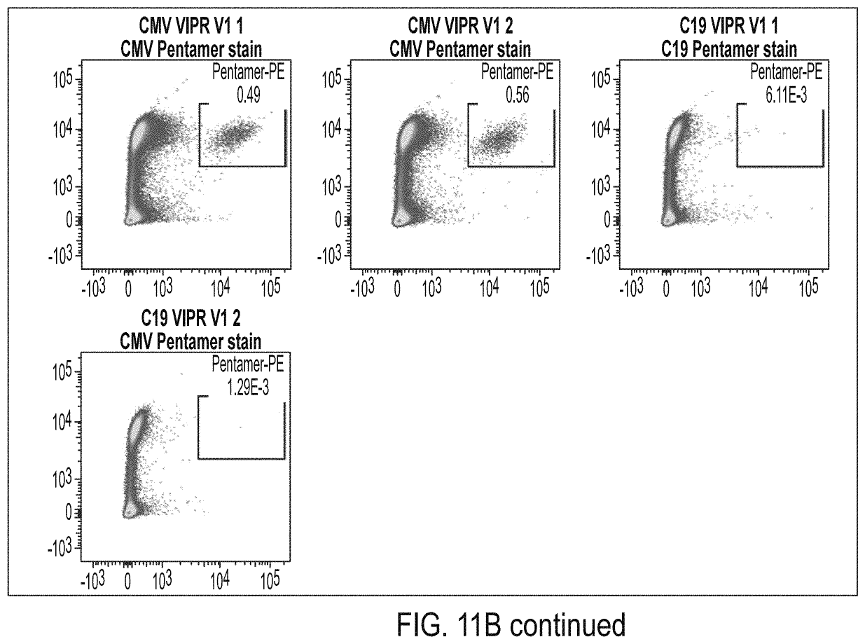

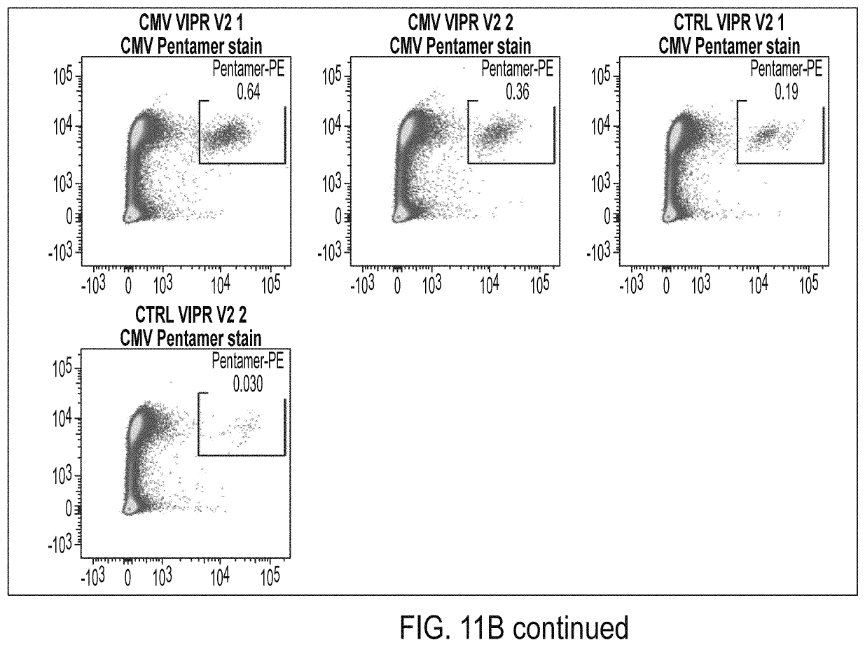

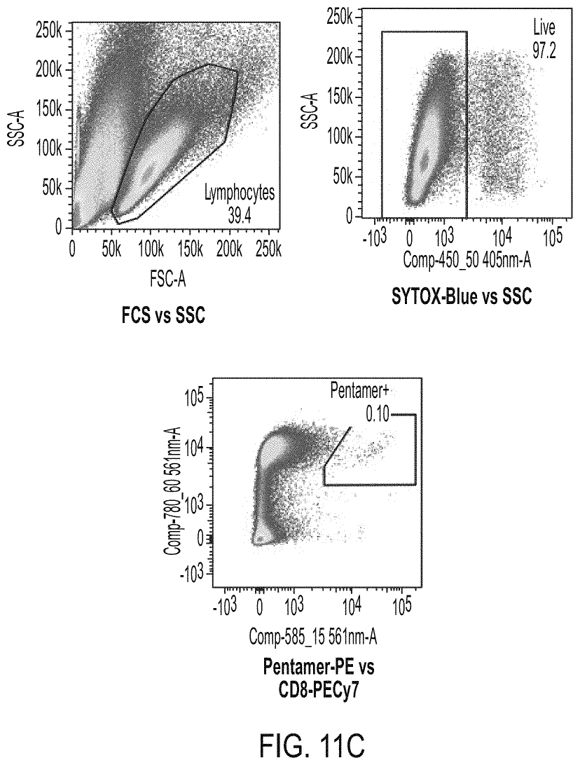

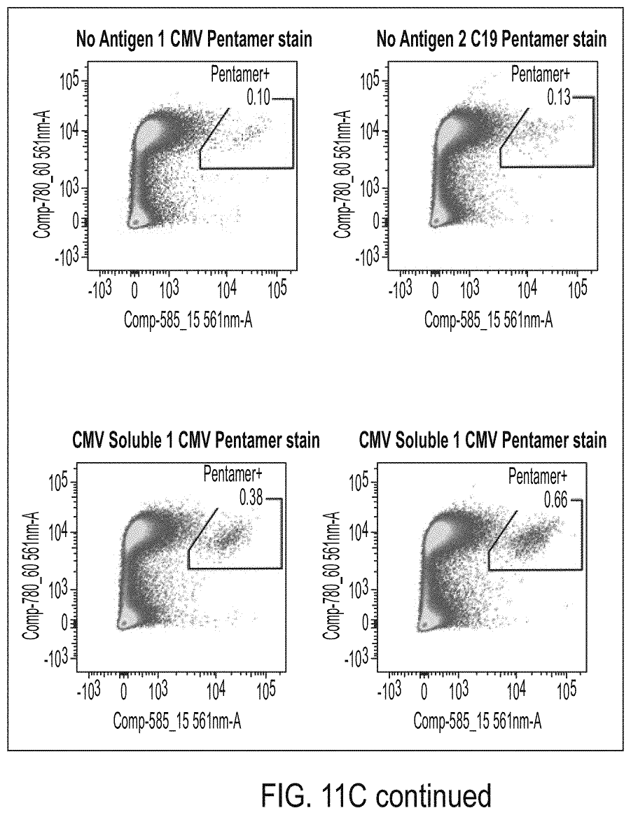

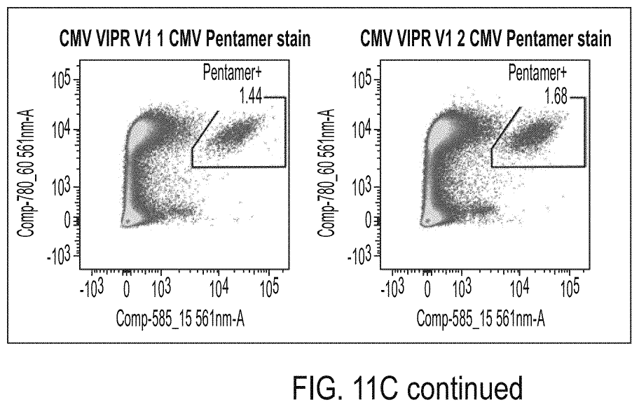

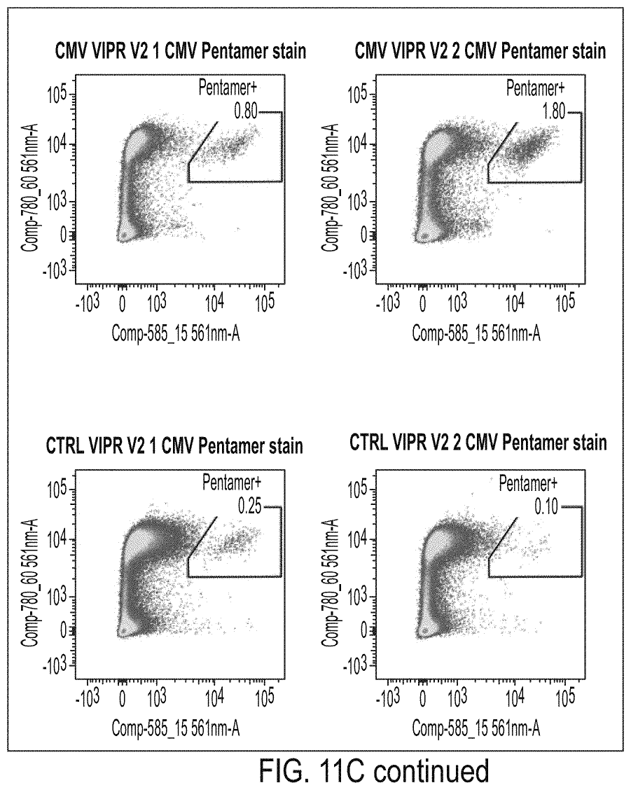

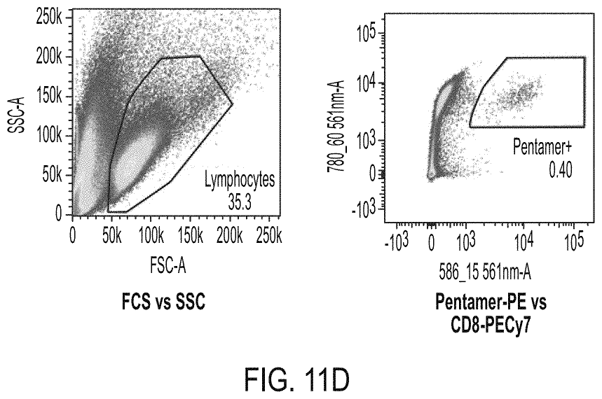

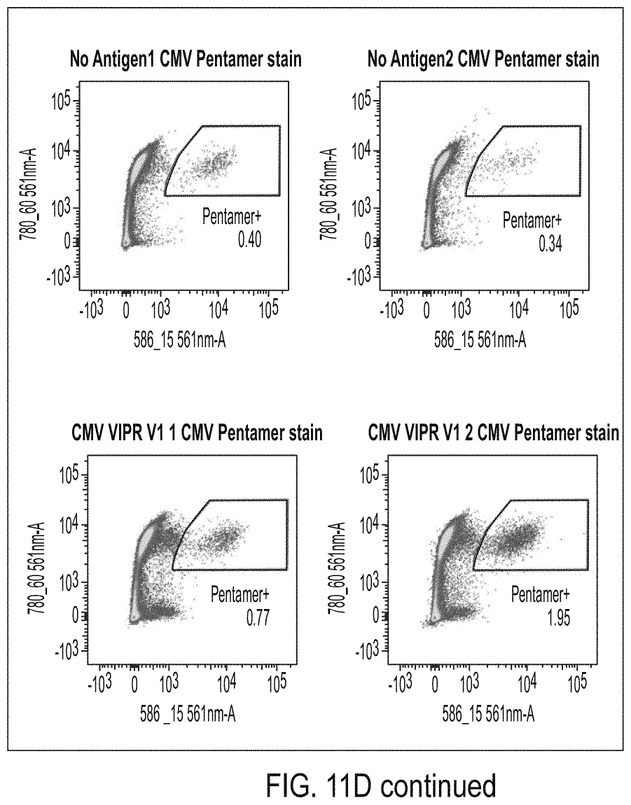

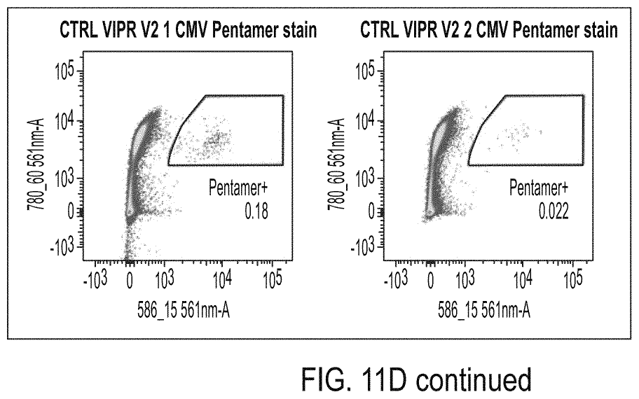

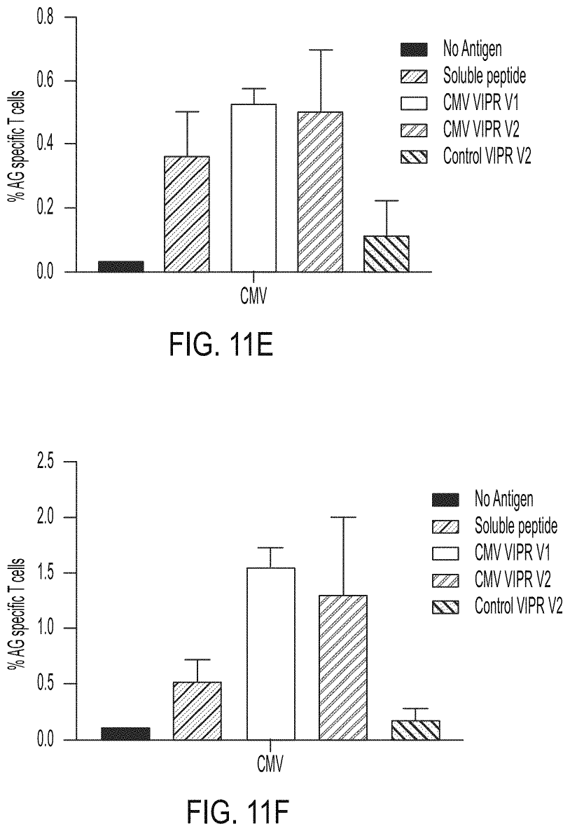

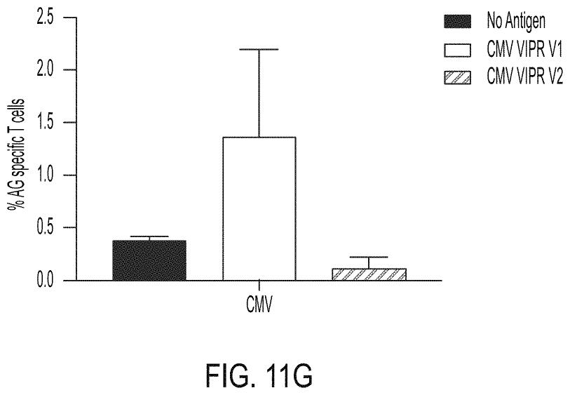

Comparative studies of V1 particles (CMV Pentamer: CD28) and V2 particles (CMV Pentamer: CD3: CD28) were performed using T cells from a CMV positive donor that was also HLA-A*02:01. Conditions that were tested include: (1) Comparison to soluble matched peptide antigen; (2) V1 particles; V2 particles; and V2 control (CD3:CD28); and (3) Covid antigen-1 as a test to see if cells are detectible (also as negative control). Briefly, DCs were pulsed with 20 ug/ml peptide (SEQ ID NO: 70 and SEQ ID NO: 72) for 2 hours (no wash) with the addition of 2×10⁵ T Cells in the presence of 6000 U/mL of IL-2. Flow cytometry utilizing MHCI-Pentamer PE was performed on day 6 (FIG. 11B), day 12 (FIG. 11C), and day 15 (FIG. 11D). A summary of day 6, day 12 and day 15 data is shown in FIG. 11E, FIG. 11F, and FIG. 11G respectively.

Results show that the Version 1 particle (Pentamer+CD28) is more efficient at expanding antigen-specific T cells than the Version 2 (Pentamer+CD28+CD3). The control beads, CD3+CD28 conjugated to M-280 beads, show very little antigen-specific expansion as expected. Fold expansion conferred by the V1 particle is as follows: Day 6: 17.5-fold, Day 12: 15.6-fold, and Day 15: 3.7-fold.

### Re-Dosing of Antigen Loaded Particles

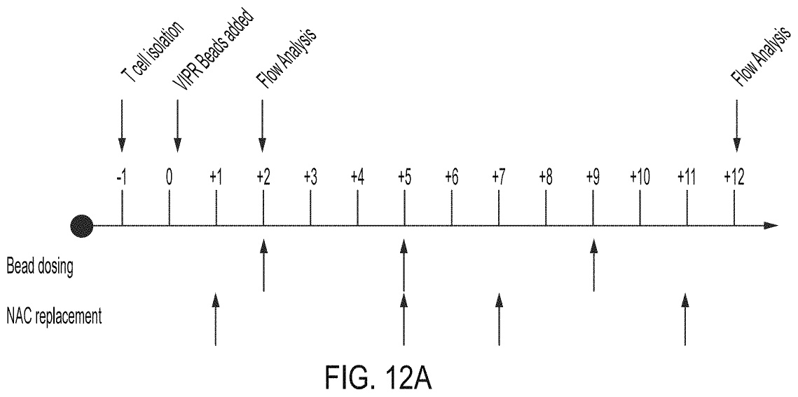

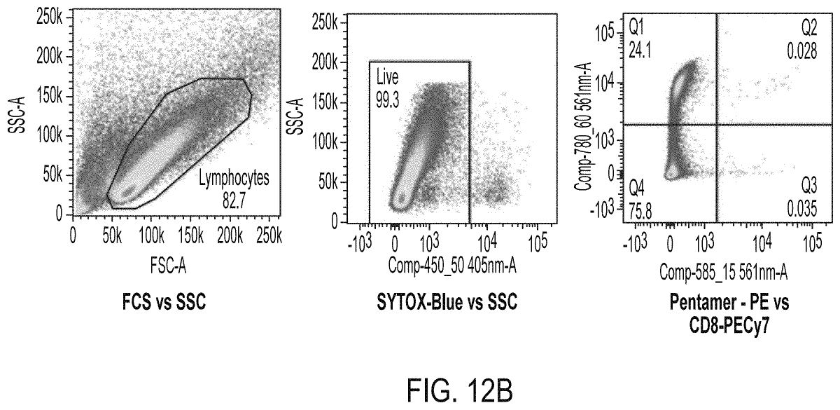

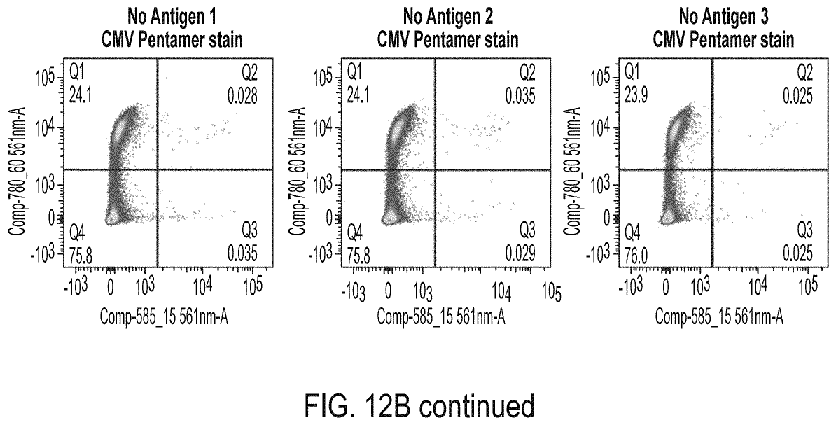

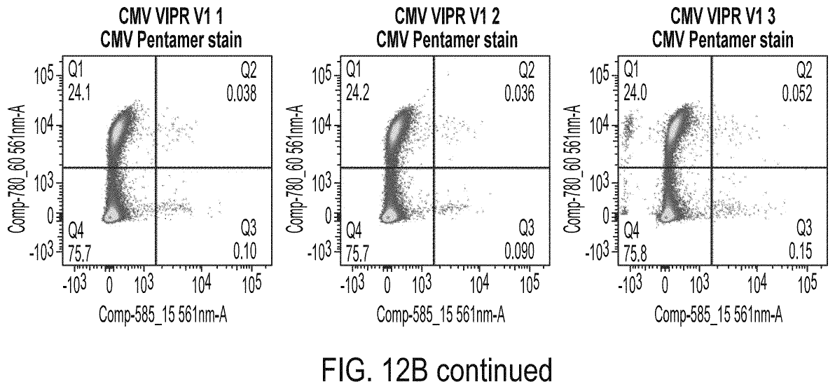

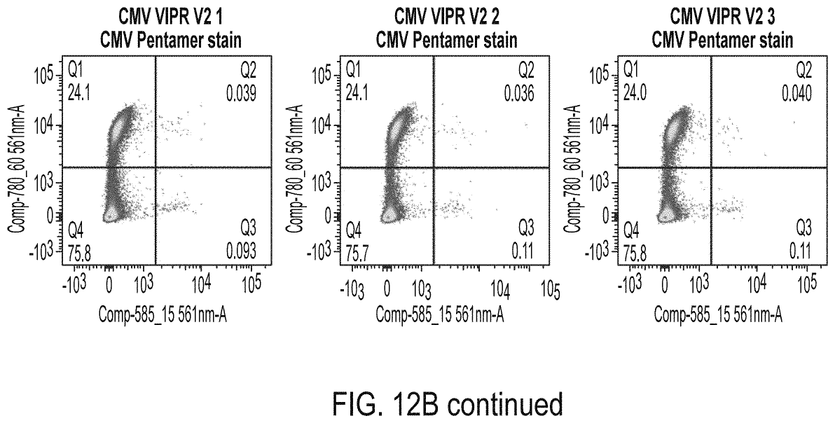

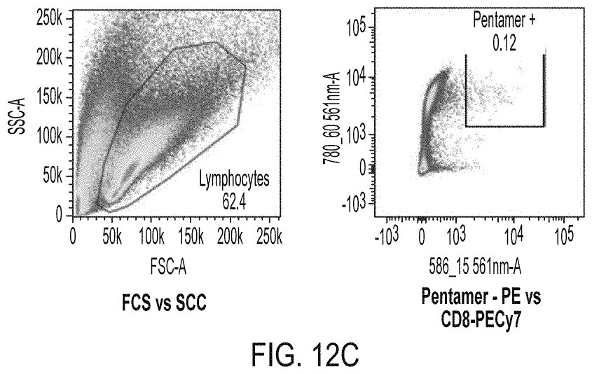

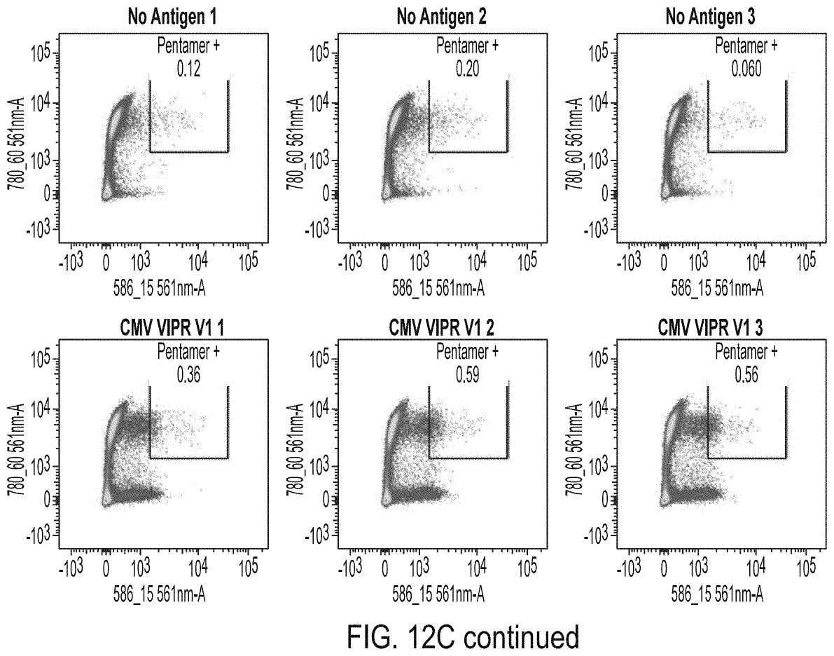

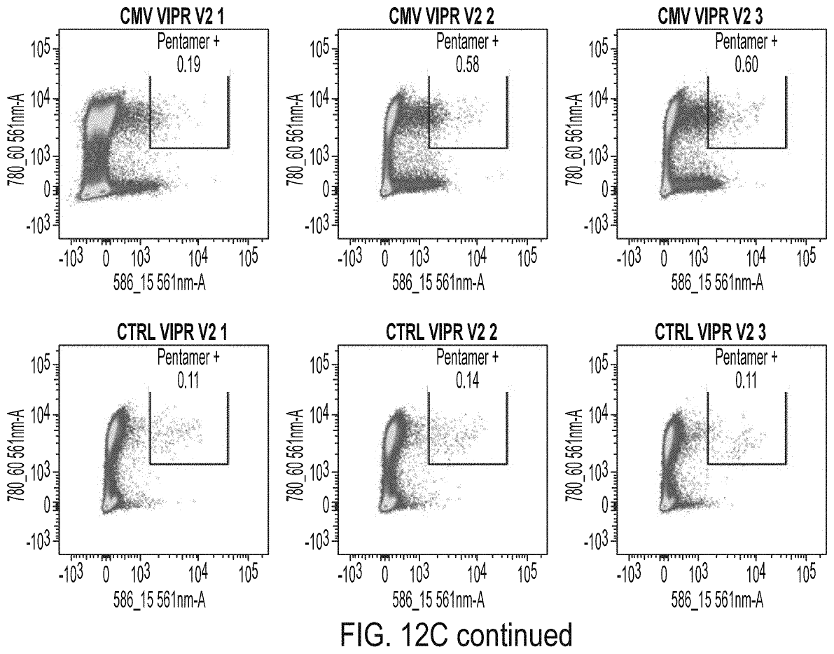

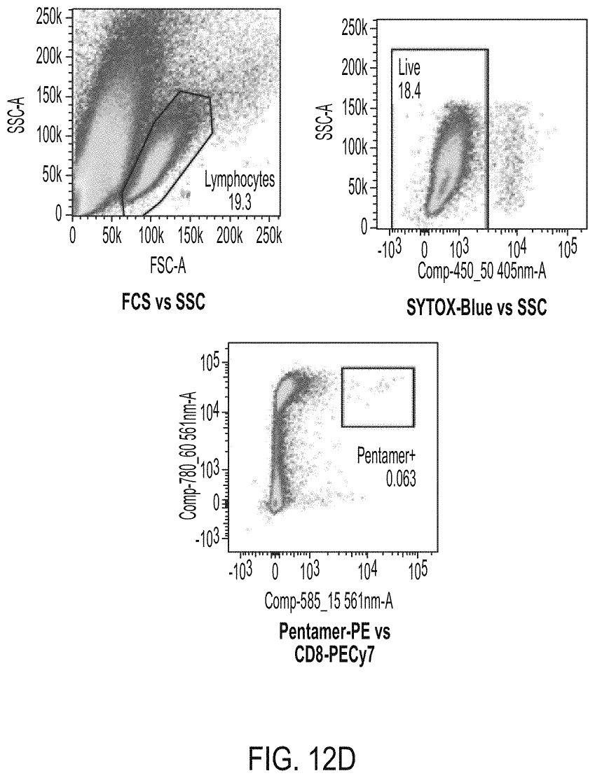

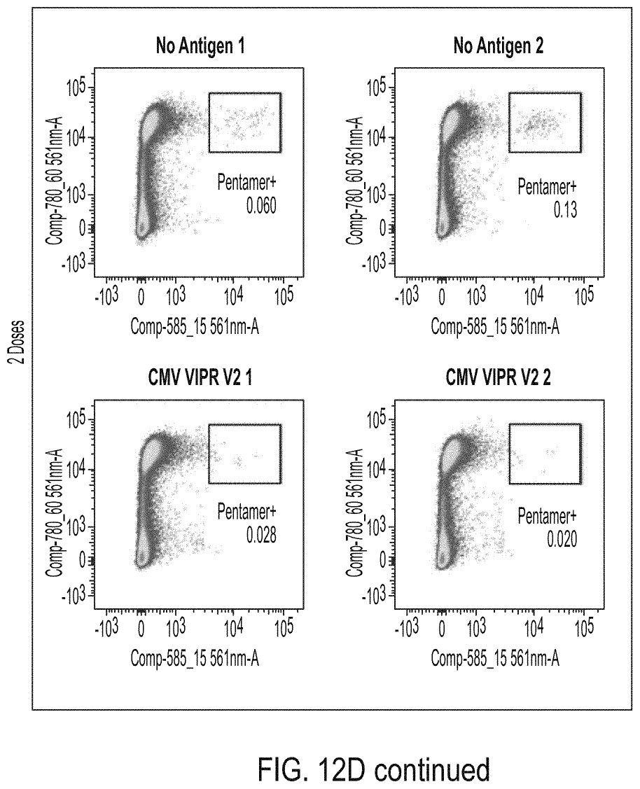

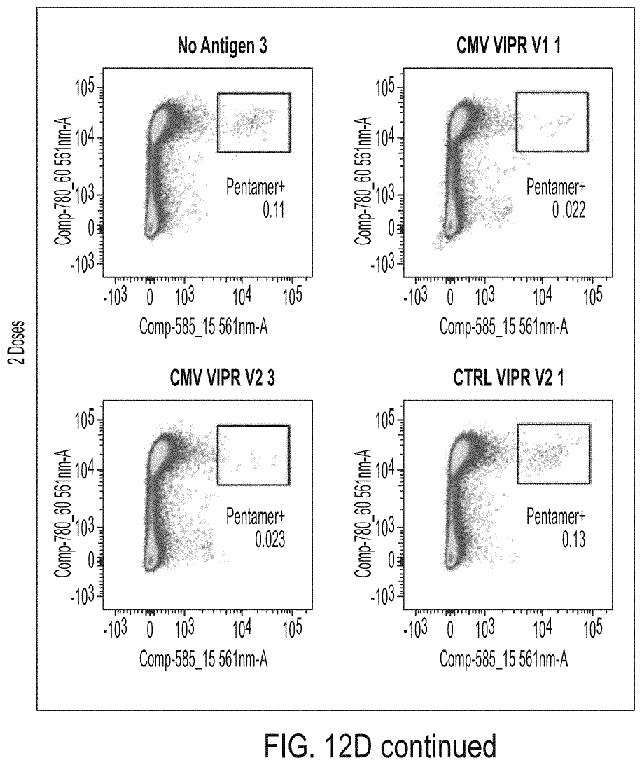

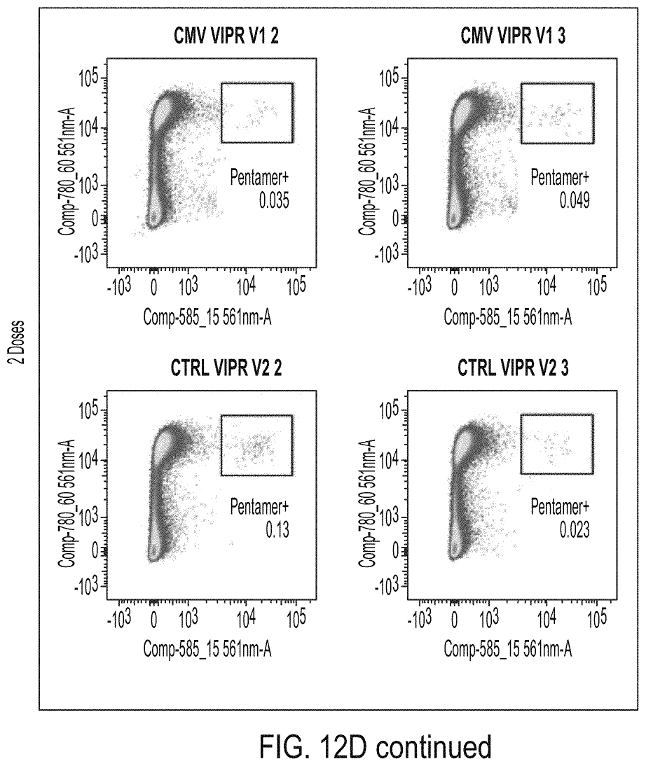

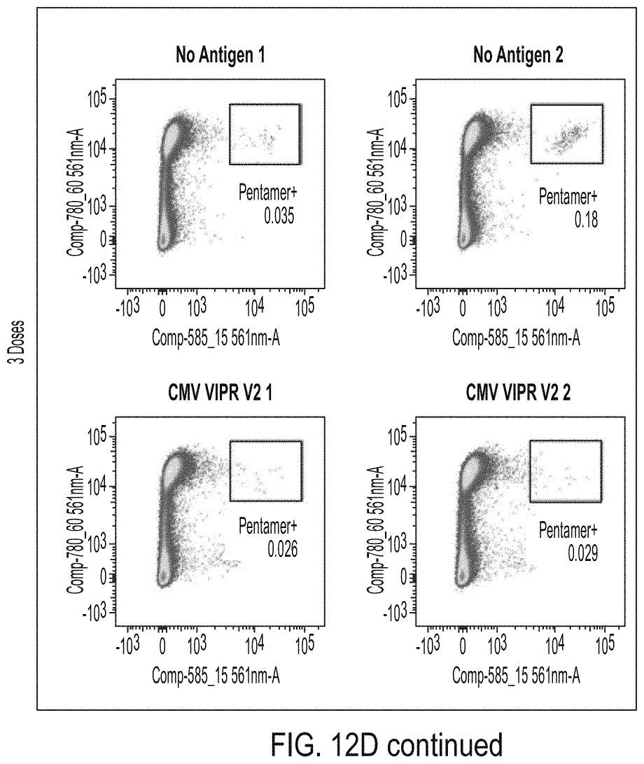

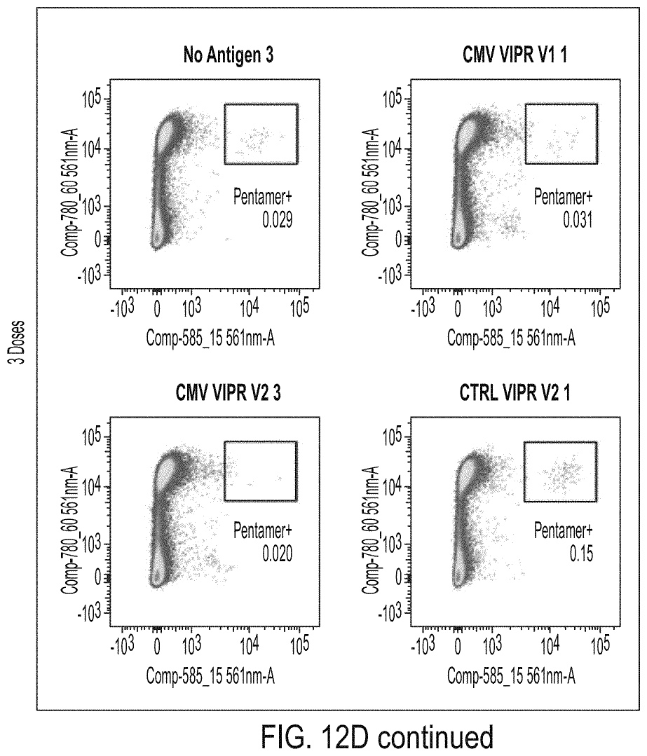

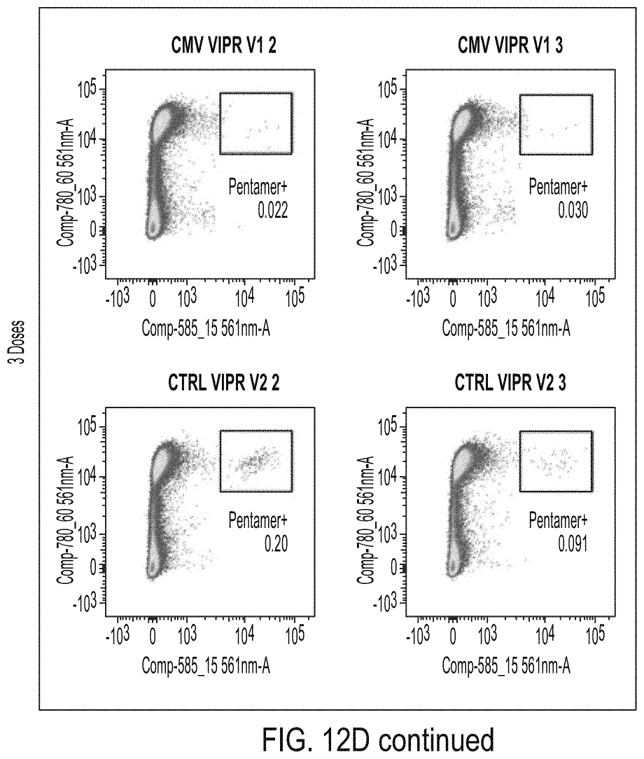

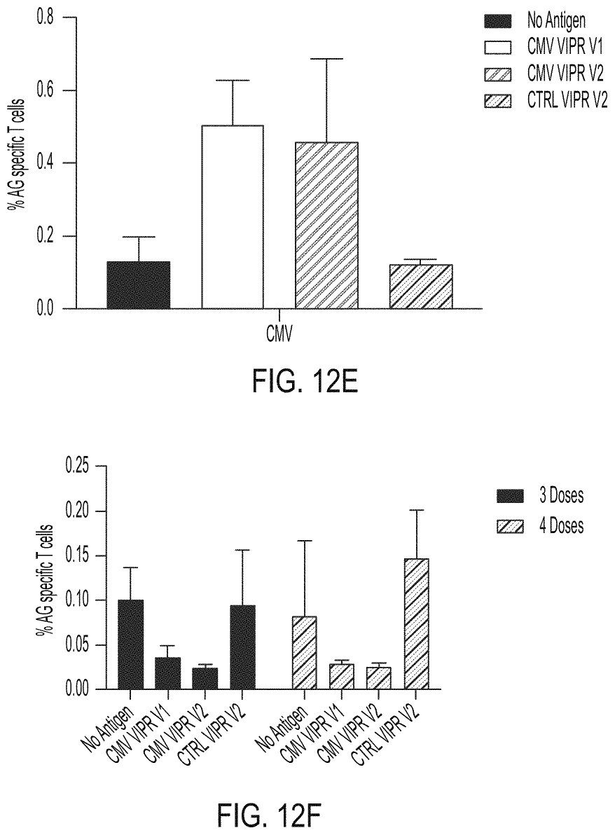

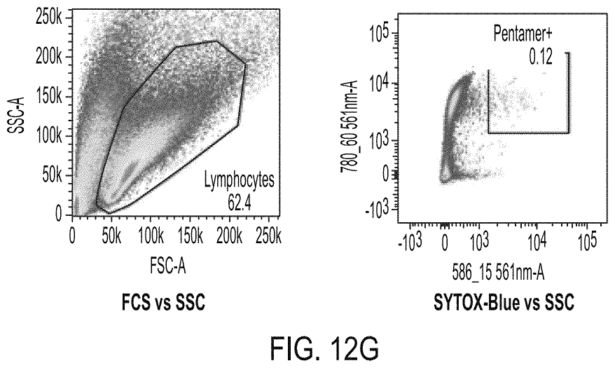

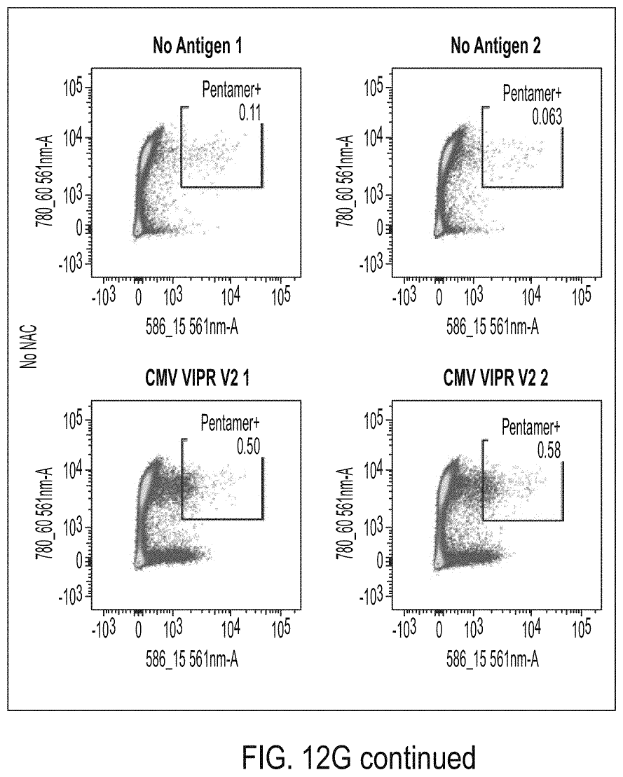

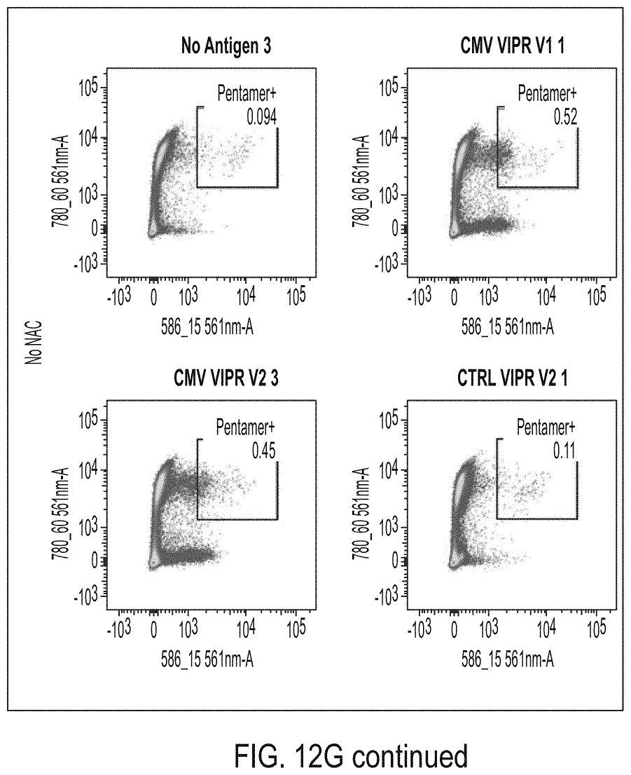

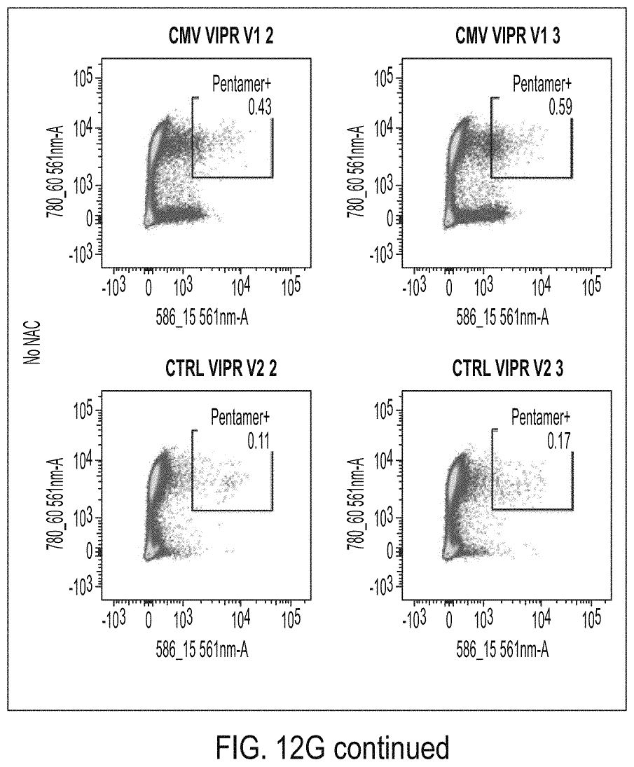

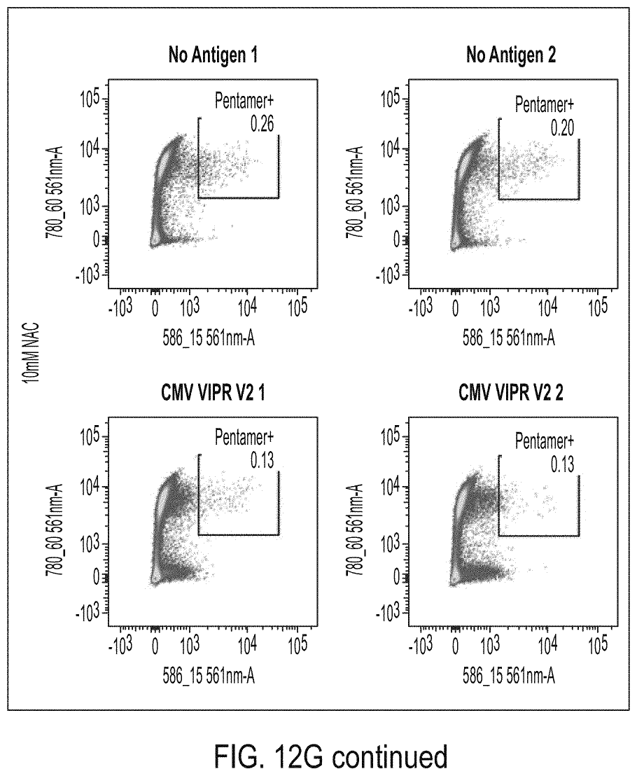

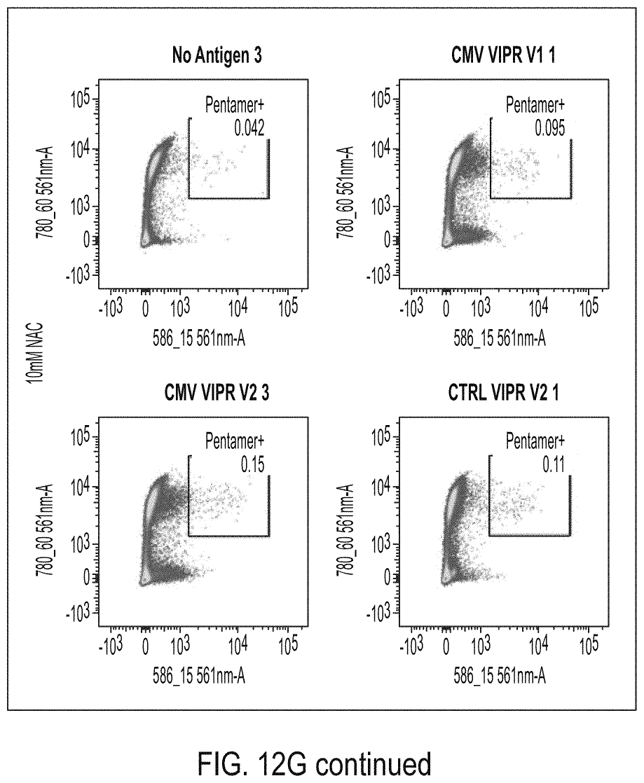

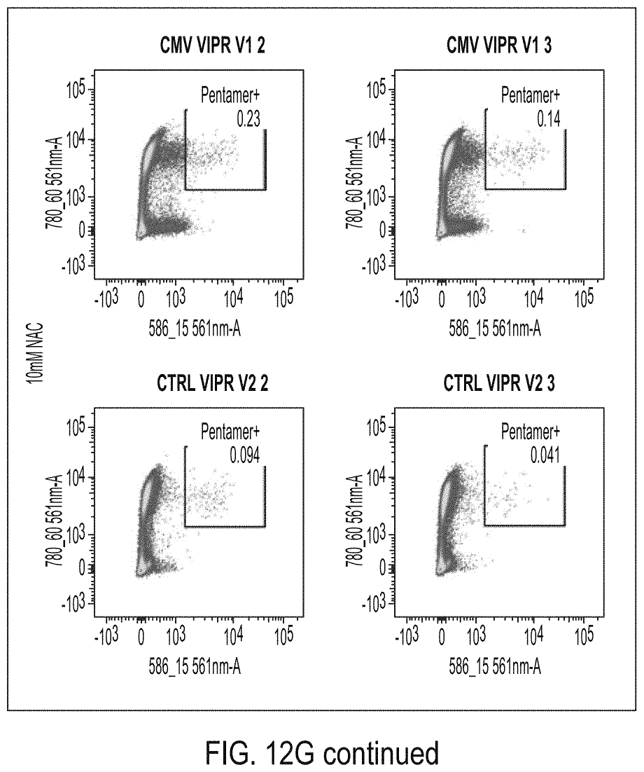

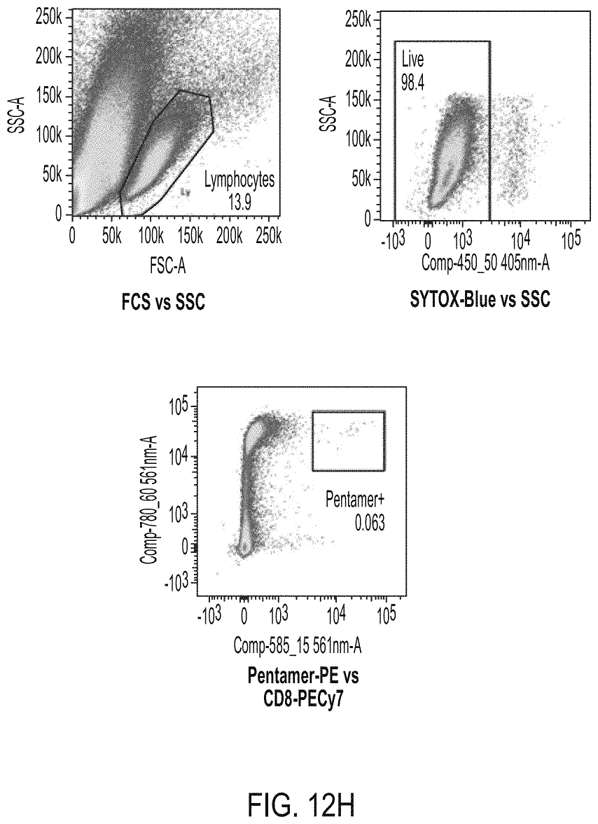

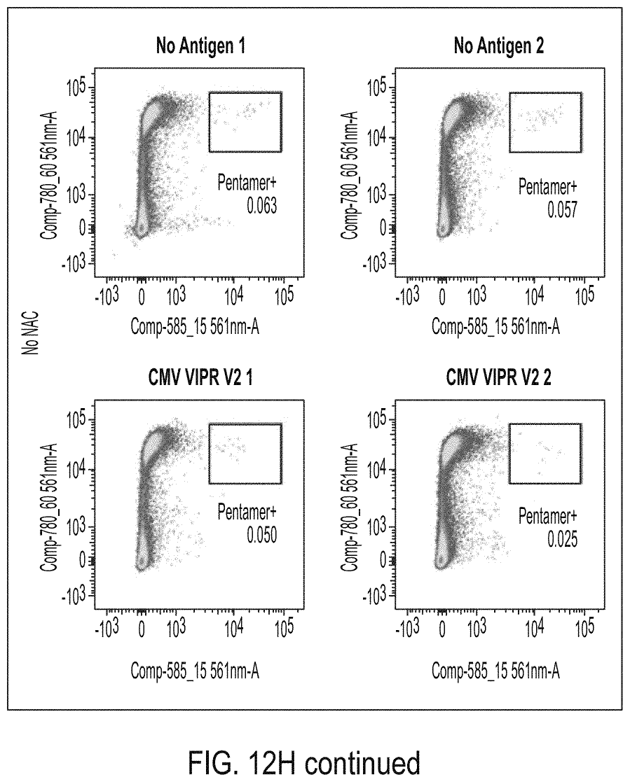

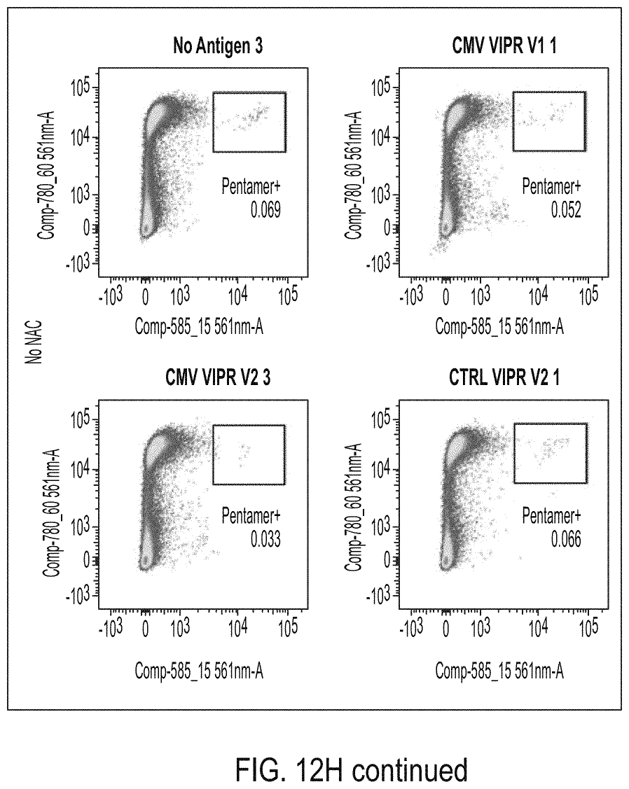

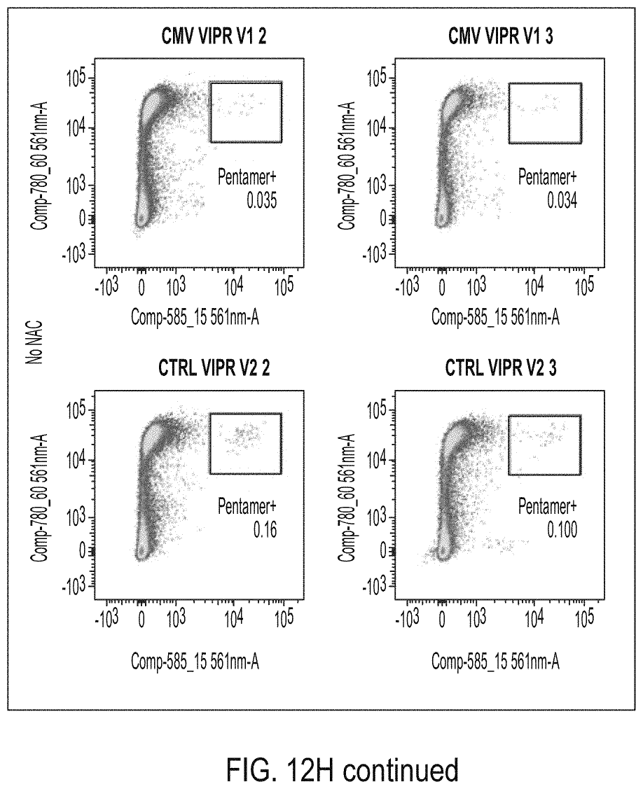

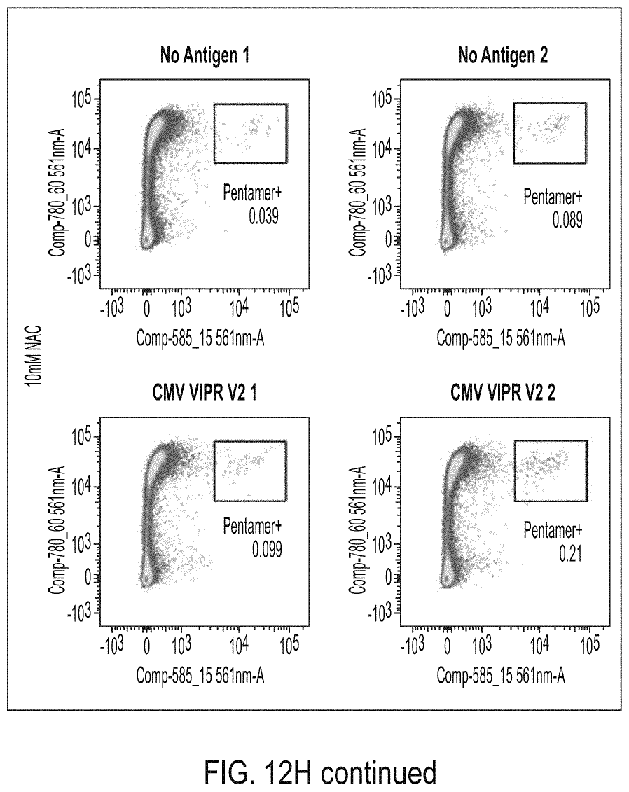

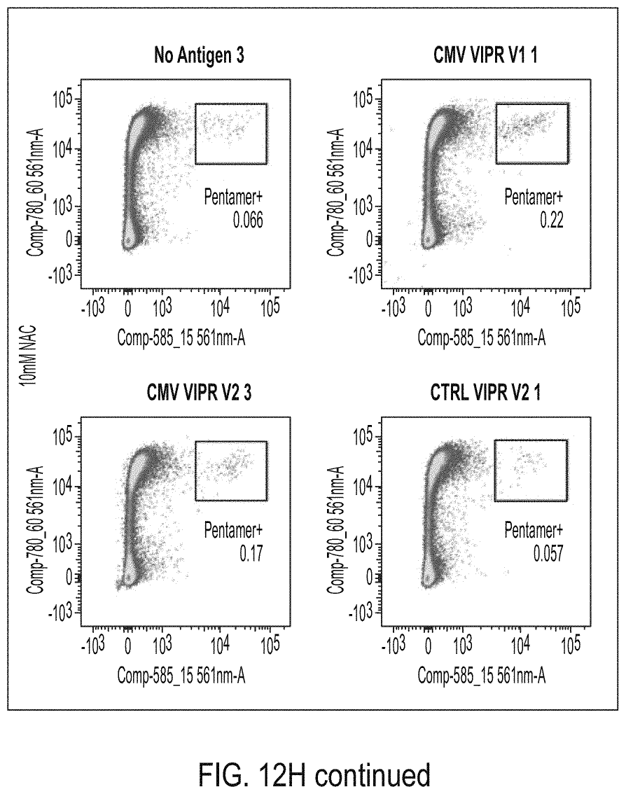

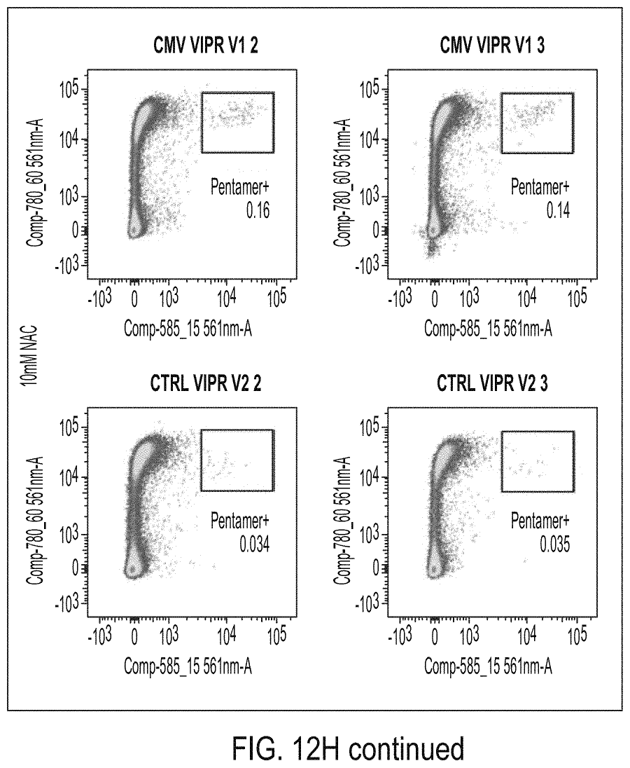

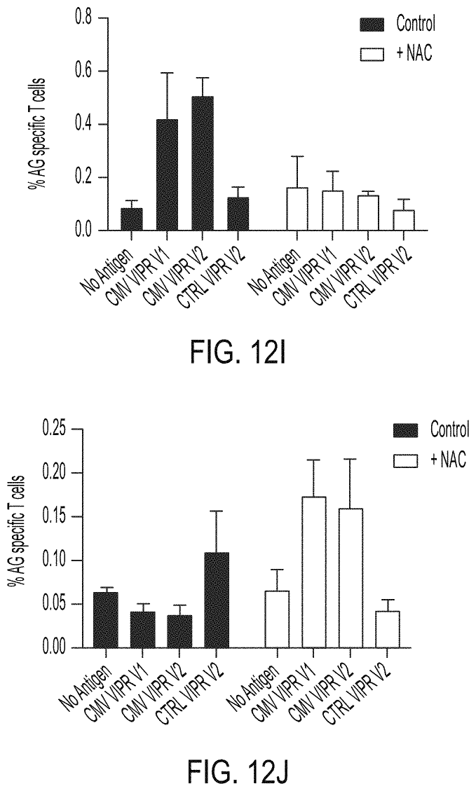

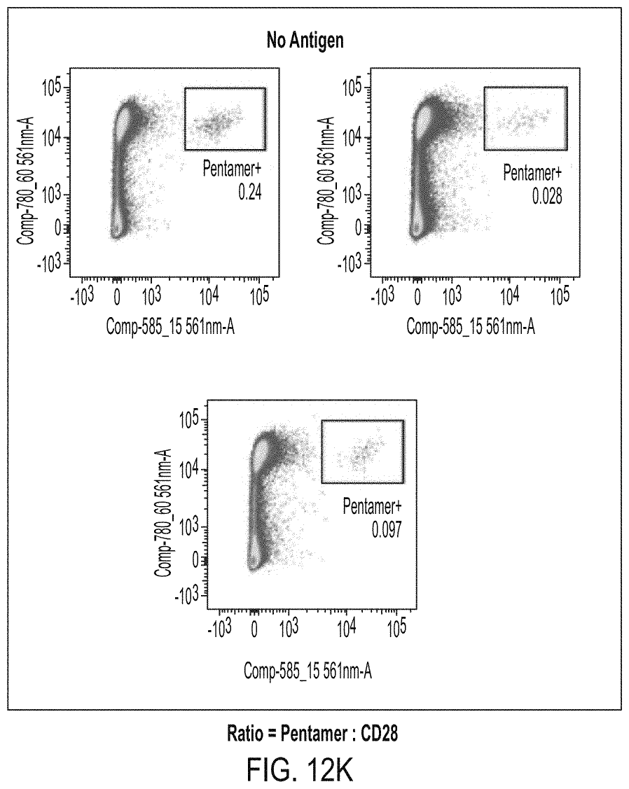

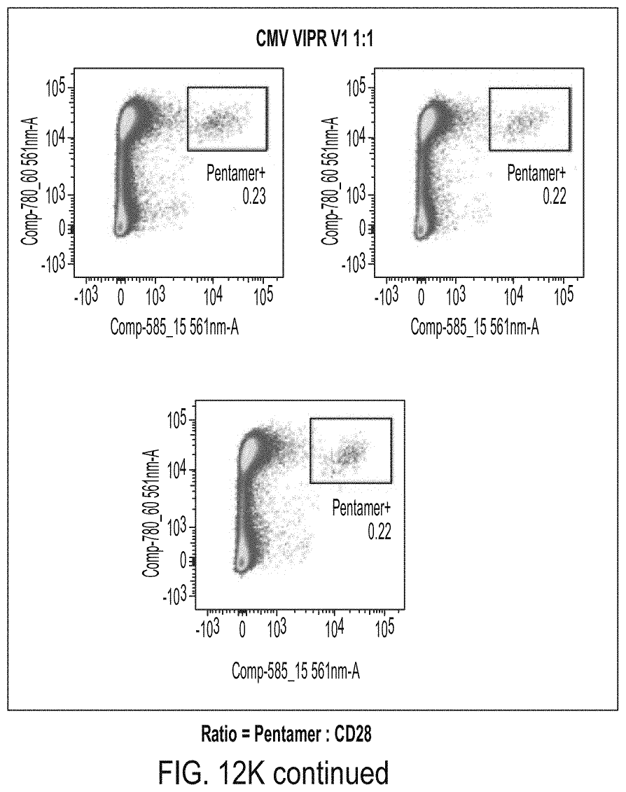

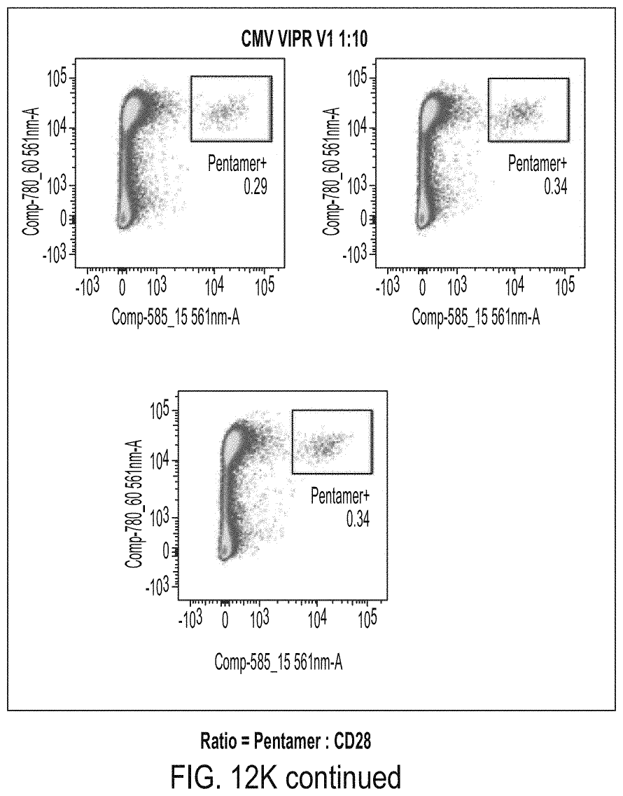

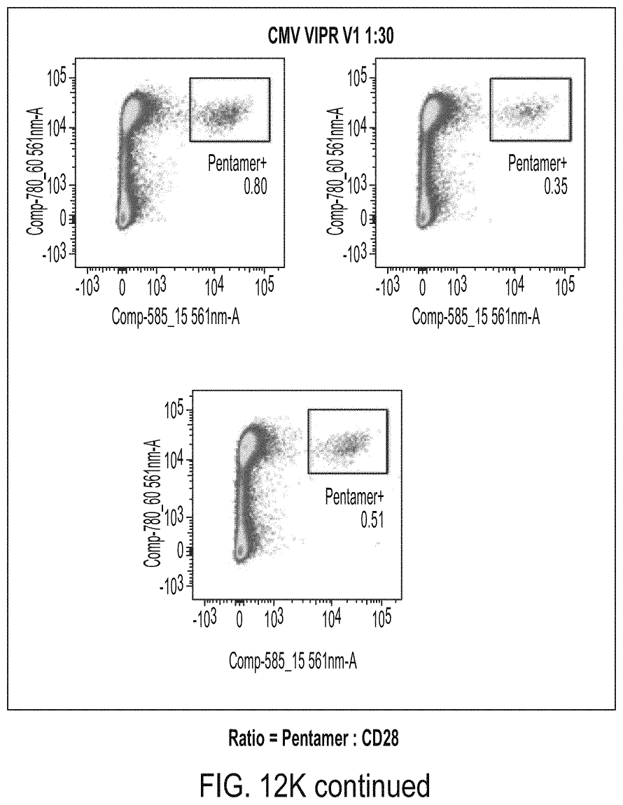

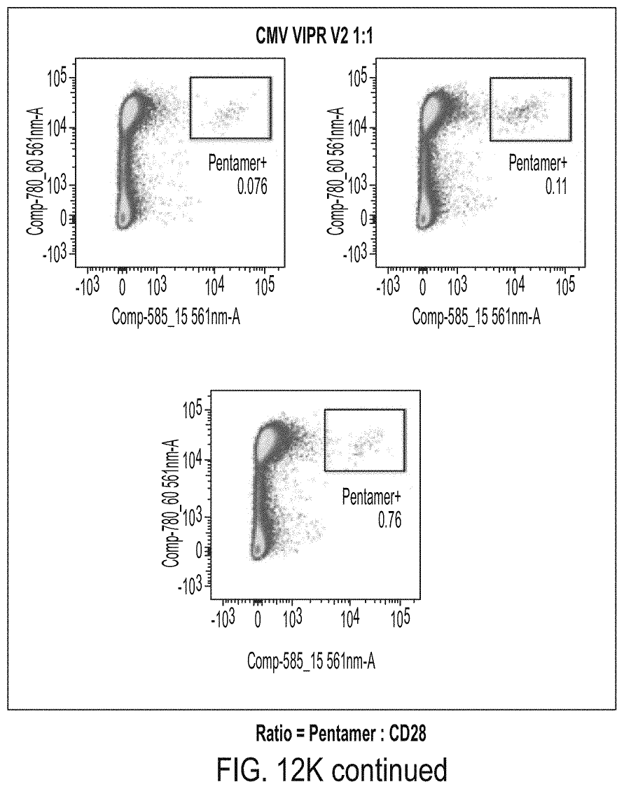

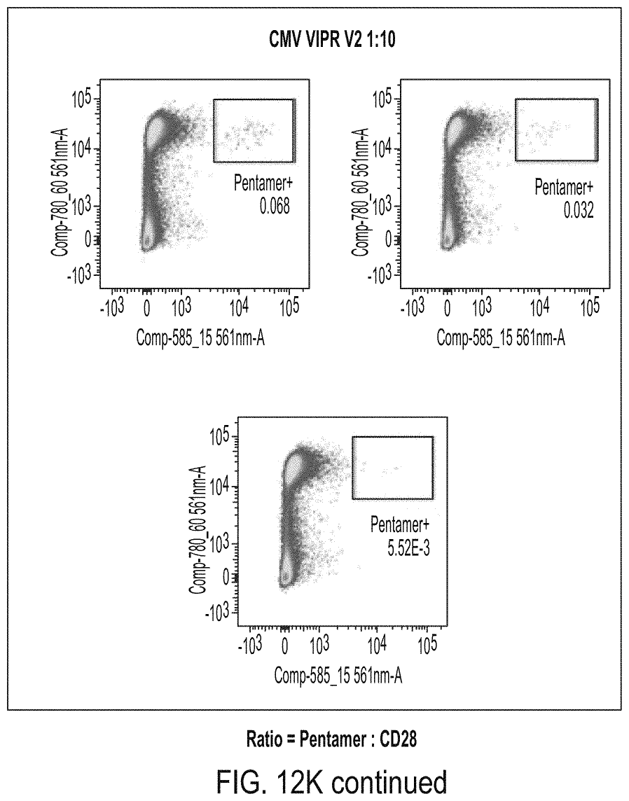

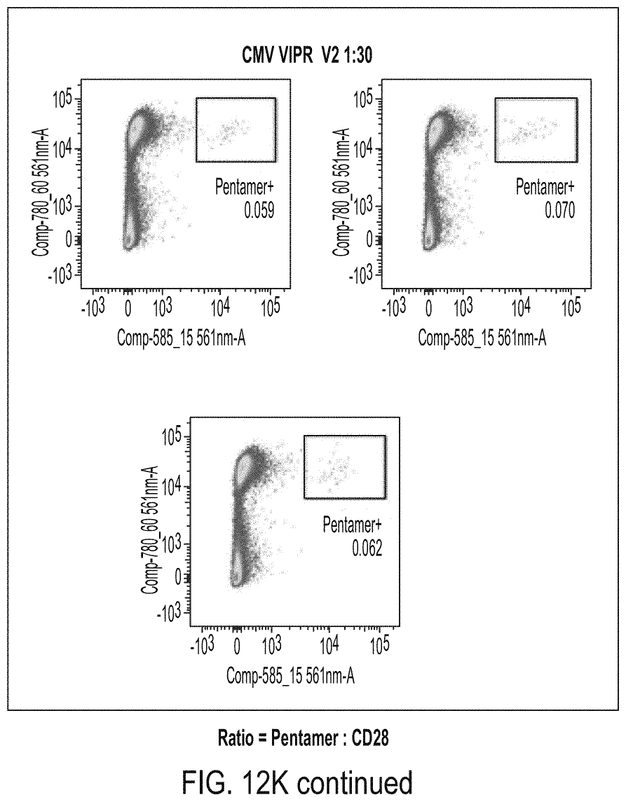

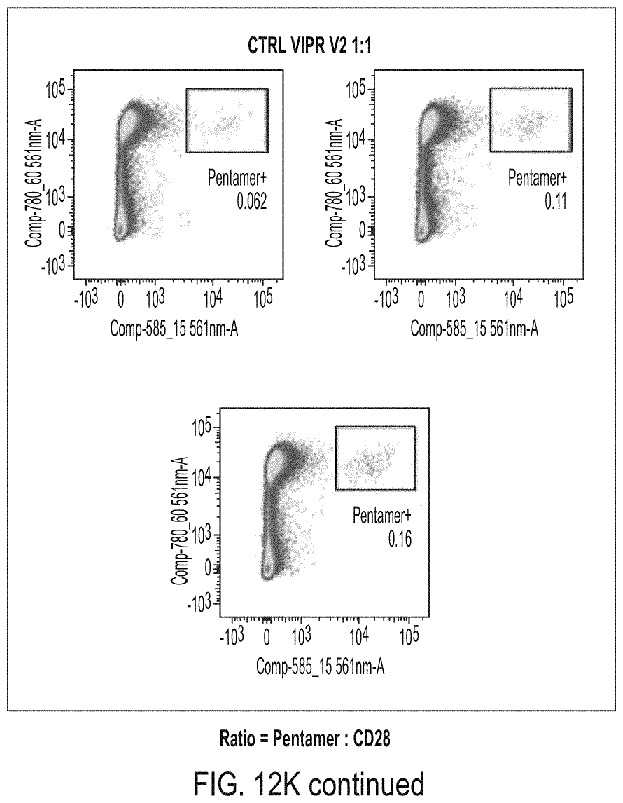

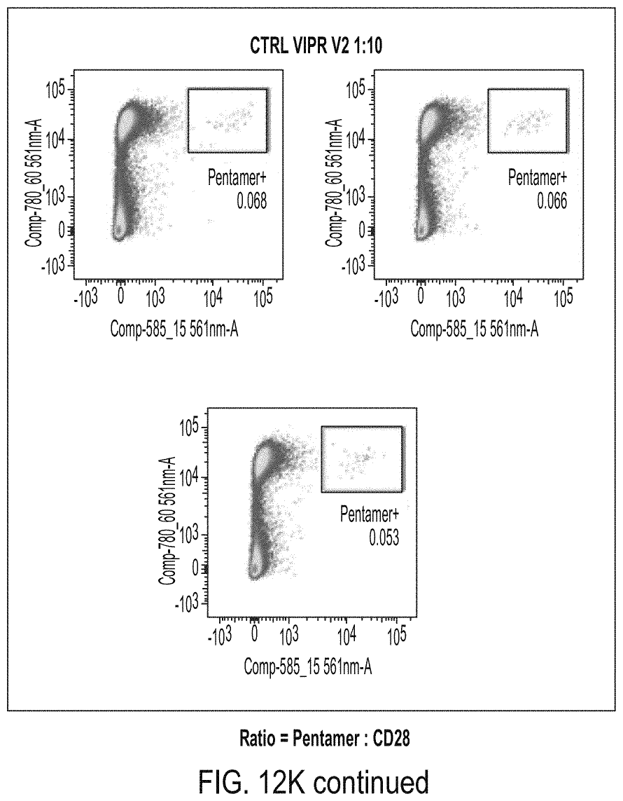

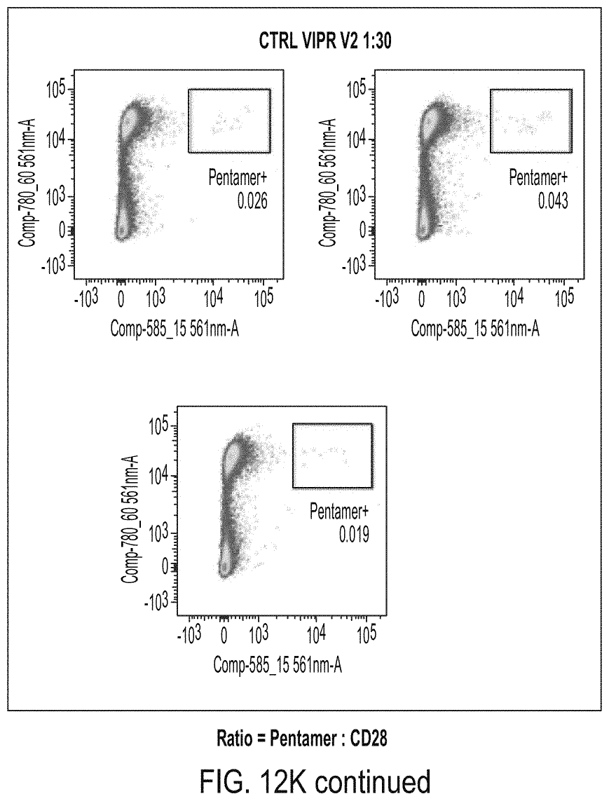

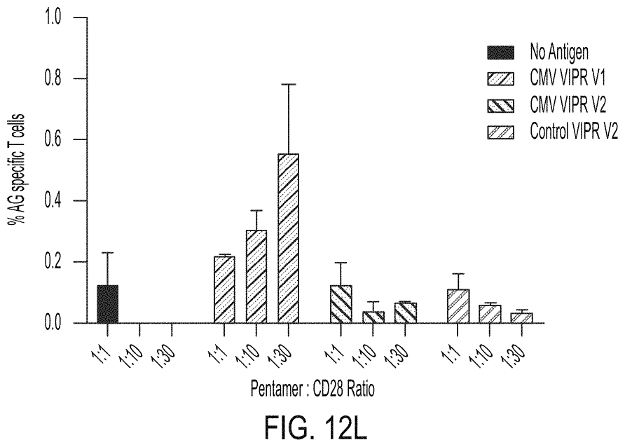

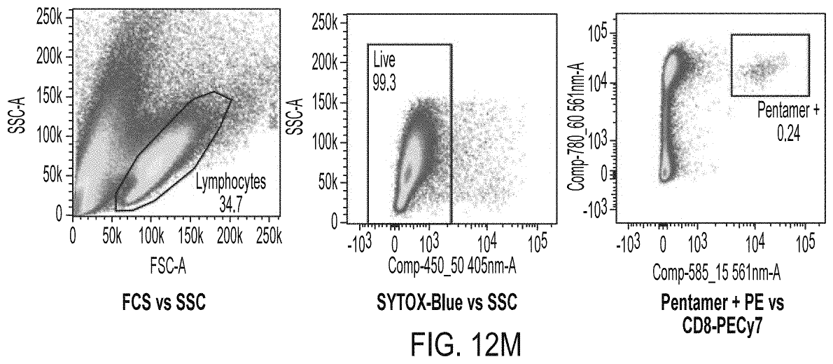

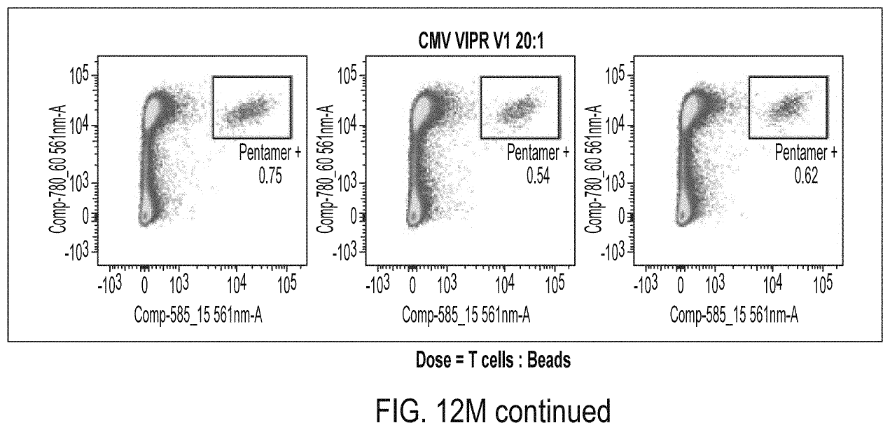

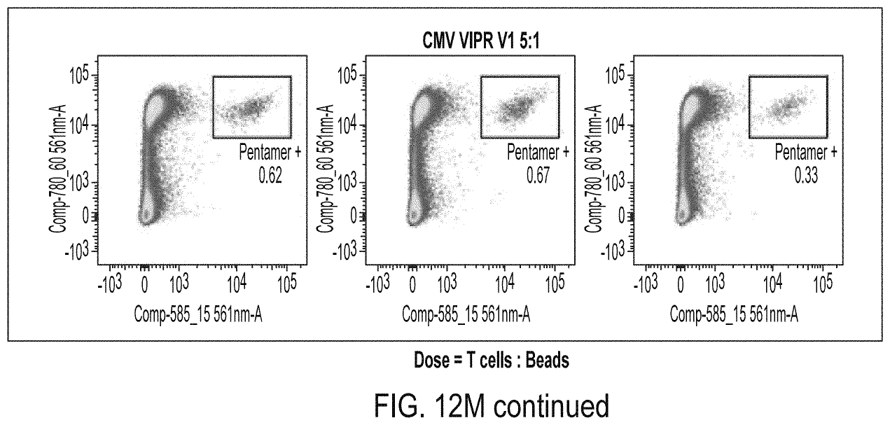

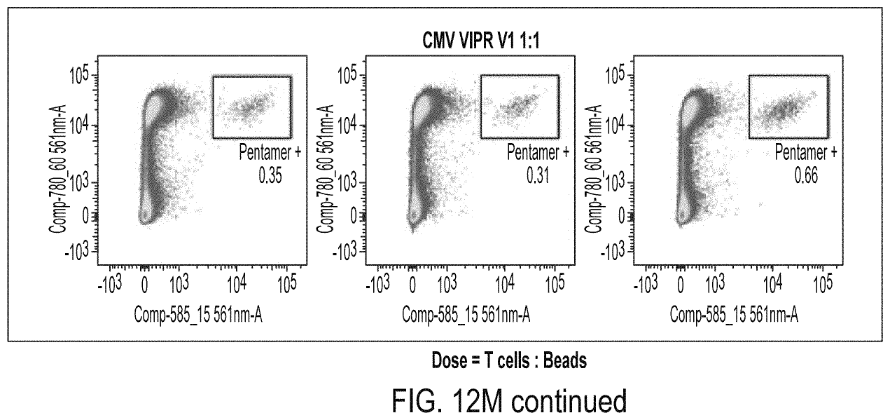

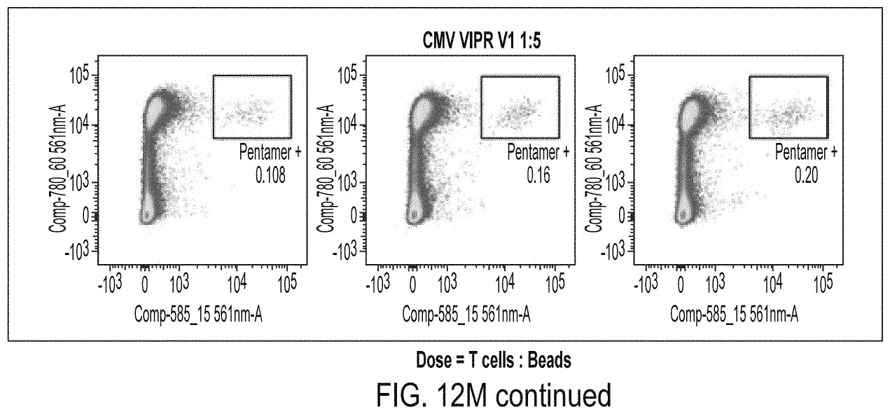

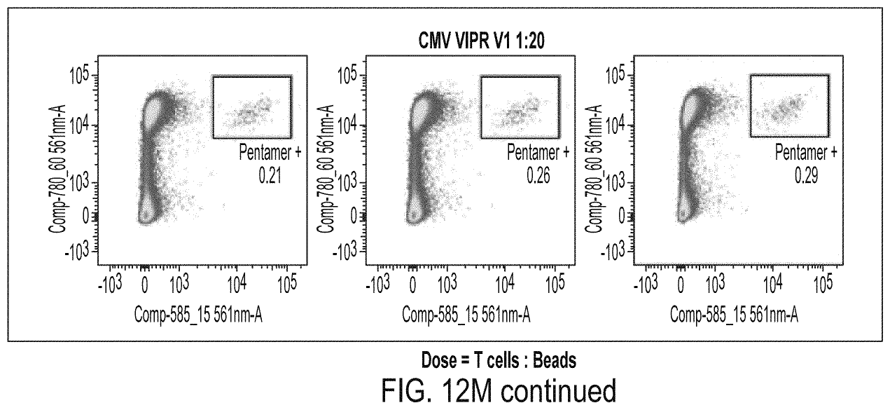

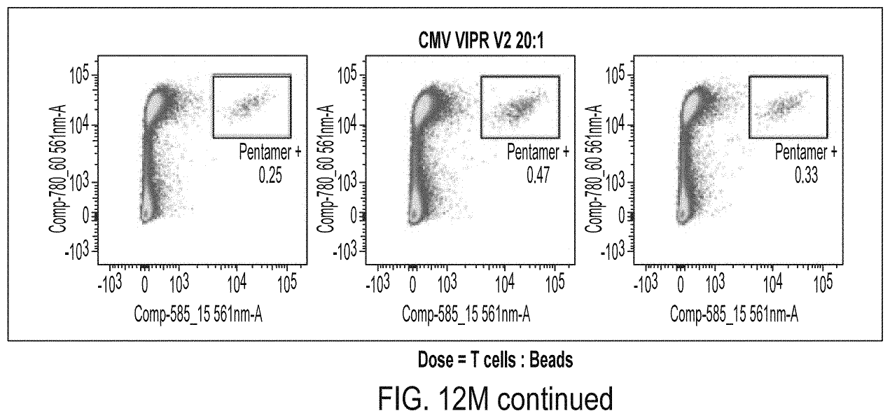

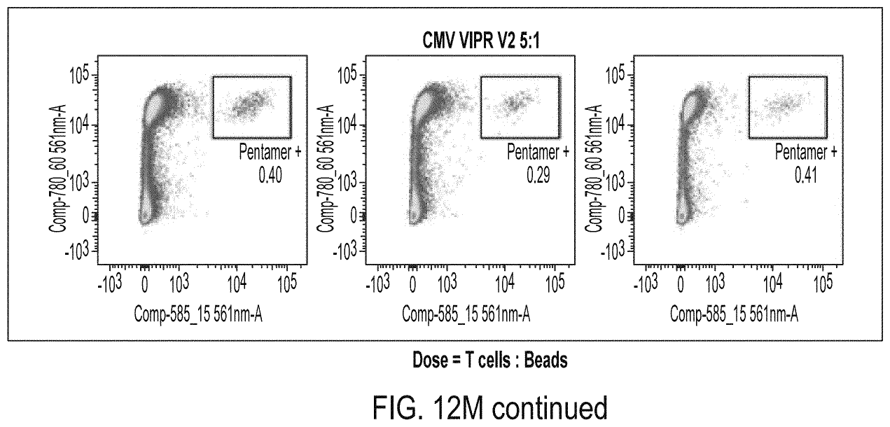

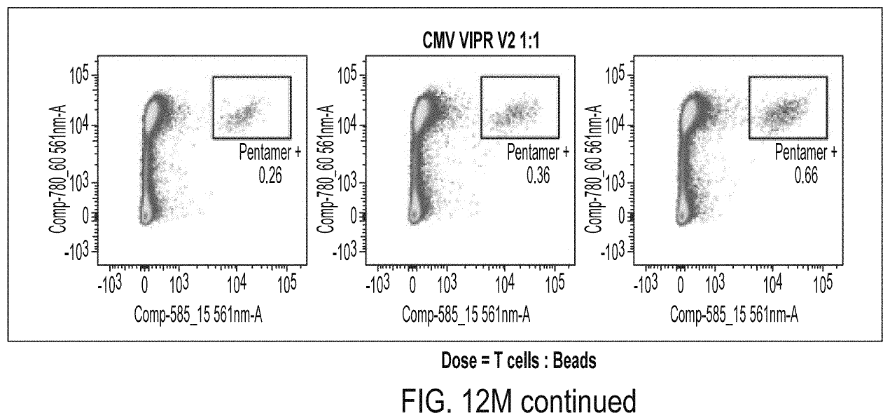

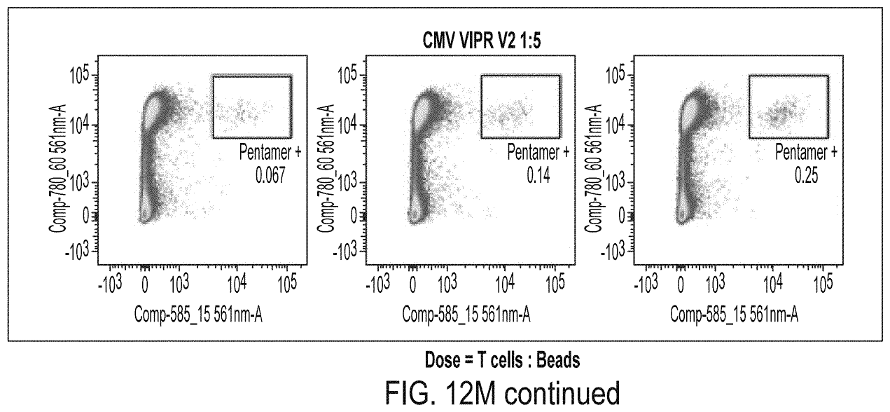

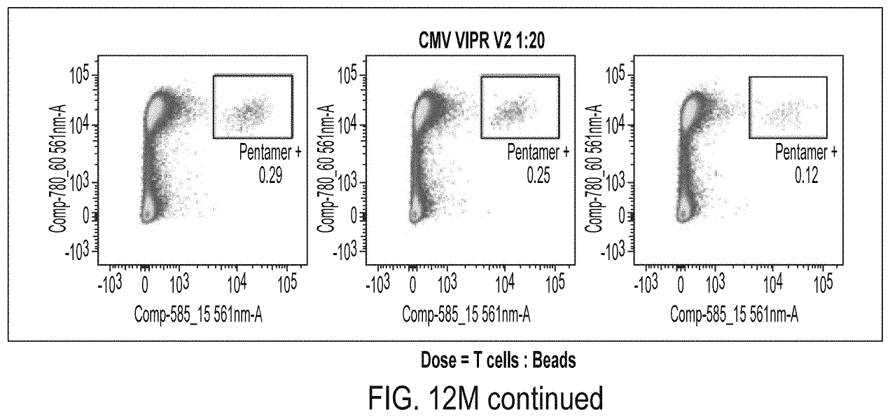

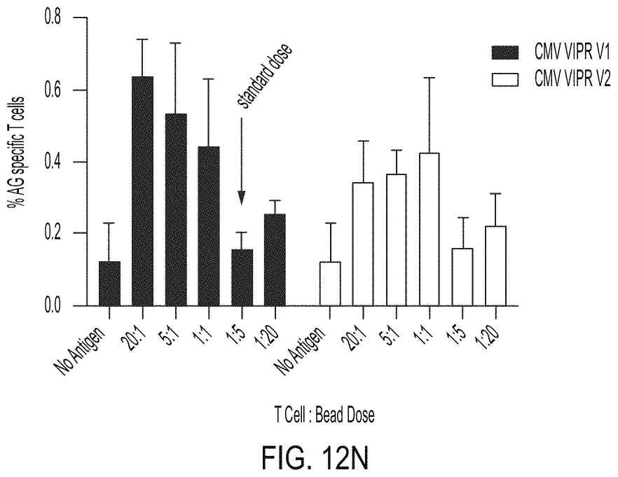

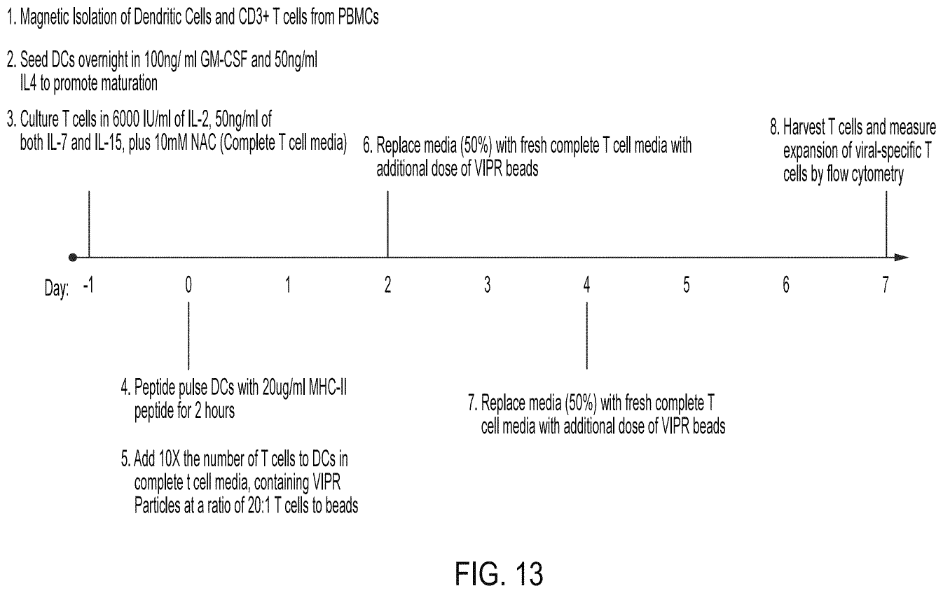

Comparative studies of particle re-dosing were done as follow. CMV pp65 (SEQ ID NO: 70) loaded particles were cultured with 2×10⁵ T Cells in the presence of 6000 U/mL of IL-2. Flow cytometry utilizing MHCI-Pentamer PE was performed on day 2 (FIG. 12B), day 4 (FIG. 12C), and day 10 (FIG. 12D). A summary of day 4 and day 10 data is shown in FIG. 12E and FIG. 12F respectively.

Further analysis to be completed due to some particles causing internalization of the TCR making it difficult to stain for antigen-specific T cells. To repeat multiple doses over the 7 days and add 3 day rest before flow analysis.

### Addition of NAC

Comparative studies of culturing in the presence of NAC (10 mM) were done as follow. CMV pp65 (SEQ ID NO: 70) loaded particles were cultured with 2×10⁵ T Cells in the presence of 6000 U/mL of IL-2 and 10 mM NAC. Flow cytometry utilizing MHCI-Pentamer PE was performed on day 4 (FIG. 12 G), and day 10 (FIG. 12H). A summary of day 4 data is shown in FIG. 121 and a summary of day 10 data is shown in FIG. 12J.

Results show that at later timepoints addition of NAC lead to a greater than 4-fold increase in the percent of antigen-specific cells, suggesting NAC supports more effective proliferation

### Particle Design: Pentamer CD28 Ratio

Comparative studies of culturing in the presence of different ratios of antigen peptide loaded particles were done as follow. CMV pp65 (SEQ ID NO: 70) loaded particles were cultured with 2×10⁵ T Cells in the presence of 6000 U/mL of IL-2. Flow cytometry utilizing MHCI-Pentamer PE was performed on day 10 (FIG. 12K). A summary of day 10 data is shown in FIG. 12L.

Results show that an increase in CD28 conjugated to bead increases antigen specific T cell expansion. Pentamer: CD28 of 1:1 gives 1.8-fold expansion over no antigen control. Pentamer: CD28 of 1:10 gives 2.5-fold expansion over no antigen control. Pentamer: CD28 of 1:30 gives 4.5-fold expansion over no antigen control (2.5-fold better than 1:1 V1 bead).

### Alternate T Cell: Particle Ratios

Comparative studies of culturing in the presence of different ratios of T cells and particles were completed as follow. CMV pp65 (SEQ ID NO: 70) loaded particles were cultured with T cell to particles ratios of: 20:1, 1:1, 1:20, 5:1, and 1:5 in the presence of 6000 U/mL of IL-2. Flow cytometry utilizing MHCI-Pentamer PE was performed on day 10 (FIG. 12M). A summary of day 10 data is shown in FIG. 12N.

Results show that higher T cell to bead ratios leads to better antigen-specific T cell expansions: T Cell: Bead=20:1=5.2 fold expansion over no antigen control, T Cell: Bead=5:1=4.4 fold expansion over no antigen control, T Cell: Bead=1:1=3.6 fold expansion over no antigen control, T Cell: Bead=20:1=1.3 fold expansion over no antigen control, T Cell Bead=20:1=2.1 fold expansion over no antigen control.

### Example 9: Rapid Expansion Protocol (REP)

T cells are incubated with OKT3 (anti-CD3) antibody (Ortho Biotech, Bridgewater, N.J.) and IL-2 in the presence of irradiated, allogeneic feeder cells at a 200:1 ratio of feeder cells to SARS-cov2 specific T cells. PBMC feeder cells obtained from normal volunteers by apheresis were thawed, washed, resuspended in CM, and irradiated (50 Gy). PBMC (2×10⁸), OKT3 antibody (30 ng/mL), CM (75 mL), AIM V media (GIBCO/BRL, 75 mL), and SARS-Cov2 specific T cells (1×10⁶) were combined, mixed, and aliquoted to a 175 cm2 tissue culture flask. Flasks were incubated upright at 37° C. in 5% C02. IL-2 was added to 6000 IU/mL on day 2. On day 5, 120 mL of culture supernatant was removed by aspiration (cells are retained on the bottom of the flask) and media was replaced with a 1:1 mixture of CM/AIM V containing 6000 IU/mL IL-2. On day 6 and every day thereafter, cell concentration is determined and cells are split into additional flasks or transferred to Baxter 3-L culture bags with additional medium containing 6000 CU/mL IL-2 as needed to maintain cell densities around 1×10⁶ cells/mL. About 14 days after initiation of the REP, cells are harvested from culture bags and prepared for patient treatment. Harvesting was accomplished using a Baxter/Fenwal continuous centrifuge cell harvester system. The cells are then washed in 0.9% sodium chloride and resuspended in 45 to 150 mL of 0.9% sodium chloride with 2.5% human albumin. Samples are removed from the infusion product for QC testing, aliquots are cryopreserved for future experimental analysis, and the remaining cells are infused into a patient by intravenous administration.

### Example 10: Optimization of Ex Vivo Expansion of Patient-Derived Viral Antigen-Specific Lymphocyte T Cells

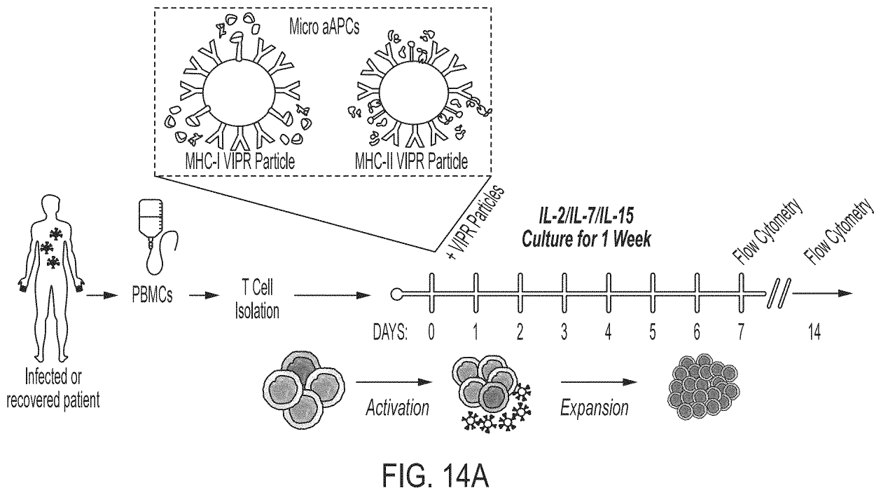

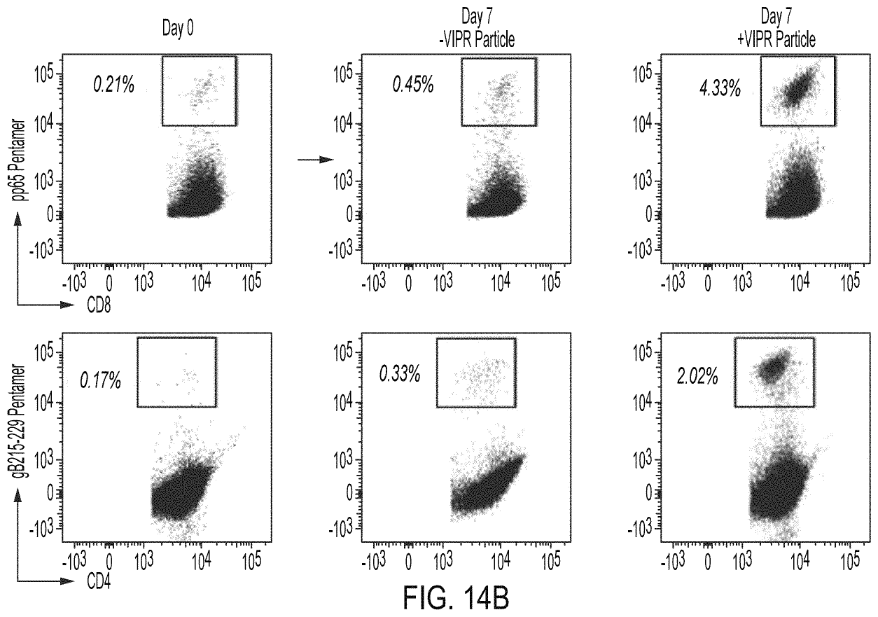

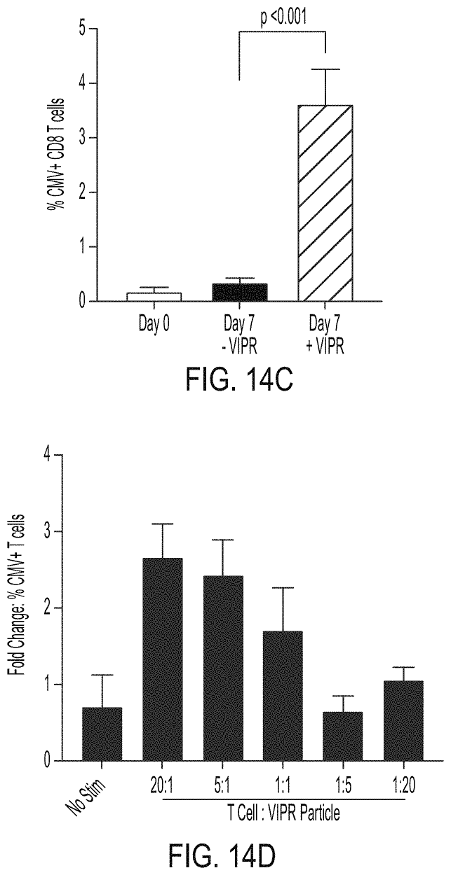

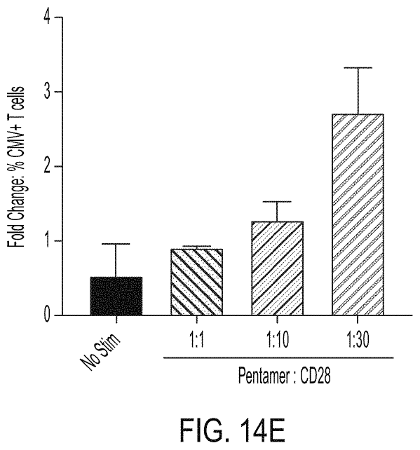

To develop a platform for robust and rapid ex vivo expansion of viral antigen-specific T cells from patients exposed to viral pathogens, a microbead aAPP VIPR particle capable of providing an immunogenic viral peptide in the context of MHC Class I or MHC Class II molecules in combination with anti-CD28 stimulation molecules was developed (FIG. 14A).

Preparation of the Microbead aAPP VIPR Particle

The microbead-based aAPPs VIPR particles comprising MHC-I pentamers or MHC-II tetramers with associated viral antigen peptide complexes, along with anti-CD28 antibodies, conjugated to 2.8 μm superparamagnetic beads were prepared as follows: biotin labelled peptide-MHC-Pentamers and biotinylated MHC class II Tetramers were conjugated to streptavidin Dynabeads in combination with biotinylated anti-CD28 antibodies. Pro5 MHC Class I Pentamers were provided by ProImmune Ltd and include the following peptide epitopes: CMV pp65 HLA A*02:01-restricted NLVPMVATV epitope (SEQ ID NO: 70); SARS-CoV-2 Spike protein 269− 277 HLA A*02:01-restricted YLQPRTFLL epitope (SEQ ID NO: 72). ProM2 MHC class II biotinylated Monomers were also obtained from ProImmune for the DRB1*07:01-restricted CMV gB 215-229 PDDYSNTHSTRYVTV epitope (SEQ ID NO: 80). These biotin-labelled MHC-peptide Pentamers and biotinylated MHC-peptide Monomer complexes, and mouse anti-human CD28 antibody (BD Biosciences) were conjugated to M270 Streptavidin Dynabeads (Thermo Scientific) at defined molar ratios of Pentamer:anti-CD28 and Monomer:anti-CD28 (calculated to account for tetramer formation of these monomers), and both in molar excess of the number of streptavidin molecules per Dynabead. Bead-conjugation was carried out at 4° C. in Phospho-buffered saline (PBS, Gibco), for 1 hour with regular agitation. Conjugated VIPR particles were centrifuged at 14,500×g for 3 minutes at 4° C. and washed three times in PBS while the beads were immobilized in an Invitrogen DynaMag-2 magnet (Thermo Scientific). VIPR particles were resuspended in sterile PBS at a concentration of 5×10⁸ particles per ml and stored at 4° C.

Immunodominant viral antigens were selected based on the known MHC specificity to demonstrate their capacity for TCR stimulation and simultaneous expansion of responding T cells. T cells isolated from donor PBMCs were cultured with these VIPR particles for 7-days in the presence of high concentrations of trophic cytokines, IL-2, IL-7 and IL-15 and addition of N-acetylcysteine (NAC), known to improve T cell proliferation. To demonstrate efficacy of the platform, an immunodominant MHC-I restricted pp65 antigen of cytomegalovirus (CMV) known to robustly stimulate the TCRs of CMV-specific T cells was selected.

The microbead aAPP VIPR particle can also be prepared using the protocol in Example 4 or its variation.

Detection and Enrichment of Antigen-Specific Virus Induced Lymphocytes (VIL)

Using fluorescently conjugated pentamers and flow cytometry, the CD3+ T cells isolated from the PBMCs of several independent CMV positive individuals were analyzed. Approximately 0.2% of T cells were CD8+ cells, demonstrating specificity for the CMV pp65 antigen (FIG. 14B). After a 7-day culture with VIPR particles, these cells enriched on average 20-fold, reaching Pentamer+/CD8+ T cell proportions of over 4% (FIGS. 14B & 14C). By comparison, donor T cells cultured in cytokine alone in the absence of VIPR particles showed a minimal enrichment in antigen-specific VIL proportions, even though the T cells proliferated robustly. In addition to the enrichment of virus-specific CD8+ T cells using MHC-I restricted antigens, CD4+ T cells could also be enriched over 20-fold using MHC-II VIPR particles carrying a well validated CMV glycoprotein antigen (FIG. 14B).

Optimization of Antigen-Specific Micro aAPPs for T Cell Expansion

The design of the VIPR particle aAPPs was further optimized to enhance the expansion of antigen-specific T cells within the rapid 7-day stimulation culture. The impact of the ratio of T cells to VIPR particles had on the proportion of CMV-specific T cells enriched at day-7 was investigated. A dose-dependent enrichment was seen with lower doses of particles and higher numbers of T cells, such that an optimal enrichment was observed with a ratio of 20:1 T cells to VIPR particles (FIG. 14D). In addition, increasing the ratio of molecules of anti-CD28 antibody to peptide-MHC-pentamer also increased the capacity of the VIPR particles for expansion of the antigen-specific VIL population (FIG. 14E).

### Example 11: Rapid Expansion of SARS-CoV-2 Antigen-Specific Virus-Induced Lymphocytes Using Microbead aAPP VIPR Particles for Cell Therapy

Provided herein are methods of rapidly expanding SARS-CoV-2 antigen-specific virus-induced lymphocytes using microbead aAPP VIPR particles for cell therapy. To demonstrate the modularity of the methods, results showing experiments using CMV antigen are presented.

### Enrichment of SARS-CoV-2-Specific T Cells in COVID-19 Convalescent Individuals

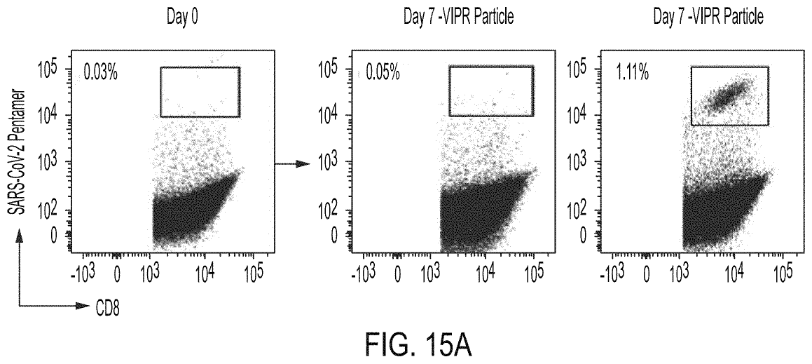

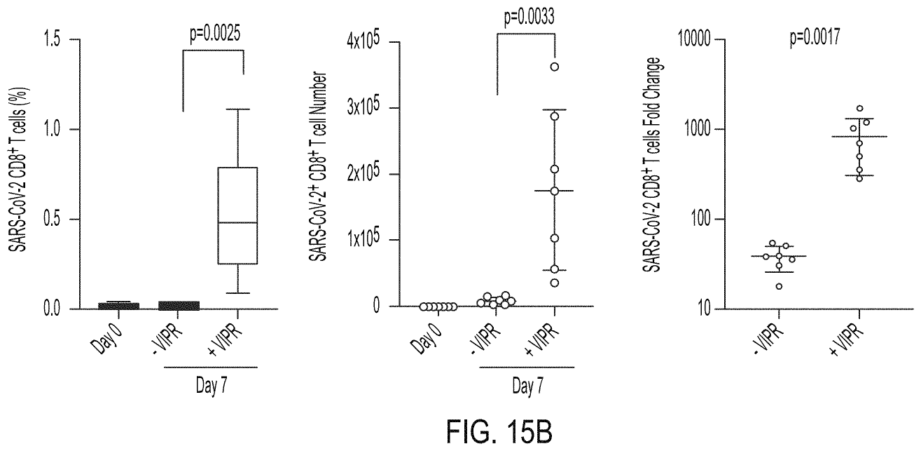

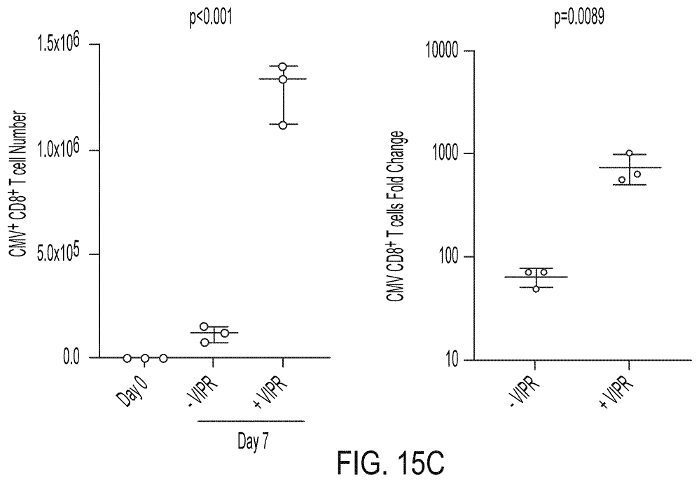

To determine whether VIPR particles can be used to enrich and expand SARS-CoV-2 specific T cells from COVID-19 patients for cell therapy, PBMCs from COVID-19 convalescent individuals were obtained 24 days or more after developing symptoms and testing positive by PCR. The frequency and proportion of VIL specific for a recently published and validated immunodominant MHC-I SARS-CoV-2 epitope YLQPRTFLL (YLQ) (SEQ ID NO: 72) was analyzed. YLQ antigen-specific VIL were barely detectible within the isolated T cell populations from these individuals (FIG. 15A and Table 8).

TABLE 8
The clinical presentation of convalescent COVID-19 donors used and the proportions of SARS-
CoV-2 specific VIL detectible before enrichment, and fold expansion by VIPR particles.

| | | | | Days post | | | | Fold |
|---|---|---|---|---|---|---|---|---|
| | | | | infection | | | | change |
| | | | Serological | PBMCs | | | % VIL | by VIPR |
| Donor | Sex | Age | Results | harvested | Symptoms | MCH-1 | (day 0) | expansion |
| 1 | F | 37 | IgG+/IgM+ | 25 | Fatigue; | HLA-A2 | 0.03 | 37 |
| | | | | | Non-productive cough; | 02:01 | | |
| | | | | | Shortness of breath; | | | |
| | | | | | Anosmia | | | |
| 2 | M | 48 | IgG+/IgM+ | 72 | Fever >100.4° F.; | HLA-A2 | 0.02 | 21 |
| | | | | | Chills; Muscle aches; | 02:01 | | |
| | | | | | Headache; Ageusia & | | | |
| | | | | | Anosmia | | | |
| 3 | M | 46 | IgG+ | 53 | Sore throat; | HLA-A2 | 0.02 | 10 |
| | | | | | Nasal congestion; | 02:01 | | |
| | | | | | Fatigue; | | | |
| | | | | | Non-productive cough | | | |
| 4 | M | 42 | IgG+ | 26 | Fever >100.4° F. | HLA-A2 | 0.01 | 48 |
| | | | | | (38° C.); | 02:01 | | |
| | | | | | Congestion; Non-productive | | | |
| | | | | | cough; Chills; Flu-like | | | |
| | | | | | symptoms; Muscle aches; | | | |
| | | | | | Fatigue | | | |
| 5 | F | 42 | IgG+/IgM+ | 24 | Headache; Fever >100.4° F.; | HLA-A2 | 0.02 | 16 |
| | | | | | Muscle aches; | 02:01 | | |
| | | | | | Ageusia & Anosmia; | | | |
| | | | | | arthralgias; Fatigue; | | | |
| | | | | | Non-productive cough | | | |
| 5 | M | 51 | ND | 28 | ND | HLA-A2 | 0.04 | 19 |
| | | | | | | 02:01 | | |
| 7 | M | 49 | ND | 26 | ND | HLA-A2 | 0.01 | 12 |
| | | | | | | 02:01 | | |

After 7-day culture with MHC-I VIPR particles, antigen-specific CD8+ T cells could be readily detected by pentamer staining and could be enriched and expanded to frequencies greater than 1% (FIGS. 15A & Table 8). While the majority of PBMCs from convalescent individuals with the YLQ matched MHC allele (HLA A*02), included T cells from which antigen specific VIL could be enriched, the overall frequency varied between individuals and did not appear to correlate with either the length of time since they were symptomatic, nor the reported severity of their symptoms (Table 8).

Rapid VIL Expansion Results in 1, 000-Fold Enrichment of SARS-CoV-2 Antigen-Specific T Cells within 7-Days

The capacity for the 7-day culture system to expand the overall quantity of virus specific VIL was evaluated. The T cell cultures were configured to include the same culturing conditions used in neoantigen TIL human clinical trials (“A Study of Metastatic Gastrointestinal Cancers Treated With Tumor Infiltrating Lymphocytes 604 in Which the Gene Encoding the Intracellular Immune Checkpoint CISH Is Inhibited Using CRISPR Genetic Engineering.” 2010, https://clinicaltrials.gov/ct2/show/NCT04426669) to enable rapid T cell expansion, thus in addition to the VIPR particles and high IL-2 (6000 IU/ml), IL-7, IL-15 and NAC, T cells were cultured in Gas Permeable Rapid Expansion (G-REX) plates. These culture plates enable gas exchange from the base of the culture well, allowing cells to be cultured with a large ratio of media per surface area and abundant access to nutrients, and have been shown to facilitate a large and rapid expansion of primary human T cells (Vera, J. F. et al., “Accelerated production of antigen-specific T cells for preclinical and clinical applications using gas-permeable rapid expansion cultureware (G-Rex).” 2010, J Immunother 33, 305-315).

Isolated human CD3+ T cells were cultured in X-VIVO-15 Basal Media (Lonza) supplemented with 10% Human AB Serum Heat Inactivated (Sigma), 6000 IU/ml Recombinant Human IL-2 (Peprotech), 5 ng/ml Recombinant Human IL-7 (Peprotech), 5 ng/ml Recombinant Human IL-15 (Peprotech) and 10 mM N-Acetyl-L-cysteine (Sigma). T cells were seeded at a density of 2×10⁵ cells per well of U-bottom 96-well plates, or at a density of 1-2×10⁶ T cells per cm² of G-REX 24-well plates (Wilson-Wolf). At the time of T cell seeding, VIPR particles were added to the relevant samples at a ratio of 20 T cells per particle, and cells were cultured for 7− days in a 37° C., 5% CO2 culture incubator. In addition, a sample of the T cells was also analyzed by flow cytometry at Day-0 to measure the starting proportion of antigen-specific T cells (see methods below). For some cultures the media was replaced every 2-3 days with fresh complete media including cytokines and NAC (but no extra addition of VIPR particles) and media in G-REX cultures was left unchanged for the duration of 7-days in some experiments to promote cell expansion. On day 7, T cell numbers were assessed by harvesting all cells, washing in PBS followed by centrifugation at 300×g for 10 minutes and then counting using a CellDrop Automated Cell Counter (DeNovix). The proportion of expanded antigen-specific T cells was assessed at Day-7 by flow cytometry. VIL can also be expanded using methods described in protocol of Example 9.

After 7-days of culture with VIPR particles, SARS-CoV-2 antigen-specific CD8+ T cells could be robustly expanded in proportion, but most importantly in absolute quantity of T cells, to an average of over 1,000-fold (FIG. 15B). Thus, the cultures seeded with 1×10⁶ total CD3+ T cells could reach expanded numbers, on average, between 2.6×10⁷ and 4.5×10⁷ total cells at day-7. This proliferative expansion coupled with the enrichment of the VIL population resulted in an average of 2.4×10⁵ SARS-CoV-2 CD8+ T cells per million CD3+ cultured (FIG. 15B). A similar robustness in the expansion in absolute number of virus-specific CD8+ VIL could also be observed with T cells from CMV-positive individuals when stimulated with MHC-I VIPR particles under these culture conditions (FIG. 15C). After 7-days, antigen-specific CD8+ T cells had increased from approximately 2×10³ cells to over 1.0×10⁶, leading to up to an average >700− fold expansion in cell number. Collectively these data demonstrate the ability of the VIPR particle expansion protocol to rapidly enrich and expand VIL from low numbers in CMV-positive individuals and near undetectable numbers in COVID-19 convalescent individuals, to significantly large numbers of virus-specific T cells.

### The Activation and T Cell Memory Phenotype of Rapidly-Expanded SARS-CoV-2 Antigen-Specific VIL

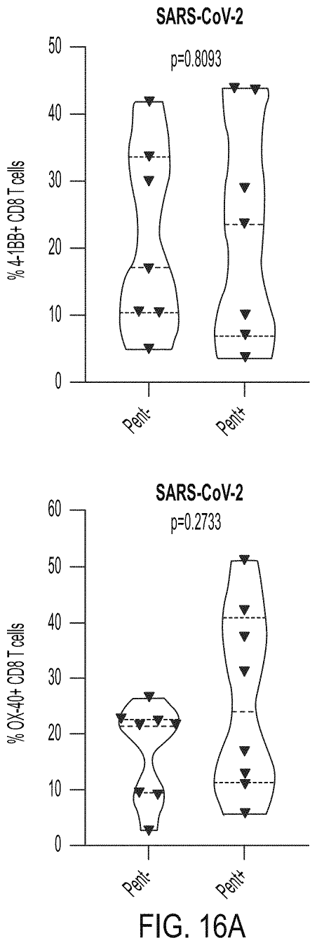

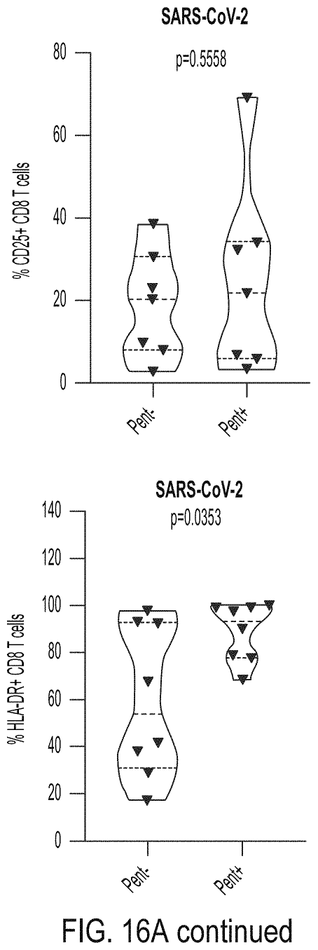

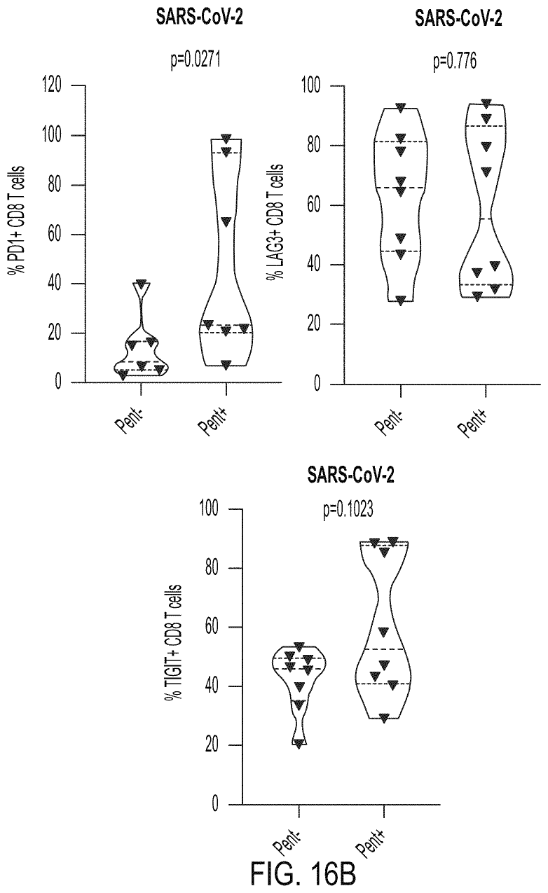

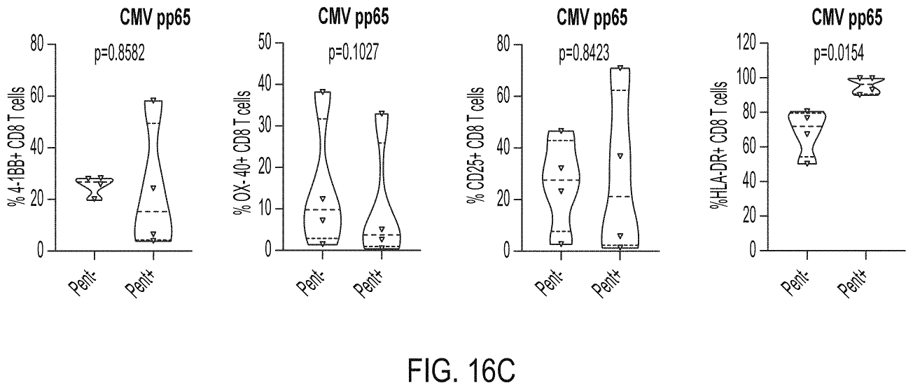

To evaluate the phenotype of SARS-CoV-2 specific T cells and CMV specific T cells that had undergone enrichment and expansion with VIPR particles, T cells were analyzed for expression of cell surface markers indicative of T cell activation. SARS-CoV-2-specific CD8+ T cells expressed co-stimulatory and activation markers 4-1BB, OX-40 and CD25, albeit variable between convalescent individuals, and an elevated level of HLA-DR when compared to that of the non-virus-specific T cells within the culture (FIG. 16A). The SARS-CoV-2 antigen-specific VIL population also showed a significant expression of the checkpoint markers PD-1, TIGIT, LAG-3, indicating these T cells have acquired a proliferative and activated functional phenotype (FIG. 16B). The same profile of activation marker and checkpoint gene expression was observed when CMV-specific VIL were stimulated after 7-day rapid expansion with VIPR particles, with a similarly observed variability between different CMV-positive individuals, indicating this culture platform is effective at rapid T cells expansion and activation with multiple viral antigens (FIGS. 16C and 16D).

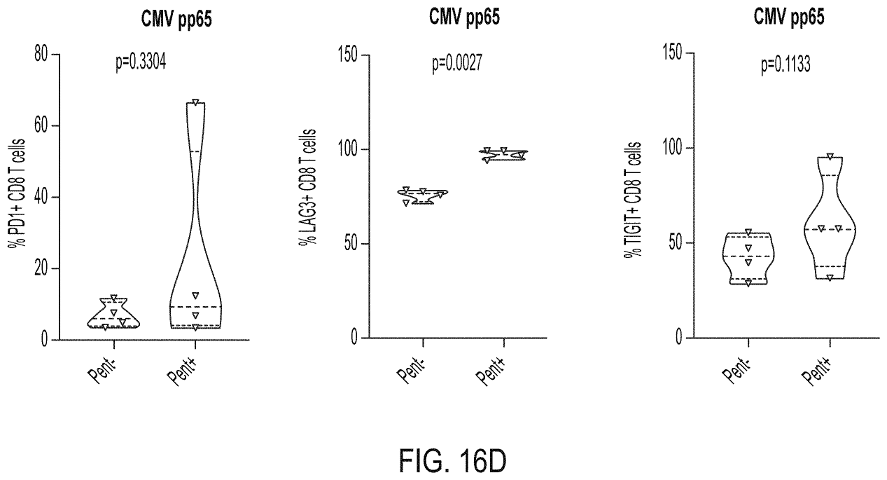

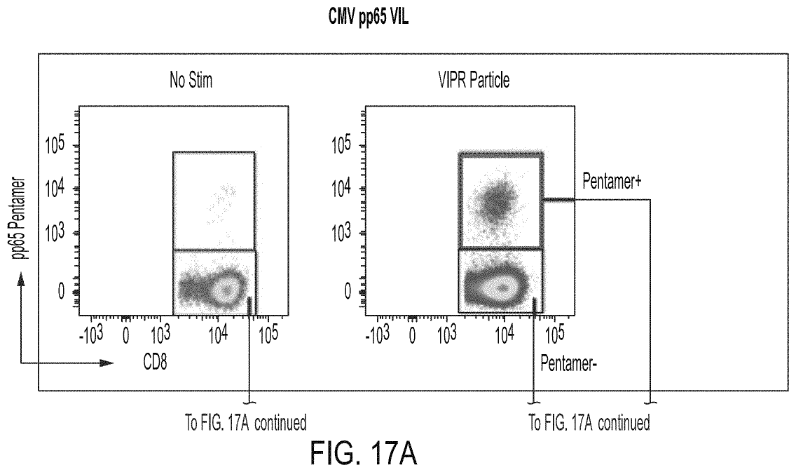

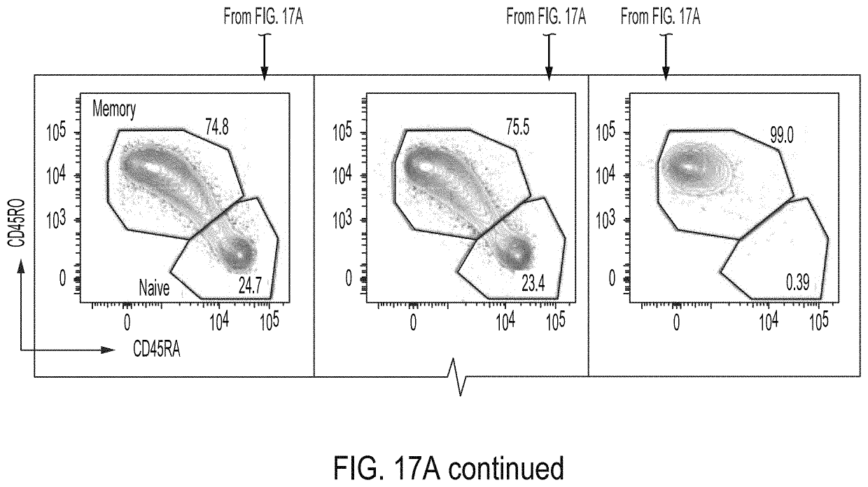

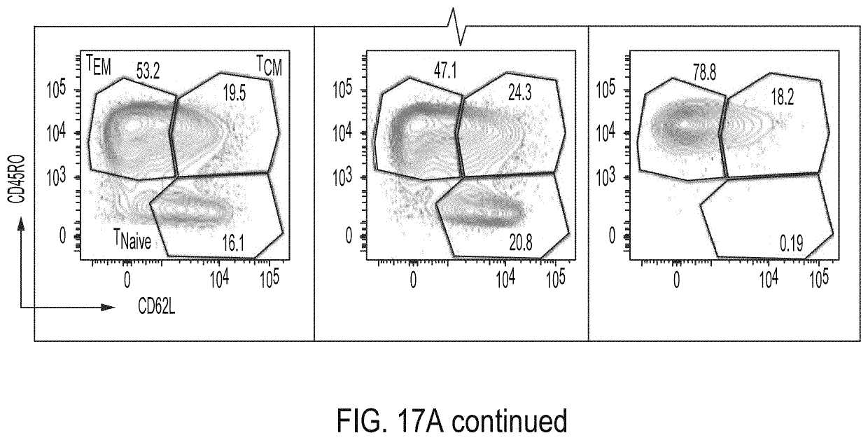

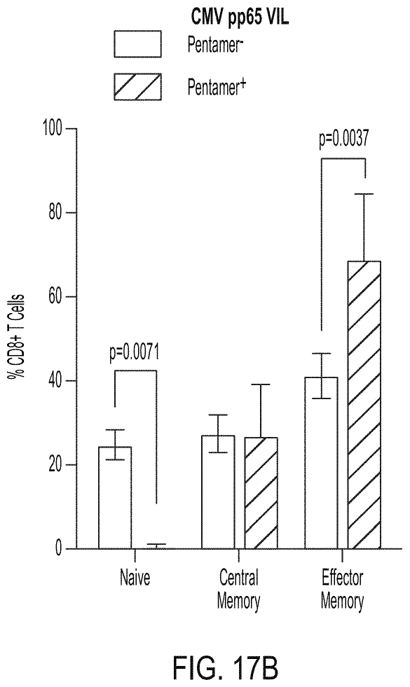

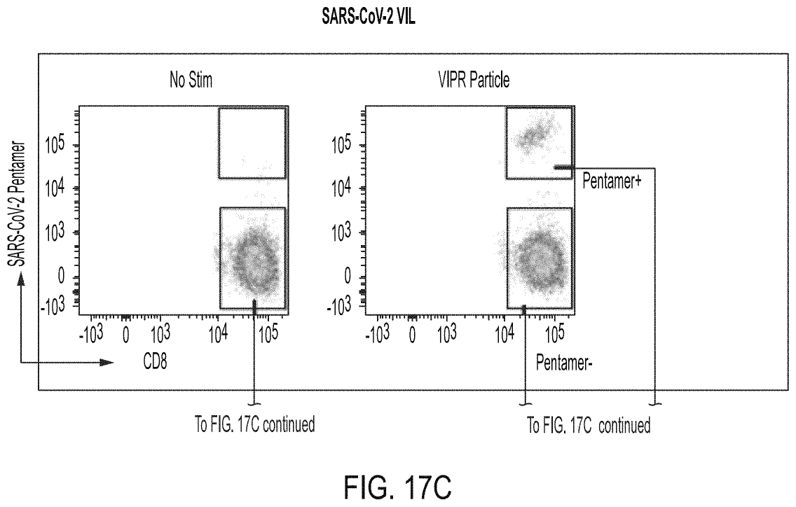

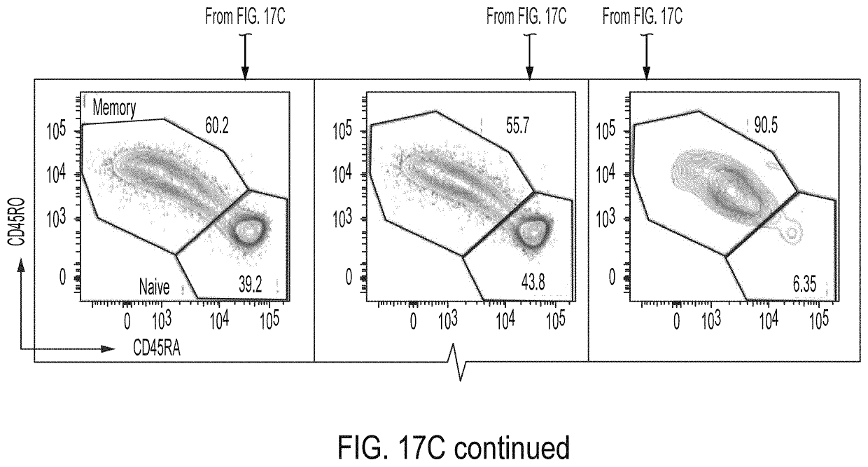

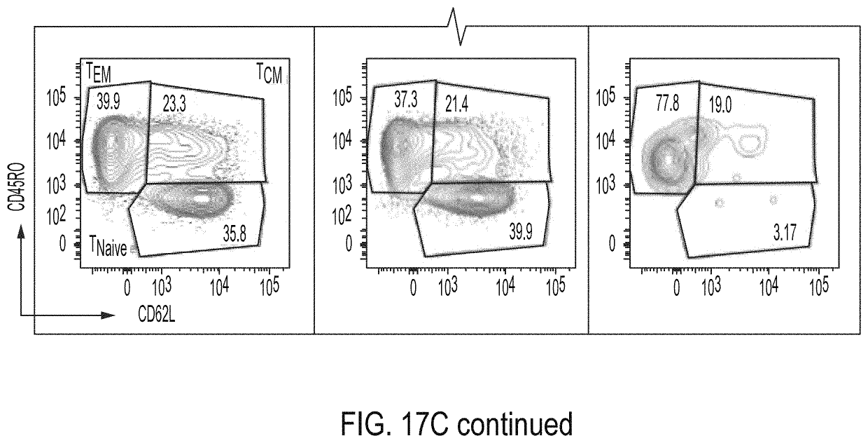

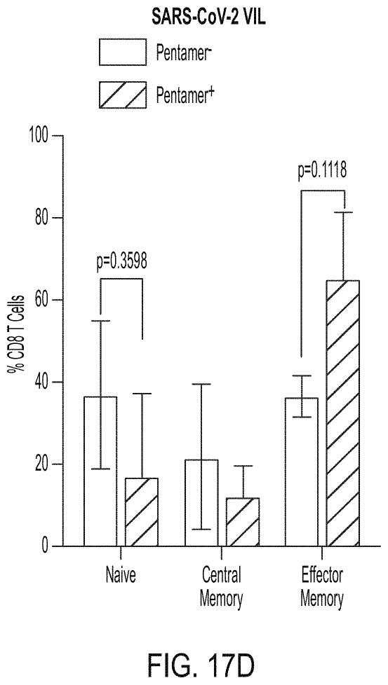

The memory phenotype of the expanded SARS-CoV-2 and CMV virus-specific cells was analyzed by measuring expression of the canonical memory markers CD45RA and CD45RO and categorized the cell populations into either a naïve (CD45RO−, CD45RA+) or memory phenotype (CD45RO+, CD45RA−). After the 7-day culture in IL-2, IL-7, IL-15 and NAC, the majority of CMV T cells had begun to adopt a memory phenotype, but the virus-specific CD8+ T cells were almost exclusively expressing the highest levels of CD45RO and completely lost CD45RA expression, indicating the antigen-specific population had uniformly transitioned into memory T cells (FIG. 17A). Further delineation of the T cell memory phenotype by analysis of CD62L expression within the CD45RO+ population revealed the virus-specific T cells had robustly differentiated into an effector memory T cell phenotype via downregulation of CD62L (FIGS. 17A & 17B). The non-virus-specific T cells within these cultures however, consisted of significantly more naïve T cells. The same profile of effector memory T cells was observed when SARS-CoV-2-specific VIL were stimulated after 7-day rapid expansion with VIPR particles, again demonstrating the antigen-specific VIPR particle platform is effective at significantly expanding activated effector memory T cells over a short time-course. (FIGS. 17C & 17D).

Polyfunctional Proinflammatory Cytokine Expression Among Rapidly-Expanded SARS-CoV-2 Antigen-Specific VIL

To further evaluate the function of the rapidly expanded virus-specific VIL, intracellular cytokine staining and flow cytometry was performed to measure the proportion of the cells that were producing IFN-γ, TNF-a and IL-2.

After 7-days of expansion with VIPR particles, the T cell cultures were stimulated for 6 hours with 20 μg/ml peptide antigen (>95% purity) specific for the VIPR particle expanded CD8+ population (CMV pp65: NLVPMVATV (SEQ ID NO: 70); SARS-CoV-2 S protein 269− 277 YLQPRTFLL (SEQ ID NO: 72)), all peptides were synthesized and obtained from ProImmune, Ltd. After 1-hour of peptide stimulation, GolgiStop solution (containing Monensin protein transport inhibitor) was added to block intracellular protein transport (BD Bioscience). As a positive control for cytokine production, T cells were also stimulated for 6 hours with 50 ng/ml PMA and 1 μg/ml Ionomycin (Sigma). T cells were then harvested, and cells fixed and permeabilized using BD Cytofix/Cytoperm Fixation/Permeabilization Solution (ThermoFisher). Cells were then stained for surface markers followed by intracellular cytokines using antibodies specific for IFN-γ (4S.B3, 1:40) (Biolegend) IL-2 (MQ1-17H12, 1:40) (BD Bioscience), or TNF-α (MAb11, 1:40) (ThermoFisher). Flow analysis was carried out on a Fortessa flow cytometer (BD Bioscience) and data analyzed using FlowJo 10 software (BD Biosciences).

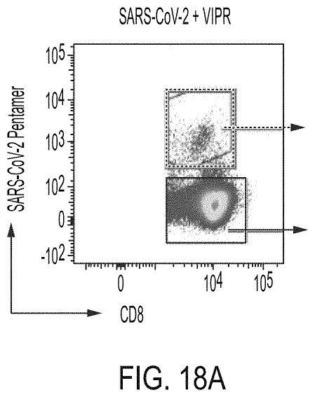

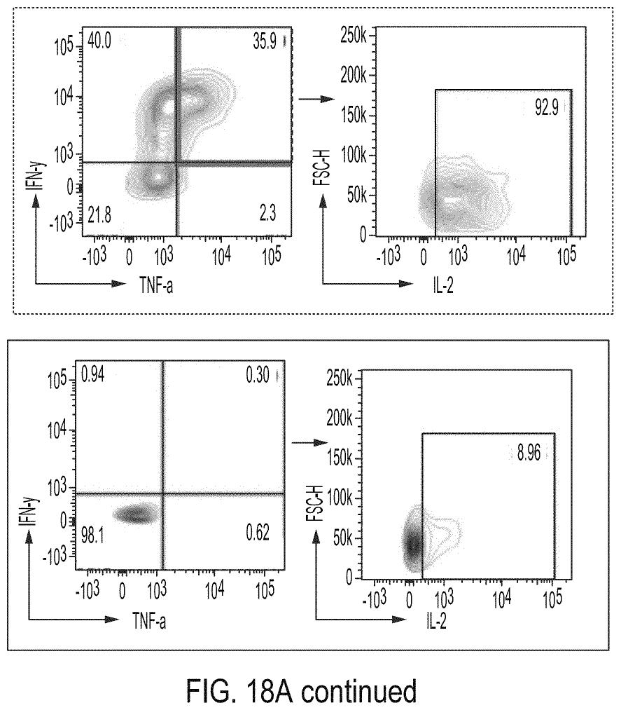

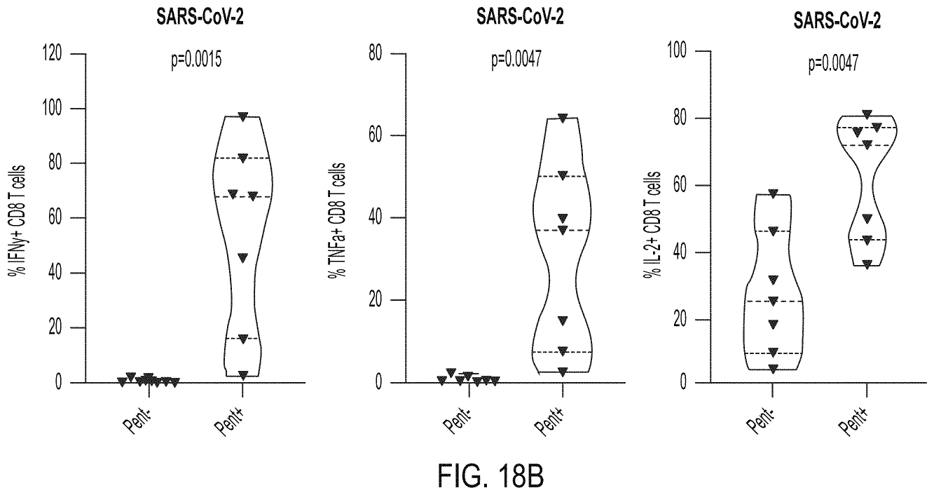

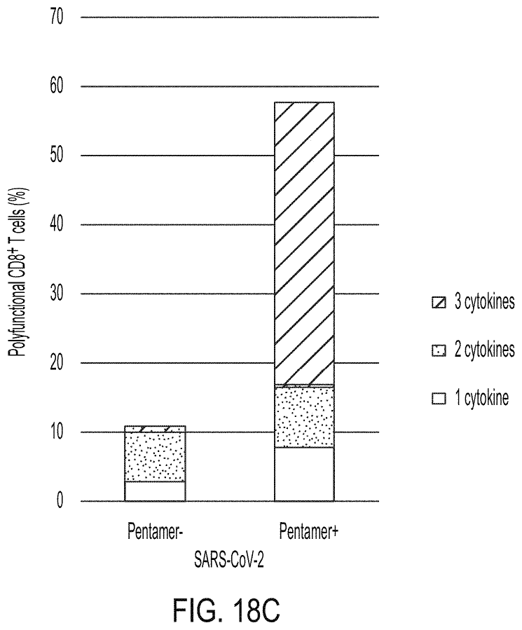

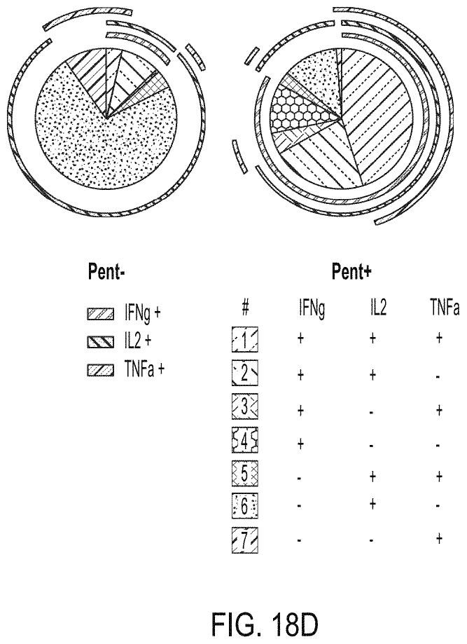

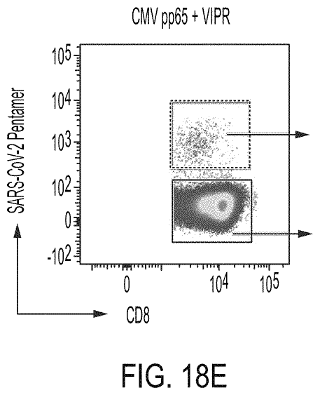

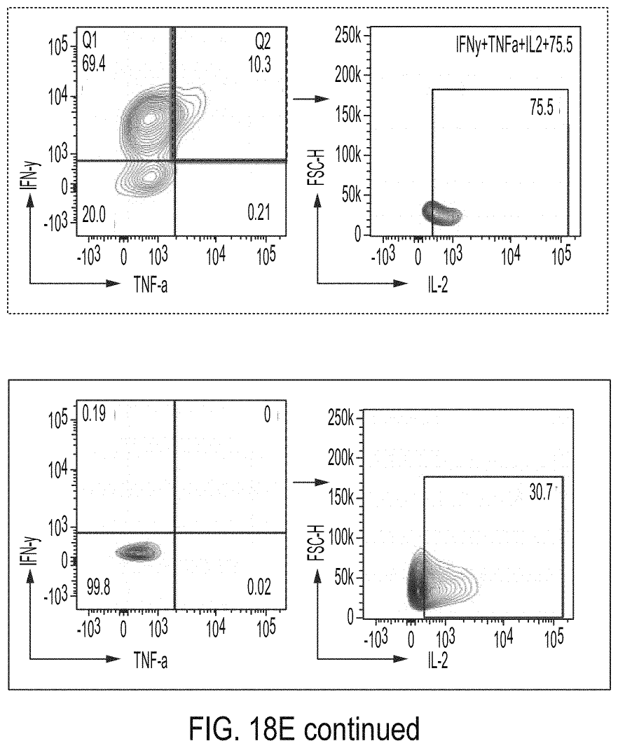

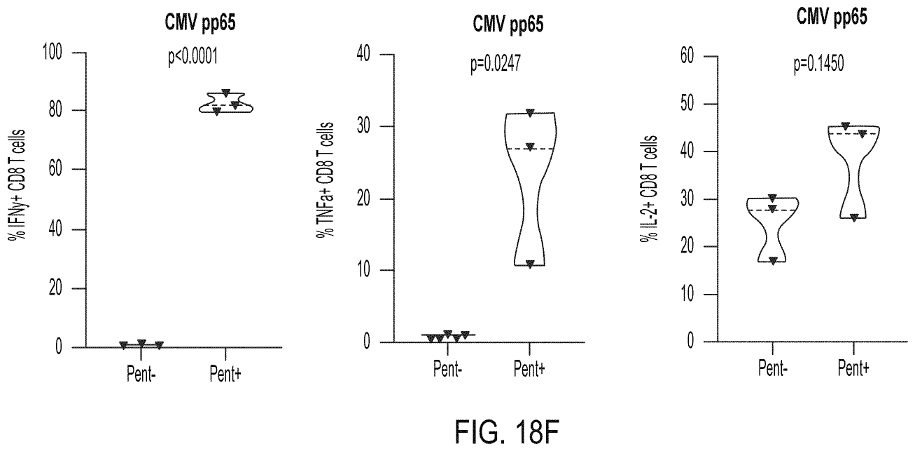

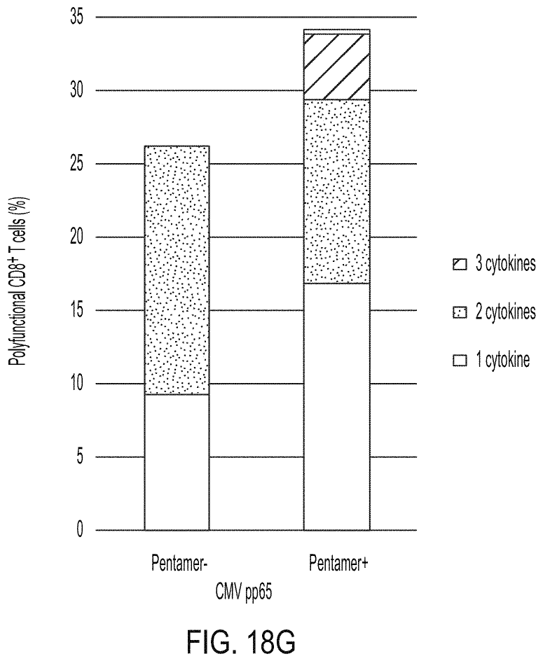

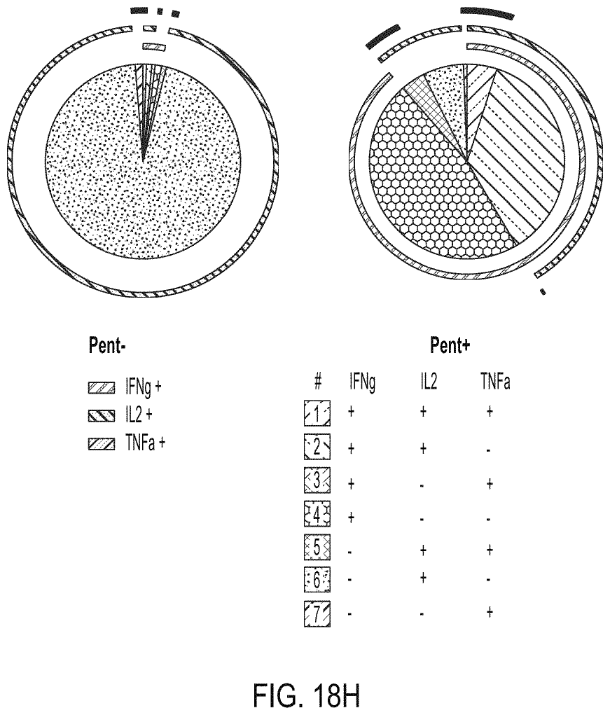

Strong expression of all three proinflammatory cytokines within the antigen-specific T cell population (identified by TCR specific pentamer staining) was observed, the expression of either IFN-γ or TNF-α in the non-antigen-specific T cell population (T cells that do not bind the TCR-specific pentamer) or within the T cells cultured for 7-days without any VIPR particle expansion was not detected (FIGS. 18A & 18B). The antigen-specific T cells also showed significantly elevated levels of IL-2 when compared to that of the non-antigen specific CD8+ population. When analyzed together, an elevated proportion of cells expressing 1, 2 or all 3 cytokines in combination was detected, when compared to that of the non-SARS-CoV-2-specific T cells within the expanded culture (FIGS. 18C & 18D). The same functional response was observed with virus-specific VIL expanded in T cells isolated from CMV-positive individuals and stimulated for 6-hours with pp65 MHC-I epitope peptide antigen. Intracellular cytokine staining revealed a robust increase in production of all cytokines in the CMV-specific CD8+ T cells when stimulated controls (FIGS. 18E & 18F). An elevated frequency of polyfunctional CD8+ T cells expressing multiple proinflammatory cytokines was also seen in the CMV-specific T cell population (FIGS. 18G & 18H). Taken together, these analyses demonstrate that elevated numbers of virus-specific VIL can be rapidly expanded in 7-days by VIPR particle culture and form robust activated, polyfunctional effector memory T cells.

Patient Samples and Preparation of T Cells

For preparing patient and T cell samples described herein and thereof, peripheral blood mononuclear cells were obtained from anonymized CMV-positive individuals and convalescent COVID-19 individuals (Caltag Medsystems, Tissue Solutions Ltd, Precision for Medicine, Inc.) and obtained, handled and stored in accordance with the Human Tissue Authority UK regulations. Genomic DNA was extracted from PBMC samples using the Gentra Puregene DNA isolation kit (Qiagen), and DNA samples were HLA-typed by sequencing at Class I (HLA-A, -B & -C) and Class II (HLA-DRB1) loci (MC Diagnostics). Total CD3+ T cells were isolated from unfractionated PBMCs using the EasySep™ Human T Cell Isolation Kit (Stem Cell Technologies) with a DynaMag™-2 magnet (ThermoFisher Scientific) according to the manufacturer's guidelines. Purity and viability of isolated T cells was assessed using flow cytometry prior to cryopreservation of isolated T cells in CryoStore CS10 cryopreservation media (Stem Cell Technologies) at a density of 1-1.5×10⁷ cells per ml.

### Flow Cytometry Analysis of T Cell Phenotype

For flow cytometric analysis of the antigen-specific T cell population and cell surface marker expression described herein and thereof, cells were harvested from culture plates and washed using PBS with 1% Bovine Serum Albumen (Thermo Scientific) and were then stained with monoclonal antibodies specific for CD8 (HIT8A, 1:100), CD4 (OKT4, 1:100), HLA-DR (L243 1:80), LAG-3 (11C3C65, 1:80), TIGIT (VSTM3, 1:40), CD45RO (UCHL1, 1:40), CD45RA (HI100, 1:80), TIM3 (F38-2E2, 1:40), CD62L (DREG-56, 1:40), CD57 (QA17A04, 1:80), PD-1 (EH12.1, 1:40), OX-40 (Ber-ACT35, 1:40), CD25 (MA2-51, 1:40), 41BB (4B4-1, 1:40) (Biolegend), or specific for CD8 (RPA-T8, 1:100) (BD Bioscience), or TNF-a (MAb11, 1:40) and CD3 (UCHT1, 1:100) (ThermoFisher). CMV pp65 and SARS-CoV-2 Spike antigen-specific T cells were detected by staining cells with R-PE-labelled Pro5 Pentamers (ProImmune, Ltd), and CMV gB215-229 specific T cells stained with R-PE labelled ProT2 Tetramer for 20 minutes at room temperature according to manufacturer's recommendation. Live/Dead Fixable Dead Cell Stains (Invitrogen) or SYTOX Blue Dead Cell Stain (Invitrogen) were included in all experiments to exclude dead cells. After staining, cells were resuspended in PBS with 2% Human Heat Inactivated AB Serum (Sigma) and 0.1M EDTA pH 8.0 (Invitrogen) before analysis on a Fortessa flow cytometer (BD Bioscience) and data analyzed using FlowJo 10 software (BD Biosciences).

Allogeneic VIL Therapy Platform: An Adoptive Cell Therapy for the Treatment of Individuals Suffering from Severe Symptoms of COVID-19

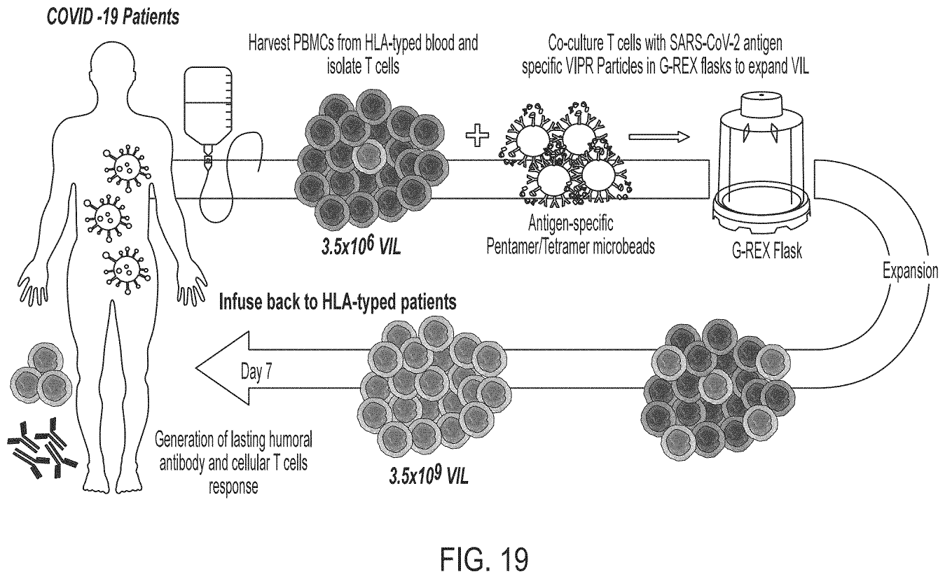

Given the paucity of therapeutic options for the treatment of COVID-19 and the data described herein and thereof suggesting the importance of the immune T cell response to viral infections, a novel potential therapeutic modality to augment the anti-viral T cell response by providing a therapeutic immune boost of virus-specific T cells (VIL) was proposed. Similar to adoptive cell therapy (ACT) methods in immuno-oncology to actively transfer Tumor Infiltrating Lymphocytes (TIL), the potential utility of Virus Induced Lymphocytes (VIL) to deliver SARS-CoV-2 immunodominant viral antigens in the setting of nascent and acute COVID-19 infection was demonstrated. Unlike T cell immuno-oncology in which T cell expansion and subsequent efficacy requires substantial critical mass of quantity, and thus time, VIL serve a catalytic immune booster function and can be isolated and expanded ex vivo both autologously and also MHC typed for allogenic delivery in a 7-day vein-to-vein time which is clinically practical and relevant (as depicted in FIG. 19).

In the setting of COVID-19 pathogenesis, studies have found individuals suffering from a more severe presentation of the disease typically require a duration of hospitalization ranging from 5 to 29 days (Rees, E. M. et al., “COVID-19 length of hospital stay: a systematic review and data synthesis.” 2020, BMC Med 18, 270; Vultaggio, A. et al., “Prompt Predicting of Early Clinical Deterioration of Moderate-to-Severe COVID-19 Patients: Usefulness of a Combined Score Using IL-6 in a Preliminary Study.” 2020, J Allergy Clin Immunol Pract 8, 2575-2581 e2572). Thus, a therapeutic treatment to improve patient outcome must be rapidly administered during the critical time window of disease progression prior to and/or early in the patient's intubation and ventilatory support. Herein lies the opportunity for a T cell therapy that can robustly expand the quantity and quality of virus-reactive T cells within this short duration to help boost the immune response and potentiate the patient's own in vivo T cell response to the viral infection. Developments in Rapid Expansion Protocols (REP) for T cell and TIL expansion in the setting of clinical oncology have enabled methods for the robust and exponential ex vivo expansion of unenriched, as well as neoantigen-enriched, T cells for the autologous treatment of solid tumors (Jin, J. et al., “Simplified method of the growth of human tumor infiltrating lymphocytes in gas-permeable flasks to numbers needed for patient treatment. J2012, Immunother 35, 283-292; Dudley, M. E. et al., “Generation of tumor-infiltrating lymphocyte cultures for use in adoptive transfer therapy for melanoma patients. 2003, J Immunother 26, 332− 342; Douglas C. et al., “Internal checkpoint regulates T cell neoantigen reactivity and susceptibility to PD1 blockade.” 2020, bioRxiv In press; Bianchi, V. et al., “Neoantigen-Specific Adoptive Cell Therapies for Cancer: Making T-Cell Products More Personal.” 2020, Front Immunol 11, 1215). In fact, large quantities of antigen-specific TIL can be expanded in just 22 days using well established protocols (Amod Sarnaik, N. I. K. et al., “Safety and efficacy of cryopreserved autologous tumor infiltrating lymphocyte therapy (LN-144, lifileucel) in advanced metastatic melanoma patients who progressed on multiple prior therapies including anti-PD-1.” 2019, Journal of Clinical Oncology 37, 2518-2518). This duration is however too long for a clinically relevant cell therapy in the setting of COVID-19, and thus a more rapid T cell expansion protocol that builds upon the principles of TIL and T cell REP is developed.

The approach described herein and thereof enabled an over 1,000-fold expansion in the numbers of viral antigen-specific T cells in just 7-days from isolation of a patient's T cells, providing a higher quantity of activated, polyfunctional effector memory cells. It demonstrated the antigen-specific expansion of SARS-CoV-2 T cells from convalescent COVID-19 patients hence the applicability for this viral pathogen, yet one important consideration is that these individuals had very low numbers of recirculating SARS-CoV-2 specific T cells in their blood due to the timeframe since their infection and recovery (Table 8). Hospitalized, symptomatic COVID-19 patients with a severe form of the disease will be undergoing a significant cellular immune response whereby the numbers of virus-specific T cells may have expanded, even if overall T cell numbers may be reduced in some individuals (Diao, B. et al., “Reduction and Functional Exhaustion of T Cells in Patients With Coronavirus Disease 2019 (COVID-19). 2020, Front Immunol 11, 827). While this T cell response needs therapeutically boosting to potentiate viral clearance and ensure positive disease outcome, we expect a more robust and significantly elevated VIL expansion can be achieved when T cells are acquired from suffering COVID-19 patients as opposed to recovered convalescent individuals. When considering the translation of this platform into the clinic as a potential cell therapy for hospitalized COVID-19 patients, based on the level of enrichment and expansion demonstrated herein and thereof, and a prediction of the number of SARS-COV-2 cells in the blood of COVID-19 individuals, an estimated capability to expand and deliver an average of approximately 3.5×10⁹ SARS-321 CoV-2 CD8+ and/or CD4+ T cells back to the patient within 7-days was calculated (Table 9).

TABLE 9
Estimations of viral-specific T cell numbers generated ex vivo for patient
infusion based on the empirical data of VIL expansion by VIPR particles

| Therapeutic Parameters | Value | |
|---|---|---|
| Volume of blood per kg of bodyweight that can be taken from a patient | 5 | ml/kg |
| Therefore, approx. total volume of blood for T cell extraction in 70 kg | 350 | ml |
| male | | |

| | |
|---|---|
| Number of total T cells per ml of blood | 1 × 10⁶ |
| Total number of T cells extracted per patient | 3.5 × 10⁸ |
| Estimated % of SARS-CoV-2 VIL during infection | 1% |
| Total number of SARS-CoV-2 VIL isolated per patient | 3.5 × 10⁶ |
| Average fold-expansion of SARS-CoV-2 VIL ex vivo after 7 days | 1,000-fold |
| Total number of expanded SARS-CoV-2 VIL for patient infusions | 3.5 × 10⁹ |

### Example 12: Autologous and Allogeneic COVID-19 Treatment

T cells from a COVID-19 positive patient are isolated, stimulated, and expanded as previously described, FIG. 1 and FIG. 5. Anti-SARS-Cov2 specific T cells are administered to the patient at a dose from about 0.22-3.34×10¹ total cells over a period of about 30 minutes. Alternatively, the cells can be cryopreserved and used as an off-the-shelf therapeutic in an allogeneic setting.

A patient is subsequently monitored for a reduction in disease burden with peripheral blood tests at 0, 14, 21, 28 and 60 days.

### Example 13: Immune Booster Vaccine

A subject's blood is collected to determine immunity to a given disease. A serologic IgG test can be used to determine if a subject has antibodies to a virus from past disease or who may be candidates for a vaccination or booster vaccination. If a titer test reveals that antibody count is lower than the acceptable immunity threshold, a subject may require a vaccine or booster to increase immunity to the virus. In some cases, multiple vaccinations may be required to achieve immunity.

### Example 14: Allogeneic Off-the-Shelf Cells

T cells from a healthy subject that has recovered from a disease, such as a viral infection, are isolated, CRISPR modified to knock out at least one of: TCRα constant (TRAC), TCRβ constant (TRBC), HLA-I, HLA heavy chains, and/or beta-2-microglobulin (B2M), expanded, and cryopreserved for future allogeneic treatment in a HLA-matched, partially-matched, or unmatched subject. Cells may also be further modified to knock out an endogenous immune checkpoint gene.

### Example 15: Suppression of an Endogenous Immune Response to a Gene Therapy Vector

Recombinant adeno-associated viruses (rAAV) are derived from small, non-enveloped, 4.7 kb DNA dependo-viruses belonging to the Parvoviridae family. They have emerged as a promising vector platform for in vivo gene delivery. Nevertheless, these successes have been tempered by rising concerns over the immunogenicity of the AAV capsid in patients, especially when the vector is systemically administered.

To address these concerns, compositions and methods provided herein can be utilized to expand AAV-specific CD4+ regulatory T cells that express the transcription factor Foxp3+ (Tregs) from the blood of treated subjects. This could offer a method by which Tregs can be isolated, expanded, or induced through ex vivo cytokine culture to then provide a modality to suppress the AAV-mediated immune response taking place in vivo in response to the gene therapy vector.

1. A method of producing a population of ex vivo antigen specific T cells, said method comprising:
(a) isolating T cells from a sample obtained from a subject;
(b) isolating dendritic cells or dendritic cell precursors from said sample obtained from said subject;
(c) culturing said dendritic cells or dendritic cell precursors with at least a first exogenous peptide to produce a population of antigen presenting dendritic cells or dendritic cell precursors that present at least one of said first exogenous peptide or a derivative thereof, and
(d) culturing said isolated T cells from (a) with
(i) said population of antigen presenting dendritic cells or dendritic cell precursors from (c); and
(ii) an artificial antigen presenting platform that comprises:
(1) a first protein that comprises a peptide binding domain of a human leucocyte antigen (HLA) protein that binds a second exogenous peptide, wherein said first protein is attached to a solid support; and
(2) a second protein that specifically binds to CD28, wherein said second protein is attached to said solid support;
to thereby produce a population of antigen specific T cells that comprises T cells that specifically recognize said first exogenous peptide and T cells that specifically recognize said second exogenous peptide.

2. The method of claim 1, wherein said population of antigen specific T cells comprises CD8+ T cells, CD4+ T cells, or both CD8+ T cells and CD4+ T cells.

3. The method of claim 1, wherein said population of antigen specific T cells comprises effector T cells, memory T cells, or both memory T cells and effector T cells.

4. (canceled)

5. (canceled)

6. The method of claim 1, wherein step (c) comprises culturing said dendritic cells or dendritic cell precursors with a plurality of different exogenous peptides.

7. The method of claim 1, wherein step (d) comprises culturing said isolated T cells in a medium that comprises a plurality of cytokines.

8. The method of claim 7, wherein said plurality of cytokines comprises IL-2, IL-7, or IL-15.

9. The method of claim 1, wherein step (d) further comprises culturing said isolated T cells in a medium that comprises N-Acetyl Cysteine (NAC).

10-24. (canceled)

25. The method of claim 1, wherein said first exogenous peptide is presented by an HLA class II protein expressed on the surface of said dendritic cells or dendritic cell precursors.

26. The method of claim 25, wherein said HLA class II protein is an HLA-DP, HLA-DQ, HLA-DM, HLA-DR, or HLA-DO protein.

27. The method of claim 1, wherein said artificial antigen presenting platform comprises a first protein that comprises a peptide binding domain of an HLA class I protein.

28. The method of claim 27, wherein said HLA class I protein is an HLA-A, HLA-B, or HLA-C protein.

29. The method of claim 1, wherein said first exogenous peptide and said second exogenous peptide are each microbial peptides.

30. The method of claim 29, wherein said microbial peptides are viral, bacterial, or parasitic peptides.

31-56. (canceled)

57. The method of claim 1, further comprising genetically modifying said population of antigen specific T cells to introduce a genomic disruption in at least one HLA gene.

58. The method of claim 57, wherein said genomic disruption inhibits expression of an HLA protein encoded by said at least one HLA gene on the surface of said cell.

59-66. (canceled)

67. A population of antigen-specific T-cells made by the method of claim 1.

68-137. (canceled)

138. A method of producing a population of ex vivo antigen specific T cells, said method comprising:
(a) isolating T cells from a sample obtained from a subject;
(b) isolating dendritic cells or dendritic cell precursors from said sample obtained from said subject;
(c) culturing said dendritic cells or dendritic cell precursors with at least a one exogenous peptide to produce a population of antigen presenting dendritic cells or dendritic cell precursors that present at least one of said first exogenous peptide or a derivative thereof, and
(d) culturing said isolated T cells from (a) with said antigen presenting dendritic cells or precursors thereof from (c);
to thereby produce a population of antigen specific T cells that specifically recognize said at least one exogenous peptide, wherein said population of antigen specific T cells comprises effector T cells, memory T cells, or both memory T cells and effector T cells.

139. The method of claim 138, wherein said population of antigen specific T cells comprises CD4+ T cells, CD8+ T cells, or both CD8+ T cells and CD4+ T cells.

140. (canceled)

141. The method of claim 138, wherein step (c) comprises culturing said dendritic cells or dendritic cell precursors with a plurality of different exogenous peptides.

142-281. (canceled)

282. The method of claim 138, wherein said at least one exogenous peptide comprises at least one microbial peptide.


SEQUENCE LISTING (89 sequences)

SEQ ID NO: 1 - protein, 9 aa, Human immunodeficiency virus 1
SLYNTVATL

SEQ ID NO: 2 - protein, 10 aa, Human immunodeficiency virus 1
KRWIILGLNK

SEQ ID NO: 3 - protein, 9 aa, Human immunodeficiency virus 1
GPGHKARVL

SEQ ID NO: 4 - protein, 11 aa, Human immunodeficiency virus 1
KAFSPEVIPMF

SEQ ID NO: 5 - protein, 9 aa, Human immunodeficiency virus 1
VLAEAMSQV

SEQ ID NO: 6 - protein, 9 aa, Human immunodeficiency virus 1
SLFNTVATL

SEQ ID NO: 7 - protein, 10 aa, Human immunodeficiency virus 1
RYPLTFGWCF

SEQ ID NO: 8 - protein, 9 aa, Human immunodeficiency virus 1
AVDLSHFLK

SEQ ID NO: 9 - protein, 10 aa, Human immunodeficiency virus 1
QVPLRPMTYK

SEQ ID NO: 10 - protein, 8 aa, Human immunodeficiency virus 1
RYPLTFGW

SEQ ID NO: 11 - protein, 10 aa, Human immunodeficiency virus 1
RFPLTFGWCF

SEQ ID NO: 12 - protein, 10 aa, Human immunodeficiency virus 1
RGPGRAFVTI

SEQ ID NO: 13 - protein, 9 aa, Human immunodeficiency virus 1
KLTPLCVTL

SEQ ID NO: 14 - protein, 15 aa, Human immunodeficiency virus 1
RIQRGPGRAFVTIGK

SEQ ID NO: 15 - protein, 9 aa, Human immunodeficiency virus 1
RAIEAQQHL

SEQ ID NO: 16 - protein, 15 aa, Human immunodeficiency virus 1
RIQRGPGRAFVTIGK

SEQ ID NO: 17 - protein, 10 aa, Human immunodeficiency virus 1
TVYYGVPVWK

SEQ ID NO: 18 - protein, 11 aa, Human immunodeficiency virus 1
RLRDLLLIVTR

SEQ ID NO: 19 - protein, 9 aa, Zaire ebolavirus
FLSFASLFL

SEQ ID NO: 20 - protein, 9 aa, Zaire ebolavirus
FQQTNAMVT

SEQ ID NO: 21 - protein, 9 aa, Zaire ebolavirus
KLTEAITAA

SEQ ID NO: 22 - protein, 9 aa, Zaire ebolavirus
RLMRTNFLI

SEQ ID NO: 23 - protein, 8 aa, Zaire ebolavirus
YQNNLEEI

SEQ ID NO: 24 - protein, 9 aa, Zaire ebolavirus
ATDVPSATK

SEQ ID NO: 25 - protein, 9 aa, Zaire ebolavirus
RLASTVIYR

SEQ ID NO: 26 - protein, 9 aa, Zaire ebolavirus
TTIGEWAFW

SEQ ID NO: 27 - protein, 9 aa, Zaire ebolavirus
GFRSGVPPK

SEQ ID NO: 28 - protein, 9 aa, Zaire ebolavirus
NQDGLICGL

SEQ ID NO: 29 - protein, 9 aa, Zika virus
IGVSNRDFV

SEQ ID NO: 30 - protein, 20 aa, Zika virus
IRCIGVSNRDFVEGMSGGTW

SEQ ID NO: 31 - protein, 20 aa, Zika virus
QPENLEYRIMLSVHGSQHSG

SEQ ID NO: 32 - protein, 20 aa, Zika virus
KGVSYSLCTAAFTFTKIPAE

SEQ ID NO: 33 - protein, 20 aa, Zika virus
FEATVRGAKRMAVLGDTAWD

SEQ ID NO: 34 - protein, 20 aa, Zika virus
HRSGSTIGKAFEATVRGAKR

SEQ ID NO: 35 - protein, 17 aa, Influenza A virus
ELLVLLENERTLDYHDS

SEQ ID NO: 36 - protein, 17 aa, Influenza A virus
ELLVLLENERTLDYHDS

SEQ ID NO: 37 - protein, 20 aa, Influenza A virus
TYVLSIIPSGPLKAEIAQRL

SEQ ID NO: 38 - protein, 11 aa, Influenza A virus
LYKKLKREITF

SEQ ID NO: 39 - protein, 16 aa, Influenza A virus
GFEMIWDPNGWTGTDN

SEQ ID NO: 40 - protein, 17 aa, Influenza A virus
GQASYKIFRIEKGKIVK

SEQ ID NO: 41 - protein, 17 aa, Influenza A virus
GWAIYSKDNSVRIGSKG

SEQ ID NO: 42 - protein, 7073 aa, Severe acute respiratory syndrome-related coronavirus 2
MESLVLGVNEKTHVQLSLPVLQVRDVLVRGFGDSVEEALSEAREHLKNGTCGLVELEKGVLPQLEQPYVFIKRSDALSTNHGHKVVELVAEMDGIQYGRSGITLGVLVPHVGETPIAYRNVLLRKNGNKGAGGHSYGIDLKSYDLGDELGTDPIEDYEQNWNTKHGSGALRELTRELNGGAVTRYVDNNFCGPDGYPLDCIKDFLARAGKSMCTLSEQLDYIESKRGVYCCRDHEHEIAWFTERSDKSYEHQTPFEIKSAKKFDTFKGECPKFVFPLNSKVKVIQPRVEKKKTEGFMGRIRSVYPVASPQECNNMHLSTLMKCNHCDEVSWQTCDFLKATCEHCGTENLVIEGPTTCGYLPTNAVVKMPCPACQDPEIGPEHSVADYHNHSNIETRLRKGGRTRCFGGCVFAYVGCYNKRAYWVPRASADIGSGHTGITGDNVETLNEDLLEILSRERVNINIVGDFHLNEEVAIILASFSASTSAFIDTIKSLDYKSFKTIVESCGNYKVTKGKPVKGAWNIGQQRSVLTPLCGFPSQAAGVIRSIFARTLDAANHSIPDLQRAAVTILDGISEQSLRLVDAMVYTSDLLTNSVIIMAYVTGGLVQQTSQWLSNLLGTTVEKLRPIFEWIEAKLSAGVEFLKDAWEILKFLITGVFDIVKGQIQVASDNIKDCVKCFIDVVNKALEMCIDQVTIAGAKLRSLNLGEVFIAQSKGLYRQCIRGKEQLQLLMPLKAPKEVTFLEGDSHDTVLTSEEVVLKNGELEALETPVDSFTNGAIVGTPVCVNGLMLLEIKDKEQYCALSPGLLATNNVFRLKGGAPIKGVTFGEDTVWEVQGYKNVRITFELDERVDKVLNEKCSVYTVESGTEVTEFACVVAEAVVKTLQPVSDLLTNMGIDLDEWSVATFYLFDDAGEENFSSRMYCSFYPPDEEEEDDAECEEEEIDETCEHEYGTEDDYQGLPLEFGASAETVRVEEEEEEDWLDDTTEQSEIEPEPEPTPEEPVNQFTGYLKLTDNVAIKCVDIVKEAQSANPMVIVNAANIHLKHGGGVAGALNKATNGAMQKESDDYIKLNGPLTVGGSCLLSGHNLAKKCLHVVGPNLNAGEDIQLLKAAYENFNSQDILLAPLLSAGIFGAKPLQSLQVCVQTVRTQVYIAVNDKALYEQVVMDYLDNLKPRVEAPKQEEPPNTEDSKTEEKSVVQKPVDVKPKIKACIDEVTTTLEETKFLTNKLLLFADINGKLYHDSQNMLRGEDMSFLEKDAPYMVGDVITSGDITCVVIPSKKAGGTTEMLSRALKKVPVDEYITTYPGQGCAGYTLEEAKTALKKCKSAFYVLPSEAPNAKEEILGTVSWNLREMLAHAEETRKLMPICMDVRAIMATIQRKYKGIKIQEGIVDYGVRFFFYTSKEPVASIITKLNSLNEPLVTMPIGYVTHGFNLEEAARCMRSLKAPAVVSVSSPDAVTTYNGYLTSSSKTSEEHFVETVSLAGSYRDWSYSGQRTELGVEFLKRGDKIVYHTLESPVEFHLDGEVLSLDKLKSLLSLREVKTIKVFTTVDNTNLHTQLVDMSMTYGQQFGPTYLDGADVTKIKPHVNHEGKTFFVLPSDDTLRSEAFEYYHTLDESFLGRYMSALNHTKKWKFPQVGGLTSIKWADNNCYLSSVLLALQQLEVKFNAPALQEAYYRARAGDAANFCALILAYSNKTVGELGDVRETMTHLLQHANLESAKRVLNVVCKHCGQKTTTLTGVEAVMYMGTLSYDNLKTGVSIPCVCGRDATQYLVQQESSFVMMSAPPAEYKLQQGTFLCANEYTGNYQCGHYTHITAKETLYRIDGAHLTKMSEYKGPVTDVFYKETSYTTTIKPVSYKLDGVTYTEIEPKLDGYYKKDNAYYTEQPIDLVPTQPLPNASFDNFKLTCSNTKFADDLNQMTGFTKPASRELSVTFFPDLNGDVVAIDYRHYSASFKKGAKLLHKPIVWHINQATTKTTFKPNTWCLRCLWSTKPVDTSNSFEVLAVEDTQGMDNLACESQQPTSEEVVENPTIQKEVIECDVKTTEVVGNVILKPSDEGVKVTQELGHEDLMAAYVENTSITIKKPNELSLALGLKTIATHGIAAINSVPWSKILAYVKPFLGQAAITTSNCAKRLAQRVFNNYMPYVFTLLFQLCTFTKSTNSRIRASLPTTIAKNSVKSVAKLCLDAGINYVKSPKFSKLFTIAMWLLLLSICLGSLICVTAAFGVLLSNFGAPSYCNGVRELYLNSSNVTTMDFCEGSFPCSICLSGLDSLDSYPALETIQVTISSYKLDLTILGLAAEWVLAYMLFTKFFYLLGLSAIMQVFFGYFASHFISNSWLMWFIISIVQMAPVSAMVRMYIFFASFYYIWKSYVHIMDGCTSSTCMMCYKRNRATRVECTTIVNGMKRSFYVYANGGRGFCKTHNWNCLNCDTFCTGSTFISDEVARDLSLQFKRPINPTDQSSYIVDSVAVKNGALHLYFDKAGQKTYERHPLSHFVNLDNLRANNTKGSLPINVIVFDGKSKCDESASKSASVYYSQLMCQPILLLDQALVSDVGDSTEVSVKMFDAYVDTFSATFSVPMEKLKALVATAHSELAKGVALDGVLSTFVSAARQGVVDTDVDTKDVIECLKLSHHSDLEVTGDSCNNFMLTYNKVENMTPRDLGACIDCNARHINAQVAKSHNVSLIWNVKDYMSLSEQLRKQIRSAAKKNNIPFRLTCATTRQVVNVITTKISLKGGKIVSTCFKLMLKATLLCVLAALVCYIVMPVHTLSIHDGYTNEIIGYKAIQDGVTRDIISTDDCFANKHAGFDAWFSQRGGSYKNDKSCPVVAAIITREIGFIVPGLPGTVLRAINGDFLHFLPRVFSAVGNICYTPSKLIEYSDFATSACVLAAECTIFKDAMGKPVPYCYDTNLLEGSISYSELRPDTRYVLMDGSIIQFPNTYLEGSVRVVTTFDAEYCRHGTCERSEVGICLSTSGRWVLNNEHYRALSGVFCGVDAMNLIANIFTPLVQPVGALDVSASVVAGGIIAILVTCAAYYFMKFRRVFGEYNHVVAANALLFLMSFTILCLVPAYSFLPGVYSVFYLYLTFYFTNDVSFLAHLQWFAMFSPIVPFWITAIYVFCISLKHCHWFFNNYLRKRVMFNGVTFSTFEEAALCTFLLNKEMYLKLRSETLLPLTQYNRYLALYNKYKYFSGALDTTSYREAACCHLAKALNDFSNSGADVLYQPPQTSITSAVLQSGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQGKFKKIVKGTHHWMLLTFLTSLLILVQSTQWSLFFFVYENAFLPFTLGIMAIAACAMLLVKHKHAFLCLFLLPSLATVAYFNMVYMPASWVMRIMTWLELADTSLSGYRLKDCVMYASALVLLILMTARTVYDDAARRVWTLMNVITLVYKVYYGNALDQAISMWALVISVTSNYSGVVTTIMFLARAIVFVCVEYYPLLFITGNTLQCIMLVYCFLGYCCCCYFGLFCLLNRYFRLTLGVYDYLVSTQEFRYMNSQGLLPPKSSIDAFKLNIKLLGIGGKPCIKVATVQSKMSDVKCTSVVLLSVLQQLRVESSSKLWAQCVQLHNDILLAKDTTEAFEKMVSLLSVLLSMQGAVDINRLCEEMLDNRATLQAIASEFSSLPSYAAYATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVVPDYGTYKNTCDGNTFTYASALWEIQQVVDADSKIVQLSEINMDNSPNLAWPLIVTALRANSAVKLQNNELSPVALRQMSCAAGTTQTACTDDNALAYYNNSKGGRFVLALLSDHQDLKWARFPKSDGTGTIYTELEPPCRFVTDTPKGPKVKYLYFIKGLNNLNRGMVLGSLAATVRLQAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNRVCGVSAARLTPCGTGTSTDVVYRAFDIYNEKVAGFAKFLKTNCCRFQEKDEEGNLLDSYFVVKRHTMSNYQHEETIYNLVKDCPAVAVHDFFKFRVDGDMVPHISRQRLTKYTMADLVYALRHFDEGNCDTLKEILVTYNCCDDDYFNKKDWYDFVENPDILRVYANLGERVRQSLLKTVQFCDAMRDAGIVGVLTLDNQDLNGNWYDFGDFVQVAPGCGVPIVDSYYSLLMPILTLTRALAAESHMDADLAKPLIKWDLLKYDFTEERLCLFDRYFKYWDQTYHPNCINCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVETPHLMGWDYPKCDRAMPNMLRIMASLVLARKHNTCCNLSHRFYRLANECAQVLSEMVMCGGSLYVKPGGTSSGDATTAYANSVFNICQAVTANVNALLSTDGNKIADKYVRNLQHRLYECLYRNRDVDHEFVDEFYAYLRKHFSMMILSDDAVVCYNSNYAAQGLVASIKNFKAVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKIGILCIMSDRDLYDKLQFTSLEIPRRNVATLQAENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSDSPCESHGKQVVSDIDYVPLKSATCITRCNLGGAVCRHHANEYRQYLDAYNMMISAGFSLWIYKQFDTYNLWNTFTRLQSLENVAYNVVNKGHFDGHAGEAPVSIINNAVYTKVDGIDVEIFENKTTLPVNVAFELWAKRNIKPVPEIKILNNLGVDIAANTVIWDYKREAPAHVSTIGVCTMTDIAKKPTESACSSLTVLFDGRVEGQVDLFRNARNGVLITEGSVKGLTPSKGPAQASVNGVTLIGESVKTQFNYFKKVDGIIQQLPETYFTQSRDLEDFKPRSQMETDFLELAMDEFIQRYKLEGYAFEHIVYGDFSHGQLGGLHLMIGLAKRSQDSPLKLEDFIPMDSTVKNYFITDAQTGSSKCVCSVIDLLLDDFVEIIKSQDLSVISKVVKVTIDYAEISFMLWCKDGHVETFYPKLQASQAWQPGVAMPNLYKMQRMLLEKCDLQNYGENAVIPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPRTKHVTKENDSKEGFFTYLCGFIKQKLALGGSIAVKITEHSWNADLYKLMGHFSWWTAFVTNVNASSSEAFLIGANYLGKPKEQIDGYTMHANYIFWRNTNPIQLSSYSLFDMSKFPLKLRGTAVMSLKENQINDMIYSLLEKGRLIIRENNRVVVSSDILVNN

SEQ ID NO: 43 - protein, 4382 aa, Severe acute respiratory syndrome-related coronavirus 2
MESLVLGVNEKTHVQLSLPVLQVRDVLVRGFGDSVEEALSEAREHLKNGTCGLVELEKGVLPQLEQPYVFIKRSDALSTNHGHKVVELVAEMDGIQYGRSGITLGVLVPHVGETPIAYRNVLLRKNGNKGAGGHSYGIDLKSYDLGDELGTDPIEDYEQNWNTKHGSGALRELTRELNGGAVTRYVDNNFCGPDGYPLDCIKDFLARAGKSMCTLSEQLDYIESKRGVYCCRDHEHEIAWFTERSDKSYEHQTPFEIKSAKKFDTFKGECPKFVFPLNSKVKVIQPRVEKKKTEGFMGRIRSVYPVASPQECNNMHLSTLMKCNHCDEVSWQTCDFLKATCEHCGTENLVIEGPTTCGYLPTNAVVKMPCPACQDPEIGPEHSVADYHNHSNIETRLRKGGRTRCFGGCVFAYVGCYNKRAYWVPRASADIGSGHTGITGDNVETLNEDLLEILSRERVNINIVGDFHLNEEVAIILASFSASTSAFIDTIKSLDYKSFKTIVESCGNYKVTKGKPVKGAWNIGQQRSVLTPLCGFPSQAAGVIRSIFARTLDAANHSIPDLQRAAVTILDGISEQSLRLVDAMVYTSDLLTNSVIIMAYVTGGLVQQTSQWLSNLLGTTVEKLRPIFEWIEAKLSAGVEFLKDAWEILKFLITGVFDIVKGQIQVASDNIKDCVKCFIDVVNKALEMCIDQVTIAGAKLRSLNLGEVFIAQSKGLYRQCIRGKEQLQLLMPLKAPKEVTFLEGDSHDTVLTSEEVVLKNGELEALETPVDSFTNGAIVGTPVCVNGLMLLEIKDKEQYCALSPGLLATNNVFRLKGGAPIKGVTFGEDTVWEVQGYKNVRITFELDERVDKVLNEKCSVYTVESGTEVTEFACVVAEAVVKTLQPVSDLLTNMGIDLDEWSVATFYLFDDAGEENFSSRMYCSFYPPDEEEEDDAECEEEEIDETCEHEYGTEDDYQGLPLEFGASAETVRVEEEEEEDWLDDTTEQSEIEPEPEPTPEEPVNQFTGYLKLTDNVAIKCVDIVKEAQSANPMVIVNAANIHLKHGGGVAGALNKATNGAMQKESDDYIKLNGPLTVGGSCLLSGHNLAKKCLHVVGPNLNAGEDIQLLKAAYENFNSQDILLAPLLSAGIFGAKPLQSLQVCVQTVRTQVYIAVNDKALYEQVVMDYLDNLKPRVEAPKQEEPPNTEDSKTEEKSVVQKPVDVKPKIKACIDEVTTTLEETKFLTNKLLLFADINGKLYHDSQNMLRGEDMSFLEKDAPYMVGDVITSGDITCVVIPSKKAGGTTEMLSRALKKVPVDEYITTYPGQGCAGYTLEEAKTALKKCKSAFYVLPSEAPNAKEEILGTVSWNLREMLAHAEETRKLMPICMDVRAIMATIQRKYKGIKIQEGIVDYGVRFFFYTSKEPVASIITKLNSLNEPLVTMPIGYVTHGFNLEEAARCMRSLKAPAVVSVSSPDAVTTYNGYLTSSSKTSEEHFVETVSLAGSYRDWSYSGQRTELGVEFLKRGDKIVYHTLESPVEFHLDGEVLSLDKLKSLLSLREVKTIKVFTTVDNTNLHTQLVDMSMTYGQQFGPTYLDGADVTKIKPHVNHEGKTFFVLPSDDTLRSEAFEYYHTLDESFLGRYMSALNHTKKWKFPQVGGLTSIKWADNNCYLSSVLLALQQLEVKFNAPALQEAYYRARAGDAANFCALILAYSNKTVGELGDVRETMTHLLQHANLESAKRVLNVVCKHCGQKTTTLTGVEAVMYMGTLSYDNLKTGVSIPCVCGRDATQYLVQQESSFVMMSAPPAEYKLQQGTFLCANEYTGNYQCGHYTHITAKETLYRIDGAHLTKMSEYKGPVTDVFYKETSYTTTIKPVSYKLDGVTYTEIEPKLDGYYKKDNAYYTEQPIDLVPTQPLPNASFDNFKLTCSNTKFADDLNQMTGFTKPASRELSVTFFPDLNGDVVAIDYRHYSASFKKGAKLLHKPIVWHINQATTKTTFKPNTWCLRCLWSTKPVDTSNSFEVLAVEDTQGMDNLACESQQPTSEEVVENPTIQKEVIECDVKTTEVVGNVILKPSDEGVKVTQELGHEDLMAAYVENTSITIKKPNELSLALGLKTIATHGIAAINSVPWSKILAYVKPFLGQAAITTSNCAKRLAQRVFNNYMPYVFTLLFQLCTFTKSTNSRIRASLPTTIAKNSVKSVAKLCLDAGINYVKSPKFSKLFTIAMWLLLLSICLGSLICVTAAFGVLLSNFGAPSYCNGVRELYLNSSNVTTMDFCEGSFPCSICLSGLDSLDSYPALETIQVTISSYKLDLTILGLAAEWVLAYMLFTKFFYLLGLSAIMQVFFGYFASHFISNSWLMWFIISIVQMAPVSAMVRMYIFFASFYYIWKSYVHIMDGCTSSTCMMCYKRNRATRVECTTIVNGMKRSFYVYANGGRGFCKTHNWNCLNCDTFCTGSTFISDEVARDLSLQFKRPINPTDQSSYIVDSVAVKNGALHLYFDKAGQKTYERHPLSHFVNLDNLRANNTKGSLPINVIVFDGKSKCDESASKSASVYYSQLMCQPILLLDQALVSDVGDSTEVSVKMFDAYVDTFSATFSVPMEKLKALVATAHSELAKGVALDGVLSTFVSAARQGVVDTDVDTKDVIECLKLSHHSDLEVTGDSCNNFMLTYNKVENMTPRDLGACIDCNARHINAQVAKSHNVSLIWNVKDYMSLSEQLRKQIRSAAKKNNIPFRLTCATTRQVVNVITTKISLKGGKIVSTCFKLMLKATLLCVLAALVCYIVMPVHTLSIHDGYTNEIIGYKAIQDGVTRDIISTDDCFANKHAGFDAWFSQRGGSYKNDKSCPVVAAIITREIGFIVPGLPGTVLRAINGDFLHFLPRVFSAVGNICYTPSKLIEYSDFATSACVLAAECTIFKDAMGKPVPYCYDTNLLEGSISYSELRPDTRYVLMDGSIIQFPNTYLEGSVRVVTTFDAEYCRHGTCERSEVGICLSTSGRWVLNNEHYRALSGVFCGVDAMNLIANIFTPLVQPVGALDVSASVVAGGIIAILVTCAAYYFMKFRRVFGEYNHVVAANALLFLMSFTILCLVPAYSFLPGVYSVFYLYLTFYFTNDVSFLAHLQWFAMFSPIVPFWITAIYVFCISLKHCHWFFNNYLRKRVMFNGVTFSTFEEAALCTFLLNKEMYLKLRSETLLPLTQYNRYLALYNKYKYFSGALDTTSYREAACCHLAKALNDFSNSGADVLYQPPQTSITSAVLQSGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQGKFKKIVKGTHHWMLLTFLTSLLILVQSTQWSLFFFVYENAFLPFTLGIMAIAACAMLLVKHKHAFLCLFLLPSLATVAYFNMVYMPASWVMRIMTWLELADTSLSGYRLKDCVMYASALVLLILMTARTVYDDAARRVWTLMNVITLVYKVYYGNALDQAISMWALVISVTSNYSGVVTTIMFLARAIVFVCVEYYPLLFITGNTLQCIMLVYCFLGYCCCCYFGLFCLLNRYFRLTLGVYDYLVSTQEFRYMNSQGLLPPKSSIDAFKLNIKLLGIGGKPCIKVATVQSKMSDVKCTSVVLLSVLQQLRVESSSKLWAQCVQLHNDILLAKDTTEAFEKMVSLLSVLLSMQGAVDINRLCEEMLDNRATLQAIASEFSSLPSYAAYATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVVPDYGTYKNTCDGNTFTYASALWEIQQVVDADSKIVQLSEINMDNSPNLAWPLIVTALRANSAVKLQNNELSPVALRQMSCAAGTTQTACTDDNALAYYNNSKGGRFVLALLSDHQDLKWARFPKSDGTGTIYTELEPPCRFVTDTPKGPKVKYLYFIKGLNNLNRGMVLGSLAATVRLQAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNGFAV

SEQ ID NO: 44 - protein, 1255 aa, Severe acute respiratory syndrome-related coronavirus 2
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 45 - protein, 274 aa, Severe acute respiratory syndrome-related coronavirus 2
MDLFMRFFTLRSITAQPVKIDNASPASTVHATATIPLQASLPFGWLVIGVAFLAVFQSATKIIALNKRWQLALYKGFQFICNLLLLFVTIYSHLLLVAAGMEAQFLYLYALIYFLQCINACRIIMRCWLCWKCKSKNPLLYDANYFVCWHTHNYDYCIPYNSVTDTIVVTEGDGISTPKLKEDYQIGGYSEDRHSGVKDYVVVHGYFTEVYYQLESTQITTDTGIENATFFIFNKLVKDPPNVQIHTIDGSSGVANPAMDPIYDEPTTTTSVPL

SEQ ID NO: 46 - protein, 154 aa, Severe acute respiratory syndrome-related coronavirus 2
MMPTTLFAGTHITMTTVYHITVSQIQLSLLKVTAFQHQNSKKTTKLVVILRIGTQVLKTMSLYMAISPKFTTSLSLHKLLQTLVLKMLHSSSLTSLLKTHRMCKYTQSTALQELLIQQWIQFMMSRRRLLACLCKHKKVSTNLCTHSFRKKQVR

SEQ ID NO: 47 - protein, 76 aa, Severe acute respiratory syndrome-related coronavirus 2
MYSFVSEETGTLIVNSVLLFLAFVVFLLVTLAILTALRLCAYCCNIVNVSLVKPTVYVYSRVKNLNSSEGVPDLLV

SEQ ID NO: 48 - protein, 221 aa, Severe acute respiratory syndrome-related coronavirus 2
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 49 - protein, 63 aa, Severe acute respiratory syndrome-related coronavirus 2
MFHLVDFQVTIAEILIIIMRTFRIAIWNLDVIISSIVRQLFKPLTKKNYSELDDEEPMELDYP

SEQ ID NO: 50 - protein, 122 aa, Severe acute respiratory syndrome-related coronavirus 2
MKIILFLTLIVFTSCELYHYQECVRGTTVLLKEPCPSGTYEGNSPFHPLADNKFALTCTSTHFAFACADGTRHTYQLRARSVSPKLFIRQEEVQQELYSPLFLIVAALVFLILCFTIKRKTE

SEQ ID NO: 51 - protein, 44 aa, Severe acute respiratory syndrome-related coronavirus 2
MNELTLIDFYLCFLAFLLFLVLIMLIIFWFSLEIQDLEEPCTKV

SEQ ID NO: 52 - protein, 39 aa, Severe acute respiratory syndrome-related coronavirus 2
MKLLIVLTCISLCSCICTVVQRCASNKPHVLEDPCKVQH

SEQ ID NO: 53 - protein, 84 aa, Severe acute respiratory syndrome-related coronavirus 2
MCLKILVRYNTRGNTYSTAWLCALGKVLPFHRWHTMVQTCTPNVTINCQDPAGGALIARCWYLHEGHQTAAFRDVLVVLNKRTN

SEQ ID NO: 54 - protein, 422 aa, Severe acute respiratory syndrome-related coronavirus 2
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 55 - protein, 98 aa, Severe acute respiratory syndrome-related coronavirus 2
MDPNQTNVVPPALHLVDPQIQLTITRMEDAMGQGQNSADPKVYPIILRLGSQLSLSMARRNLDSLEARAFQSTPIVVQMTKLATTEELPDEFVVVTAK

SEQ ID NO: 56 - protein, 1281 aa, Severe acute respiratory syndrome-related coronavirus 2
MGILPSPGMPALLSLVSLLSVLLMGCVAETGTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNEVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGRENLYFQGGGGSGYIPEAPRDGQAYVRKDGEWVLLSTFLGHHHHHHHH

SEQ ID NO: 57 - protein, 6 aa, synthetic construct
  REGION 1..6: note = Description of Artificial Sequence: Syntheticpeptide
GGGTSG

SEQ ID NO: 58 - protein, 6 aa, synthetic construct
  REGION 1..6: note = Description of Artificial Sequence: Syntheticpeptide
GSLGGS

SEQ ID NO: 59 - protein, 9 aa, Severe acute respiratory syndrome-related coronavirus 2
FIAGLIAIV

SEQ ID NO: 60 - protein, 9 aa, Severe acute respiratory syndrome-related coronavirus 2
ALNTLVKQL

SEQ ID NO: 61 - protein, 9 aa, Severe acute respiratory syndrome-related coronavirus 2
LITGRLQSL

SEQ ID NO: 62 - protein, 9 aa, Severe acute respiratory syndrome-related coronavirus 2
NLNESLIDL

SEQ ID NO: 63 - protein, 9 aa, Severe acute respiratory syndrome-related coronavirus 2
RLNEVAKNL

SEQ ID NO: 64 - protein, 9 aa, Severe acute respiratory syndrome-related coronavirus 2
VLNDILSRL

SEQ ID NO: 65 - protein, 9 aa, Severe acute respiratory syndrome-related coronavirus 2
VVFLHVTYV

SEQ ID NO: 66 - protein, 16 aa, Severe acute respiratory syndrome-related coronavirus 2
GAALQIPFAMQMAYRF

SEQ ID NO: 67 - protein, 16 aa, Severe acute respiratory syndrome-related coronavirus 2
MAYRFNGIGVTQNVLY

SEQ ID NO: 68 - protein, 18 aa, Severe acute respiratory syndrome-related coronavirus 2
QLIRAAEIRASANLAATK

SEQ ID NO: 69 - protein, 17 aa, Severe acute respiratory syndrome-related coronavirus 2
QALNTLVKQLSSNFGAI

SEQ ID NO: 70 - protein, 9 aa, Cytomegalovirus sp.
NLVPMVATV

SEQ ID NO: 71 - protein, 10 aa, unidentified
  REGION 1..10: note = Description of Unknown:MART1 antigen sequence
ELAGIGILTV

SEQ ID NO: 72 - protein, 9 aa, Severe acute respiratory syndrome-related coronavirus 2
YLQPRTFLL

SEQ ID NO: 73 - protein, 13 aa, Influenza A virus
PKYVKQNTLKLAT

SEQ ID NO: 74 - protein, 10 aa, Severe acute respiratory syndrome-related coronavirus 2
TTDPSFLGRY

SEQ ID NO: 75 - protein, 9 aa, Severe acute respiratory syndrome-related coronavirus 2
PTDNYITTY

SEQ ID NO: 76 - protein, 9 aa, Severe acute respiratory syndrome-related coronavirus 2
LLLDRLNQL

SEQ ID NO: 77 - protein, 9 aa, Severe acute respiratory syndrome-related coronavirus 2
YLQPRTFLL

SEQ ID NO: 78 - protein, 9 aa, Severe acute respiratory syndrome-related coronavirus 2
RLQSLQTYV

SEQ ID NO: 79 - protein, 10 aa, Severe acute respiratory syndrome-related coronavirus 2
MEVTPSGTWL

SEQ ID NO: 80 - protein, 419 aa, Severe acute respiratory syndrome-related coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 81 - DNA, 3822 bp, synthetic construct
  misc_feature 1..3822: note = Description of Artificial Sequence: Syntheticpolynucleotide
  source 1..3822: mol_type = other DNA
atggcattcgtgtttctggtgctgctgcctctggtgtccagccagtgcgtgaacttcaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgccaatcctgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcggggactgcctcagggcttttctgctctggaacccctggtggatctgcccatcggcatcaacatcacccggtttcagaccctgcaccggtcctatctgacacccggcgattcttctagcggatggacagctggcgccgctgcctactatgtgggatacctgcagcctcggaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagaccggcaatatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgcaatggcgtgaagggctttaactgctacttcccactgcagtcctacggcttccagccaacatacggcgtgggctaccagccttacagagtggtggtgctgagcttcgagctgctgcatgctcctgccacagtgtgcggccctaagaaaagcaccaatctcgtgaagaacaaatgcgtcaacttcaatttcaacggcctgaccggcaccggcgtgctgacagagagcaacaagaagttcctgccattccagcagttcggccgggacattgccgataccacagatgctgtcagagatccccagacactggaaatcctggacatcaccccatgcagcttcggcggagtgtctgtgatcacccctggcaccaacaccagcaatcaggtggcagtgctgtaccagggcgtcaactgtacagaggtgccagtggccattcacgccgatcagctgacccctacttggcgggtgtactccacaggcagcaatgtgttccagaccagagccggctgtctgatcggagccgagcacgtgaacaatagctacgagtgcgacatccccatcggcgctggcatctgtgccagctaccagacacagacaaacagccccggcggcagcggatctgtggccagccagagcatcattgcctacacaatgtctctgggcgtcgagaacagcgtggcctactccaacaactctatcgctatccccaccaatttcaccatcagcgtgaccacagagatcctgcctgtgtccatgaccaagaccagcgtggactgcaccatgtacatctgcggcgatagcaccgagtgctccaacctgctgctgcagtacggcagcttctgcacccagctgaatagagccctgaccggaatcgccgtggaacaggacaagaacacccaagaggtgttcgcccaagtgaagcagatctacaagacccctcctatcaaggacttcggcggcttcaacttcagccagattctgcccgatcctagcaagcccagcaagcggagcttcatcgaggacctgctgttcaacaaagtgacactggccgacgccggcttcatcaagcagtatggcgattgtctgggcgacattgcagcccgggatctgatttgcgcccagaagtttaacggactgaccgtgctgcctcctctgctgaccgatgagatgatcgcccagtacacatctgccctgctggccggcacaatcacaagcggctggacatttggagctggcgctgccctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagccagcgctctgggaaaactgcaggacgtggtcaaccagaacgcccaggctctgaataccctggtcaagcagctgtcctccaacttcggcgccatcagctctgtgctgaacgatatcctgagcagactggaccctcctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcaccacacggcgtggtgtttctgcacgtgacatacgtgcccgctcaagagaagaacttcacaacagcccctgccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccttggtacatctggctgggctttatcgccggactgattgccatcgtgatggtcacaatcatgctgtgctgtatgaccagctgctgtagctgcctgaagggctgttgcagctgtggctcctgctgcaagttcgacgaggacgacagtgagccggtgcttaagggcgtaaaacttcattacacttccggatga

SEQ ID NO: 82 - protein, 15 aa, Cytomegalovirus sp.
PDDYSNTHSTRYVTV

SEQ ID NO: 83 - protein, 30 aa, synthetic construct
  REGION 1..30: note = Description of Artificial Sequence: Syntheticpolypeptide
  VARIANT 1..30: note = This sequence may encompass 1-6 "Gly Gly Pro Gly Gly"repeating units
GGPGGGGPGGGGPGGGGPGGGGPGGGGPGG

SEQ ID NO: 84 - protein, 30 aa, synthetic construct
  REGION 1..30: note = Description of Artificial Sequence: Syntheticpolypeptide
  VARIANT 1..30: note = This sequence may encompass 1-6 "Gly Gly Ser Gly Gly"repeating units
GGSGGGGSGGGGSGGGGSGGGGSGGGGSGG

SEQ ID NO: 85 - protein, 8 aa, synthetic construct
  REGION 1..8: note = Description of Artificial Sequence: Syntheticpeptide
SIYRYYGL

SEQ ID NO: 86 - protein, 8 aa, Gallus gallus
SIINFEKL

SEQ ID NO: 87 - protein, 9 aa, unidentified
  REGION 1..9: note = Description of Unknown:melanocyte GP100 protein sequence
KVPRNQDWL

SEQ ID NO: 88 - protein, 9 aa, Influenza A virus
ASNENMETH

SEQ ID NO: 89 - protein, 9 aa, Influenza A virus
GILGFVFTL